(19) United States
(12) Patent Application Publication
(10) Pub. No.: US 2003/0158245 A1
(43) Pub. Date: Aug. 21, 2003

(54) Fused heterocyclic derivatives, their production and use

(76) Inventors: Tsuneo Yasuma, Ibaraki-shi (JP); Akira Mori, Kyoto-shi (JP); Masahiro Kawase, Kawabe-gun (JP); Masayuki Takizawa, Tsukuba-shi (JP); Shokyo Miki, Toyonaka-shi (JP); Mitsuhiro Takeda, Osaka-shi (JP)

Correspondence Address: FITZPATRICK CELLA HARPER & SCINTO, 30 ROCKEFELLER PLAZA, NEW YORK, NY, 10112, US

(21) Appl. No.: 10/204,472

(22) PCT Filed: Mar. 29, 2001
(86) PCT No.: PCT/JP01/02614

(30) Foreign Application Priority Data
Mar. 31, 2000 (JP) 2000-101373
Mar. 31, 2000 (JP) 2000-101374
Dec. 25, 2000 (JP) 2000-392843

(51) Int. Cl. C07D 333/76; A61K 31/407; A61K 31/381

(52) U.S. Cl. 514/411; 514/443; 548/430; 549/43

(57) ABSTRACT

There is disclosed novel fused thiophene derivatives as prophylactic and therapeutic drugs for bone or articular diseases, industrially advantageous processes for production thereof, and novel production intermediates. Said fused thiophene derivatives are represented by the general formula (I):

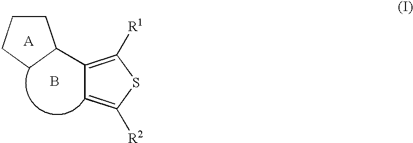

wherein R¹ is an optionally substituted hydrocarbon, heterocyclic, sulfinyl, sulfonyl, hydroxyl, thiol or amino group,; R² is cyano, formyl, thioformyl, etc.; ring A is any of

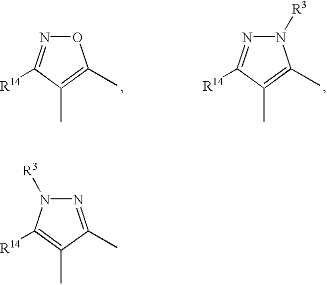

(wherein R³ is hydrogen or an optionally substituted hydrocarbon, heterocyclic, hydroxyl, amino, sulfonyl or acyl; R¹⁴ is hydrogen, halogen, optionally subsituted hydrocarbon group, optionally subsituted heterocyclic group etc.; and ring B represents an optionally substituted 5- to 7-membered hydrocarbon ring.

### FIELD OF THE INVENTION

The present invention relates to fused heterocyclic derivatives or salts thereof, their production and their use. The fused thiophene derivatives or salt thereof of the present invention have activity to induce differentiation of undifferentiated cells such as osteoblast precursor cells and chondrocyte precursor cells, and are useful in the field of medicine as prophylactic or therapeutic drugs for osteopathy and articular diseases whose representative examples include osteopathy and chondropathy, and further for diseases caused by nerve degeneration. The present invention also relates to the novel intermediates useful for the production of the fused thiophene derivatives of the present invention.

### PRIOR ART

Osteopathy includes non-metabolic osteopathy such as bone fracture, bone deformity and spondylosis deformans, osteosarcoma, myeloma, osteogenseis imperfecta, scoliosis, and the like, and metabolic osteopathy such as osteoporosis, osteomalacia, rickets, fibrous ostitis, renal osteodystrophy, Paget's disease of bone, and the like. Among them, metabolic osteopathy has become a matter of concern in recent years. For example, osteoporosis, a metabolic osteopathy, is a systemic disease characterized by the increase in bone fragility and easy bone fracture induced by low bone mass and the changes in microstructures of the osseous tissue. The major clinical symptoms thereof are hunchback and fracture of dorsolumbar bone, vertebral body, femoral neck, distal end of radius, rib, proximal end of humerus, and the like. In the osseous tissue, osteogenesis and osteolysis by bone resorption occur repeatedly in a balanced way, where osteoblasts including osteoblast precursor cells, and osteoclasts in bone resorption play a central role in osteogenesis. when the osteogenesis and osteolysis by bone resorption get out of balance, bone mass reduction is resulted. Heretofore, bone resorption suppressants such as estrogens, calcitonin and bisphosphonates, etc. have mainly been used as the prophylactic and therapeutic drugs of osteoporosis.

Articular diseases are characterized by the degeneration of articular cartilage (for example, chronic rheumatoid arthritis, osteoarthritis, or the like) as the major lesion. Cartilage is a tissue composed of collagen and proteoglycan, where release of proteoglycan from cartilage tissue is accelerated and decrease in proteoglycan synthesis in the tissue is initiated by a variety of causes. At the same time, release and activation of matrix metalloprotease such as collagenase-3 are stimulated, leading to the degradation of collagen in the cartilage tissue. These series of reactions promote hardening and destruction of the cartilage tissue, followed by the progression of the lesion leading to synovial hyperplasia, destruction of subchondral bone, and hypertrophy or bone neogenesis at the peripheral region of the joint, which are further followed by joint deformation, eventually resulting in dysfunction in serious cases. Articular diseases are most frequently observed in the knee joints but are also observed in the joints of elbow, hip, foot and finger. Among the articular diseases, osteoarthritis is the commonest disease suffered by the largest number of patients, while it is expected that the number of patients with the disease will increase in the aging society in future, because aging is considered to be one of the etiologic factor of this disease. For treating this disease, antiphlogistic analgesic drugs and hyaluronic acid preparations are used to alleviate the pain that accompanies the cartilage degeneration and hardening and destruction of subchondral bone. However, these drugs are used only for symptomatic treatment and are insufficient for providing the desired effects. Stimulation of chondrogenesis, suppression of cartilage destruction, and induction and stimulation of differentiation of chondrocytes including chondrocyte precursor cells are expected to be effective in preventing and treating chondropathy.

As for fused thiophene derivatives, 4,5-dihydro-8-(methylthio)isoxazolo[5,4-d]benzo[c]thiophene-6-carboxamide is described in a product catalog (Volume 241, published in October, 1991) of Maybridge Company (Trevillett, Tintagel, North Cornwall, PL34 OHW, England).

JP 8-245386 A discloses an enhancer of cell differentiation inducer activity comprising, for example, 4,5-dihydro-8-(methylthio)isoxazolo[5,4-d]benzo[c]-thiophene-6-carboxamide, or the like.

JP 10-130271 A (WO98/09958) discloses fused thiophene derivatives which have cell differentiation inducer-enhancing activity and an anti-matrix metalloprotease activity, and are useful for prevention and treatment of osteopathy such as osteoporosis, bone fracture, osteoarthritis, chronic rheumatoid arthritis, and the like, arteriosclerosis, cancer metastasis, and diseases caused by nerve degeneration. The production processes hereof are also disclosed.

Furthermore, fused thiophene derivatives and their production processes are disclosed in GB 2336589 A, Liebigs Ann., pages 239-245, 1996, and WO98/09958. Further, thiophene derivatives are disclosed in Heterocycles, Vol. 43, page 367 (1996).

Among fused thiophene derivatives, in order to produce a compound represented by the general formula (I):

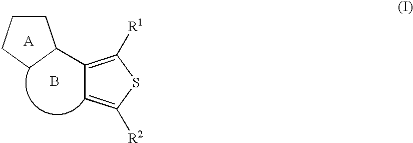

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); ring A represents an optionally substituted aromatic 5-membered heterocyclic ring represented by any of

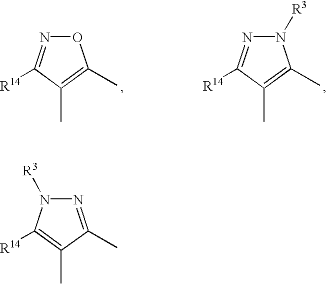

(wherein R³ represents hydrogen atom or an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring, or a salt thereof, in particular, a compound represented by the general formula (I-II):

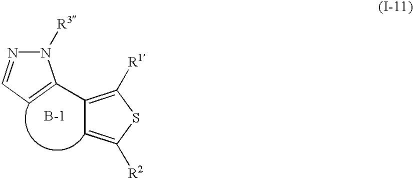

wherein R^{1′} represents, among an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group, a group represented by the formula: —X′—W′ (wherein X′ represents a bond, an optionally substituted carbon atom, an optionally substituted nitrogen atom, oxygen atom or an optionally oxidized sulfur atom; and W′ represents an optionally substituted cyclic group or carbon or nitrogen atom having 2 or more substituents); R² represents cyano group, formyl group, thioformyl group, or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R^{3″} represents an optionally substituted hydrocarbon group, an optionally substituted heterocyclic group, an optionally substituted sulfonyl group or an optionally substituted acyl group; and ring B-1 represents an optionally substituted 5- to 7-membered hydrocarbon ring, or a salt thereof, for example, the following compound 20 is produced, it can be synthesized by combining reactions with reference to papers by D. Prim et al. (Synth. Commun., vol. 25, page 2449, 1995), A. Nangia et al. (Indian J. Chem., vol. 35B, page 49, 1996), etc., according to the following scheme.

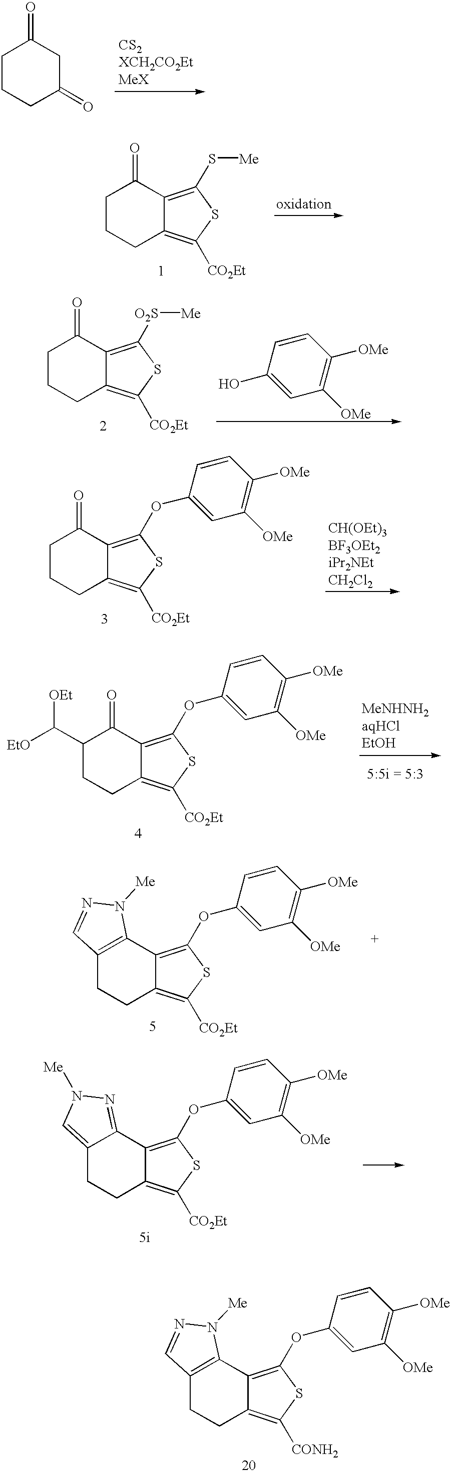

### OBJECTS OF THE INVENTION

In the clinical field of bone or articular diseases (for example, cartilage diseases), excellent drugs for preventing and treating the bone or articular diseases have been still desired. In particular, excellent drugs for preventing and treating articular diseases, for example, cartilage diseases have been desired. Further, when compound 20 has been synthesized actually according to the above synthetic route, there have been raised problems such as (1) the pyrazole-cyclization reaction of intermediate 4 is inefficient because of no position selectivity, and further complicated purification operations such as column chromatography are required to obtain the desired intermediate 5; (2) boron trifluoride ether complex, which has a possibility of making it difficult to conduct the reaction in a common production equipment owing to a corrosion problem, is used in the step to obtain intermediate 4 from 3, etc. Then, it is required to develop an industrially advantageous process overcoming these problems. In particular, no experiment succeeded in solving the problem of the cyclization-position selectivity of the above (1) has been found.

An object of the present invention is to provide a novel fused thiophene derivative useful as a drug for preventing or treating bone or articular diseases.

Another object of the present invention is to provide an industrially advantageous process for producing the fused thiophene derivative.

Another object of the present invention is to provide a novel intermediate to produce the fused thiophene derivative.

These objects as well as other objects and advantages of the present invention will become apparent to those skilled in the art from the following description.

### SUMMARY OF THE INVENTION

Under the above circumstances, the present inventors have studied intensively. As a result, the present inventors have found that novel fused thiophene derivatives represented by the following formula (I) which are characteristic in ring A, or salts thereof, have activity to induce differentiation of undifferentiated cells such as osteoblast precursor cells and chondrocyte precursor cells, thereby being employed as prophylactic and therapeutic drugs for bone or articular diseases. Further, the present inventors have found novel synthetic intermediates useful for the production of the novel fused thiophene derivatives represented by the formula (I) which are characteristic in ring A, or salts thereof.

Furthermore, the present inventors have found that, in the above synthetic route, intermediate 7, which is easily obtained by the reaction of intermediate 1 with trisdimethylaminomethane, becomes a good precursor for the pyrazole cyclization and gives intermediate 8 by the reaction with MeNHNH₂, a hydrazine derivative, thereby producing compound 20 in good yield from the thus-obtained 8.

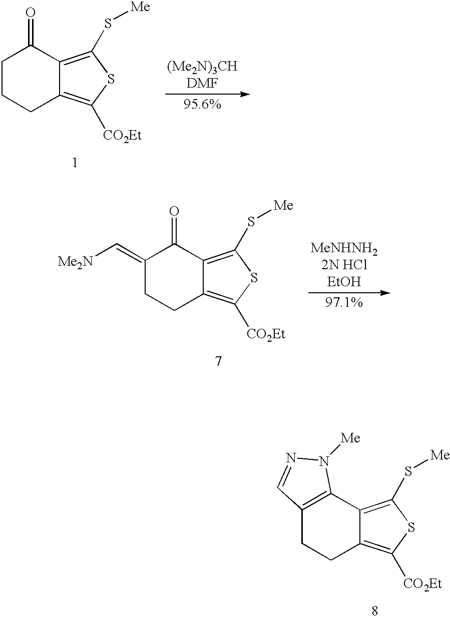

Moreover, it has been found that, although the following intermediate 9 having a bulky substituent (an aryloxy group in this case) at the 3-position is difficult to be cyclized with the position selectivity, unexpectedly the position selectivity is improved to obtain 5 in good yield when the reaction is carried out in the presence of an acid under substantially anhydrous conditions.

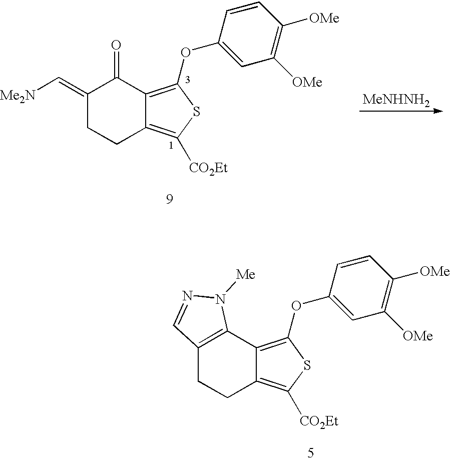

The present inventors have further studied based on these findings to complete the present invention.

That is, the present invention relates to

[1] A compound represented by the general formula (I):

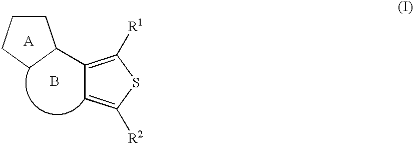

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

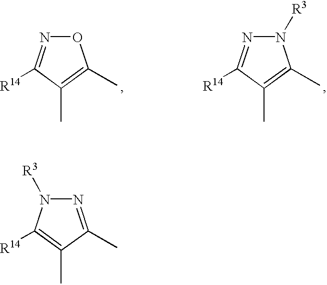

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than the compound represented by the formula:

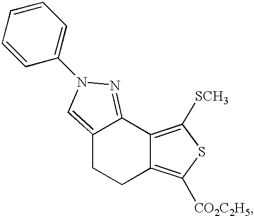

and a compound wherein, when ring A is

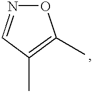

Z¹ of -Z¹-Z² is —CO— and Z² thereof is optionally substituted amino group, or a salt thereof;

[2] The compound according to the above [1], wherein ring B represents a 5- to 7-membered hydrocarbon ring having 1 to 3 C₁₋₈ alkyl groups;

[3] The compound according to the above [1], wherein ring B represents an unsubstituted 5- to 7-membered hydrocarbon ring;

[4] The compound according to the above [1], wherein ring B represents an unsubstituted 6-membered hydrocarbon ring;

[5] The compound according to the above [1], wherein ring A is

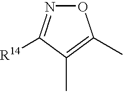

wherein R¹⁴ is as defined in the above [1];

[6] The compound according to the above [1], wherein ring A is

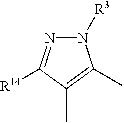

wherein each symbol is as defined in the above [1];

[7] The compound according to the above [1], wherein ring A is

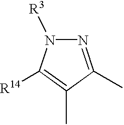

wherein each symbol is as defined in the above [1];

[8] The compound according to the above [1], wherein R³ is (1) hydrogen atom, (2) a C₁₋₈ alkyl group optionally substituted with 1 to 3 substituents selected from the group consisting of C₁₋₆ alkoxy, carboxyl, C₁₋₆ alkoxy-carbonayl, halogen and hydroxyl, (3) a C₇₋₁₄ aralkyl group, (4) a C₂₋₈ alkanoyl group, (5) a C₁₋₆ alkoxy-carbonyl group, (6) carbamoyl group optionally substituted with C₁₋₆ alkyl, (7) C₁₋₈ alkylsulfinyl or (8) C₁₋₈ alkylsulfonyl, and R¹⁴ is (1) hydrogen atom, (2) halogen, (3) C₁₋₈ alkyl or (4) i) C₆₋₁₀ aryl, ii) a 5- to 7-membered heterocyclic group containing one sulfur atom, one nitrogen atom or one oxygen atom, iii) a 5- to 6-membered heterocyclic group containing 2 to 4 nitrogen atoms, iv) a 5- to 6-membered heterocyclic group containing 1 to 2 nitrogen atoms and one sulfur atom or oxygen atom, or v) a heterocyclic group of ii) to iv) which is fused with a 5- to 6-membered heterocyclic ring containing 2 or less nitrogen atom, benzene ring or a 5-membered heterocyclic ring, each of which may have 1 to 3 substituents selected from the group consisting of C₁₋₆ alkyl, C₁₋₆ haloalkyl, C₁₋₆ alkoxy, C₁₋₆ haloalkoxy, C₁₋₆ alkoxy-carbonyl, amino and C₁₋₆ alkylthio;

[9] The compound according to the above [1], wherein R³ is a C₁₋₈ alkyl group and R¹⁴ is hydrogen atom;

[10] The compound according to the above [1], wherein R¹ is an optionally substituted hydrocarbon group;

[11] The compound according to the above [1], wherein R¹ is an optionally substituted heterocyclic group;

[12] The compound according to the above [1], wherein R¹ is an optionally substituted sulfinyl group, an optionally substituted sulfonyl group or an optionally substituted thiol group;

[13] The compound according to the above [1], wherein R¹ is an optionally substituted hydroxyl group;

[14] The compound according to the above [1], wherein R¹ is an optionally substituted amino group;

[15] The compound according to the above [1], wherein R¹ is:

(1) sulfinyl group, sulfonyl group or thiol group each of which may be substituted with C₁₋₈ alkyl or C₇₋₁₄ aralkyl,

(2) i) a C₆₋₁₀ aryloxy group,

ii) an aromatic 5- or 6-membered heterocyclic-oxy group having 1 to 4 hetero atoms selected from the group consisting of nitrogen atom, oxygen atom and sulfur atom, or

iii) a fused bicyclic heterocyclic-oxy group formed by aromatic 5- or 6-membered heterocyclic ring having 1 to 3 hetero atoms selected from the group consisting of nitrogen atom, oxygen atom and sulfur atom fused with benzene ring or aromatic 5- or 6-membered heterocyclic ring having 1 to 3 hetero atoms selected from the group consisting of nitrogen atom, oxygen atom and sulfur atom,

each of which may be substituted with 1 or 2 substituents selected from the group consisting of C₁₋₈ alkyl, C₆₋₁₄ aryl, C₇₋₁₄ aralkyl, hydroxyl, C₁₋₆ alkoxy, C₁₋₆ alkylenedioxy, C₆₋₁₄ aryloxy, C₁₋₆ alkylthio, morpholinosulfonyl, formyl, C₁₋₆ alkyl-carbonyl, carboxyl, C₁₋₆ alkoxy-carbonyl, C₆₋₁₄ aryloxy-carbonyl, C₇₋₁₄ aralkyloxy-carbonxyl, amino, mono- or di-C₁₋₆ alkylamino, mono- or di-C₁₋₆ alkyl-carbonylamino, toluenesulfonylamino, C₁₋₆ alkylaminothiocarbonylamino, -nitro, halogen, C₁₋₆ haloalkyl, C₁₋₆ haloalkoxy, C₁₋₆ alkoxy-C₆₋₁₄ aryl, 2-amino-3-(4-morpholinyl)-3-oxo-C₁₋₆ alkyl, C₁₋₆ alkyl having one or two di(C₁₋₆ alkyoxy)phosphoryl, an aromatic 5- or 6-membered heterocyclic group having 1 to 4 hetero atoms selected from the group consisting of nitorgen atom, oxygen atom and sulfur atom, an aromatic 5- or 6-membered heterocyclic-oxy group having 1 to 4 hetero atoms selected from the group consisting of nitrogen atom, oxygen atom and sulfur atom, and a fused bicyclic heterocyclic-oxy group formed by an aromatic 5- or 6-membered heterocyclic ring having 1 to 3 hetero atoms selected from the group consisting of nitorgen atom, oxygen atom and sulfur aton fused with benzene ring or an aromatic 5- or 6-membered heterocyclic ring having 1 to 3 hetero atoms selected from the group consisting of nitrogen atom, oxygen atom and sulfur atom,

(3) a C₁₋₈ alkyloxy group, a C₃₋₇ cycloalkyloxy group, a C₇₋₁₄ aralkyloxy group or dihydrobenzofuranyloxy group,

(4) amino group which may be substituted with 1 or 2 substituents selected from the group consisting of C₁₋₈ alkyl, C₆₋₁₀ aryl, C₇₋₁₄ aralkyl, halo C₆₋₁₀ aryl, C₁₋₆ alkyl-carbonyl, C₆₋₁₀ aryl-carbonyl and C₇₋₁₄ aralkyl-oxycarbonyl, or

(5) C₁₋₆ alkyl or C₇₋₁₄ aralkyl;

[16] The compound according to the above [1], wherein R¹ is:

(1) sufinyl group, sulfonyl group or thiol group each of which is substituted with C₁₋₈ alkyl,

(2) (a) C₁₋₆ alkylenedioxy, (b) di(C₁₋₆ alkoxy)phosphoryl-C₁₋₆ alkyl, (c) C₁₋₆ haloalkyl, (d) C₇₋₁₄ aralkyloxy, (e) aromatic 5- or 6-membered heterocyclic-oxy having 1 to 4 hetero atoms selected from the group consisting of nitrogen atom, oxygen atom and sulfur atom or (f) a C₆₋₁₀ aryloxy group which may be subsituted with fused bicyclic heterocyclic-oxy formed by an aromatic 5- or 6-membered heterocyclic ring having 1 to 3 hetero atoms selected from the group consisting of nitrogen atom, oxygen atom and sulfur atom fused with benzene ring or an aromatic 5- or 6-membered heterocyclic ring having 1 to 3 hetero atoms selected from the group consisting of nitrogen atom, oxygen atom and sulfur atom,

(3) an aromatic 5- or 6-membered heterocyclic-oxy group having 1 to 4 hetero atoms selected from the group consisting of nitrogen atom, oxygen atom and sulfur atom, or

(4) a concensed bicyclic heterocyclic-oxy group formed by an aromatic 5- or 6-membered heterocyclic ring having 1 to 3 hetero atoms selected from the group consisting or nitrogen atom, oxygen atom and sulfur atom fused with benzene ring or an aromatic 5- or 6-membered heterocyc ring having 1 to 3 hetero atom selected from the group consisting of nitrogen atom, oxygen atom and sulfur atom;

[17] The compound according to the above [1], wherein R is represented by

—CO-Z^{2′}

wherein Z² represents hydrogen atom or an optionally substituted hydrocarbon group, an optionally substituted heterocyclic group, an optionally substituted amino group or an optionally substituted hydroxyl group;

[18] The compound according to the above [17], wherein Z^{2′} is (1) amino group which may be substituted with 1 or 2 substituents selected from the group consisting of C₁₋₈ alkyl, C₆₋₁₄ aryl, C₇₋₁₄ aralkyl, hydroxyl, C₁₋₆ alkoxy, amino, mono- or di-C₁₋₆ alkyl amino, C₁₋₆ alkoxy-carbonylamino, an aromatic 5- or 6-membered heterocyclic group having 1 to 4 hetero atoms selected from the group consisting of nitorgen atom, oxygen atom and sulfur atom, C₁₋₆ haloalkyl, carboxy C₁₋₆ alkyl, C₁₋₆ alkoxy-carbonyl-C₁₋₆ alkyl, carbamoyl C₁₋₆ alkyl, aromatic 5- or 6-membered heterocyclic-C₁₋₆ alkyl having 1 to 4 hetero atoms selected from the group consisting or nitrogen atom, oxygen atom and sulfur atom, aromatic 5- or 6-membered heterocyclic-C₁₋₆ alkyl-C₆₋₁₄ aryl having 1 to 4 hetero atoms selected from the group consisting of nitrogen atom, oxygen atom and sulfur atom, and mono- or di-C₁₋₆ alkylamino C₁₋₆ alkyl, or (2) morpholino group;

[19] The compound according to the above [17], wherein Z^{2′} is amino group optionally substituted with 1 or 2 C₁₋₆ alkyls;

[20] The compound according to the above [1], wherein R¹⁴ is hydrogen atom;

[21] The compound according to the above [1], wherein R¹ is:

(1) sulfinyl group, sulfonyl group or thiol group each of which may be substituted with C₁₋₈ alkyl or C₇₋₁₄ aralkyl,

(2) i) a C₆₋₁₀ aryloxy group,

ii) an aromatic 5- or 6-membered heterocyclic-oxy group having 1 to 4 hetero atoms selected from the group consisting of nitrogen atom, oxygen atom and sulfur atom, or

iii) a fused bicyclic heterocyclic-oxy group formed by aromatic 5- or 6-membered heterocyclic ring having 1 to 3 hetero atoms selected from the group consisting of nitrogen atom, oxygen atom and sulfur atom fused with benzene ring or aromatic 5- or 6-membered heterocyclic ring having 1 to 3 hetero atoms selected from the group consisting of nitrogen atom, oxygen atom and sulfur atom,

each of which may be substituted with 1 to 2 substituents selected from the group consisting of C₁₋₈ alkyl, C₆₋₁₄ aryl, C₇₋₁₄ aralkyl, hydroxyl, C₁₋₆ alkoxy, C₁₋₆ alkylenedioxy, C₆₋₁₄ aryloxy, C₁₋₆ alkylthio, morpholinosulfonyl, formyl, C₁₋₆ alkyl-carbonyl, carboxyl, C₁₋₆ alkoxy-carbonyl, C₆₋₁₄ aryloxy-carbonyl, C₇₋₁₄ aralkyloxy-carbonxyl, amino, mono- or di-C₁₋₆ alkylamino, mono- or di-C₁₋₆ alkyl-carbonylamino, toluenesulfonylamino, C₁₋₆ alkylaminothiocarbonylamino, nitro, halogen, C₁₋₆ haloalkyl, C₁₋₆ haloalkoxy, C₁₋₆ alkoxy-C₆₋₁₄ aryl, 2-amino-3-(4-morpholinyl)-3-oxo-C₁₋₆ alkyl, C₁₋₆ alkyl having one or two di(C₁₋₆ alkyoxy)phosphoryl, an aromatic 5- or 6-membered heterocyclic group having 1 to 4 hetero atoms selected from the group consisting of nitorgen atom, oxygen atom and sulfur atom, an aromatic 5- or 6-membered heterocyclic-oxy group having 1 to 4 hetero atoms selected from the group consisting of nitrogen atom, oxygen atom and sulfur atom, and a fused bicyclic heterocyclic-oxy group formed by an aromatic 5- or 6-membered heterocyclic ring having 1 to 3 hetero atoms selected from the group consisting of nitorgen atom, oxygen atom and sulfur aton fused with benzene ring or an aromatic 5- or 6-membered heterocyclic ring having 1 to 3 hetero atoms selected from the group consisting of nitrogen atom, oxygen atom and sulfur atom,

(3) a C₁₋₈ alkyloxy group, a C₃₋₇ cycloalkyloxy group, a C₆₋₁₄ aryloxy group, a C₇₋₁₄ aralkyloxy group or dihydrobenzofuranyloxy group,

(4) amino group which may be substituted with 1 or 2 substituents selected from the group consisting of C₁₋₈ alkyl, C₆₋₁₀ aryl, C₇₋₁₄ aralkyl, halo C₆₋₁₀ aryl, C₁₋₆ alkyl-carbonyl, C₆₋₁₀ aryl-carbonyl and C₇₋₁₄ aralkyl-oxycarbonyl, or

(5) C₁₋₈ alkyl or C₇₋₁₄ aralkyl

R¹ is —CO-Z^{2′} and Z^{2′} is:

(1) amino group which may be substituted with 1 or 2 substituents selected from the group consisting of C₁₋₈ alkyl, C₆₋₁₄ aryl, C₇₋₁₄ aralkyl, hydroxyl, C₁₋₆ alkoxy, amino, mono- or di-C₁₋₆ alkyl amino, C₁₋₆ alkoxy-carbonylamino, an aromatic 5- or 6-membered heterocyclic group having 1 to 4 hetero atoms selected from the group consisting of nitorgen atom, oxygen atom and sulfur atom, C₁₋₆ haloalkyl, carboxy C₁₋₆ alkyl, C₁₋₆ alkoxy-carbonyl-C₁₋₆ alkyl, carbamoyl C₁₋₆ alkyl, aromatic 5- or 6-membered heterocyclic-C₁₋₆ alkyl having 1 to 4 hetero atoms selected from the group consisting or nitrogen atom, oxygen atom and sulfur atom, aromatic 5- or 6-membered heterocyclic-C₁₋₆ alkyl-C₆₋₁₄ aryl having 1 to 4 hetero atoms selected from the group consisting of nitrogen atom, oxygen atom and sulfur atom, and mono- or di-C₁₋₆ alkylamino C₁₋₆ alkyl, or (2) morpholino group,

R³ is:

(1) hydrogen atom, (2) a C₁₋₈ alkyl group optionally substituted with 1 to 3 substituents selected from the group consisting of C₁₋₆ alkoxy, carboxyl, C₁₋₆ alkoxy-carbonayl, halogen and hydroxyl, (3) a C₇₋₁₄ aralkyl group, (4) a C₂₋₈ alkanoyl group, (5) a C₁₋₆ alkoxy-carbonyl group, (6) carbamoyl group optionally substituted with C₁₋₆ alkyl, (7) C₁₋₈ alkylsulfinyl or (8) C₁₋₈ alkylsulfonyl, and R¹⁴ is (1) hydrogen atom, (2) halogen, (3) C₁₋₈ alkyl or (4) i) C₆₋₁₀ aryl, ii) a 5- to 7-membered heterocyclic group containing one sulfur atom, one nitrogen atom or one oxygen atom, iii) a 5- to 6-membered heterocyclic group containing 2 to 4 nitrogen atoms, iv) a 5- to 6-membered heterocyclic group containing 1 to 2 nitrogen atoms and one sulfur atom or oxygen atom, or v) a heterocyclic group of ii) to iv) which is fused with a 5- to 6-membered heterocyclic ring containing 2 or less nitrogen atom, benzene ring or a 5-membered heterocyclic ring, each of which may have 1 to 3 substituents selected from the group consisting of C₁₋₆ alkyl, C₁₋₆ haloalkyl, C₁₋₆ alkoxy, C₁₋₆ haloalkoxy, C₁₋₆ alkoxy-carbonyl, amino and C₁₋₆ alkylthio, and

ring B is a 5- to 7-membered hydrocarbon ring optionally substituted with 1 to 3 C₁₋₈ alkyl groups;

[22] The compound according to the above [1], wherein R¹ is (a) C₁₋₆ alkylenedioxy, (b) di(C₁₋₆ alkoxy)phosphoryl-C₁₋₆ alkyl, (c) C₁₋₆ haloalkyl, (d) C₇₋₁₄ aralkyloxy, (e) aromatic 5- or 6-membered heterocyclic-oxy having 1 to 4 hetero atoms selected from the group consisting of nitrogen atom, oxygen atom and sulfur atom or (f) a C₆₋₁₀ aryloxy group which may be subsituted with fused bicyclic heterocyclic-oxy formed by an aromatic 5- or 6-membered heterocyclic ring having 1 to 3 hetero atoms selected from the group consisting of nitrogen atom, oxygen atom and sulfur atom fused with benzene ring or an aromatic 5- or 6-membered heterocyclic ring having 1 to 3 hetero atoms selected from the group consisting of nitrogen atom, oxygen atom and sulfur atom,

R² is —CO-Z^{2′} and Z^{2′} is amino group optionally substituted with 1 or 2 C₁₋₆ alkyl groups,

R³ is a C₁₋₈ alkyl group,

R¹⁴ is hydrogen atom, and

ring B is cyclohexane ring;

[23] The compound according to the above [1], wherein

9-methylthio-4,5-dihydro-6H-1-oxa-8-thia-2-aza-cyclopenta[e]azulene-7-carboxamide,

4,5-dihydro-8-methylthio-4-phenylisoxazolo[4,5-e]benzo[c]thiophene-6-carboxamide,

4,5-dihydro-4-methyl-8-(methylthio)isoxazolo[4,5-e]benzo[c]thiophene-6-carboxamide, and

4,5-dihydro-4,4-dimethyl-8-(methylthio)-isoxazolo[4,5-e]benzo[c]thiophene-6-carboxamide are further excluded from the compound represented by the formula (I);

[24] The compound according to the above [1], wherein a compound wherein, when ring B is a 6-membered hydrocarbon ring substituted with a lower alkyl group or phenyl, R¹ is a lower alkylthio group, and a compound wherein, when ring B is an unsubstituted 7-membered hydrocarbon ring, R¹ is a lower alkylthio group are further excluded from the compound represented by the formula (I);

[25] The compound according to the above [1], wherein the compound represented by the formula (I) is:

4,5-dihydro-1-methyl-8-propylsulfanyl-1H-thieno [3,4-g]indazole-6-carboxamide;

4,5-dihydro-1-methyl-8-propylsulfinyl-1H-thieno [3,4-g]indazole-6-carboxamide;

4,5-dihydro-1-methyl-8-propylsulfonyl-1H-thieno[3,4-g]indazole-6-carboxamide;

4,5-dihydro-8-(3,4-methylenedioxyphenoxy)-1-methyl-1H-thieno[3,4-g]indazole-6-carboxamide;

4,5-dihydro-8-phenoxy-1-methyl-1H-thieno [3,4-g]indazole-6-carboxamide;

4,5-dihydro-8-(3,4-methylenedioxyphenoxy)thieno-[3,4-g]-1,2-benzisoxazole-6-carboxamide;

8-[4-[(diethoxyphosphoryl)methyl]phenoxy]-4,5-dihydro-2-methyl-2H-thieno[3,4-g]indazole-6-carboxamide;

8-[4-[(diethoxyphosphoryl)methyl]phenoxy]-4,5-dihydro-1-methyl-1H-thieno[3,4-g]indazole-6-carboxamide;

N-ethyl-4,5-dihydro-8-(3,4-methylenedioxy-phenoxy)-1-methyl-1H-thieno[3,4-g]indazole-6-carboxamide;

4,5-dihydro-1-methyl-8-(4-trifluoromethyl-phenoxy)-1H-thieno[3,4-g]indazole-6-carboxamide;

4,5-dihydro-1-methyl-8-(6-quinolinyloxy)-1H-thieno[3,4-g]indazole-6-carboxamide;

4,5-dihydro-1-methyl-8-(3-pyridinyloxy)-1H-thieno[3,4-g]indazole-6-carboxamide;

8-[4-(benzyloxy)phenoxy]-4,5-dihydro-1-methyl-1H-thieno[3,4-g]indazole-6-carboxamide; or

4,5-dihydro-1-methyl-8-[4-(2-quinolinylmethoxy)phenoxy]-1H-thieno[3,4-g]indazole-6-carboxamide;

[26] A prodrug of the compound according to the above [1] or a salt thereof;

[27] A pharmaceutical composition which comprises a compound represented by the general formula (I):

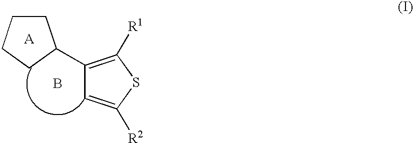

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

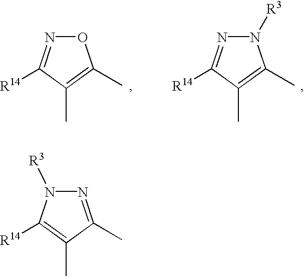

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than, when ring A is

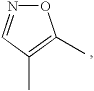

Z¹ of -Z¹-Z² is —CO— and Z² thereof is optionally substituted amino group, or a pharmaceutically acceptable salt thereof or a prodrug thereof;

[28] The composition according to the above [27], wherein

9-methylthio-4,5-dihydro-6H-1-oxa-8-thia-2-aza-cyclopenta[e]azulene-7-carboxamide,

4,5-dihydro-8-methylthio-4-phenylisoxazolo[4,5-e]benzo[c]thiophene-6-carboxamide,

4,5-dihydro-4-methyl-8-(methylthio)isoxazolo[4,5-e]benzo[c]thiophene-6-carboxamide, and

4,5-dihydro-4,4-dimethyl-8-(methylthio)isoxazolo[4,5-e]benzo[c]thiophene-6-carboxamide are further excluded from the compound represented by the formula (I);

[29] The composition according the above [27], wherein a compound wherein, when ring B is a 6-membered hydrocarbon ring substituted with a lower alkyl group or phenyl, R¹ is a lower alkylthio group, and a compound wherein, when ring B is an unsubstituted 7-membered hydrocarbon ring, R¹ is a lower alkylthio group are further excluded from the compound represented by the formula (I);

[30] A cell differentiation inducing drug which comprises a compound represented by the general formula (I):

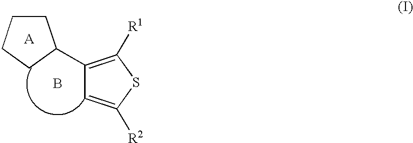

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

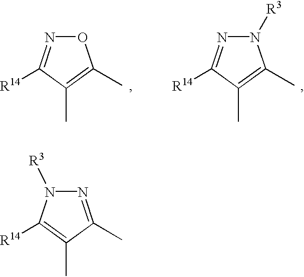

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula:

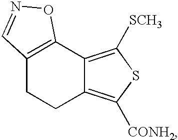

or a pharmaceutically acceptable salt thereof or a prodrug thereof;

[31] The cell differentiation inducing drug according to the above [30], wherein 9-methylthio-4,5-dihydro-6H-1-oxa-8-thia-2-aza-cyclopenta[e]azulene-7-carboxamide,

4,5-dihydro-8-methylthio-4-phenylisoxazolo[4,5-e]benzo[c]thiophene-6-carboxamide,

4,5-dihydro-4-methyl-8-(methylthio)isoxazolo[4,5-e]benzo[c]thiophene-6-carboxamide, and

4,5-dihydro-4,4-dimethyl-8-(methylthio)isoxazolo[4,5-e]benzo[c]thiophene-6-carboxamide are further excluded from the compound represented by the formula (I);

[32] The cell differentiation inducing drug according to the above [30], wherein a compound wherein, when ring B is a 6-membered hydrocarbon ring substituted with a lower alkyl group or phenyl, R¹ is a lower alkylthio group, and a compound wherein, when ring B is an unsubstituted 7-membered hydrocarbon ring, R¹ is a lower alkylthio group are further excluded from the compound represented by the formula (I);

[33] A a prophylactic or therapeutic drug for bone or articular diseases which comprises a compound represented by the general formula (I):

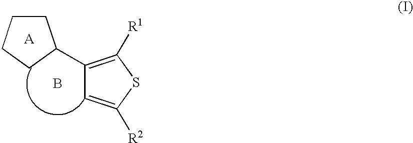

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

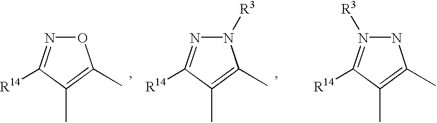

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula:

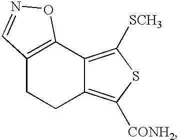

or a pharmaceutically acceptable salt thereof or a prodrug thereof;

[34] The prophylactic or therapeutic drug for bone or articular diseases according to the above [33], wherein the bone or articular diseases are osteoporosis, bone fracture, osteoarthritis or chronic rheumatoid arthritis;

[35] A compound represented by general formula (II):

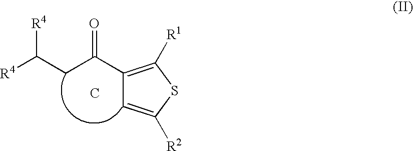

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R⁴ represents a substituted hydroxyl group; and ring C represents an optionally substituted 5- to 7-membered hydrocarbon ring, or a salt thereof;

[36] A compound represented by the general formula (IX):

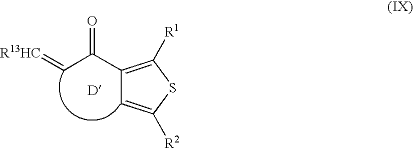

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group, or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group, or an optionally substituted amino group); R¹³ represents an optionally substituted amino group or an optionally substituted hydroxyl group; and ring D′ represents an optionally substituted 5- to 7-membered hydrocarbon ring, or a salt thereof;

[37] A process for producing a compound represented by the general formula (IX-1):

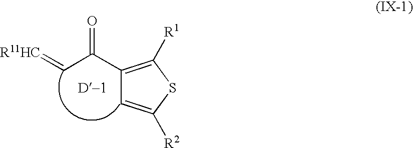

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R¹¹ represents an optionally substituted amino group; ring D′-1 represents an optionally substituted 5- to 7-membered hydrocarbon, or a salt thereof, which comprises reacting a compound of the general formula (IV)

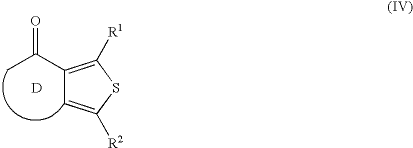

wherein ring D represents an optionally substituted 5- to 7-membered hydrocarbon ring; and R¹ and R² are as defined above, with an amide acetal;

[38] A process for producing a compound of the general formula (IX-2):

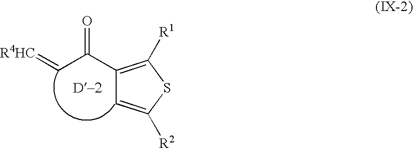

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R⁴ represents an optionally substituted hydroxyl group; ring D′-2 represents an optionally substituted 5- to 7-membered hydrocarbon ring, or a salt thereof, which comprises subjecting a compound of the general formula (II):

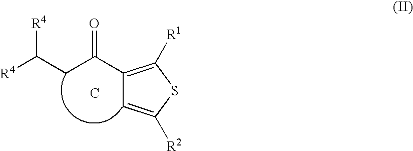

wherein ring C represents an optionally substituted 5- to 7-membered hydrocarbon ring; and R¹, R² and R⁴ are as defined above, to a de-alcoholization reaction;

[39] A process for producing a compound represented by the general formula (I):

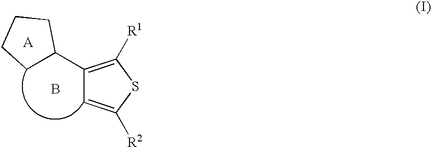

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

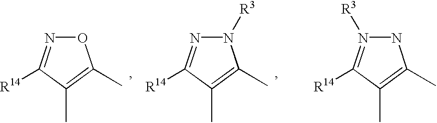

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring, or a salt thereof, which comprises subjecting a compound represented by the general formula (IX)

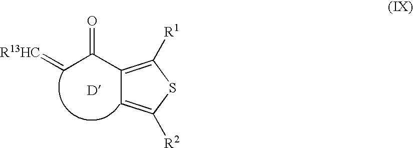

wherein R¹³ represents an optionally substituted amino group or an optionally substituted hydroxyl group; ring D′ represents an optionally substituted 5- to 7-membered hydrocarbon ring; and R¹ and R² are as defined above, or a salt thereof, and hydroxylamine or its salt, or a compound represented by the formula: R^{3′}NHNH₂ (wherein R^{3′} represents hydrogen atom or an optionally substituted hydrocarbon group, an optionally substituted heterocyclic group, an optionally substituted sulfonyl group, or an optionally substituted acyl group), or a salt thereof to a cyclization reaction, if desired, followed by conversion into an optionally substituted hydroxyl group or an optionally substituted amino group;

[40] A process for producing a compound represented by the general formula (I-II):

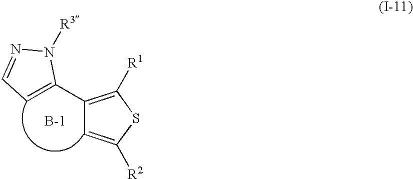

wherein R^{1′} represents, among an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group, a group represented by the formula: —X′—W′ (wherein X′ represents a bond, an optionally substituted carbon atom, an optionally substituted nitrogen atom, oxygen atom or an optionally oxidized sulfur atom; R^{3″} represents an optionally substituted hydrocarbon group, an optionally substituted heterocyclic group, an optionally substituted sulfonyl group or an optionally substituted acyl group; and W′ represents an optionally substituted cyclic group or carbon or nitrogen atom having 2 or more substituents); R² represents cyano group, formyl group, thioformyl group, or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); ring B-1 represents an optionally substituted 5- to 7-membered hydrocarbon ring, or a salt thereof, which comprises subjecting a compound represented by the general formula (IX-3):

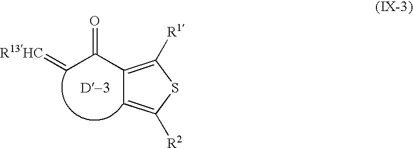

wherein R^{13′} represents an optionally substituted amino group or an optionally substituted hydroxyl group; ring D′-3 represents an optionally substituted 5- to 7-membered hydrocarbon ring; and R^{1′} and R² are as defined above, or a salt thereof, and a compound resented by formula: R^{3″}NHNH₂ (wherein R^{3″} is as defined above), or a salt thereof to a cyclization reaction in the presence of an acid under substantially anhydrous conditions;

[41] The process according to the above [40], wherein R^{1′} is the group represented by —X′—W′, wherein, X′ represents oxygen atom or an optionally oxidized sulfur atom; and W′ represents an optionally substituted cyclic group;

[42] A method for inducing cell differentiation in a mammal in need thereof which comprises administering an effective amount of a compound represented by the general formula (I):

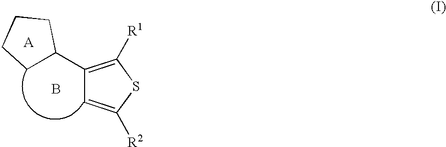

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

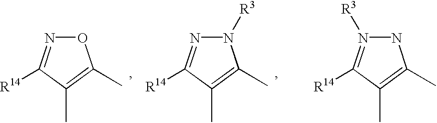

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula:

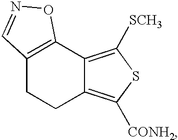

or a pharmaceutically acceptable salt thereof or a prodrug thereof, to the mammal;

[43] A method for preventing or treating bone or articular diseases in a mammal in need thereof which comprises administering an effective amount of a compound represented by the general formula (I):

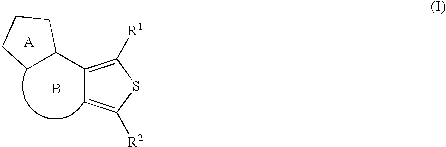

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

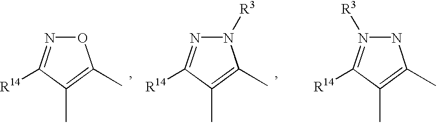

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula:

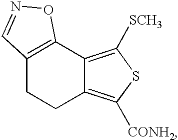

or a pharmaceutically acceptable salt thereof or a prodrug thereof, to the mammal;

[44] Use of a compound represented by the general formula (I):

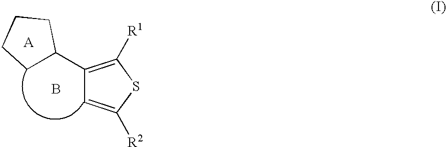

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

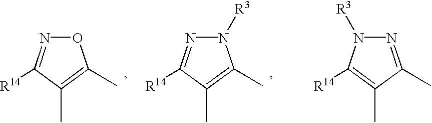

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula:

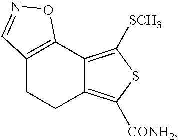

or a pharmaceutically acceptable salt thereof or a prodrug thereof, for manufacturing a cell defferentiation inducing drug; and

[45] Use of a compound represented by the general formula (I):

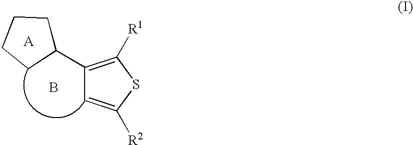

wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of

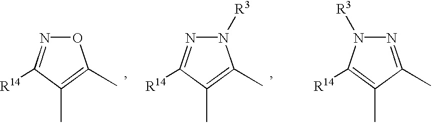

(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula:

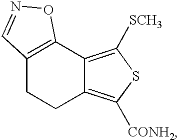

or a pharmaceutically acceptable salt thereof or a prodrug thereof, for manufacturing a prophylactic or therapeutic drug for bone or articular diseases which comprises.

### DETAILED DESCRIPTION OF THE INVENTION

In the above formulas, R¹ represents an optionally substituted hydrocarbon group, an optionally substituted heterocyclic group, an optionally substituetd sulfinyl group, an optionally substituted sulfonyl group, an optionally substituted hydroxyl group, an optionally substituted thiol group, or an optionally substituted amino group.

Examples of the hydrocarbon group in an optionally substituted hydrocarbon group of R¹ include an optionally substituted aliphatic hydrocarbon group, an optionally substituted alicyclic hydrocarbon group, an optionally substituted alicyclic-aliphatic hydrocarbon group, an optionally substituted aromatic hydrocarbon group, an optionally substituted aromatic-aliphatic hydrocarbon group (an aralkyl group), and the like.

Examples of said aliphatic hydrocarbon group include a saturated aliphatic hydrocarbon group having 1-8 carbon atoms (e.g., alkyl group) such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.; and an unsaturated aliphatic hydrocarbon group having 2-8 carbon atoms (e.g., alkenyl group, alkynyl group, alkadienyl group, alkadiynyl group, etc.) such as vinyl, allyl, 1-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl, 2,4-hexadienyl, 1-heptenyl, 1-octenyl, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 2,4-hexadiynyl, 1-heptynyl, 1-octynyl, etc.

Examples of said alicyclic hydrocarbon group include a saturated alicyclic hydrocarbon group having 3-7 carbon atoms (e.g., cycloalkyl group, etc.) such as cyclopropyl, cyclobutyl, cyclopentyl, cyclohexyl, cycloheptyl, and the like; an unsaturated alicyclic hydrocarbon group having 3-7 carbon atoms (e.g., cycloalkenyl group, cycloalkadienyl group, etc.) such as 1-cyclopentenyl, 2-cyclopentenyl, 3-cyclopentenyl, 1-cyclohexenyl, 2-cyclohexenyl, 3-cyclohexenyl, 1-cycloheptenyl, 2-cycloheptenyl, 3-cycloheptenyl, 2,4-cycloheptadienyl, etc.; a partly saturated and fused bicyclic hydrocarbon group [preferably, C₉₋₁₀ partly saturated and fused bicyclic hydrocarbon group, etc. (including those where the benzene ring is combined to 5- or 6-membered non-aromatic cyclic hydrocarbon group)] such as 1-indenyl, 2-indenyl, 1-indanyl, 2-indanyl, 1,2,3,4-tetrahydro-1-naphthyl, 1,2,3,4-tetrahydro-2-naphthyl, 1,2-dihydro-1-naphthyl, 1,2-dihydro-2-naphthyl, 1,4-dihydro-1-naphthyl, 1,4-dihydro-2-naphthyl, 3,4-dihydro-1-naphthyl, 3,4-dihydro-2-naphthyl, etc.; and the like.

Examples of said alicyclic-aliphatic hydrocarbon group include those where the above-mentioned alicyclic hydrocarbon group and the above-mentioned aliphatic hydrocarbon group are combined, for example, those having 4-14 carbon atoms such as cyclopropylmethyl, cyclopropylethyl, cyclobutylmethyl, cyclobutylethyl, cyclopentylmethyl, 2-cyclopentenylmethyl, 3-cyclopentenylmethyl, cyclopentylethyl, cyclohexylmethyl, 2-cyclohexenylmethyl, 3-cyclohexenylmethyl, cyclohexylethyl, cycloheptylmethyl, cycloheptylethyl, 2-(3,4-dihydro-2-naphtyl)ethyl, 2-(1,2,3,4-tetrahydro-2-naphtyl)ethyl, 2-(3,4-dihydro-2-naphtyl)ethenyl, etc. (e.g., C₃₋₇ cycloalkyl-C₁₋₄ alkyl group, C₃₋₇ cycloalkenyl-C₁₋₄ alkyl group, C₃₋₇ cycloalkyl-C₂₋₄ alkenyl group, C₃₋₇ cycloalkenyl-C₂₋₄ alkenyl group, C₉₋₁₀ partly saturated and fused bicyclic hydrocarbon-C₁₋₄ alkyl group, C₉₋₁₀ partly saturated and fused bicyclic hydrocarbon-C₁₋₄ alkenyl groups, etc.).

Examples of said aromatic hydrocarbon group include an aryl group having 6-10 carbon atoms (including that where a 5- to 6-membered non-aromatic hydrocarbon ring is fused with phenyl group) such as phenyl, α-naphthyl, β-naphthyl, 4-indenyl, 5-indenyl, 4-indanyl, 5-indanyl, 5,6,7,8-tetrahydro-1-naphthyl, 5,6,7,8-tetrahydro-2-naphthyl, 5,6-dihydro-1-naphthyl, 5,6-dihydro-2-naphthyl, 5,6-dihydro-3-naphthyl, 5,6-dihydro-4-naphthyl, etc.; and the like.

Examples of said aromatic-aliphatic hydrocarbon group include an aralkyl group having 7-14 carbon atoms (C₆₋₁₀ aryl-C₂₋₄ alkyl group) such as phenyl-C₁₋₄ alkyl group, e.g., benzyl, phenethyl, 1-phenylethyl, 1-phenylpropyl, 2-phenylpropyl, 3-phenylpropyl, etc.; naphthyl-C₁₋₄ alkyl group such as α-naphthylmethyl, α-naphthylethyl, β-naphthylmethyl, β-naphthylethyl, etc.; C₆₋₁₀ aryl-C₂₋₄ alkenyl group such as phenyl-C₂₋₄ alkenyl group, e.g., styryl, cinnamyl, etc.; and the like.

Examples of the heterocyclic group in an optionally substituted heterocyclic group of R¹ include (i) a 5- to 7-membered heterocyclic group containing one sulfur atom, one nitrogen atom, or one oxygen atom, (ii) a 5- to 6-membered heterocyclic group containing 2-4 nitrogen atoms,

(iii) a 5- to 6-membered heterocyclic group containing 1-2 nitrogen atoms and one sulfur or oxygen atom, or the like; and (iv) these heterocyclic groups may be fused with a 5-to 6-membered ring containing 2 or less nitrogen atoms, benzene ring, or a 5-membered ring containing one sulfur atom. In addition, each of the heterocyclic groups exemplified in (i) to (iv) may be a saturated or unsaturated heterocyclic group and the unsaturated heterocyclic group may be either aromatic or non-aromatic.

Examples of the heterocyclic group in an optionally substituted heterocyclic group of R¹ include an aromatic monocyclic heterocyclic group, an aromatic fused heterocyclic group, and a non-aromatic heterocyclic group.

Specific examples of the heterocyclic group in an optionally substituted heterocyclic group of R¹ include (i) an aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, etc.); (ii) an aromatic fused heterocyclic group (e.g., benzofuranyl, isobenzofuranyl, benzo[b]thienyl, indolyl, isoindolyl, 1H-indazolyl, benzimidazolyl, benzoxazolyl, 1,2-benzisothiazolyl, 1H-benztriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.); and (iii) a non-aromatic, heterocyclic group (e.g., oxiranyl, azetidinyl, oxetanyl, thietanyl, pyrrolidinyl, tetrahydrofuryl, thiolanyl, piperidyl, tetrahydropyranyl, morpholinyl, thiomorpholinyl, piperazinyl, etc.).

Examples of sulfinyl group in an optionally substituted sulfinyl group of R¹ include that where —SO— is combined with “the hydrocarbon group” or “the heterocyclic group” in “an optionally substituted hydrocarbon group or an optionally substituted heterocyclic group” of the above R¹.

Preferred examples include a C₁₋₈ alkylsulfinyl group where sulfinyl group is combined with a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.; a C₆₋₁₀ arylsulfinyl group where sulfinyl group is combined with a C₆₋₁₀ aryl group such as phenyl, α-naphthyl, β-naphthyl, 4-indenyl, 5-indenyl, 4-indanyl, 5-indanyl, 5,6,7,8-tetrahydro-1-naphthyl, 5,6,7,8-tetrahydro-2-naphthyl, 5,6-dihydro-1-naphthyl, 5,6-dihydro-2-naphthyl, 5,6-dihydro-3-naphthyl, 5,6-dihydro-4-naphthyl, etc.; a group where sulfinyl group is combined with an aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, etc.); and a group where sulfinyl group is combined with an aromatic fused heterocyclic group (e.g., benzofuranyl, isobenzofuranyl, benzo[b]thienyl, indolyl, isoindolyl, 1H-indazolyl, benzimidazolyl, benzoxazolyl, 1,2-benzisothiazolyl, 1H-benztriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.).

More preferred examples include a C₁₋₈ alkylsulfinyl group where sulfinyl group is combined with a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.

Examples of sulfonyl group in an optionally substituted sulfonyl group of R¹ include a group where —SO²— is combined with “the hydrocarbon group” or “the heterocyclic group” in “an optionally substituted hydrocarbon group or an optionally substituted heterocyclic group” of the above R¹.

Preferred examples include a C₁₋₈ alkylsulfonyl group where sulfonyl group is combined with a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.; a C₆₋₁₀ arylsulfonyl group where sulfonyl group is combined with a C₆₋₁₀ aryl group such as phenyl, α-naphthyl, β-naphthyl, 4-indenyl, 5-indenyl, 4-indanyl, 5-indanyl, 5,6,7,8-tetrahydro-1-naphthyl, 5,6,7,8-tetrahydro-2-naphthyl, 5,6-dihydro-1-naphthyl, 5,6-dihydro-2-naphthyl, 5,6-dihydro-3-naphthyl, 5,6-dihydro-4-naphthyl, etc.; a group where sulfonyl group is combined with an aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pirazinyl, triazinyl, or the like); and a group where the sulfonyl group is combined with an aromatic, fused heterocyclic group (e.g., benzofuranyl, isobenzofuranyl, benzo[b]thienyl, indolyl, isoindolyl, 1H-indazolyl, benzimidazolyl, benzoxazolyl, 1,2-benzisothiazolyl, 1H-benztriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.).

More preferared examples include a C₁₋₈ alkylsulfonyl group where sulfonyl group is combined with a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.

Examples of an optionally substituted hydroxyl group of R¹ include hydroxyl group and that having an appropriate substituent, for example, “an optionally substituted hydrocarbon group or an optionally substituted heterocyclic group” represented by R¹.

Preferred examples include a C₁₋₈ alkyloxy group whose substituent is a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.; a C₆₋₁₀ aryloxy group whose substituent is a C₆₋₁₀ aryl group such as phenyl, α-naphthyl, β-naphthyl, 4-indenyl, 5-indenyl, 4-indanyl, 5-indanyl, 5,6,7,8-tetrahydro-1-naphthyl, 5,6,7,8-tetrahydro-2-naphthyl, 5,6-dihydro-1-naphthyl, 5,6-dihydro-2-naphthyl, 5,6-dihydro-3-naphthyl, 5,6-dihydro-4-naphthyl, etc.; a hydroxyl group substituted with an aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, etc.); a hydroxyl group substituted with an aromatic fused heterocyclic group (e.g., benzofuranyl, isobenzofuranyl, benzo[b]thienyl, indolyl, isoindolyl, 1H-indazolyl, benzimidazolyl, benzoxazolyl, 1,2-benzisothiazolyl, 1H-benztriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.).

More preferred examples include a C₆₋₁₀ aryloxy group (in particular, phenyloxy) or a hydroxyl group substituted with an aromatic monocyclic heterocyclic group (in particular, pyridyl) or an aromatic fused heterocyclic group (in particular, quinolyl).

“The hydrocarbon group” or “the heterocyclic group” as the substituent of the substituted hydroxyl group exemplified above may have the same substituent as that of “the hydrocarbon group” or “the heterocyclic group” in “an optionally substituted hydrocarbon group or an optionally substituted heterocyclic group” of the above R¹.

Examples of an optionally substituted thiol group of R¹ include thiol group and that substituted with an appropriate group such as “an optionally substituted hydrocarbon group or an optionally substituted heterocyclic group represented by R¹.

Preferred examples include a C₁₋₈ alkylthio group, whose substituent is a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.; a C₆₋₁₀ arylthio group, whose substituent is a C₆₋₁₀ aryl group such as phenyl, α-naphthyl, β-naphthyl, 4-indenyl, 5-indenyl, 4-indanyl, 5-indanyl, 5,6,7,8-tetrahydro-1-naphthyl, 5,6,7,8-tetrahydro-2-naphthyl, 5,6-dihydro-1-naphthyl, 5,6-dihydro-2-naphthyl, 5,6-dihydro-3-naphthyl, 5,6-dihydro-4-naphthyl, etc.; a thiol group substituted with an aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, etc.); and a thiol group substituted with an aromatic fused heterocyclic groups (e.g., benzofuranyl, isobenzofuranyl, benzo[b]thienyl, indolyl, isoindolyl, 1H-indazolyl, benzimidazolyl, benzoxazolyl, 1,2-benzisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.).

“The hydrocarbon group” or “the heterocyclic group” as the substituent of the substituted thiol group exemplified above may have the same substituent as that of “the hydrocarbon group” or “the heterocyclic group” in “an optionally substituted hydrocarbon group or an optionally substituted heterocyclic group” of the above-mentioned R¹.

More preferred examples include a C₁₋₈ alkylthio group substituted with a C₁₋₈ alkyl such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, or the like.

Examples of an optionally substituted amino group include amino group, an N-mono-substituted amino group, and an N,N-disubstituted amino group. Examples of said substituted amino groups include that having one or two substituents of an optionally substituted hydrocarbon group (e.g., the same group as an optionally substituted hydrocarbon group of R¹, more specifically, a C₁₋₈ alkyl group, a C₃₋₇ cycloalkyl group, a C₂₋₈ alkenyl group, a C₂₋₈ alkynyl group, a C₃₋₇ cycloalkenyl group, a C₆₋₁₀ aryl group that may have a C₁₋₄ alkyl group, etc.), an optionally substituted heterocyclic group (e.g., the same group as an optionally substituted heterocyclic group of R¹), or the formula: —COR′ (wherein R′ represents hydrogen atom or an optionally substituted hydrocarbon group or an optionally substituted heterocyclic group. As for “the hydrocarbon group” or “the heterocyclic group” in “an optionally substituted hydrocarbon group” or “an optionally substituted heterocyclic group” of R′ may have the same substituent as that of “the hydrocarbon group” or “the heterocyclic group” in “an optionally substituted hydrocarbon group” or “an optionally substituted heterocyclic group” of the above R¹.), preferably a C₁₋₁₀ acyl group (e.g., a C₂₋₇ alkanoyl, benzoyl, nicotinoyl, etc.). Specific examples thereof include methylamino, dimethylamino, ethylamino, diethylamino, dipropylamino, dibutylamino, diallylamino, cyclohexylamino, phenylamino, N-methyl-N-phenylamino, acetylamino, propionylamino, benzoylamino, nicotinoylamino, and the like.

In addition, the two groups in said substituted amino groups may be combined to form a nitrogen-containing 5- to 7-membered ring (e.g., piperidino, piperadino, morpholino, thiomorpholino, etc.).

“The hydrocarbon group”, “the heterocyclic group”, “the sulfinyl group”, or “the sulfonyl group” in “an optionally substituted hydrocarbon group, an optionally substituted heterocyclic group, an optionally substituted sulfinyl group, or an optionally substituted sulfonyl group” represented by R¹ may be substituted with 1-3 substituents. Examples of said substituents include a lower (C₁₋₆) alkyl group (e.g., methyl, ethyl, propyl, isopropyl, butyl, isobutyl, sec-butyl, tert-butyl, pentyl, isopentyl, neopentyl, hexyl, etc.); a lower (C₂₋₆) alkenyl group (e.g., vinyl, allyl, 1-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 4-hexenyl, 5-hexenyl, etc.); a lower (C₂₋₆) alkynyl group (e.g., ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, etc.); a C₃₋₇ cycloalkyl group (e.g., cyclopropyl, cyclobutyl, cyclopentyl, cyclohexyl, cycloheptyl, etc.); a C₆₋₁₀ aryl group (e.g., phenyl, α-naphthyl, β-naphthyl, etc.); an aromatic heterocyclic group [e.g., (i) an aromatic 5- or 6-membered heterocyclic group having 1-4 heteroatoms selected from nitrogen atom, oxygen atom, and sulfur atom; (ii) a fused bicyclic heterocyclic group formed by condensation of an aromatic 5- or 6-membered heterocyclic group having 1-3 heteroatoms selected from nitrogen atom, oxygen atom, and sulfur atom with benzene ring or an aromatic 5- or 6-membered heterocyclic group having 1-3 heteroatoms selected from nitrogen atom, oxygen atom, and sulfur atom; (iii) a fused tricyclic heterocyclic group formed by condensation of [1] an aromatic, 5- or 6-membered heterocyclic group having 1-3 heteroatoms selected from nitrogen atom, oxygen atom and sulfur atom, [2] benzene ring, and [3] an aromatic 5- or 6-membered heterocyclic group having 1-3 heteroatoms selected from nitrogen atom, oxygen atom and sulfur atom or benzene ring, such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, benzofuranyl, isobenzofuranyl, benzo[b]thienyl, indolyl, isoindolyl, 1H-indazolyl, benzimidazolyl, benzoxazolyl, 1,2-benzisothiazolyl, 1H-benztriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.]; a heterocyclic-oxy group formed by combining each of the above heterocyclic groups (i), (ii) and (iii) with oxy group; a non-aromatic heterocyclic group (e.g., a non-aromatic, 4- or 7-membered heterocyclic group having 1-3 heteroatoms selected from nitrogen atom, oxygen atom and sulfur atom, such as oxiranyl, azetidinyl, oxetanyl, thietanyl, pyrrolidinyl, tetrahydrofuryl, thiolanyl, piperidinyl, tetrahydropyranyl, morpholinyl, thiomorpholinyl, piperazinyl, etc.); a C₇₋₁₄ aralkyl group (e.g., a C₆₋₁₀ aryl-C₁₋₄ alkyl group such as benzyl, phenethyl, 1-phenylethyl, 1-phenylpropyl, 2-phenylpropyl, 3-phenylpropyl, α-naphthylmethyl, α-naphthylethyl, β-naphthylmethyl, β-naphthylethyl, etc.); amino group; a N-mono-substituted amino group [e.g., a N-(C₁₋₆ alkyl)amino group such as methylamino, ethylamino, allylamino, cyclohexylamino, phenylamino, a N-(C₂₋₆ alkenyl)amino group, a N-(C₃₋₇ cycloalkyl)amino group, a N-(C₆₋₁₀ aryl)amino group, etc.]; a N,N-disubstituted amino group [e.g., an amino group substituted with two substituents selected from a C₁₋₆ alkyl group, a C₂₋₆ alkenyl group, a C₃₋₇ cycloalkenyl group, and a C₆₋₁₀ aryl group, such as dimethylamino, diethylamino, dibutylamino, diallylamino, N-methyl-N-phenylamino, etc.]; amidino group; an acyl group (e.g., a C₂₋₈ alkanoyl group such as formyl, acetyl, propionyl, butyryl, isobytyryl, valeryl, isovaleryl, pivaloyl, hexanoyl, heptanoyl, octanoyl, cyclopropanecarbonyl, cyclobutanecarbonyl, cyclopentanecarbonyl, cyclohexanecarbonyl, crotonyl, 2-cyclohexenecarbonyl, benzoyl, nicotinoyl, etc.; a C₃₋₈ alkenoyl group; a C₃₋₇ cycloalkyl-carbonyl group; a C₃₋₇ cycloalkenyl-carbonyl group; a C₆₋₁₀ aryl-carbonyl group; a heterocyclic-carbonyl group formed by binding of an aromatic or non-aromatic 5- or 6-membered heterocyclic group having 1-3 heteroatoms selected from nitrogen atom, oxygen atom and sulfur atom with carbonyl group, etc.); carbamoyl group; a mono-substituted carbamoyl group [e.g., a N-(C₁₋₆ alkyl)carbamoyl group such as methylcarbamoyl, ethylcarbamoyl, cyclohexylcarbamoyl, phenylcarbamoyl, etc.]; a N-(C₂₋₆ alkenyl)carbamoyl group; a N-(C₃₋₇ cycloalkyl)carbamoyl group; a N-(C₆₋₁₀ aryl)carbamoyl group; etc.]; a N,N-disubstituted carbamoyl group [e.g., a carbamoyl group substituted with two substituents selected from a C₁₋₆ alkyl group, a C₂₋₆ alkenyl group, a C₃₋₇ cycloalkyl group, and a C₆₋₁₀ aryl group, such as dimethylcarbamoyl, diethylcarbamoyl, dibutylcarbamoyl, diallylcarbamoyl, N-methyl-N-phenylcarbamoyl, etc.]; sulfamoyl group, a N-mono-substituted sulfamoyl group [e.g., a N-(C₁₋₆ alkyl)sulfamoyl group such as methylsulfamoyl, ethylsulfamoyl, cyclohexylsulfamoyl, phenylsulfamoyl, etc.; a N-(C₂₋₆ alkenyl)sulfamoyl group; a N-(C₃₋₇ cycloalkyl)sulfamoyl group; a N-(C₆₋₁₀ aryl)sulfamoyl group; etc.], a N,N-disubstituted sulfamoyl group [e.g., sulfamoyl group substituted with two substituents selected from a C₁₋₆ alkyl group, a C₂₋₆ alkenyl group, a C₃₋₇ cycloalkyl group, and a C₆₋₁₀ aryl group, such as dimethylsulfamoyl, diethylsulfamoyl, dibutylsulfamoyl, diallylsulfamoyl, N-methyl-N-phenylsulfamoyl, etc.]; carboxyl group; a lower (C₁₋₆) alkoxy-carbonyl group (e.g., methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl, etc.); hydroxyl group; a lower (C₁₋₆) alkoxy group (e.g., methoxy, ethoxy, propoxy, isopropoxy, butoxy, isobutoxy, sec-butoxy, tert-butoxy, pentyloxy, hexyloxy, etc.); a lower (C₂₋₁₀) alkenyloxy group (e.g., allyloxy, 2-butenyloxy, 2-pentenyloxy, 3-hexenyloxy, etc.); a C₃₋₇ cycloalkyloxy group (e.g., cyclopropyloxy, cyclobutyloxy, cyclopentyloxy, cyclohexyloxy, cycloheptyloxy, etc.); a C₆₋₁₀ aryloxy group (e.g., phenoxy, naphthyloxy, etc.); a C₇₋₁₄ aralkyloxy group (e.g., a C₆₋₁₀ aryl-C₁₋₄ alkyloxy group such as phenyl-C₁₋₄ alkyloxy, naphthyl-C₁₋₄ alkyloxy, etc.); mercapto group; a lower (C₁₋₆)alkylthio group (e.g., methylthio, ethylthio, propylthio, isopropylthio, butylthio, isobutylthio, sec-butylthio, tert-butylthio, pentylthio, isopentylthio, neopentylthio, hexylthio, etc.), a C₇₋₁₄ aralkylthio group (e.g., a C₆₋₁₀ aryl-C₁₋₄ alkylthio group such as phenyl-C₁₋₄ alkylthio, naphthyl-C₁₋₄ alkylthio, etc.); a C₆₋₁₀ arylthio group (e.g., phenylthio, naphtylthio, etc.), a lower (C₁₋₆) alkylsulfinyl group (e.g., methylsulfinyl, ethylsulfinyl, propylsulfinyl, isopropylsulfinyl, butylsulfinyl, isobutylsulfinyl, sec-butylsulfinyl, tert-butylsulfinyl, pentylsulfinyl, isopentylsulfinyl, neopentylsulfinyl, hexylsulfinyl, etc.); a C₇₋₁₄ aralkylsulfinyl group (e.g., a C₆₋₁₀ aryl-C₁₋₄ alkylsulfinyl group such as phenyl-C₁₋₄ alkylsulfinyl, naphthyl-C₁₋₄ alkylsulfinyl, etc.); a C₆₋₁₀ arylsulfinyl group (e.g., phenylsulfinyl, naphtylsulfinyl, etc.); a lower (C₁₋₆) alkylsulfonyl group (e.g., methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, isopentylsulfonyl, neopentylsulfonyl, hexylsulfonyl, etc.), a C₇₋₁₄ aralkylsulfonyl group (e.g., a C₆₋₁₀ aryl-C₁₋₄ alkylsulfonyl group such as phenyl-C₁₋₄ alkylsulfonyl, naphthyl-C₁₋₄ alkylsulfonyl, etc.), a C₆₋₁₀ arylsulfonyl group (e.g., phenylsulfonyl, naphtylsulfonyl, etc.); sulfo group; cyano group; azido group; a halogen atom (e.g., fluorine, chlorine, bromine, iodine, etc.); nitro group; nitroso group; an optionally esterified phosphono group [e.g., phosphono group, a (C₁₋₆ alkoxy)phosphoryl group such as ethoxyphosphoryl, a di-(C₁₋₆ alkoxy)phosphoryl group such as diethoxyphosphoryl, etc.]; a lower (C₁₋₆) alkyl group substituted with an optionally esterified phosphono group (e.g., a phosphono-C₁₋₆ alkyl group, a C₁₋₆ alkoxyphosphoryl-C₁₋₆ alkyl group, a di-(C₁₋₆ alkoxy)phosphoryl-C₁₋₆ alkyl group such as diethoxyphosphorylmethyl, etc.); a C₁-6 haloalkyl group (e.g., a C₁₋₆ alkyl group substituted with 1 to 4 halogen such as trifluoromethyl, etc.); a C₁₋₆ haloalkoxy group (e.g., a C₁₋₆ alkoxy group substituted with 1 to 4 halogen such as trifluoromethoxy, etc.); and the like.

Among the above substituents, when hydroxyl group is located adjacent to an lower (C₁₋₆) alkoxy group, they may form C₁₋₆ alkylenedioxy groups such as methylenedioxy, ethylenedioxy, or the like.

The above C₆₋₁₀ aryl group, the C₆₋₁₀ aryl group as a substituent of the aromatic heterocyclic group and the N-mono-substituted amino group, the C₆₋₁₀ aryl group as a substituent of the N,N-disubstituted amino group, the C₆₋₁₀ aryl group as a substituent of the N-mono-substituted carbamoyl group, the C₆₋₁₀ aryl group as a substituent of the N,N-disubstituted carbamoyl group, the C₆₋₁₀ aryl as a substituent of the N-mono-substituted sulfamoyl group, the C₆₋₁₀ aryl group as a substituent of the N,N-disubstituted sulfamoyl group, the C₆₋₁₀ aryl group as a substituent of the C₆₋₁₀ aryloxy group, the C₆₋₁₀ aryl group of the C₇₋₄ aralkyloxy group, the C₆₋₁₀ aryl group of the C₇₋₁₄ aralkylthio groups, the C₆₋₁₀ aryl group of the C₆₋₁₀ arylthio groups, the C₆₋₁₀ aryl group of the C₇₋₄ aralkylsulfinyl groups, the C₆₋₁₀ aryl group of the C₆₋₁₀ arylsulfinyl group, the C₆₋₁₀ aryl group of the C₇₋₁₄ aralkylsulfonyl groups, and the C₆₋₁₀ aryl group in the C₆₋₁₀ arylsulfonyl group may be substituted further with 1-3 substituent. Examples of said substituent include a lower (C₁₋₆) alkyl group, amino group, a N-(C₁₋₆ alkyl)amino group, a N,N-di-(C₁₋₆ alkyl)amino group, amidino group, carbamoyl group, a N-(C₁₋₆ alkyl)carbamoyl group, a N,N-di-(C₁₋₆ alkyl)carbamoyl group, sulfamoyl group, a N-(C₁₋₆ alkyl)sulfamoyl group, a N,N-di-(C₁₋₆ alkyl)sulfamoyl group, carboxyl group, a lower (C₂₋₇) alkoxycarbonyl group, hydroxyl group, a lower (C₁₋₆) alkoxy group, mercapto group, a lower (C₁₋₆) alkylthio group, sulfo group, cyano group, azido group, a halogen atom, nitro group, nitroso group, an optionally substituted phosphono group [e.g., phosphono group, a C₁₋₆ alkoxyphosphoryl group, a di-(C₁₋₆ alkoxy)phosphoryl group, etc.], a lower (C₁₋₆) alkyl group substituted with an optionally esterified phosphono group [e.g., a phosphono-C₁₋₆ alkyl group, a C₁₋₆ alkoxyphosphoryl-C₁₋₆ alkyl group, a di-(C₁₋₆ alkoxy)phosphoryl-C₁₋₆ alkyl group such as diethoxyphosphorylmethyl, etc.], and the like.

Among the above substituted, when hydroxyl group is located adjascen to a lower (C₁₋₆) alkoxyl group, they may form a C₁₋₆ alkylenedioxy group such as methylenedioxy, ethylenedioxy, or the like.

Preferred R¹ is an optionally substituted sulfinyl group, an optionally substituted sulfonyl group, an optionally substituted hydroxyl group, or an optionally substituted thiol group. That is, a group represented by the formula: —SR, —SOR, —SO₂R, or —OR (wherein R represents an optionally substituted hydrocarbon group or an optionally substituted heterocyclic group) is preferred as R¹. Examples of “an optionally substituted hydrocarbon group or an optionally substituted heterocyclic group” represented by R include the same group as “an optionally substituted hydrocarbon group or an optionally substituted heterocyclic group” represented by R¹. However, as for R, a group having 2 or more constituent carbon atoms is preferred with an optionally substituted cyclic group being more preferred. In particular, an optionally substituted aromatic group (more preferably, a nitrogen-containing heterocyclic ring) is preferred.

R^{1′} represents, among an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group, a group represented by the formula: —X′—W′ (wherein X′ represents a bond, an optionally substituted carbon atom, an optionally substituted nitrogen atom, oxygen atom, or an optionally oxidized sulfur atom; and W′ represents an optionally substituted cyclic group, or a carbon or nitrogen atom having 2 or more substituent).

An optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group is the same group as an optionally substituted heterocyclic group, an optionally substituted sulfinyl group, an optionally substituted sulfonyl group, an optionally substituted hydroxyl group, an optionally substituted thiol group, or an optionally substituted amino group of R¹.

In a group represented by the formula: —X′—W′ (wherein X′ represents a bond, an optionally substituted carbon atom, an optionally substituted nitrogen atom, oxygen atom, or an optionally oxidized sulfur atom; and W′ represents an optionally substituted cyclic group, or a carbon or nitrogen atom having 2 or more substituents) of R^{1′}, “an optionally substituted carbon atom” represented by X′ is a bivalent group having two hydrogen atoms, one hydrogen atom and one substituent, or two substituents on the carbon atom, and “an optionally substituted nitrogen atom” represented by X′ is a bivalent group having one hydrogen atom and one substituent on the nitrogen atom. Examples of said substituent include the same substituent as that of “the hydrocarbon group” or “the heterocyclic group” in “an optionally substituted hydrocarbon group or an optionally substituted heterocyclic group” of the above R¹ as well as the same group as “an optionally substituted hydrocarbon group or an optionally substituted heterocyclic group” as R¹. “An an optionally oxidized sulfur atom” representd by X′ represents a bivalent sulfur atom represented by —S—, —SO—, or —SO₂—.

Examples of “a cyclic group” in “an optionally substituted cyclic group, or a carbon or nitrogen atom having 2 or more substituents” represented as W′ include a cyclic group in “an optionally substituted hydrocarbon group or an optionally substituted heterocyclic group” of the above R¹, for example, an optionally substituted alicyclic hydrocarbon group, an optionally substituted aromatic hydrocarbon group, and an optionally substituted heterocyclic group (an aromatic, monocyclic heterocyclic group, an aromatic fused heterocyclic group, and a non-aromatic heterocyclic group). Examples of the optional substituent of said “cyclic group” include the same substituent as that of “the hydrocarbon group” or “the heterocyclic group” in “an optionally substituted hydrocarbon group or an optionally substituted heterocyclic group” of R¹.

Examples of “a carbon atom having 2 or more substituents” in “an optionally substituted cyclic group or a carbon or nitrogen atom having 2 or more substituents” represented by W′ include a group where the same or different 2-3 substituents are attached to the carbon atom, such as t-butyl or i-propyl (in other words, a group where said atom has 0-1 hydrogen atom). Examples of the substituents include the same substituent as that of “the hydrocarbon group” or “the heterocyclic group” in “an optionally substituted hydrocarbon group or an optionally substituted heterocyclic group” of the above R¹ and the same group as “an optionally substituted hydrocarbon group or an optionally substituted heterocyclic group” of R¹.

As the above-mentioned nitrogen atom having 2 or more substituents, there are an N,N-disubstituted amino group. Examples of the substituent of the “N,N-disubstituted amino group” include the same substituent as that of “the amino group” in “an optionally substituted amino group” as the above-mentioned R¹.

As for R^{1′}, among an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group, a group represented by the formula: —X′—W′ (wherein X′ represents oxygen atom or an optionally oxidized a sulfur atom and W′ represents an optionally substituted cyclic group) is preferred.

R² is cyano group, formyl group, thioformyl group, or a group of the formula: -Z¹-Z² (wherein, Z¹ represents —CO—, —CS—, —SO—, or —SO₂—, and Z² represents an optionally substituted hydrocarbon group, an optionally substituted heterocyclic group, an optionally substituted amino group, or an optionally substituted hydroxyl group).

As for Z¹ in R², —CO— or —SO— is preferred with —CO— being more preferred.

As for R², a group represented by —CO-Z^{2′} (wherein Z² represents an optionally substituted hydrocarbon group, an optionally substituted heterocyclic group, an optionally substituted amino group, or an optionally substituted hydroxyl group).

Examples of an optionally substituted hydrocarbon group of Z² include the same group as an optionally substituted hydrocarbon group of R¹. Preferably, an optionally substituted hydrocarbon group of Z² is an aliphatic hydrocarbon group, more preferably, a saturated aliphatic hydrocarbon group having 1-8 carbon atoms (e.g., alkyl) such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, or the like.

Examples of an optionally substituted heterocyclic group of Z² include the same group as an optionally substituted heterocyclic group of R¹.

Examples of an optionally substituted amino group of Z² include the same group as an optionally substituted amino group of R¹ as well as a group represented by —NHOR, —NHNHR, or —NHNRR′ (wherein R is as defined above and R¹ represents an optionally substituted hydrocarbon group or an optionally substituted heterocyclic group). Examples of “an optionally substituted hydrocarbon group or an optionally substituted heterocyclic group” represented by R′ include the same group as that represented by R. Preferred examples of an optionally substituted amino group of Z² include amino group and an amino group having one or two C₁₋₈ alkyl groups as the substituent(s) (e.g., methylamino, dimethylamino, ethylamino, diethylamino, dibutylamino, etc.).

Examples of an optionally substituted hydroxyl group of Z² include the same group similar as an optionally substituted hydroxyl group of R¹. Preferred examples of an optionally substituted hydroxyl group of Z² include hydroxyl group and a C₁₋₈ alkyloxy group substituted with a C₁₋₈ alkyl group such as methyl, ethyl, propyl, isopropyl, butyl, isobutyl, sec-butyl, tert-butyl, pentyl, isopentyl, neopentyl, tert-pentyl, hexyl, isohexyl, heptyl, or octyl.

As for Z² of R², an optionally substituted amino group or an optionally substituted hydroxyl group is preferred. More preferably, Z² of R² is an amino group that may be substituted. In particular, Z in R² is amino group or an amino group having one or two C₁₋₈ alkyl groups as the substituent(s) (e.g., methylamino, dimethylamino, ethylamino, diethylamino, dibutylamino, etc.).

R^{3′} represents hydrogen atom or an optionally substituted hydrocarbon group, an optionally substituted heterocyclic group, an optionally substituted sulfonyl group, or an optionally substituted acyl group.

Examples of an optionally substituted hydrocarbon group, an optionally substituted heterocyclic group, an optionally substituted sulfonyl group or an optionally substituted acyl group of R^{3′} include the same group as an optionally substituted hydrocarbon group, an optionally substituted heterocyclic group, an optionally substituted sulfonyl group or an optionally substituted acyl group of R³.

R^{3″} represents an optionally substituted hydrocarbon group, an optionally substituted heterocyclic group, an optionally substituted sulfonyl group or an optionally substituted acyl group.

Examples of an optionally substituted hydrocarbon group, an optionally substituted heterocyclic group, an optionally substituted sulfonyl group or an optionally substituted acyl group of R^{3″} include the same group as an optionally substituted hydrocarbon group, an optionally substituted heterocyclic group, an optionally substituted sulfonyl group or an optionally substituted acyl group of R³.

R⁴ represents a substituted hydroxyl group.

Examples of a substituted hydroxyl group of R⁴ include a hydroxyl group of an optionally substituted hydroxyl group of R¹, for example, a hydroxyl group where this hydroxyl group is substituted with an appropriate group such as “an optionally substituted hydrocarbon group or an optionally substituted heterocyclic group” represented by R¹.

Preferred examples thereof include C₁₋₈ alkyloxy substituted with C₁₋₈ alkyl such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, etc.; C₆₋₁₀ aryloxy substituted with C₆₋₁₀ aryl such as phenyl, α-naphthyl, β-naphthyl, 4-indenyl, 5-indenyl, 4-indanyl, 5-indanyl, 5,6,7,8-tetrahydro-1-naphthyl, 5,6,7,8-tetrahydro-2-naphthyl, 5,6-dihydro-1-naphthyl, 5,6-dihydro-2-naphthyl, 5,6-dihydro-3-naphthyl, 5,6-dihydro-4-naphthyl, etc.; hydroxyl group substituted with an aromatic, monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl, etc.); and hydroxyl group substituted with an aromatic fused heterocyclic group (e.g., benzofuranyl, isobenzofuranyl, benzo[b]thienyl, indolyl, isoindolyl, 1H-indazolyl, benzimidazolyl, benzoxazolyl, 1,2-benzisothiazolyl, 1H-benztriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxatinyl, thianthrenyl, phenanthredinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.).

More preferred examples include C₁₋₈ alkyloxy substituted with C₁₋₈ alkyl such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, eetc.; in particular, C₁₋₃ alkyloxy substituted with methyl, ethyl, propyl or isopropyl.

The “hydrocarbon group” or the “heterocyclic group” as the substituents of the substituted hydroxyl group may have the same substituent as that of the “hydrocarbon group” or the “heterocyclic group” of “an optionally substituted hydrocarbon group or an optionally substituted heterocyclic group” of the above-mentioned R¹.

R⁵ represents an optionally substituted sulfinyl group or an optionally substituted sulfonyl group of R¹.

R⁶ is the same as an optionally substituted thiol group of R¹.

R⁷ is the same as an optionally substituted amino group of R¹.

R⁸ is the same as R².

R⁹ is the same as the above-mentioned Z².

R¹⁰ represents a protective group for carboxyl group. Examples of a protective group for carboxyl group represented by R¹⁰ include the same group as an optionally substituted hydrocarbon group of R¹, or the like.

R¹¹ represents an optionally substituted amino group.

Examples of an optionally substituted amino group represented by R¹¹ include the same group as an optionally substituted amino group represented by R¹ such as amino group, an N-mono-substituted amino group, or an N,N-disubstituted amino group. Examples of said substituted amino group include amino group having one or two substituents of an optionally substituted hydrocarbon group (e.g., the same group as an optionally substituted hydrocarbon group represented by R¹, more specifically, a C₁₋₈ alkyl group, a C₃₋₁₇ cycloalkyl group, a C₂₋₈ alkenyl group, a C₂₋₈ alkynyl group, a C₃₋₇ cycloalkenyl group, a C₆₋₁₄ aryl group that may have a C₁₋₄ alkyl group); an optionally substituted heterocyclic group (e.g., the same group as an optionally substituted heterocyclic group represented by R¹) or a group of the formula: —COR′ (wherein R′ represents hydrogen atom, an optionally substituted hydrocarbon group or an optionally substituted heterocyclic group whose “hydrocarbon group” or “heterocyclic group” as R′ may have the same substituent as that of the “hydrocarbon group” or the “heterocyclic group” of “an optionally substituted hydrocarbon group or an optionally substituted heterocyclic group” of the above-mentioned R¹); or, preferably a C₁₋₁₀ acyl group (e.g., a C₂₋₇ alkanoyl, benzoyl, nicotinoyl, etc.) or the like), for example, methylamino, dimethylamino, ethylamino, diethylamino, propylamino, dipropylamino, dibutylamino, diallylamino, cyclohexylamino, phenylamino, N-methyl-N-phenylamino, acetylamino, propionylamino, benzoylamino, nicotinoylamino, and the like. More preferred examples thereof include a N,N-disubstituted amino group (e.g., dimethylamino, diethylamino, dipropylamino, dibutylamino, diallylamino, N-methyl-N-phenylamino, etc.), in particular, a N,N-di-C₁₋₃-alkylamino group (e.g., dimethylamino, diethylamino, dipropylamino, etc.).

In addition, two groups of said substituted amino group may be combined to form a nitrogen-containing 5- to 7-membered ring (e.g., piperidino, morpholino, thiomorpholino, etc.).

R¹³ represents an optionally substituted hydrocarbon group, an optionally substituted amino group or an optionally substituted hydroxyl group. Examples of R¹³ include the same group as the above-mentioned R⁴ or R¹¹.

R^{13′} represents an optionally substituted hydrocarbon group, an optionally substituted amino group or an optionally substituted hydroxyl group. Examples of R^{13′} include hydroxyl group or the same group as the above-mentioned R⁴ or R¹¹.

X represents a halogen atom such as fluorine, chlorine, bromine or iodine.

Z⁵ represents —CO—.

Z⁶ is the same as an optionally substituted amino group of Z².

Z⁷ represents —CO—.

Z⁸ is the same as an optionally substituted hydrocarbon group or an optionally substituted heterocyclic group of Z².

Ring A represents an aromatic 5-membered heterocyclic ring represented by

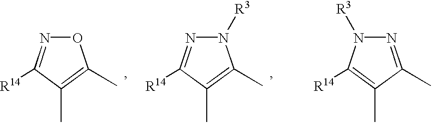

wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; and R¹⁴ represents hydrogen atom, a halogen, an optionally substituted hydrocarbon group, an optionally substituted heterocyclic group, an optionally substituted hydroxyl group, an optionally substituted amino group, an optionally substituted sulfonyl group, or an optionally substituted acyl group. Preferably, ring A is an aromatic 5-membered heterocyclic ring represented by

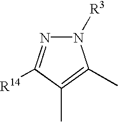

wherein R³ and R¹⁴ are as defined above).

R³ represents hydrogen atom or an optionally substituted hydrocarbon group, an optionally substituted heterocyclic group, an optionally substituted hydroxyl group, an optionally substituted amino group, an optionally substituted sulfonyl group, or an optionally substituted acyl group.

Examples of an optionally substituted hydrocarbon group of R³ include the same group similar as an optionally substituted hydrocarbon group of R¹. Preferred examples of an optionally substituted hydrocarbon group of R³ include an aliphatic hydrocarbon group, more preferably, a saturated aliphatic hydrocarbon group having 1-8 carbon atoms (e.g., an alkyl group) such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, or the like.

Examples of an optionally substituted heterocyclic group of R³ include the same group as an optionally substituted heterocyclic group of R¹.

Examples of an optionally substituted hydroxyl group of R³ include the same group as an optionally substituted hydroxyl group of R¹.

Examples of an optionally substituted amino group of R³ include the same group as an optionally substituted amino group of R¹.

Examples of an optionally substituted sulfonyl group of R³ include the same group as an optionally substituted sulfonyl group of R¹.

Examples of an optionally substituted acyl group of R³ include the same group as that where carbonyl group is combined with “an optionally substituted hydrocarbon group or an optionally substituted heterocyclic group” of R¹ or the like. Preferred examples of the optionally substituted acyl group include the same acyl group as that of the substituent of the hydrocarbon group, the heterocyclic group, sulfinyl group or sulfonyl group represented by R¹.

R¹⁴ is hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group. Examples of a halogen atom of R¹⁴ include fluorine, chlorine, bromine or iodine. Examples of an optionally substituted hydrocarbon group, an optionally substituted heterocyclic group, an optionally substituted hydroxyl group, an optionally substituted amino group, an optionally substituted sulfonyl group or an optionally substituted acyl group of R¹⁴ include the same group as that represented by R³. Hydrogen atom is preferred as R¹⁴.

Ring B, B-1, C, D, D-2, D-3, D-4, D′, D′-1, D′-2, or D′-3 represents an optionally substituted 5- to 7-membered hydrocarbon ring.

The 5- to 7-membered hydrocarbon ring of an optionally substituted 5- to 7-membered hydrocarbon group may be either an aliphatic or an aromatic 5- to 7-membered hydrocarbon ring.

Examples of said alicyclic 5- to 7-membered, hydrocarbon ring include a C₅₋₇ saturated alicyclic hydrocarbon ring (e.g., C₅₋₇ cycloalkane such as cyclopentane, cyclohexane, cycloheptane, etc.); C₅₋₇ unsaturated alicyclic hydrocarbon ring (e.g., C₅₋₇ cycloalkene and C₅₋₇ cycloalkadiene such as 1-cyclopentene, 2-cyclopentene, 3-cyclopentene, 1-cyclohexene, 2-cyclohexene, 3-cyclohexene, 1-cycloheptene, 2-cycloheptene, 3-cycloheptene, 2,4-cycloheptadiene, etc.); and the like.

As said aromatic hydrocarbon group, for example, there is benzene ring.

Preferred examples include a C₅₋₇ saturated alicyclic hydrocarbon ring, with a C₆ saturated alicyclic hydrocarbon ring (cyclohexane) being more preferred.

Examples of an optionally substituted 5- to 7-membered hydrocarbon ring of ring B, B-1, C, D, D-2, D-3, D-4, D′, D′-1, D′-2, or D′-3 include the same group as the substituent of “an optionally substituted hydrocarbon group” of R¹ and the like. Preferably, the ring is substituted with 1 to 3 substituents. Preferred examples of a substituent of said optionally substituted alicyclic 5- to 7-membered hydrocarbon ring include an aliphatic hydrocarbon group, more preferably, a saturated, aliphatic hydrocarbon group having 1-8 carbon atoms (e.g., an alkyl group) such as methyl, ethyl, propyl, isopropyl, butyl, isobutyl, sec-butyl, tert-butyl, pentyl, isopentyl, neopentyl, tert-pentyl, hexyl, isohexyl, heptyl, octyl, or the like.

Ring B, B-1, C, D, D-2, D-3, D-4, D′, D′-1, D′-2, or D′-3 represents an optionally substituted 5- to 7-membered hydrocarbon ring. Preferred examples thereof include an unsubstituted 5- to 7-membered hydrocarbon ring, more preferably, an unsubstituted 5- to 7-membered saturated hydrocarbon ring. In particular, an substituted 6-membered saturated hydrocarbon ring is preferred.

In the above-mentioned formula (I), a compound wherein R¹ is an optionally substituted sulfinyl group, an optionally substituted sulfonyl group, an optionally substituted hydroxyl group or an optionally substituted thiol group; R² is -Z¹-Z² (wherein Z¹ represents —CO— or —CS—; Z² represents an optionally substituted hydroxyl group, or an optionally substituted amino group); ring A is

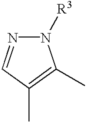

wherein R³ is as defined above; and ring B is an optionally substituted 5- to 7-membered hydrocarbon ring, or a salt thereof is preferred.

In the above-mentioned formula (I), a compound wherein R¹ is sulfinyl group or sulfonyl group attached through a C₁₋₈ alkyl, thiol group optionally substituted with a C₁₋₈ alkyl, or hydroxyl group optionally substituted with C₆₋₁₀ aryl (in particular, phenyl), an aromatic monocyclic heterocyclic group (in particular, pyridyl), or an aromatic fused heterocyclic group (in particular), each of which may have 1-3 substituents; R² is -Z¹-Z² (wherein Z¹ represents —CO— and Z² represents an optionally substituted hydroxyl group or an optionally substituted amino group); R³ of ring A

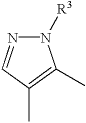

wherein R³ is as defined above, is a saturated aliphatic hydrocarbon group (e.g., an alkyl group); and ring B is a C₅₋₇ saturated aliphatic hydrocarbon group, or a salt thereof is more preferred.

Preferred examples of a compound represented by formula (I) are:

4,5-dihydro-1-methyl-8-propylsulfanyl-1H-thieno[3,4-g]indazole-6-carboxamide;

4,5-dihydro-1-methyl-8-propylsulfinyl-1H-thieno[3,4-g]indazole-6-carboxamide;

4,5-dihydro-1-methyl-8-propylsulfonyl-1H-thieno[3,4-g]indazole-6-carboxamide;

4,5-dihydro-8-(3,4-methylenedioxyphenoxy)-1-methyl-1H-thieno[3,4-g]indazole-6-carboxamide;

4,5-dihydro-8-phenoxy-1-methyl-1H-thieno[3,4-g]indazole-6-carboxamide;

4,5-dihydro-8-(3,4-methylenedioxyphenoxy)thieno[3,4-g]-1,2-benzisoxazole-6-carboxamide;

8-[4-[(diethoxyphosphoryl)methyl]phenoxy]-4,5-dihydro-2-methyl-2H-thieno[3,4-g]indazole-6-carboxamide;

8-[4-[(diethoxyphosphoryl)methyl]phenoxy]-4,5-dihydro-1-methyl-1H-thieno[3,4-g]indazole-6-carboxamide;

N-ethyl-4,5-dihydro-8-(3,4-methylenedioxyphenoxy)-1-methyl-1H-thieno[3,4-g]indazole-6-carboxamide;

4,5-dihydro-1-methyl-8-(4-trifluoromethyl-phenoxy)-1H-thieno[3,4-g]indazole-6-carboxamide;

4,5-dihydro-1-methyl-8-(6-quinolinyloxy)-1H-thieno[3,4-g]indazole-6-carboxamide;

4,5-dihydro-1-methyl-8-(3-pyridinyloxy)-1H-thieno[3,4-g]indazole-6-carboxamide;

8-[4-(benzyloxy)phenoxy]-4,5-dihydro-1-methyl-1H-thieno[3,4-g]indazole-6-carboxamide; and

4,5-dihydro-1-methyl-8-[4-(2-quinolinylmethoxy)phenoxy]-1H-thieno[3,4-g]indazole-6-carboxamide.

In the above-mentioned formula (II), a compound wherein R¹ is an optionally substituted sulfinyl group, an optionally substituted sulfonyl group, an optionally substituted hydroxyl group or an optionally substituted thiol group; R² is -Z¹-Z² (wherein Z¹ represents —CO— or —CS— and Z² represents an optionally substituted hydroxyl group or an optionally substituted amino group); R⁴ is C₁₋₈ alkoxy; and ring C is an optionally substituted 5- to 7-membered hydrocarbon ring, or a salt thereof is preferred.

In the above-mentioned formula (II), a compound wherein R¹ is sulfinyl group or sulfonyl group attached through C₁₋₈ alkyl, thiol optionally substituted with a C₁₋₈ alkyl, or hydroxyl group optionally substituted with C₆₋₁₀ aryl (in particular, phenyl), an aromatic monocyclic heterocyclic group (in particular, pyridyl), or an aromatic fused heterocyclic group (in particular, quinolyl), each of which may have 1-3 substituents; R² is -Z¹-Z² (wherein Z¹ represents —CO— and Z² represents an optionally substituted hydroxyl group or an optionally substituted amino group); R⁴ is C₁₋₃ alkoxy; and ring C is a C₅₋₇ saturated alicyclic hydrocarbon ring, or a salt thereof is more preferred.

As for a salt of a starting compound for producing the compound represented by formula (I) of the present invention (referred to as compound (I)) or a salt thereof, a pharmaceutically acceptable salt is preferred and examples thereof include a salt with an inorganic base, a salt with an organic base, a salt with an inorganic acid, a salt with an organic acid, a salt with a basic or acidic amino acid, or the like. Preferred examples of a salt with an inorganic base include an alkali metal salt such as sodium salt, potassium salt, or the like; an alkaline earth metal salt such as calcium salt, magnesium salt, or the like; and aluminum salt; ammonium salt; or the like. Preferred examples of a salt with an organic base include a salt with trimethylamine, triethylamine, pyridine, picoline, ethanolamine, diethanolamine, triethanolamine, dicyclohexylamine, N,N′-dibenzylethylenediamine, or the like. Preferred examples of a salt with an inorganic acid include a salt with hydrochloric acid, hydrobromic acid, nitric acid, sulfuric acid, phosphoric acid, or the like. Preferred examples of a salt with an organic acid include a salt with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, or the like. Preferred examples of a salt with a basic amino acid include a salt with arginine, lysine, ornithine or the like. Preferred examples of a salt with an acidic amino acid include a salt with aspartic acid, glutamic acid, or the like.

Compound (I) or its salt may be in the form of a prodrug thereof. The prodrug of compound (I) or its salt refers to a compound that is converted into compound (I) or its salt by a reaction with an enzyme, gastric acid, or the like under a physiological condition in the living body, namely, [1] a compound that is converted into compound (I) or its salt by an enzymatic oxidation, reduction, hydrolysis, or the like and [2] a compound that is converted into compound (I) or its salt by hydrolysis with gastric acid or the like. Examples of a prodrug of compound (I) or its salt to be used include a compound or its salt wherein hydroxyl group in compound (I) or its salt is acylated, alkylated, phosphorylated, or converted into borate (e.g., a compound or its salt wherein hydroxyl group in compound (I) or its salt is converted into acetyloxy, palmitoyloxy, propanoyloxy, pivaloyloxy, succinyloxy, fumaryloxy, alanyloxy, dimethylaminomethylcarbonyloxy, etc.), a compound or its salt wherein carboxyl group in compound (I) or its salt is esterified or amidated (e.g., a compound or its salt wherein carboxyl group in compound (I) or its salt is subjected to ethyl esterification, phenyl esterification, carboxyoxymethyl esterification, dimethylaminomethyl esterification, pivaloyloxymethyl esterification, ethoxycarbonyloxyethyl esterification, phthalidyl esterification, (5-methyl-2-oxo-1,3-dioxolan-4-yl)methyl esterification, cyclohexyloxycarbonyl esterification, or conversion into the methyl amide, etc.), or the like. These prodrugs can be produced according to a per se known method or its modified method.

Further, a prodrug of compound (I) or its salt may be a compound or its salt that is converted into compound (I) or its salt under physiological conditions as described in “Development of Drugs”, Volume 7, Molecular Design, Hirokawa Shoten, 1990; pages 163-198.

Compound (I) or its salt may be labeled with an isotope (for example, ²H, ³H, ¹⁴C, ³⁵S, ¹²⁵I, or the like) or the like.

In the above-mentioned formula (IX), a compound wherein R¹ is an optionally substituted sulfinyl group, an optionally substituted sulfonyl group, an optionally substituted hydroxyl group, or an optionally substituted thiol group; R² is -Z¹-Z² (wherein Z¹ represents —CO— or —CS— and Z² represents an optionally substituted hydroxyl group or an optionally substituted amino group); R¹³ is C₁₋₈ alkoxy or an N,N-di-substituted amino group; and ring D is an optionally substituted 5- to 7-membered hydrocarbon ring, or a salt thereof is preferred.

In the above-mentioned formula (IX), a compound wherein R¹ is sulfinyl group or sulfonyl group attached through a C₁₋₈ alkyl, thiol group optionally substituted with C₁₋₈ alkyl, or hydroxyl group optionally substituted with C₆₋₁₀ aryl (in particular, phenyl), an aromatic monocyclic heterocyclic group (in particular, pyridyl), or an aromatic fused heterocyclic group (in particular, quinolyl) each or which may have 1-3 substituents; R² is -Z¹-Z² (wherein, Z¹ represents —CO— and Z² represents an optionally substituted hydroxyl group or an optionally substituted amino group); R¹³ is an N,N-di-C₁₋₃-alkylamino group or C₁₋₃ alkoxy; and ring D is a C₅₋₇ saturated alicyclic hydrocarbon ring, or a salt thereof is more preferred.

Preferred examples of a compound represented by formula (IX) is:

3-(3,4-dimethoxyphenoxy)-5-ethoxymethylidene-4,5,6,7-tetrahydro-4-oxobenzo[c]thiophene-6-carboxylic acid ethyl ester;

3-(3,4-dimethoxyphenoxy)-5-dimethylamino-methylidene-4,5,6,7-tetrahydro-4-oxobenzo[c]thiophene-6-carboxylic acid ethyl ester; or

5-dimethylaminomethylidene-4,5,6,7-tetrahydro-3-methylthio-4-oxobenzo[c]thiophene-6-carboxylic acid ethyl ester.

As for a salt of a starting compound for producing the compound or a salt thereof described herein whose representative example is a compound represented by formula (IX) [referred to as compound (IX)] or its salt, a pharmaceutically acceptable salt is preferred. Examples thereof include a salt with an inorganic base, a salt with an organic base, a salt with an inorganic acid, a salt with an organic acid, a salt with a basic or acidic amino acid, or the like. Preferred examples of a salt with an inorganic base include an alkali metal salt such as sodium salt, potassium salt, and the like; an alkaline earth metal salt such as calcium salt, magnesium salt, or the like; and aluminum salt, ammonium salt, or the like. Preferred examples of a salt with an organic base include a salt with trimethylamine, triethylamine, pyridine, picoline, ethanolamine, diethanolamine, triethanolamine, dicyclohexylamine, N,N′-dibenzylethylenediamine, or the like. Preferred examples of a salt with an inorganic acid include a salt with hydrochloric acid, hydrobromic acid, nitric acid, sulfuric acid, phosphoric acid, or the like. Preferable examples of a salt with an organic acid include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, or the like. Preferable examples of a salt with a basic amino acid include a salt with arginine, lysine, ornithine or the like. Preferred examples of a salt with an acidic amino acid include a salt with aspartic acid, glutamic acid, or the like.

Compound (IX) or its salt may be labeled with an isotope (for example, ²H, ³H, ¹⁴C, ³⁵S, ¹²⁵I, or the like) or the like.

When the compound obtained by the present invention or a salt thereof has a double bond in its molecule and a steric configuration of Z or E exsits, each of the stereoisomers and a mixture thereof are included in the present invention.

When a steric configuration exsits due to an asymmetric carbon in the molecule of the compound obtained by the present invention or a salt thereof, each of them and a mixture thereof are included in the present invention.

Hereinafter, production of the compound of the present invention will be illustrated.

Compound (I) or a salt thereof can be produced, for example, by the following Process A to Process F or its modification.

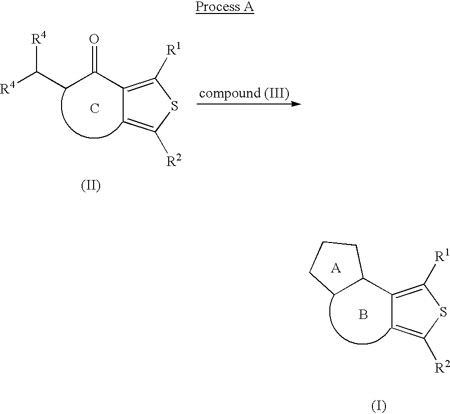

wherein compound (III) represents hydroxylamine or mono-substituted hydrazine (R^{3′}NHNH₂) or a salt thereof and the other symbols are as defined above.

In this reaction, compound (I) is produced by the reaction of compound (II) with compound (III).

This reaction is carried out under neutral conditions or in the presence of an acid or a base in a solvent that does not adversely affect the reaction according to a conventional method.

Examples of an acid include a mineral acid such as hydrochloric acid, sulfuric acid, etc.; and an organic acid such as methanesulfonic acid, p-toluenesulfonic acid, benzoic acid, acetic acid, trifluoroacetic acid, etc. Examples of a base include an inorganic base such as sodium hydride, sodium hydroxide, potassium hydride, etc.; and an organic base such as potassium t-butoxide, sodium acetate, triethylamine, pyridine, 1,8-diazabicyclo[5.4.0]-7-undecene, sodium methoxide, etc.

The amounts of the acid and compound (III) to be used are preferably about 1- to about 5-molar equivalents for compound (II).

Examples of the solvent that does not adversely affect the reaction include water, alcohol such as methanol, ethanol, propanol, etc.; ether such as ethyl ether, tetrahydrofuran, dioxane, etc.; halogenated hydrocarbon such as chloroform, dichloromethane, etc.; aromatic hydrocarbon such as benzene, toluene, xylene, etc.; amide such as N,N-dimethylformamide, 1-methylpyrrolidone, etc.; sulfoxide such as dimethyl sulfoxide, etc., or the like. These solvents may be used as a mixture thereof in an appropriate ratio.

The reaction temperature is usually about −50 to about 150° C., preferably about 0 to about 100° C.

The reaction time is usually about 0.5 to about 20 hours.

R^{3′} on ring A produced by this reaction can be converted into an optionally substituted hydroxyl group or an optionally substituted amino group described as R³ by using a per se known method.

The thus-obtained compound (I) can be isolated and purified according to a known separation and purification means such as concentration, concentration under reduced pressure, solvent extraction, crystallization, recrystallization, trans-solubilization, chromatography, or the like.

Compound (II) to be employed as the starting compound in the above-mentioned Process A is a novel compound and is produced by reacting compound (IV):

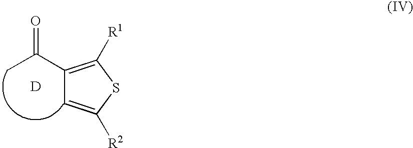

wherein each symbol is as defined above, with an orthoformic acid ester. That is, compound (IV) is subjected to a known process such as the process described in Indian J. Chem. Vol. Sec. B, 35, pages 49-51 (1996) or a modified process thereof. That is, normally, this reaction is carried out in the presence of an acid and a base in a solvent that does not adversely affect the reaction.

The amount of orthoformic acid ester is preferably about 1- to about 10-molar equivalents for compound (IV).

As the acid, for example, there is boron trifluoride-ether complex or the like.

The amount of the acid to be used is preferably about 1- to about 10-molar equivalents for compound (IV).

Examples of the base include triethylamine, diisobutylethylamine, 1,8-diazabicyclo[5.4.0]-7-undecene, or the like.

The amount of the base to be used is preferably about 1- to about 10-molar equivalents for compound (IV).

Examples of the solvent that does not adversely affect the reaction include halogenated hydrocarbon such as chloroform, dichloromethane, etc.; aromatic hydrocarbon such as benzene, toluene, xylene, etc.; and the like. These solvents may be used as a mixture thereof in an appropriate ratio.

The reaction temperature is usually about −100 to about 150° C., preferably about −70 to about 0° C.

The reaction time is usually about 0.5 to about 20 hours.

The thus-obtained compound (II) can be isolated and purified according to a known separation and purification means such as concentration, concentration under reduced pressure, solvent extraction, crystallization, recrystallization, trans-solubilization, chromatography, or the like.

Among compound (IV) to be used as the starting compound, compound (IV-1):

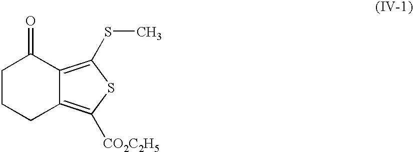

is a known compound and has been described in Liebigs Ann., 1996, pages 239-245 or Synth. Commun., 1995, pages 2449-2455.

Further, compound (IV-2, 3) to be used as the starting compound for producing compound (I) wherein R¹ is an optionally substituted amino group in the above-mentioned Process A can be produced by the following synthetic process.

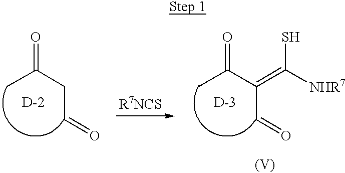

wherein each symbol is as defined above.

In this reaction, compound (V) is produced by reacting a 1,3-cycloalkanedione with an isothiocyanic acid alkyl ester or an isothiocyanic acid aryl ester in the presence of a base.

Examples of the base include alkali metal salt such as sodium hydroxide, sodium hydrogen carbonate, potassium carbonate, etc; amine such as pyridine, triethylamine, N,N-dimethylaniline, 1,8-diazabicyclo[5.4.0]undec-7-ene, etc.; metal hydride such as potassium hydride, sodium hydride, etc.; and alkali metal alkoxide such as sodium methoxide, sodium ethoxide, potassium t-butoxide, etc.

The amounts of the reagents to be used are preferably about 1- to about 10-molar equivalents for the 1,3-cycloalkanedione.

The amount of the base to be used is preferably about 1- to about 10-molar equivalents for the 1,3-cycloalkanedione.

The reaction temperature is usually about −50 to about 150° C., preferably about 0 to about 100° C.

The reaction time is usually about 0.5 to about 20 hours.

The thus-obtained compound (V) can be isolated and purified according to a known separation and purification means such as concentration, concentration under reduced pressure, solvent extraction, crystallization, recrystallization, trans-solubilization, chromatography, and the like.

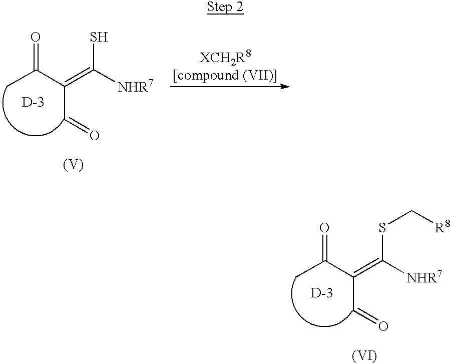

wherein each symbol is as defined above.

In this present reaction, compound (VI) is produced by the reaction of compound (V) with compound (VII). This reaction is carried out in the presence of a base in a solvent that does not adversely affect the reaction according to a conventional method.

Examples of compound (VII) include a haloacetic acid ester, a halomethyl nitrile, or halomethyl ketone, and specifically by ethyl chloroacetate, ethyl bromoacetate, t-butyl bromoacetate, chloroacetone, chloroacetylbenzene, chloroacetonitrile, etc.

The amount of compound (VII) to be used is preferably about 1- to about 10-molar equivalents for compound (V).

Examples of the base include alkali metal salt such as potassium hydroxide, sodium hydroxide, sodium hydrogen carbonate, potassium carbonate, etc.; amine such as pyridine, triethylamine, N,N-dimethylaniline, 1,8-diazabicyclo[5.4.0]undec-7-ene, etc.; metal hydride such as potassium hydride, sodium hydride, etc.; and alkali metal alkoxide such as sodium methoxide, sodium ethoxide, potassium t-butoxide, etc.

The amount of the base to be used is preferably about 1- to about 5-molar equivalents for compound (V).

Examples of the solvent that does not adversely affect the reaction include aromatic hydrocarbon such as benzene, toluene, xylene, etc.; ether such as tetrahydrofuran, dioxane, diethyl ether, etc.; halogenated hydrocarbon such as chloroform, dichloromethane, etc.; amide such as N,N-dimethylformamide, etc.; sulfoxide such as dimethyl sulfoxide, etc.; and the like. These solvents may be used as a mixture thereof in an appropriate ratio.

The reaction temperature is usually about −50 to about 150° C., preferably about −10 to about 100° C.

The reaction time is usually about 0.5 to about 20 hours.

The thus-obtained compound (VI) can be isolated and purified according to a known separation and purification means such as concentration, concentration under reduced pressure, solvent extraction, crystallization, recrystallization, trans-solubilization, chromatography, or the like.

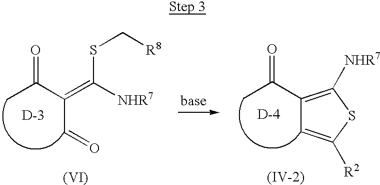

wherein each symbol is as defined above.

In this reaction, compound (VI-2) is produced from compound (VI). This reaction is carried out in the presence of a base in a solvent that does not adversely affect the reaction according to a conventional method.

Examples of the base include alkali metal salt such as potassium hydroxide, sodium hydroxide, sodium hydrogen carbonate, potassium carbonate, etc.; amine such as pyridine, triethylamine, N,N-dimethylaniline, 1,8-diazabicyclo[5.4.0]undec-7-ene, etc.; metal hydride such as potassium hydride, sodium hydride, etc.; and alkali metal alkoxide such as sodium methoxide, sodium ethoxide, potassium t-butoxide, etc.

The amount of the base to be used is preferably about 1- to about 5-molar equivalents for compound (VI).

Examples of the solvent that does not adversely affect the reaction include aromatic hydrocarbon such as benzene, toluene, xylene, etc.; ether such as tetrahydrofuran, dioxane, diethyl ether, etc.; halogenated hydrocarbons such as chloroform, dichloromethane, etc.; amide such as N,N-dimethylformamide, etc.; sulfoxide such as dimethyl sulfoxide, etc.; or the like. These solvents may be used as a mixture thereof in an appropriate ratio.

The reaction temperature is usually about −50 to about 150° C., preferably about −10 to about 100° C.

The reaction time is usually about 0.5 to about 20 hours.

The thus-obtained compound (IV-2) can be isolated or purified purification according to a known separation and purification means such as concentration, concentration under reduced pressure, solvent extraction, crystallization, recrystallization, trans-solubilization, chromatography, or the like.

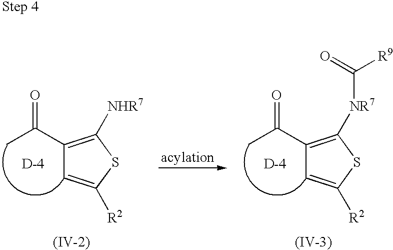

wherein each symbol is as defined above.

In this reaction, compound (VI-3) is produced by acylation of compound (IV-2). This reaction is carried out by employing a process where compound (IV-2) is appropriately reacted with an acylating agent, or the like.

Examples of the acylating agent include an acid anhydride, an acid halide (an acid chloride or an acid bromide), an imidazolide, a mixed acid anhydride (e.g., an anhydride with methyl carbonate, ethyl carbonate, or isobutyl carbonate, etc.). The amount of the acylating agent to be used is preferably about 1- to about 5-molar equivalents for compound (IV-2).

Examples of the solvent that does not adversely affect the reaction include aromatic hydrocarbon such as benzene, toluene, xylene, etc.; ether such as tetrahydrofuran, dioxane, diethyl ether, etc.; halogenated hydrocarbon such as chloroform, dichloromethane, etc.; amide such as N,N-dimethylformamide, etc.; sulfoxide such as dimethyl sulfoxide, etc.; and the like. These solvents may be used as a mixture thereof in an appropriate ratio.

The reaction temperature is usually about −50 to about 150° C., preferably about −10 to about 100° C.

The reaction time is usually about 0.5 to about 20 hours.

The thus-obtained compound (IV-3) can be isolated and purified according to a known separation and purification means such as concentration, concentration under reduced pressure, solvent extraction, crystallization, recrystallization, trans-solubilization, chromatography, or the like.

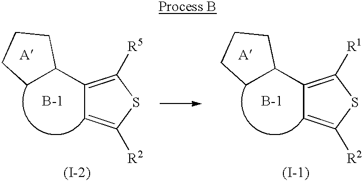

wherein ring A′ represents

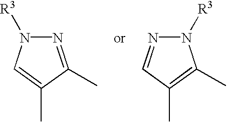

and each symbol is as defined above.

In this process, compound (I-1) is produced by the reaction of compound (I-2) with a nucleophilic reagent.

This reaction is carried out by a per se known process, for example, the process described in WO98/18792 or a modified process thereof.

Examples of the nucleophilic reagent include a metal phenolate, a metal alcoholate, a Grignard reagent, an alkali metal reagent, an aryl metal reagent, a thioalcoholate, an amine, or the like.

The amount of the nucleophilic reagent to be used is preferably about 1- to about 5-molar equivalents for compound (I-2).

Examples of the solvent that does not adversely affect the reaction include ether such as diethyl ether, tetrahydrofuran, dioxane, etc.; halogenated hydrocarbon such as chloroform, dichloromethane, etc.; aromatic hydrocarbon such as benzene, toluene, xylene, etc.; amide such as N,N-dimethylformamide, N-methylpyrrolidine, etc.; sulfoxide such as dimethyl sulfoxide, etc.; or the like. These solvents may be used as a mixture thereof in an appropriate ratio.

The reaction temperature is usually about −50 to about 150° C., preferably about −10 to about 100° C.

The reaction time is usually about 0.5 to about 20 hours.

The thus-obtained compound (I-1) can be isolated and purified according to a known separation and purification means such as concentration, concentration under reduced pressure, solvent extraction, crystallization, recrystallization, trans-solubilization, chromatography, Or or the like.

Compound (I-2) to be employed as the starting compound in the above-mentioned Process B can be produced by the following process.

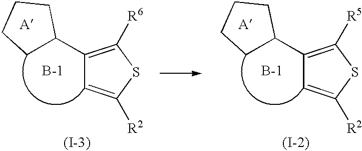

wherein each symbol is as defined above.

In this process, compound (I-2) is produced from compound (I-3) by using an oxidizing agent. This reaction is carried out according to a per se known process such as a process using as the oxidizing agent, manganese dioxide, permanganic acid, chromic acid, lead tetraacetate, halogen, ozone, hydrogen peroxide, an organic peroxide, an organic peracid, hydrogen peroxide-sodium tungstate, oxygen, an N-halocarboxamide, a hypohalogenic acid ester, an iodosyl compound, nitric acid, dinitrogen tetraoxide, dimethyl sulfoxide, ethyl azodicarboxylate, chloroauric acid, etc.; anodic oxidation; or a modified process thereof. That is, this reaction is carried out usually in the presence of an oxidizing agent in a solvent that does not adversely affect the reaction.

Preferred examples of the oxidizing agent include m-chloroperbenzoic acid, peracetic acid, etc.

Examples of the solvent that does not adversely affect the reaction include ether such as diethyl ether, tetrahydrofuran, dioxane, etc.; halogenated hydrocarbon such as chloroform, dichloromethane, etc.; aromatic hydrocarbon such as benzene, toluene, xylene, etc.; amide such as N,N-dimethylformamide, N-methylpyrrolidine etc.; and the like. These solvents may be used as a mixutre thereof in an appropriate ratio.

The reaction temperature is usually about −50 to about 150° C., preferably about −10 to about 100° C.

The reaction time is usually about 0.5 to about 20 hours.

The thus-obtained compound (I-2) can be isolated and purified according to a known separation and purification means such as concentration, concentration under reduced pressure, solvent extraction, crystallization, recrystallization, trans-solubilization, chromatography, or the like.

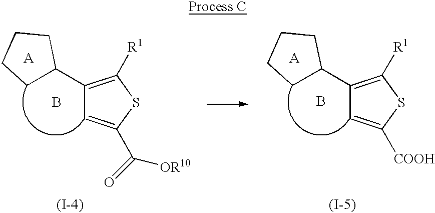

wherein each symbol is as defined above.

In this process, compound (I-5) is produced by removal of a carboxyl protecting group.

In this reaction, there can be used any conventional method employing for removal of a carboxyl protecting group, for examples, hydrolysis, reduction, removal with a Lewis acid, or like. When a carboxyl protecting group is an ester, it can be removed by hydrolysis or with a Lewis acid. In the hydrolysis, a protecting group can be removed by using a base or a Lewis acid. Preferably, the hydrolysis is carried out in the presence of a base or an acid. Preferred examples of the base include an inorganic base such as alkali metal hydroxide (e.g., sodium hydroxide, calcium hydroxide, etc.), alkaline earth metal hydroxide (e.g., magnesium hydroxide, calcium hydroxide, etc.), alkali metal carbonate (e.g., sodium carbonate, potassium carbonate, etc.), alkaline earth metal carbonate (e.g., magnesium carbonate, calcium carbonate, etc.), alkali metal bicarbonate (e.g., sodium bicarbonate, potassium bicarbonate, etc.), alkali metal acetate (e.g., sodium acetate, potassium acetate, etc.), alkaline earth metal phosphate (e.g., magnesium phosphate, calcium phosphate, etc.), alkali metal hydrogen phosphate (e.g., disodium hydrogen phosphate, dipotassium hydrogen phosphate, etc.); and an organic base such as trialkylamine (e.g., trimethylamine, triethylamine, etc.), picoline, N-methylpyrrolidine, N-methylmorpholine, 1,5-diazabicyclo[4.3.2]non-5-ene, 1,4-diazabicyclo[2.2.2]non-5-ene, 1,8-diazabicyclo[5.4.0]-7-undecene, and the like. The hydrolysis using a base is carried out in water or a hydrophilic organic solvent or a mixed solvent in many cases. Preferred examples of an acid include an organic acid (e.g., formic acid, hydrobromic acid, sulfuric acid, etc.).

This hydrolysis is carried out usually in an organic solvent, water, or a mixed solvent thereof. The reaction temperature is not specifically limited and is appropriately selected depending on a particular kind of a carboxyl protecting group and a particular removing method. The removal with a Lewis acid is carried out by reacting compound (I-4) or a salt thereof with a Lewis acid such as boron trihalide (e.g., boron trichloride, boron trifluoride, etc.), titanium tetrahalide (e.g., titanium tetrachloride, titanium tetrabromide, etc.), aluminum halide (e.g., aluminum chloride, aluminum bromide, etc.), trihaloacetic acid (e.g., trichloroacetic acid, trifluoroacetic acid, etc.), or the like. This removing reaction is carried out preferably in the presence of a cation scavenger (e.g., anisole, phenol, etc.) and, also, is carried out usually in a solvent such as nitroalkane (e.g., nitromethane, nitroethane, etc.), alkylene halide (e.g., methylene chloride, ethylene chloride, etc.), diethyl ether, carbon disulfide, or another solvent that does not adversely affect the reaction, or the like. These solvents may be used as a mixture thereof.

The removal by reduction is applied preferably to that of a protecting group such as halogenated alkyl (e.g., 2-iodoethyl, 2,2,2,-trichlorethyl, etc.) ester, aralkyl (e.g., benzyl, etc.) ester, or the like. The reduction method to be employed for this removing reaction is, for example, a combination of metal (e.g., zinc, zinc amalgam, etc.) or a salt of chrome compound (e.g., chromous chloride, chromous acetate, etc.) with an organic or inorganic salt (e.g., acetic acid, propionic acid, hydrochloric acid, etec.); a conventional hydrogenation in the presence of a conventional metal catalyst (e.g., palladium carbon, Raney nickel, etc.), or the like. The reaction temperature is not specifically limited and the reaction is carried out usually under cooling, at room temperature, or with warming.

The thus-obtained compound (I-5) can be isolated and purified according to a known separation and purification means such as concentration, concentration under reduced pressure, solvent extraction, crystallization, recrystallization, trans-solubilization, chromatography, or the like.

Compound (I-6) represented by the general formula (I) wherein R² represents the formula: -Z^{5-Z}⁶ (wherein -Z⁵ represents —CO— and -Z⁶ represents an optionally substituted amino group) is produced by the following Process D.

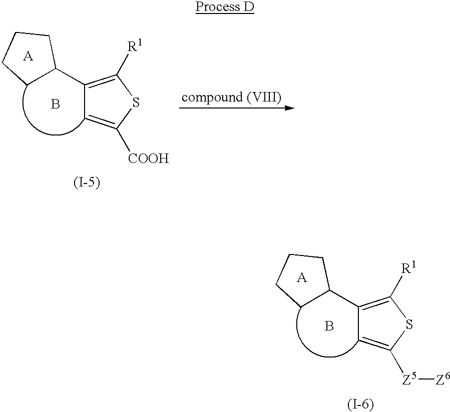

wherein, compound (VIII) represents Z ⁶H and the other symbols are as defined above.

In this process, compound (I-6) is produced by reacting compound (I-5) or its reactive derivative at the carboxyl group or a salt thereof with the above-mentioned compound (VIII) or its reactive derivative at the amino group or a salt thereof. Preferred examples of a reactive derivative at amino group or a salt thereof of compound (III) include a Schiff base-type imino or its enamine-type tautomer formed by the reaction of compound (III) with a carbonyl compound such as an aldehyde, a ketone, etc.; a silyl derivative formed by the reaction of compound (III) with a silyl compound such as bis(trimethylsilyl)acetamide, mono(trimethylsilyl)acetamide, bis(trimethylsilyl)urea, etc.; and a derivative formed by the reaction of compound (III) with phosphorus trichloride or phosgene.

Specifically, a preferred reactive derivative at carboxyl group of compound (I-5) include an acid halide, an acid anhydride, an activated amide, an activated ester, or the like. Preferred examples of the derivative is an acid chloride; an acid azide; a mixed acid anhydride with a substituted phosphoric acid such as a dialkylphosphoric acid, phenylphosphoric acid, diphenylphosphoric acid, dibenzylphosphoric acid, a halogenated phosphoric acid, etc.; a dialkylphosphorus acid; sulfurous acid; thiosulfuric acid; sulfuric acid; a sulfonic acid such as methanesulfonic acid, etc.; an aliphatic carboxylic acid such as acetic acid, propionic acid, butyric acid, isobutyric acid, pivalic acid, pentanoic acid, isopentanoic acid, trichloroacetic acid, etc.; or an aromatic carboxylic acid such as benzoic acid, etc.; a symmetric acid anhydride; an active amide with imidazole; a 4-substituted imidazole; dimethylpyrazole; triazole, or tetrazole; or an activated ester such as a cyanomethyl ester, a methoxymethyl ester, a dimethyliminomethyl ester, a vinyl ester, a propargyl ester, a p-nitrophenyl ester, a trichlorophenyl ester, a pentachlorophenyl ester, a mesylphenyl ester, a phenylazophenyl ester, a phenylthio ester, a p-cresylthio ester, a carboxymethylthio ester, a pyranyl ester, a pyridyl ester, a piperidyl ester, an 8-quinolylthio ester, etc.; or an ester with an N-hydroxyl compound such as N,N-dimethylhydroxylamine, 1-hydroxy-2-(1H)-pyridone, N-hydroxysuccinimide, N-hydroxyphthalimide, 1-hydroxy-1H-benzotriazole, etc.; or the like. These reactive derivatives may be selected optionally depending on a particular kind of compound (I-5) to be employed. Examples of a preferred salt of the reactive derivative of compound (I-5) include a basic salt, for example an alkali metal salt such as a sodium salt, a potassium salt, etc., an alkaline earth metal salt such as a calcium salt, a magnesium salt, etc., an ammonium salt, an organic base salt such as a trimethyl amine salt, a triethylamine salt, a picoline salt, a dicyclohexylamine salt, N,N-dibenzylethylenediamine salt, etc. The reaction is carried out usually in a conventional solvent such as water, alcohol, for example, methanol, ethanol, or the like, acetone, dioxane, acetonitrile, chloroform, methylene chloride, ethylene chloride, tetrahydrofuran, ethyl acetate, N,N-dimethylformamide, or pyridine. However, the reaction can be carried out in any other organic solvent in so far as the solvent does not adversely affect the reaction. These conventional solvents may be used as a mixture with water.

In this reaction, when compound (I-5) is employed in the form of a free acid or in the form of its salt, it is desirable to carry out the reaction in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diethylcarbodiimide, N,N′-diisopropylcarbodiimide, N-ethyl-N′-(3-dimetylaminopropyl)carbodiimide; N,N′-carbonylbis(2-methylimidazole); pentamethyleneketene-N-cyclohexylimine; diphenylketene-N-cyclohexylimine; ethoxyacetylene; 1-alkoxy-1-chloroethylene; trialkylphosphite; ethyl polyphosphate; isopropyl polyphosphate; phosphorus oxychloride; diphenylphosphoryl azide; thionyl chloride; oxalyl chloride; a lower alkyl haloformate such as ethyl chloroformate, isopropyl chloroformate, etc.; triphenylphosphine; 2-ethyl-7-hydroxybenzisoxazolium salt; 2-ethyl-5-(m-sulfophenyl)isoxazolium hydroxide inner salt; N-hydroxybenzotriazole; 1-(p-chlorobenzenesulfonyloxy)-6-chloro-1H-benzotriazole; a so-called Vilsmeier reagent prepared by reaction of N,N′-dimethylformamide with thionyl chloride, phosgene, trichloromethyl chloroformate, phosphorus oxychloride, etc.; or the like. The reaction may be carried out also in the presence of an inorganic base or an organic base such as an alkali metal hydrogen carbonate, a tri(lower)alkylamine, pyridine, N-(lower)alkylmorpholine, N,N-di(lower)alkylbenzylamine, or the like. Although the reaction temperature is not specifically limited, the reaction is carried out usually under cooling or with warming.

The amount of compound (VIII) to be used is 1-10 molar equivalents, preferably 1-3 molar equivalents for compound (I-5).

The reaction temperature is usually −30° C. to 100° C.

The reaction time is usually about 0.5 to about 20 hours.

Further, when a mixed acid anhydride is used, compound (I-5) is reacted with a chlorocarbonic acid ester (e.g., methyl chlorocarbonate, ethyl chlorocarbonate, isobutyl chlorocarbonate, etc.) in the presence of a base (e.g., triethylamine, N-methylmorpholine, N,N-dimethylaniline, sodium hydrogen carbonate, sodium carbonate, potassium carbonate, etc.) and further is reacted with compound (VIII).

The amount of compound (VIII) to be used is 1-10 molar equivalents, preferably 1-3 molar equivalents for compound (I-5).

The reaction temperature is usually −30° C. to 100° C.

The reaction time is usually about 0.5 to about 20 hours.

The thus-obtained compound (I-6) can be isolated and purified according to a known separation and purification means such as concentration, concentration under reduced pressure, solvent extraction, crystallization, recrystallization, trans-solubilization, chromatography, or the like.

Compound (I-5) to be employed as the starting compound in the above-mentioned Process D can be produced by the above-mentioned Process C.

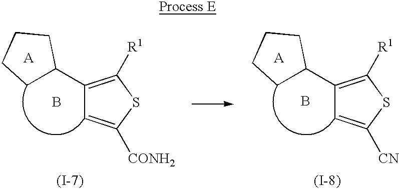

wherein each symbol is as defined above.

In this process, compound (I-8) is produced from compound (I-7) in the presence of a dehydrating agent.

This reaction is carried out by employing a method wherein compound (I-7) is appropriately reacted with a dehydrating agent, or the like. Examples of the dehydrating agent to be used here include acetic anhydride, trifluoroacetic anhydride, phosphorus pentaoxide, thionyl chloride, or the like.

The amount of the dehydrating agent to be used is 0.1-100 molar equivalents, preferably 1-10 molar equivalents for compound (I-7).

The reaction temperature is usually −30° C. to 100° C.

The reaction time is usually about 0.5 to about 20 hours.

The thus-obtained compound (I-8) can be isolated and purified according to a known separation and purification means such as concentration, concentration under reduced pressure, solvent extraction, crystallization, recrystallization, trans-solubilization, chromatography, or the like.

Compound (I-7) to be employed as the starting compound in the above-mentioned Process E can be produced by the above-mentioned Process D or Process B.

For example, when an acid chloride is used, the reaction is carried out in the presence of a base in a solvent that does not adversely affect the reaction. Examples of the solvent that does not adversely affect the reaction include ether such as diethyl ether, tetrahydrofuran, dioxane, etc.; halogenated hydrocarbon such as chloroform, dichloromethane, etc.; aromatic hydrocarbon such as benzene, toluene, xylene, etc.; amide such as N,N-dimethylformamide, N-methylpyrrolidine, etc.; sulfoxide such as dimethyl sulfoxide, etc.; or the like. These solvents may be used as a mixture thereof in an appropriate ratio.

Among compound (I), compound (I-10) wherein R² is -Z⁷-Z⁸ (wherein Z⁷ is —CO—, Z⁸ is an optionally substituted hydrocarbon group, an optionally subsituted heterocyclic group or an optionally subsituted hydroxyl group) can be produced by, for example, the following Process F.

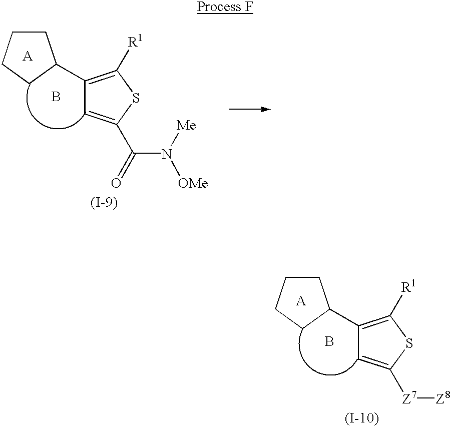

wherein each symbol is as defined above.

In this process, compound (I-10) is produced by the reaction of a nucleophilic reagent with compound (I-9). Examples of said nucleophilic reagent to be used include a metal phenolate, a metal alcoholate, a Grignard reagent, an alkyl metal reagent, an aryl metal reagent, a thioalcoholate, or the like.

The amount of the nucleophilic agent to be used is preferably 1-5 molar equivalents for compound (I-9).

This reaction is carried out usually in a solvent that does not adversely affect the reaction. Examples of the solvent that does not adversely affect the reaction include ether such as diethyl ether, tetrahydrofuran, dioxane, etc.; halogenated hydrocarbon such as chloroform, dichloromethane, etc.; aromatic hydrocarbon such as benzene, toluene, xylene, etc.; amide such as N,N-dimethylformamide, N-methylpyrrolidine, etc.; sulfoxide such as dimethyl sulfoxide, etc.; or the like. These solvents may be used as a mixture thereof in an appropriate ratio.

The reaction temperature is usually about −30 to about 150° C., preferably about −70 to about 0° C.

The reaction time is usually about 0.5 to about 20 hours.

The thus-obtained compound (I-10) can be isolated and purified according to a known separation and purification means such as concentration, concentration under reduced pressure, solvent extraction, crystallization, recrystallization, trans-solubilization, chromatography, or the like.

Compound (I-9) to be employed as the starting compound in the above-mentioned Process F can be produced by Process D.

Compound (I) can be obtained by the following production process other than the above-mentioned production processes.

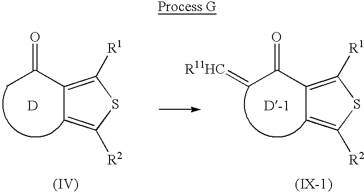

wherein each symbol is as defined above.

Compound (IX-1) is produced by the reaction of compound (IV) with an amide acetal.

Examples of the amide acetal to be used include an active acetal of an N,N-dialkylformamide, preferably, an active acetal compound of dimethylformamide such as N,N-dimethylformamide dimethyl acetal, N,N-dimethylformamide diethyl acetal, methoxybis(dimethylamino)methane, ethoxybis(dimethylamino)methane, t-butoxybis(dimethylamino)methane, tris(dimethylamino)methane, N,N-dimethylformamide dipropyl acetal, N,N-dimethylformamide bis(2-trimethylsilylethyl) acetal, N,N-dimethylformamide dibenzyl acetal, N,N-dimethylformamide di-t-butyl acetal, N,N-dimethylformamide dineopentyl acetal, N,N-dimethylformamide dicyclohexyl acetal, N,N-dimethylformamide diisopropyl acetal, or the like, more preferably, N,N-dimethylformamide dimethyl acetal, N,N-dimethylformamide diethyl acetal, methoxybis-(dimethylamino)methane, ethoxybis(dimethylamino)methante, t-butoxybis(dimethylamino)methane, tris(dimethylamino)methane, N,N-dimethylformamide dipropyl acetal, N,N-dimethylformamide diisopropyl acetal or the like.

The amount of said amide acetal to be used is 1 mole to 50 moles, preferably 1 mole to 30 moles for 1 mole of compound (IV).

The solvent to be used for this reaction may be any solvent in so far as it does not adversely affect the reaction. Examples thereof include hydrocarbon (e.g., n-hexane, n-heptane, benzene, toluene, xylene, etc.), halogenated hydrocarbon (e.g., dichloromethane, etc.), ether (e.g., diethyl ether, diisopropyl ether, ethylene glycol dimethyl ether, tetrahydrofuran, dioxane, etc.), amide (e.g., N,N-di-C₁₋₃-alkylformamide such as N,N-dimethylformamide, etc., N,N-dimethylacetamide, N-methylpyrrolidone, etc.), ester (e.g., ethyl acetate, methyl acetate, etc.), nitrile (e.g., acetonitrile, etc.), sulfoxide (e.g., dimethyl sulfoxide, etc.), and the like. These solvents can be used alone or in combination thereof.

This reaction is carried out at a temperature of 0 to 150° C., preferably 50 to 120° C. for about 30 minutes to 24 hours, preferably for 1 to 6 hours.

All of the amide acetals described herein are per se known and easily available as commercially available products. In addition, compound (IV) is produced by a known process, for example, the process described in a paper by D. Prim et al. (Synth. Commun., vol. 25, page 2449, 1995) or a modified process thereof.

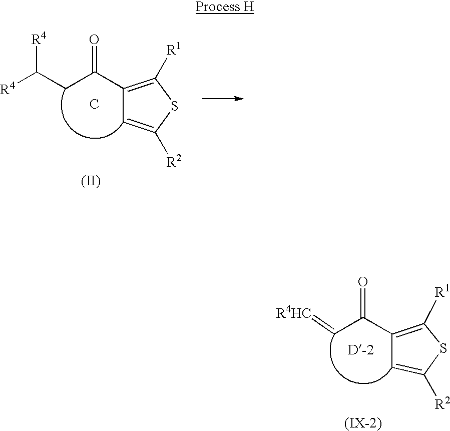

wherein each symbol is as defined above.

Compound (IX-2) is produced by de-alcoholization of compound (II).

This present reaction is carried out by using an acid or a base. Examples of the acid include inorganic acids such as hydrochloric acid, hydrobromic acid, sulfuric acid, phosphoric acid, and the like and organic acids such as trifluoroacetic acid, methanesulfonic acid, benzenesulfonic acid, p-toluenesulfonic acid, and the like. Examples of the base include alkali metal or alkaline earth metal hydrides such as sodium hydride and the like; alkali metal or alkaline earth metal amides such as lithium diisopropylamide, lithium hexamethyldisilazide, sodium hexamethyldisilazide, and the like; alkali metal or alkaline earth metal lower alkoxides such as sodium methoxide, sodium ethoxide, potassium t-butoxide, and the like; alkali metal or alkaline earth metal hydroxides such as potassium hydroxide, sodium hydroxide, and the like; carbonates such as potassium carbonate, sodium carbonate, cesium carbonate, and the like, alkali metal or alkaline earth metal hydrogen carbonates such as potassium hydrogen carbonate, sodium hydrogen carbonate, and the like; and organic bases such as triethylamine, diisopropylamine, pyridine, dimethylaminopyridine, 1,8-diazabicyclo[5.4.0]-7-undecene, and the like. Among them, inorganic acids such as hydrochloric acid, hydrobromic acid, sulfuric acid, phosphoric acid, or the like and organic acids exemplified by trifluoroacetic acid, methanesulfonic acid, benzenesulfonic acid, p-toluenesulfonic acid, and the like are preferred and the acid is used in 0.1 mole to 100 moles, preferably 1 mole to 30 moles for 1 mole of compound (II).

In this reaction, if necessary, any solvent that does not adversely affect said reaction can be used. In particular, alcohol (e.g., C₁₋₃ alcohol such as methanol, ethanol, propanol, etc.) is preferred. In addition, the above-mentioned acid or base may also be used as a solvent.

This reaction is carried out at a reaction temperature of 0 to 50° C., preferably 0 to 30° C. for about 10 minutes to 6 hours, preferably for 30 minutes to 3 hours.

Compound (II) is produced from compound (IV) by a known process, for example, the process described in a paper by A. Nangia et al. (Indian J. Chem., vol. 35B, page 49, 1996) or a modified process thereof.

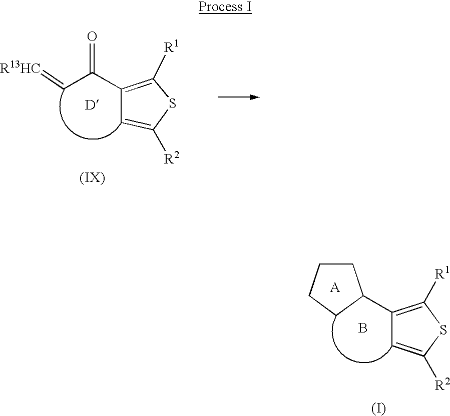

wherein each symbol is as defined above.

A compound represented by the general formula (I) is produced by subjecting compound (VII) to a ring closure reaction with hydroxylamine or its salt, or a hydrazine derivative represented by R³NHNH₂ (R^{3′} is as defined above) or its salt.

In this reaction, hydroxylamine or the hydrazine derivative is used in 1 mole to 10 moles, preferably 1 mole to 5 moles for 1 mole of compound (IX).

As for a solvent to be used in this reaction, any solvent can be used in so far as it does not adversely affect said reaction. Preferred examples thereof include alcohol (e.g., C₁₋₃ alcohol such as methanol, ethanol, propanol, etc.) or a mixture thereof with another appropriate solvent or water.

This reaction can be carried out in the presence of an acid so to control the reaction rate, regioselectivity, solubility, and the like. Examples of said acid include an inorganic acid such as hydrochloric acid, hydrobromic acid, sulfuric acid, phosphoric acid, or the like and an organic acid such as trifluoroacetic acid, methanesulfonic acid, benzenesulfonic acid, p-toluenesulfonic acid, or the like. They are used in 0.1 mole to 100 moles, preferably 1 mole to 30 moles for 1 mole of compound (IX). In addition, they can also be used as a solvent.

This reaction is carried out at a reaction temperature of 0 to 120° C., preferably 50 to 100° C. for about 10 minutes to 6 hours, preferably for 1 hour to 3 hours.

R^{3′} on ring A formed by this reaction can be converted into an optionally substituted hydroxyl group or an optionally substituted amino group of R³ according to a per se known method.

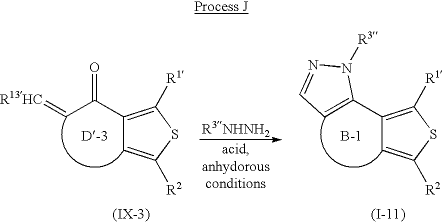

wherein, each symbol is as defined above.

In this reaction, compound (I-11) is produced by subjecting compound (IX-3) to a ring closure reaction with a hydrazine derivative represented by R^{3″}NHNH₂. The hydrazine derivative is used in 1 mole to 10 moles, preferably 1 mole to 5 moles for 1 mole of compound (IX-3).

As for the solvent to be used in this reaction, any solvent can be used in so far as it does not adversely affect said reaction. Preferred examples thereof include alcohol (e.g., C₁₋₃ alcohol such as methanol, ethanol, propanol, etc.).

The anhydrous condition in this reaction means to carry out the reaction under substantially anhydrous conditions, specifically using a solvent to which water is not positively added such as a solvent having a water content of less than about 5%, preferably less than about 3%, and more preferably less than about 1%.

Examples of the acid to be used in this reaction include an inorganic acid such as hydrochloric acid, hydrobromic acid, sulfuric acid, phosphoric acid, or the like and an organic acid such as trifluoroacetic acid, methanesulfonic acid, benzenesulfonic acid, p-toluenesulfonic acid, or the like, with methanesulfonic acid being particularly preferred. The amount thereof to be used is 0.1 mole to 100 moles, preferably 1 mole to 30 moles for 1 mole of compound (IX-3). In addition, they may also be used as a solvent.

This reaction is carried out at a reaction temperature of 0 to 120° C., preferably 40 to 70° C. for about 10 minutes to 6 hours, preferably for 1 hour to 3 hours.

When the compound wherein R^{13′} of the general formula (IX-3) is hydroxyl group substituted with a cyclic group, it is produced from compound (IV) by a known process, for example, the process described in a paper by D. Prim et al. (Synth. Commun., vol. 25, page 2449, 1995) or a modified process thereof. Alternatively, said compound may be produced by subjecting compound (IX) to a common acid hydrolysis reaction (e.g., a reaction using a mixed solvent system of alcohol or an amide with water and using the same acid as that described above in 0.1 mole to 10 moles for compound (IX) at a reaction temperature of 0 to 120° C. for about 10 minutes to 6 hours).

Further, by using “an optionally substituted amino group” as R^{13′} in the general formula (IX-3), the objective compound can be produced industrially and advantageously without the use of a compound such as the boron trifluoride-ether complex, which involves a corrosive problem in the production of compound (IX-3).

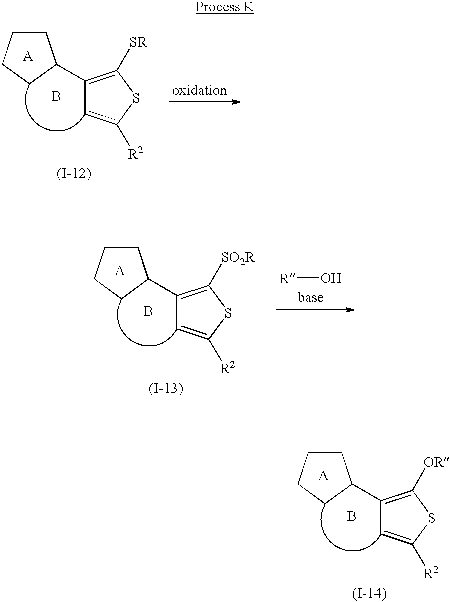

Compound (I-12) wherein R¹ in the general formula (I) is an optionally substituted thiol is converted into compound (I-13) in the following manner.

First, compound (I-12) is subjected to the oxidation reaction to prepare compound (I-13). As an oxidizing agent, there can be used a peracid such as metachloroperbenzoic acid, peracetic acid, performic acid, trifluoroperacetic acid, or the like, a peroxide such as dioxysilane or the like, hydrogen peroxide in the presence of a metal catalyst, Oxone (trade name), or the like in 2 mole to 10 moles for 1 mole of compound (I-12).

In the case of the oxidation with peracetic acid or the like, it is desirable to add an acid such as hydrochloric acid, sulfuric acid, or the like, in an amount of 1 mole to 10 moles, preferably 2 to 5 moles for 1 mole of compound (I-12) to accelerate the reaction.

As for the solvent to be used for this reaction, any solvent can be used in so far as it does not adversely affect the reaction. Examples thereof include hydrocarbons (e.g., n-hexane, n-heptane, benzene, toluene, xylene, etc.), halogenated hydrocarbons (e.g., dichloromethane, etc.), ethers (e.g., diethyl ether, diisopropyl ether, ethylene glycol dimethyl ether, tetrahydrofuran, dioxane, etc.), alcohol (e.g., C₁₋₃ alcohol such as methanol, ethanol, propanol, etc.), amides (e.g., N,N-di-C₁₋₃-alkylformamide such as N,N-dimethylformamide, etc., N,N-dimethylacetamide, N-methylpyrrolidone, and the like), esters (e.g., ethyl acetate, methyl acetate, etc.), nitrites (e.g., acetonitrile, etc.), sulfoxides (e.g., dimethyl sulfoxide, etc.), ketones (e.g., acetone, 2-butanone, 4-methyl-2-pentanone, cyclohexanone, etc.), carboxylic acids (e.g., formic acid, acetic acid, trifluoroacetic acid, etc.), and the like. They can be used alone or in combination thereof.

The reaction temperature and time in this reaction differ depending on a particular oxidizing agent to be used. For example, when peracetic acid is used, the reaction is carried out at a temperature of 0 to 100° C., preferably 30 to 60° C. for about 1 hour to 24 hours, preferably for 2 to 5 hours.

The thus-obtained compound (I-13) is subjected to a substitution reaction to produce compound (I-14). In this reaction, R″—OH is used in 1 mole to 2 moles, preferably 1 mole to 1.5 moles for 1 mole of compound (I-13).

The R″—O moiety of R″—OH corresponds to an optionally substituted hydroxyl group of R¹.

The same base as that exemplified with respect to Process H can be used in this reaction. Among them, preferred examples include sodium methoxide, sodium ethoxide, potassium t-butoxide, potassium carbonate, sodium carbonate, cesium carbonate, potassium hydrogen carbonate, sodium hydrogen carbonate, or the like. It is used in an amount of 1 mole to 3 moles, preferably 1 mole to 2 moles for 1 mole of compound (I-13).

As for the solvent to be used for this reaction, any solvent can be used in so far as it does not adversely affect on the reaction. Examples thereof include hydrocarbons (e.g., n-hexane, n-heptane, benzene, toluene, xylene, etc.), halogenated hydrocarbons (e.g., dichloromethane, etc.), ethers (e.g., diethyl ether, diisopropyl ether, ethylene glycol dimethyl ether, tetrahydrofuran, dioxane, etc.), amides (e.g., N,N-dimethylformamide, N,N-dimethylacetamide, N-methylpyrrolidone, etc.), esters (e.g., ethyl acetate, methyl acetate, etc.), nitrites (e.g., acetonitrile, etc.), sulfoxides (e.g., dimethyl sulfoxide, etc.), ketones (acetone, 2-butanone, 4-methyl-2-pentanone, cyclohexanone, etc.), and the like. In particular, preferred examples include hydrocarbons (e.g., n-hexane, n-heptane, benzene, toluene, xylene, etc.), amides (e.g., N,N-dimethylformamide, N,N-dimethylacetamide, N-methylpyrrolidone, etc.), esters (e.g., ethyl acetate, methyl acetate, etc.), ketones (e.g., acetone, 2-butanone, 4-methyl-2-pentanone, cyclohexanone, etc.), and ethers (e.g., diethyl ether, diisopropyl ether, ethylene glycol dimethyl ether, tetrahydrofuran, dioxane, etc.). These solvents can be used alone or in combination thereof.

This reaction is carried out at a temperature of 20 to 120° C., preferably 70 to 100° C. for about 1 hour to 24 hours, preferably for 2 to 6 hours.

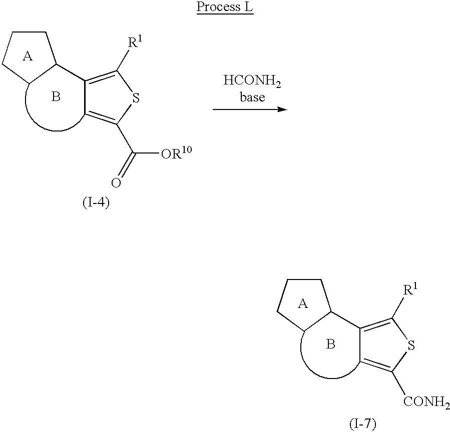

Compound (I-4) wherein R¹⁰ represents a protecting group of carboxyl group can be converted into compound (I-7) in a single step by reacting it with formamide in the presence of a base.

Usually, formamide is also used as a solvent, and is used in an amount of 1 ml to 30 ml, preferably 2 to 10 ml for 1 g of compound (I-4).

The same base as that exemplified with respect to Process H can be used in this reaction. Among them, preferred examples include sodium methoxide, sodium ethoxide, potassium t-butoxide, potassium carbonate, sodium carbonate, cesium carbonate, potassium hydrogen carbonate, sodium hydrogen carbonate, or the like. It is used in an amount of 1 mole to 10 moles, preferably 1 mole to 5 moles for 1 mole of compound (I-4).

As for the solvent to be used for this reaction, any solvent can be used in so far as it does not adversely affect the reaction. Preferred examples thereof include alcohol (e.g., C₁₋₃ alcohol such as methanol, ethanol, propanol, etc.), an amide (e.g., N,N-dimethylformamide, N,N-dimethylacetamide, N-methylpyrrolidone, formamide, etc.) and the like.

This reaction is carried out at a temperature of 20 to 120° C., preferably 70 to 100° C. for about 1 hour to 12 hours, preferably for 1 hour to 3 hours.

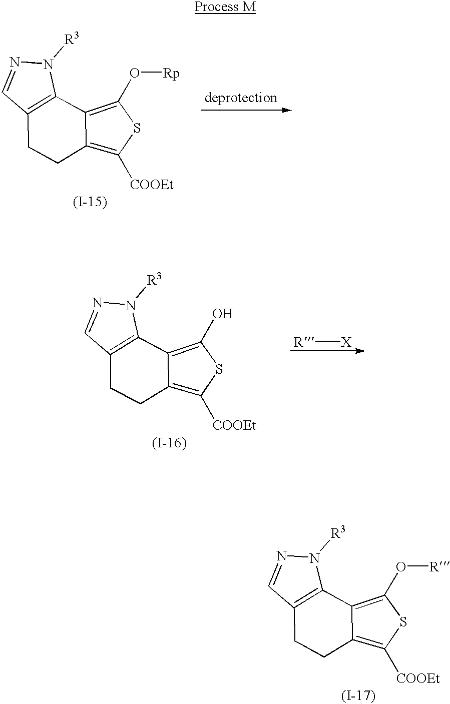

wherein, R′″ represents a hydrocarbon group or a heterocyclic group each of which may be substituted; and Rp represents a protecting group of hydroxyl group and the other symbols are as defined above.

Compound (I-15) wherein Rp is a protecting group of hydroxyl group is converted into compound (I-16) by a deprotection reaction, followed by a substitution reaction to produce compound (I-17).

Examples of Rp in compound (I-15) to be used in the first reaction include C₁₋₆ alkyl (e.g., methyl, ethyl, propyl, isopropyl, butyl, isobutyl, tert-butyl, etc.), phenyl, trityl, C₇₋₁₄ aralkyl (e.g., benzyl, etc.), formyl, C₁₋₆ alkyl-carbonyl (e.g., acetyl, propionyl, etc.), benzoyl, C₇₋₁₀ aralkyl-carbonyl (e.g., benzylcarbony, etc.), 2-tetrahydropyranyl, 2-tetrahydrofuranyl, silyl (e.g., trimethylsilyl, triethylsilyl, dimethyphenylsilyl, tert-butyldimethylsilyl, tert-butyldiethylsilyl, etc.), C₂₋₆ alkenyl (e.g., 1-allyl, etc.), and the like. These groups may be substituted with 1 to 3 substituents such as a halogen atom (e.g., fluorine, chlorine, bromine, iodine, etc.), C₁₋₆ alkyl (e.g., methyl, ethyl, propyl, etc.), C₁₋₆ alkoxy (e.g., methoxy, ethoxy, propoxy, etc.), nitro group, or the like. The removal of the protecting group to be used in this reaction is carried out according to a per se known methods, for examples, the methods described in Protective Groups in Organic Synthesis, John Wiley and Sons (1980), and the like. For examples, there are employed a method using an acid, a base, a ultraviolet light, hydrazine, phenylhydrazine, sodium N-methyldithiocarbamate, tetrabutylammonium fluoride, palladium acetate, a trialkylsilyl halide (e.g., trimethylsilyl iodide, trimethylsilyl bromide, etc.), or the like. A reduction method, or the like, can also be employed.

Examples of “the hydrocarbon group or heterocyclic group each of which may be substituted” represented by R′″ included those represented by R¹.

The thus-obtained compound (I-16) is subjected to a substitution reaction to produce compound (I-17). In thist reaction, R′″-X is used in an amount of 1 mole to 2 moles, preferably 1 mole to 1.5 moles for 1 mole of compound (I-16).

The same base as that exemplified with respect to Process H can be used in this reaction. Among them, preferred examples include sodium fluoride, potassium fluoride, cesium fluoride, or the like. It is used in an amount of 1 mole to 3 moles, preferably 1 mole to 2 moles for 1 mole of compound (I-16).

As for the solvent to be used for this reaction, any solvent can be used in so far as it does not adversely affect the reaction. Examples thereof include hydrocarbons (e.g., n-hexane, n-heptane, benzene, toluene, xylene, and the like), halogenated hydrocarbons (for examples, dichloromethane, etc.), ethers (e.g., diethyl ether, diisopropyl ether, ethylene glycol dimethyl ether, tetrahydrofuran, dioxane, etc.), amides (e.g., N,N-dimethylformamide, N,N-dimethylacetamide, N-methylpyrrolidone, etc.), esters (e.g., ethyl acetate, methyl acetate, etc.), nitriles (e.g., acetonitrile, etc.), sulfoxides (e.g., dimethyl sulfoxide, etc.), ketones (e.g., acetone, 2-butanone, 4-methyl-2-pentanone, cyclohexanone, etc.), and the like. In particular, preferred examples include hydrocarbons (e.g., n-hexane, n-heptane, benzene, toluene, xylene, etc.), amides (e.g., N,N-dimethylformamide, N,N-dimethylacetamide, N-methylpyrrolidone, etc.), esters (e.g., ethyl acetate, methyl acetate, etc.), ketones (e.g., acetone, 2-butanone, 4-methyl-2-pentanone, cyclohexanone, etc.), and ethers (e.g., diethyl ether, diisopropyl ether, ethylene glycol dimethyl ether, tetrahydrofuran, dioxane, etc.). These solvents are used alone or in combination thereof.

This reaction is carried out at a temperature of 20 to 120° C., preferably 70 to 90° C. for about 1 hour to 24 hours, preferably for 2 to 6 hours.

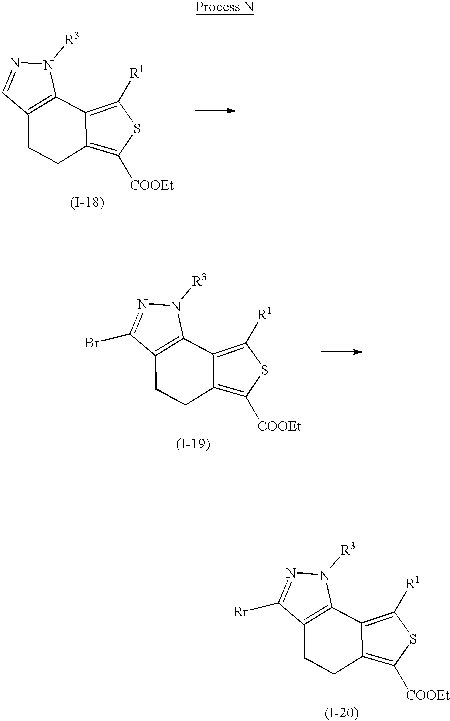

wherein Rr represents an optionally substituted hydrocarbon group. Examples of “an optionally substituted hydrocarbon group” represented by Rr include that represented by R¹.

In this process, compound (I-20) is produced by reacting compound (I-19) obtained by bromination of compound (I-18) with a nucleophilic reagent; by treatment of compound (I-19) with a metal reagent, followed by the reaction with an electrophilic reagent; or by a coupling reaction with, for example, a boronic acid reagent in the presence of a metal catalyst and a base. As said nucleophilic reagent to be used, for example, there is a metal phenolate, a metal alcoholate, a Grignard reagent, an alkyl metal reagent, an aryl metal reagent, a thioalcoholate, or the like. As said electrophilic reagent, for example, there is an alkyl halide, an aralkyl halide, an aldehyde, a ketone, or the like. On the other hand, as the metal catalyst, for example, there is bis(triphenylphosphine)palladium chloride or the like and, as the base, for example, there is sodium carbonate, sodium hydrogen carbonate, cesium fluoride, or the like.

The amounts of the nucleophilic agent and the electrophilic agent to be used are preferably about 1 to about 5 molar equivalents for compound (I-19). The amount of the metal catalyst to be used is preferably about 0.01 to about 0.1 molar equivalent for compound (I-19) and, also, the amount of the base to be used is preferably about 1 to about 5 molar equivalents for compound (I-19).

This reaction is carried out usually in a solvent that does not adversely affect the reaction. Examples of the solvent that does not adversely affect the reaction include ethers such as diethyl ether, tetrahydrofuran, dioxane, and the like; halogenated hydrocarbons such as chloroform, dichloromethane, and the like; aromatic hydrocarbons such as benzene, toluene, xylene, and the like; amides such as N,N-dimethylformamide, N-methylpyrrolidine, and the like; sulfoxides such as dimethyl sulfoxide and the like. These solvents may be used as a mixture in an appropriate ratio.

The reaction temperature is usually about −70 to about 150° C., preferably about −70 to about 0° C.

The reaction time is usually about 0.5 to about 20 hours.

The thus-obtained compound (I-20) can be isolated and purified according to a known separation and purification means such as concentration, concentration under reduced pressure, solvent extraction, crystallization, recrystallization, trans-solubilization, chromatography, or the like.

Compound (I-9) to be employed as the starting compound in the above-mentioned Process F can be produced by Process D.

All the compounds used or obtained in the present invention include corresponding salts, even if specifically stated, and they can be exchanged to one another by a per se known method or modified methods thereof.

When the compounds or salts thereof obtained by the present invention are asymmetric molecules, they can be separated into d form isomer and 1 form isomer according to a conventional optical resolution means.

The compounds or salts thereof to be obtained by the present invention can be isolated and purified according to a means such as solvent extraction, concentration under reduced pressure, crystallization, recrystallization, distillation, chromatography, or the like.

The compound or its salt obtained by the present invention may be used in a next step as its reaction mixture without sufficient purification.

Compound (I) or its salt of the present invention has an excellent activity to induce alkaline phosphatase and an action to promote the chondromodulin production and/or an action to enhance the expression. Then, the compound is expected to have a potent action to promote osteogenesis, actions to induce and promote differentiation of osteoblasts including osteoblast precursor cells, an action to promote chondrogenesis, and actions to induce and promote differentiation of chondrocytes including chondrocyte precursor cells, and further an action to enhance the BMP action. These differentiation induction and differentiation inducing and promoting actions not only affect the differentiation of osteoblasts and chondrocytes but also affect the induction of differentiation of various cells.

Further, compound (I) or its salt is expected to enhance the activity of neurotrophic factors.

Furthermore, compound (I) or its salt is expected to have anti-matrix metalloprotease (anti-MMP) activity.

In addition, it is excellent in clinically useful properties such as stability, absorbability (oral absorption in particular), and bioavailability.

Moreover, the toxicity is low. Therefore, compound (I) or its salt may be safely administered to mammalian animals (for example, human, rat, mouse, dog, rabbit, cat, cow, horse, pig, and the like).

Compound (I) or its salt is expected to have a potent action to promote osteogenesis, actions to induce and promote differentiation of osteoblasts including osteoblast precursor cells, an action to promote chondrogenesis, and actions to induce and promote differentiation of chondrocytes including chondrocyte precursor cells, and further an action to enhance the BMP action. Then, prophylactic and therapeutic drugs for articular disease containing compound (I) or its salt may be used, for example, as promoters of osteogenesis, prophylactic and therapeutic drugs of bone diseases, prophylactic and therapeutic drugs of bone fracture, promoters of chondrogenesis, and prophylactic and therapeutic drugs of chondropathy, specifically as prophylactic and therapeutic drugs of non-metabolic bone diseases in orthopedics such as bone fracture, bone deformation and spondylosis deformans, osteosarcoma, myeloma, osteogenesis imperfecta, scoliosis, and the like; as prophylactic and therapeutic drugs of metabolic diseases such as bone loss, osteoporosis, osteomalacia, rickets, fibrous ostitis, renal osteodystrophy, Paget's disease of bone, ankylosing spondylarthritis, and the like; or as prophylactic and therapeutic drugs of articular diseases, which are represented by chondropathy such as osteoarthritis and chronic rheumatoid arthritis, or as post-surgery repairing agents for multiple myeloma, lung carcinoma, breast carcinoma, and the like. In the dentistry field, the compound is expected to be applied to treatment of periodontal disease, repair of defect in the periodontal tissue in periodontal disease, stabilization of artificial dental root, alveolar crest formation, repair of cleft palate, and the like.

Further, since compound (I) or its is expected to have activity to enhance the action of neurotrophic factors, it is expected to be used in treatment and prevention of diseases, which are caused by various nerve degenerations such as Alzheimer's dementia, senile dementia in general, motor neuron dysfunction (e.g., amyotrophic lateral sclerosis, etc.), and diabetic peripheral neuropathy, and the like.

Furthermore, since pharmaceutical compositions containing compound (I) or its salt of the present invention are expected to have an anti-MMP activity, they are expected to be useful in the treatment and prevention of diseases, in which MMP is involved, such as osteoarthritis, chronic rheumatoid arthritis, arteriosclerosis, tumor metastasis, and the like.

The dosage of compound (I) or its salt can be selected in various ways depending on the administration route and the symptom of a patient to be treated. The dosage as compound (I) per an adult (a body weight of 50 kg) can be usually selected in a range of about 0.1 mg to about 500 mg, preferably about 1 mg to about 100 mg in the case of oral administration and in a range of about 0.01 mg to about 100 mg, further preferably about 0.1 mg to about 10 mg in the case of parenteral administration. The dosage can be administered with being divided in 1-3 times daily.

The objective compound (I) or its salt of the present invention can be formulated with a pharmaceutically acceptable carrier and can be orally or parenterally administered as solid formulations such as tablets, capsules, granules, powders, or the like; or liquid formulations such as syrups, injections, or the like. Also, there can be prepared formulations for transdermal administration such as patchings, cataplasms, ointments (including creams), plasters, tapes, lotions, liquids and solutions, suspensions, emulsions, sprays, and the like.

As for a pharmaceutically acceptable carrier, a variety of organic or inorganic carrier substances, which have been conventionally employed as formulation materials, is used and compounded as a bulking agent, a lubricant, a binding agent, and a disintegrator in solid formulations; a vehicle, a solubilizing agent, a suspending agent, an isotonicity agent, a buffering agent, and an analgesic in liquid formulations. If necessary, formulation excipients such as a preservative, an antioxidant, a stabilizer, a coloring agent, a sweetening agent, and the like can be used.

Preferred examples of the bulking agent include lactose, sucrose, D-mannitol, starch, crystalline cellulose, light anhydrous silicic acid, and the like. Preferred examples of the lubricant include magnesium stearate, potassium stearate, talc, colloidal silica, and the like. Preferred examples of the binding agent include crystalline cellulose, α-starch, sucrose, D-mannitol, dextrin, hydroxypropyl cellulose, hydroxypropylmethyl cellulose, polyvinyl pyrrolidone, and the like. Preferred examples of the disintegrator include starch, carboxymethyl cellulose, calcium carboxymethyl cellulose, croscarmellose sodium, sodium carboxymethyl starch, low-substituted hydroxypropyl cellulose, and the like. Preferred examples of the vehicle include water for injection, alcohol, propylene glycol, macrogol, sesame oil, corn oil, and the like.

If necessary, for the purpose of taste masking, enteric coating, or prolonged action, oral formulations can be prepared by coating by a per se known method. Examples of this coating agent include hydroxypropylmethyl cellulose, ethyl cellulose, hydroxymethyl cellulose, hydroxypropyl cellulose, polyoxyethylene glycol, Tween 80, Pluronic F68 [polyoxyethylene (160) polyoxypropylene (30) glycol], cellulose acetate phthalate, hydroxypropylmethyl cellulose phthalate, hydroxymethyl cellulose acetate phthalate, Eudragit (manufactured by Rohm Company, methacrylic acid-acrylic acid copolymer), and the like.

Preferred examples of the solubilizing agent include polyethylene glycol, propylene glycol, benzyl benzoate, ethanol, trisamiomethane, cholesterol, triethanolamine, sodium carbonate, sodium citrate, and the like. Preferred examples of the suspending agent include surface active agents such as stearyltriethanolamine, sodium lauryl sulfate, laurylaminopropionic acid, lecithin, benzalkonium chloride, benzethonium chloride, glycerin monostearate, and the like; hydrophilic, high molecular substances such as polyvinyl alcohol, polyvinyl pyrrolidone, sodium carboxymethyl cellulose, methyl cellulose, hydroxymethyl cellulose, hydroxyethyl cellulose, hydroxypropyl cellulose, and the like; and so on. Preferred examples of the isotonicity agent include sodium chloride, glycerin, D-mannitol, and the like. Preferred examples of the buffering agent include buffer solutions of a phosphate, an acetate, a carbonate, a citrate, or the like. Preferable examples of the analgesic include benzyl alcohol and the like. Preferred examples of the preservative include paraoxybenzoic acid esters, chlorobutanol, benzyl alcohol, phenethyl alcohol, dehydroacetic acid, sorbic acid, and the like. Preferred examples of the antioxidant include sulfites, ascorbic acid, and the like.

Further, compound (I) or its salt can be administered as a single formulation, or simultaneously or at temporal intervals together with [1] a cyclooxygenase inhibitor (a Cox-I, Cox-II inhibitor), [2] a disease-modifying anti-rheumatic drug and an immunodepressant, [3] a biological preparation, [4] an analgesic and an antiphlogistic, [5] a therapeutic drug for bone disease, and the like.

[1] Examples of cyclooxygenase inhibitors (Cox-I, Cox-II inhibitors) include celecoxib, rofecoxib, salicylic acid derivatives such as aspirin, diclofenac, indomethacin, loxoprofen, and the like. The oral doses of these drugs are, for example, about 100-200 mg/day for celecoxib, about 10-30 mg/day for rofecoxib, 1000-4500 mg/day for salicylic acid derivatives such as aspirin, about 25-75 mg/day for diclofenac, about 50-150 mg/day for indomethacin, and about 60-180 mg/day for loxoprofen.

[2] Disease-modifying anti-rheumatic drugs and immunodepressants include, for example, methotrexate, leflunomide, Prograf, sulfasalazine, D-penicillamine, oral gold compounds, and the like. The oral doses of these drugs are, for example, about 2.5-7.5 mg/week for methotrexate, about 20-100 mg/day for leflunomide, about 1-5 mg/day for Prograf, about 500-2000 mg/day for sulfasalazine, about 100-600 mg/day for D-penicillamine, and about 3-6 mg/day for oral gold compounds.

[3] Biological preparations include, for example, monoclonal antibodies (for examples, anti-TNF-a antibody, anti-IL-12 antibody, anti-IL-6 antibody, anti-ICAM-1 antibody, anti-CD4 antibody, and the like), soluble receptors (for examples, soluble TNF-α receptor and the like), and protein ligands (IL-1 receptor antagonist and the like). The oral doses of these drugs are, for example, about 0.1-50 mg/kg/day, preferably 0.5-20 mg/kg/day.

[4] Analgesics and antiphlogistics include, for example, centrally acting analgesics (for examples, morphine, codeine, pentazocine, and the like), steroids (for examples, prednisolone, dexamethasone, betamethazone, and the like), and antiphlogistic enzyme agents (for examples, bromelain, lysozyme, proctase, and the like).

The oral doses of these drugs are, for example, about 1-1000 mg/day, preferably about 5-300 mg/day, for centrally acting analgesics, about 0.1-400 mg/day, preferably about 0.5-100 mg/day, for steroids, and about 1-100 mg/day, preferably about 5-40 mg/day, for antiphlogistic enzyme agents.

[5] Prophylactic/therapeutic drugs for other bone diseases [for examples, bone fracture, refracture, osteoporosis, osteomalacia, Paget's disease of bone, ankylosing spondylitis, chronic rheumatoid arthritis, degenerative gonarthritis, destruction of joint tissues in related diseases, post-surgery repairing agents for multiple myeloma, lung carcinoma, breast carcinoma, and the like, and so on] include, for example, calcium preparations (for examples, calcium carbonate and the like), calcitonin preparations (for examples, eel calcitonin, salmon calcitonin, swine calcitonin, avicatonine, and the like), vitamin D₃ derivatives (for examples, 1α-hydroxy vitamin D₃, 1α,25-dihydroxy vitamin D₃, flocarcitriol, secarciferol, and the like), sex hormone-related compounds (for examples, tibolone, estrogen, estradiol, oxatelone, raloxifene, droloxifene, ormeloxifene, tamoxifen, mifepristone, and the like), prostaglandin A₁, bisphosphonates (for examples, etidronate, simadronate, alendronate, tyrudronate, risedronate, clodronate, and the like), ipriflavons, fluorine compounds (for examples, sodium fluoride and the like), vitamin K₂, bone morphogenic protein (BMP), fibroblast growth factor (FGF), platelet-derived growth factor (PDGF), transforming growth factor-β (TGF-β), insulin-like growth factor-1 and -2 (IGF-1, -2), parathyroid hormones (PTH) (for examples, PTH (1-34), PTH (1-84), PTH (1-36), and the like), and so on.

The following Reference Examples, Examples, and Test Examples further illustrate the present invention in more detail but are not to be construed to limit the scope of the present invention.

In the following Reference Examples, Examples, Test Examples, Me represents methyl, Et represents ethyl, n-Pr represents n-propyl, i-Pr represents isopropyl, tBu and t-Bu represent tertiary butyl, Ph represents phenyl, Cbz represents benzyloxycarbonyl, and Ac represents acetyl, respectively. Also, AcOEt represents ethyl acetate, HOBt represents 1-hydroxybenzotriazole, hexane represents n-hexane, THF represents tetrahydrofuran, ether represents diethyl ether, WSC represents 1-ethyl-3-(3-dimethylaminopropyl)carbonyldiimide hydrochloride salt, DMF represents N,N-dimethylformamide, and Pd-C represents palladium carbon, respectively.

### REFERENCE EXAMPLE 1

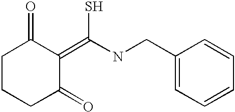

2-[Benzylamino(sulfanyl)methylene]-1,3-cyclohexanedione:

1,3-Cyclohexanedione (19.5 g) and benzyl isocyanate (26.0 g) were dissolved in acetonitrile (200 ml). To this solution was added triethylamine (24.3 ml) and the resulting solution was stirred at room temperature for 1 hour and further at 80° C. for 4 hours. The reaction solution was evaporated under reduced pressure and the residual oily substance was subjected to column chromatography on silica gel. From the fractions eluted with ethyl acetate-hexane (1:3), the title compound (15.0 g, 33%) was obtained as light yellow needles. Melting point: 47-48° C.

### REFERENCE EXAMPLE 2

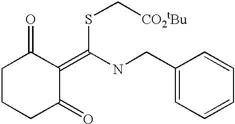

2-[[Benzylamino(2,6-dioxocyclohexylidene)methyl]sulfanyl]-acetic Acid t-butyl Ester:

A solution of 2-[benzylamino(sulfanyl)methylene]-1,3-cyclohexanedione (8.0 g), bromoacetic acid t-butyl ester (5.7 ml), and potassium carbonate (5.4 g) in N,N-dimethylformamide (DMF) (40 ml) was stirred under ice cooling for 4 hours. The reaction solution was poured into water and extracted with ethyl acetate. The organic layer was washed with water and then the solvent was evaporated under reduced pressure. The thus-obtained crystals were recrystallized from ethyl acetate-hexane to obtain the title compound (8.15 g, 71%) as colorless needles. Melting point: 142-143° C.

### REFERENCE EXAMPLE 3

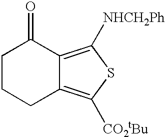

3-Benzylamino-4,5,6,7-tetrahydro-4-oxobenzo[c]thiophene-1-carboxylic Acid t-butyl Ester:

A solution of 2-[[benzylamino(2,6-dioxocyclohexylidene)methyl]sulfanyl]acetic acid t-butyl ester (10.0 g) and potassium t-butoxide (4.5 g) in 2-propanol (110 ml) was stirred at 50° C. for 70 minutes. The reaction solution was concentrated under reduced pressure and the residual oily substance was poured into water and extracted with ethyl acetate. The organic layer was dried (MgSO₄) and then the solvent was evaporated under reduced pressure. The thus-obtained oily substance was subjected to column chromatography on silica gel. From the fractions eluted with ethyl acetate-hexane (1:4), the title compound (3.5 g, 37%) was obtained. Recrystallization from ethyl acetate-hexane gave colorless needles. Melting point: 125-126° C.

According to the same manner as those in Reference Examples 1-3, compounds in Reference Examples 4 to 10 were obtained.

TABLE 1

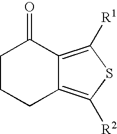

| Ref. | | | Melting point |
|---|---|---|---|
| Ex. | | | (recrystallization |
| No. | —R¹ | —R² | solvent) |
| 4 | 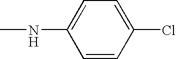 | —CO₂^{t}Bu | 142-143° C. (AcOEt—hexane) |
| 5 | —NH—Ph | —CO₂Et | 139—139° C. |
| | | | (AcOEt—hexane) |
| 6 | —NH—Ph | —CO₂^{t}Bu | 91-92° C. |
| | | | (i-Pr₂O) |
| 7 | —NH—Ph | —CO₂CH₂Ph | 117-118° C. |
| | | | (AcOEt—hexane) |
| 8 | —NH—Ph | —CN | 140-141° C. |
| | | | (AcOEt—hexane) |
| 9 | —NH—Me | —CO₂^{t}Bu | 125-126° C. |
| | | | (hexane) |
| 10 | —NH-(n-Pr) | —CO₂Et | 92-93° C. |
| | | | (AcOEt—hexane) |

### REFERENCE EXAMPLE 11

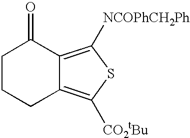

3-[(N-Benzoyl-N-benzyl)amino]-4,5,6,7-tetrahydro-4-oxobenzo[c]thiophene-1-carboxylic Acid t-butyl Ester:

To a solution of 3-benzylamino-4,5,6,7-tetrahydro-4-oxobenzo[c]thiophene-1-carboxylic acid t-butyl ester (3.0 g) in DMF (40 ml) was added 60% sodium hydride (0.47 g) under ice cooling and the resulting solution was stirred for 15 minutes. To this solution was added benzoyl chloride (1.2 ml) and the resulting solution was stirred under ice cooling for 1 hour and further at room temperature for 2 hours. The reaction solution was poured into an aqueous solution of citric acid and extracted with ethyl acetate. The organic layer was washed successively with water and an aqueous, saturated solution of sodium chloride, then dried (MgSO₄) and concentrated under reduced pressure. The thus-obtained, residual oily substance was subjected to column chromatography on silica gel. From the fractions eluted with ethyl acetate-hexane (1:4), the title compound (2.83 g, 73%) was obtained as a colorless amorphous solid. ¹H NMR (δ ppm in CDCl₃): 1.52 (9H, s), 1.7-1.9 (2H, m), 2.23 (2H, t, J=6.4 Hz), 2.99 (2H, t, J=6.4 Hz), 5.11 (2H, brs), 7.1-7.5 (10H, m).

According to the same manner as that in Reference Example 11, compounds in Reference Examples 12 to 15 were synthesized.

### REFERENCE EXAMPLE 12

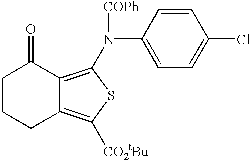

3-[[N-Benzoyl-N-(4-chlorophenyl)]amino]-4,5,6,7-tetrahydro-4-oxobenzo[c]thiophene-1-carboxylic Acid t-butyl Ester:

Light yellow prisms (yield: 79%). Melting point: 220-221° C. (recrystallization solvent: ether-hexane)

### REFERENCE EXAMPLE 13

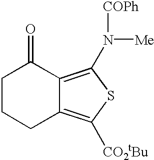

3-[(N-Benzoyl-N-methyl)amino]-4,5,6,7-tetrahydro-4-oxobenzo[c]thiophene-1-carboxylic Acid t-butyl Ester:

Colorless prisms (yield: 77%). Melting point: 135-136° C. (recrystallization solvent: AcOEt-hexane)

### REFERENCE EXAMPLE 14

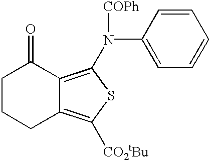

3-[(N-Benzoyl-N-phenyl)amino]-4,5,6,7-tetrahydro-4-oxobenzo[c]thiophene-1-carboxylic Acid t-butyl Ester:

Light yellow needles (yield: 80%). Melting point: 192-193° C. (recrystallization solvent: AcOEt-hexane)

### REFERENCE EXAMPLE 15

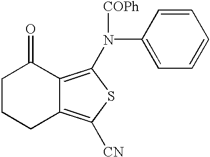

3-[(N-Benzoyl-N-phenyl)amino]-4,5,6,7-tetrahydro-4-oxobenzo[c]thiophene-1-carbonitrile:

Colorless prisms (yield: 82%). Melting point: 137-138° C. (recrystallization solvent: AcOEt-hexane)

### REFERENCE EXAMPLE 16

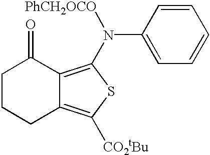

3-[(N-Benzyloxycarbonyl-N-phenyl)amino]-4,5,6,7-tetrahydro-4-oxobenzo[c]thiophene-1-carboxylic Acid t-butyl Ester:

To a solution of 3-phenylamino-4,5,6,7-tetrahydro-4-oxobenzo[c]thiophene-1-carboxylic acid t-butyl ester (6.0 g) in DMF (40 ml) was added 60% sodium hydride (1.0 g) under ice cooling and the resulting solution was stirred for 20 minutes. To this solution was added benzyl chloroformate (3.5 ml) and the resulting solution was stirred under ice cooling for 3 hours. The reaction solution was poured into an aqueous solution of citric acid and extracted with ethyl acetate. The organic layer was washed successively with water and an aqueous, saturated solution of sodium chloride, then dried (MgSO₄) and concentrated under reduced pressure. The thus-obtained residual oily substance was subjected to column chromatography on silica gel. From the fractions eluted with ethyl acetate-hexane (1:3), the title compound (9.2 g, 95%) was obtained as a light yellow, oily substance.

¹H NMR (δ ppm in CDCl₃): 1.53 (9H, s), 1.9-2.0 (2H, m), 2.46 (2H, t, J=6.4 Hz), 3.18 (2H, t, J=6.4 Hz), 5.19 (2H, s), 7.2-7.5 (10H, m).

According to the same manner as that in Reference Example 16, compounds in Reference Examples 17 to 18 were synthesized.

### REFERENCE EXAMPLE 17

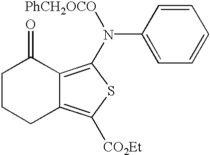

3-[(N-Benzyloxycarbonyl-N-phenyl)amino]-4,5,6,7-tetrahydro-4-oxobenzo[c]thiophene-1-carboxylic Acid Ethyl Ester:

Light yellow, oily substance (yield: 90%). ¹H NMR (δ ppm in CDCl₃): 1.32 (3H, t, J=7.2 Hz), 1.9-2.1 (2H, m), 2.47 (2H, t, J=6.4 Hz), 3.21 (2H, t, J=6.4 Hz), 4.29 (2H, q, J=7.2 Hz), 5.20 (2H, s), 7.2-7.4 (10H, m).

### REFERENCE EXAMPLE 18

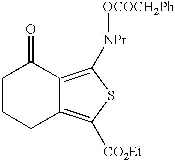

3-[(N-Benzyloxycarbonyl-N-propyl)amino]-4,5,6,7-tetrahydro-4-oxobenzo[c]thiophene-1-carboxylic Acid Ethyl Ester:

Light yellow, oily substance (yield: 100%). ¹H NMR (δ ppm in CDCl₃): 0.89 (3H, t, J=7.2 Hz), 1.36 (3H, t, J=7.2 Hz), 1.5-1.8 (2H, m), 1.9-2.1 (2H, m), 2.42 (2H, t, J=6.2 Hz), 3.20 (2H, t, J=6.2 Hz), 3.68 (2H, t, J=7.4 Hz), 4.33 (2H, q, J=7.2 Hz), 5.12 (1H, s), 7.1-7.4 (5H, m)

### REFERENCE EXAMPLE 19

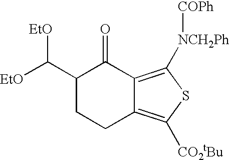

3-[(N-Benzoyl-N-benzyl)amino]-5-diethoxymethyl-4,5,6,7-tetrahydro-4-oxobenzo[c]thiophene-1-carboxylic Acid t-butyl Ester:

A solution of boron trifluoride ether complex (3.2 ml) in dichloromethane (15 ml) was added dropwise to triethyl orthoformate (3.4 g) that was cooled at −40° C. This solution was stirred under ice cooling for 15 minutes and then cooled to −70° C. To this solution were added dropwise a solution of 3-[(N-benzoyl-N-benzyl)amino-4,5,6,7-tetrahydro-4-oxobenzo[c]thiophene-1-carboxylic acid t-butyl ester (2.6 g) in dichloromethane (25 ml) and then diisopropylethylamine (4.9 ml). After being stirred at −70° C. for 1 hour, the reaction solution was poured into an aqueous solution of sodium hydrogen carbonate and extracted with chloroform. The organic layer was washed successively with water, dilute hydrochloric acid, and an aqueous, saturated solution of sodium chloride, then dried (MgSO₄) and concentrated under reduced pressure. The thus-obtained, residual oily substance was subjected to column chromatography on silica gel. From the fractions eluted with ethyl acetate-hexane (1:5), the title compound (2.9 g, 91%) was obtained as a light yellow, oily substance. ¹H NMR (δ ppm in CDCl₃): 1.11 (3H, t, J=7.0 Hz), 1.51 (9H, s), 1.8-2.2 (2H, m), 2.3-2.5 (1H, m), 2.6-2.9 (1H, m), 3.2-3.9 (5H, m), 4.99 (1H, d, J=3.2 Hz), 5.0-5.2 (2H, m), 7.1-7.5 (10H, m).

According to the same manner as that in Reference Example 19, compounds in Reference Examples 20 to 25 were obtained.

### REFERENCE EXAMPLE 20

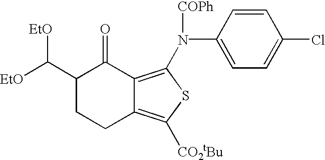

3-[[N-Benzoyl-N-(4-chlorophenyl)]amino]-5-diethoxymethyl-4,5,6,7-tetrahydro-4-oxobenzo[c]thiophene-1-carboxylic Acid t-butyl Ester:

Light yellow, amorphous solid (yield: 73%), elemental analysis: calculated [C, 63.74; H, 5.87; N, 2.40 (C₃₁H₃₄NO₆SCl)], observed [C, 63.65; H, 5.59; N, 2.40].

### REFERENCE EXAMPLE 21

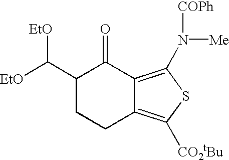

3-[(N-Benzoyl-N-methyl)amino]-5-diethoxymethyl-4,5,6,7-tetrahydro-4-oxobenzo[c]thiophene-1-carboxylic Acid t-butyl Ester:

Light yellow, oily substance (yield: 100%). ¹H NMR (δ ppm in CDCl₃): 1.13 (3H, t, J=7.0 Hz), 1.23 (3H, t, J=7.0 Hz), 1.54 (9H, s), 1.9-2.2 (2H, m), 2.4-2.6 (1H, m), 2.79 (1H, ddd, J=5.2, 10.4, 18.0 Hz), 3.41 (3H, s), 3.4-3.8 (5H, m), 5.02 (1H, d, J=3.4 Hz), 7.2-7.5 (5H, m).

### REFERENCE EXAMPLE 22

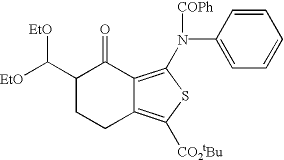

3-[(N-Benzoyl-N-phenyl)amino]-5-diethoxymethyl-4,5,6,7-tetrahydro-4-oxobenzo[c]thiophene-1-carboxylic Acid t-butyl Ester:

Light yellow, oily substance (yield: 93%). ¹H NMR (δ ppm in CDCl₃): 1.11 (3H, t, J=7.2 Hz), 1.21 (3H, t, J=7.2 Hz), 1.53 (9H, s), 2.0-2.3 (2H, m), 2.5-2.7 (1H, m), 2.90 (1H, ddd, J=5.8, 10.0, 18.0 Hz), 3.4-3.8 (5H, m), 5.04 (1H, d, J=3.4 Hz), 7.1-7.7 (10H, m).

### REFERENCE EXAMPLE 23

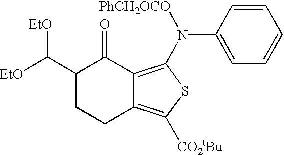

3-[(N-Benzyloxycarbonyl-N-phenyl)amino]-5-diethoxymethyl-4,5,6,7-tetrahydro-4-oxobenzo[c]thiophene-1-carboxylic Acid t-butyl Ester:

Light yellow, oily substance (yield: 92%). ¹H NMR (δ ppm in CDCl₃): 1.07 (3H, t, J=7.2 Hz), 1.21 (3H, t, J=7.2 Hz), 1.53 (9H, s), 2.1-2.4 (2H, m), 2.5-2.7 (1H, m), 2.8-3.0 (1H, m), 3.4-3.8 (5H, m), 5.06 (1H, d, J=3.4 Hz), 5.13 (1H, d, J=12.4 Hz), 5.26 (1H, d, J=12.4 Hz), 7.2-7.4 (10H, m).

### REFERENCE EXAMPLE 24

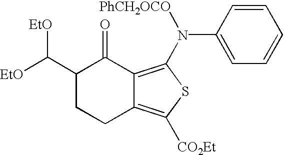

3-[(N-Benzyloxycarbonyl-N-phenyl)amino]-5-diethoxymethyl-4,5,6,7-tetrahydro-4-oxobenzo[c]thiophene-1-carboxylic Acid Ethyl Ester:

Light yellow, oily substance (yield: 86%). ¹H NMR (δ ppm in CDCl₃): 1.07 (3H, t, J=7.2 Hz), 1.23 (3H, t, J=7.2 Hz), 1.32 (3H, t, J=7.2 Hz), 2.0-2.4 (2H, m), 2.5-2.7 (1H, m), 2.92 (1H, ddd, J=5.4, 10.2, 18.0 Hz), 3.4-3.8 (5H, m), 5.05 (1H, d, J=3.4 Hz), 5.13 (1H, d, J=12.4 Hz), 5.26 (1H, d, J=12.4 Hz), 7.2-7.5 (10H, m).

### REFERENCE EXAMPLE 25

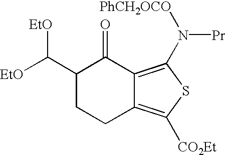

3-[(N-Benzyloxycarbonyl-N-propyl)amino]-5-diethoxymethyl-4,5,6,7-tetrahydro-4-oxobenzo[c]thiophene-1-carboxylic Acid Ethyl Ester:

Light yellow, oily substance (yield: 94%). ¹H NMR (δ ppm in CDCl₃): 0.89 (3H, t, J=7.4 Hz), 1.07 (3H, t, J=7.2 Hz), 1.23 (3H, t, J=7.2 Hz), 1.36 (3H, t, J=7.2 Hz), 1.5-1.7 (2H, m), 2.0-2.3 (2H, m), 2.5-2.7 (1H, m), 2.90 (1H, ddd, J=5.4, 10.6, 18.0 Hz), 3.4-3.8 (7H, m), 4.33 (2H, q, J=7.2 Hz), 5.04 (1H, d, J=3.2 Hz), 5.06 (1H, d, J=12.4 Hz), 5.17 (1H, d, J=12.4 Hz), 7.1-7.4 (5H, m).

### REFERENCE EXAMPLE 26

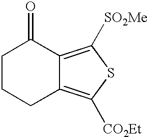

4,5,6,7-Tetrahydroxy-3-methylsulfonyl-4-oxobenzo[c]thiophene-1-carboxylic Acid Ethyl Ester:

To a solution of 4,5,6,7-tetrahydroxy-3-methylsulfanyl-4-oxobenzo[c]thiophene-1-carboxylic acid ethyl ester (5.0 g) in dichloromethane (150 ml) was added under ice cooling m-chloroperbenzoic acid (16.0 g) and the resulting solution was stirred at room temperature for 24 hours. The reaction solution was concentrated under reduced pressure and the residue was diluted with ethyl acetate. The precipitated crystals were collected by filtration, washed with an aqueous solution of sodium hydrogen carbonate and water and then further washed with ethyl acetate-hexane to obtain the title compound (5.5 g, 98%) as light yellow crystals. The thus-obtained crystals were recrystallized from ethyl acetate-hexane to obtain light yellow prisms. Melting point: 199-200° C.

### REFERENCE EXAMPLE 27

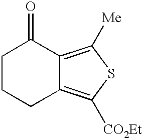

4,5,6,7-Tetrahydro-3-methyl-4-oxobenzo[c]thiophene-1-carboxylic Acid Ethyl Ester:

To a solution of 4,5,6,7-tetrahydro-3-methylsulfonyl-4-oxobenzo[c]thiophene-1-carboxylic acid ethyl ester (27.4 g) in anhydrous tetrahydrofuran (500 ml) was added dropwise a 3 M solution of methylmagnesium bromide in ether (31 ml). The resulting mixture was stirred at room temperature for 3 hours and then an aqueous solution of citric acid was added to the reaction solution. THF was concentrated under reduced pressure and the residue was extracted with ethyl acetate. The organic layer was washed with an aqueous, saturated solution of sodium chloride, then dried (MgSO₄), and concentrated under reduced pressure. The thus-obtained, oily substance was subjected to column chromatography on silica gel. From the fractions eluted with ethyl acetate-hexane (1:3), the title compound (5.9 g, 27%) was obtained as light yellow crystals. Recrystallization from ethyl acetate-hexane gave colorless crystals. Melting point: 76-77° C.

According to the same manner as that in Reference Example 27, compounds in Reference Examples 28 to 30 were obtained.

### REFERENCE EXAMPLE 28

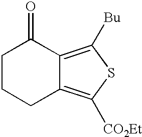

3-n-Butyl-4,5,6,7-tetrahydro-4-oxobenzo[c]thiophene-1-carboxylic Acid Ethyl Ester:

Colorless needles (yield: 30%). Melting point: 48-49° C. (recrystallization solvent: AcOEt-hexane)

### REFERENCE EXAMPLE 29

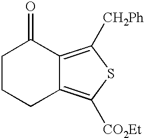

3-Benzyl-4,5,6,7-tetrahydro-4-oxobenzo[c]thiophene-1-carboxylic Acid Ethyl Ester:

Light yellow needles (yield: 21%). Melting point: 96-97° C. (recrystallization solvent: AcOEt-hexane).

### REFERENCE EXAMPLE 30

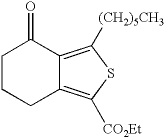

3-n-Hexyl-4,5,6,7-tetrahydro-4-oxobenzo[c]thiophene-1-carboxylic Acid Ethyl Ester:

Light yellow, oily substance (yield: 47%). ¹H NMR (δ ppm in CDCl₃): 0.89 (3H, t, J=6.4 Hz), 1.2-1.8 (8H, m), 1.38 (3H, t, J=7.2 Hz), 2.0-2.2 (2H, m), 2.54 (2H, t, J=6.4 Hz), 3.21 (2H, t, J=6.4 Hz), 3.25 (2H, t, J=6.8 Hz), 4.34 (2H, q, J=7.2 Hz).

According to the same manner as that in Reference Example 19, compounds in Reference Examples 31 to 36 were obtained.

### REFERENCE EXAMPLE 31

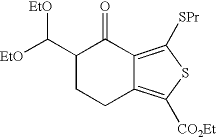

5-Diethoxymethyl-4,5,6,7-tetrahydro-4-oxo-3-n-propylsulfanylbenzo[c]thiophene-1-carboxylic Acid Ethyl Ester:

Light yellow, oily substance (yield: 97%). ¹H NMR (δ ppm in CDCl₃): 1.11 (3H, t, J=7.2 Hz), 1.14 (3H, t, J=7.2 Hz), 1.25 (3H, t, J=7.2 Hz), 1.37 (3H, t, J=7.2 Hz), 1.7-2.0 (2H, m), 2.1-2.4 (2H, m), 2.71 (1H, ddd, J=3.2, 5.0, 10.6 Hz), 2.88 (1H, ddd, J=5.7, 10.6, 18.0 Hz), 3.03 (2H, t, J=7.2 Hz), 3.5-3.9 (5H, m), 4.33 (2H, q, J=7.2 Hz), 5.15 (1H, d, J=3.2 Hz).

### REFERENCE EXAMPLE 32

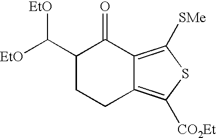

5-Diethoxymethyl-4,5,6,7-tetrahydro-3-methylsulfanyl-4-oxobenzo[c]thiophene-1-carboxylic Acid Ethyl Ester:

Light yellow, oily substance (yield: 93%). ¹H NMR (δ ppm in CDCl₃): 1.14 (3H, t, J=7.0 Hz), 1.25 (3H, t, J=7.0 Hz), 1.38 (3H, t, J=7.0 Hz), 2.0-2.4 (2H, m), 2.61 (3H, s), 2.72 (1H, ddd, J=3.0, 5.0, 10.4 Hz), 2.90 (1H, ddd, J=5.0, 10.4, 18.0 Hz), 3.5-3.9 (5H, m), 4.34 (2H, q, J=7.0 Hz), 5.15 (1H, d, J=3.0 Hz).

### REFERENCE EXAMPLE 33

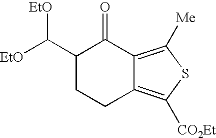

5-Diethoxymethyl-4,5,6,7-tetrahydro-3-methyl-4-oxobenzo[c]thiophene-1-carboxylic Acid Ethyl Ester:

Light yellow, oily substance (yield: 99%). ¹H NMR (δ ppm in CDCl₃): 1.15 (3H, t, J=7.0 Hz), 1.24 (3H, t, J=7.0 Hz), 1.37 (3H, t, J=7.0 Hz), 2.0-2.4 (2H, m), 2.70 (1H, ddd, J=3.6, 4.8, 10.6 Hz), 2.79 (3H, s), 2.90 (1H, ddd, J=4.8, 10.6, 17.8 Hz), 3.5-3.9 (5H, m), 4.33 (2H, q, J=7.0 Hz), 5.09 (1H, d, J=3.6 Hz).

### REFERENCE EXAMPLE 34

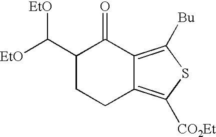

3-n-Butyl-5-diethoxymethyl-4,5,6,7-tetrahydro-4-oxobenzo[c]thiophene-1-carboxylic Acid Ethyl Ester:

Light yellow, oily substance (yield: 100%). ¹H NMR (δ ppm in CDCl₃): 0.95 (3H, t, J=7.2 Hz), 1.15 (3H, t, J=7.2 Hz), 1.37 (3H, t, J=7.2 Hz), 1.3-1.8 (4H, m), 2.0-2.4 (2H, m), 2.70 (1H, ddd, J=4.0, 4.8, 10.4 Hz), 2.91 (1H, ddd, J=4.8, 10.4, 18.0 Hz), 3.24 (2H, dd, J=7.0, 8.2 Hz), 3.5-3.9 (5H, m), 4.33 (2H, q, J=7.2 Hz), 5.10 (1H, d, J=3.6 Hz).

### REFERENCE EXAMPLE 35

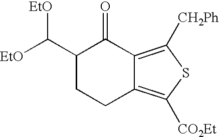

3-Benzyl-5-diethoxymethyl-4,5,6,7-tetrahydro-4-oxobenzo[c]thiophene-1-carboxylic Acid Ethyl Ester:

Light yellow, oily substance (yield: 100%). ¹H NMR (δ ppm in CDCl₃): 1.14 (3H, t, J=7.4 Hz), 1.25 (3H, t, J=7.4 Hz), 1.28 (3H, t, J=7.4 Hz), 2.0-2.4 (2H, m), 2.74 (1H, dt, J=4.4, 9.8 Hz), 2.92 (1H, ddd, J=5.6, 10.4, 18.0 Hz), 3.5-3.9 (5H, m), 4.28 (2H, q, J=7.4 Hz), 4.59 (2H, s), 5.11 (1H, d, J=3.6 Hz), 7.2-7.4 (5H, m).

### REFERENCE EXAMPLE 36

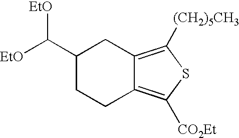

5-Diethoxymethyl-3-n-hexyl-4,5,6,7-tetrahydro-4-oxobenzo[c]thiophene-1-carboxylic Acid Ethyl Ester:

Light yellow, oily substance (yield: 91%). ¹H NMR (δ ppm in CDCl₃): 0.88 (3H, t, J=6.6 Hz), 1.14 (3H, t, J=6.4 Hz), 1.24 (3H, t, J=7.2 Hz), 1.37 (3H, t, J=7.2 Hz), 1.3-1.8 (6H, m), 2.0-2.3 (2H, m), 2.6-2.7 (1H, m), 2.90 (1H, ddd, J=5.6, 10.4, 18.0 Hz), 3.23 (2H, t, J=7.6 Hz), 3.5-3.9 (6H, m), 4.1-4.3 (1H, m), 4.33 (2H, q, J=7.2 Hz), 5.10 (1H, d, J=3.2 Hz).

### REFERENCE EXAMPLE 37

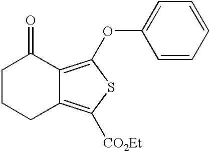

4,5,6,7-Tetrahydro-4-oxo-3-phenoxybenzo[c]thiophene-1-carboxylic Acid Ethyl Ester:

To a solution of 4,5,6,7-tetrahydro-3-methylsulfonyl-4-oxobenzo[c]thiophene-1-carboxylic acid ethyl ester (6.0 g) and phenol (2.2 g) in tetrahydrofuran (50 ml) was added sodium hydride (1.0 g) and the resulting mixture was stirred at room temperature for 14 hours. After an aqueous solution of citric acid was added to the reaction solution, the resulting mixture was concentrated under reduced pressure and the residual oily substance was subjected to addition of water and extraction with ethyl acetate. The organic layer was dried (MgSO₄) and concentrated under reduced pressure to remove the solvent to obtain crude crystals. Recrystallization from ethyl acetate-diisopropyl ether gave the title compound (5.0 g, 80%) as colorless prisms. Melting point: 125-127° C.

According to the same manner as that in Reference Example 37, compounds in Reference Examples 38 to 56 were synthesized.

TABLE 2

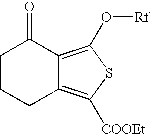

| Reference | | Melting |
|---|---|---|
| Example No. | —Rf | point (° C.) |
| 38 | 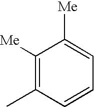 | 62-64 |
| 39 | 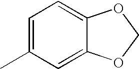 | 150-152 |
| 40 | 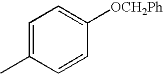 | 133-134 |
| 41 | 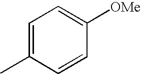 | 103-104 |
| 42 | 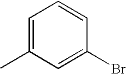 | 104-105 |
| 43 | 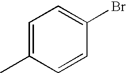 | 109-110 |
| 44 | 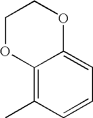 | 152-153 |
| 45 | 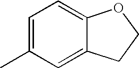 | 144-146 |
| 46 | 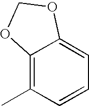 | 149-151 |
| 47 | 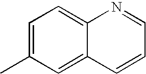 | 120-121 |

TABLE 3

| | | |
|---|---|---|
| 48 | 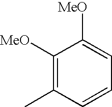 | Oily substance¹⁾ |
| 49 | 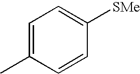 | 108-110 |
| 50 | 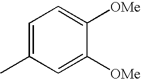 | 152-153 |
| 51 | 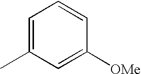 | 107-108 |
| 52 | 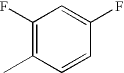 | 112-113 |
| 53 | 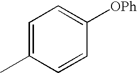 | 84-85 |
| 54 | 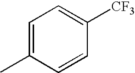 | 111-112 |
| 55 | 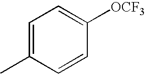 | Oily substance²⁾ |
| 56 | 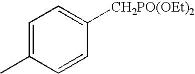 | 141-142 |

¹⁾¹H NMR(δ ppm in CDCl₃): 1.28(3H, t, J=7.0Hz), 2.00-2.20(2H, m), 2.57(2H, t, J=6.4 Hz), 3.22(2H, t, J=6.4Hz), 3.88(3H, s), 3.91(3H, s), 4.24(2H, q, J=7.0Hz), 6.80-7.20(3H, m).
²⁾¹H NMR(δ ppm in CDCl₃): 1.32(3H, t, J=7.0Hz), 2.00-2.20(2H, m), 2.57(2H, t, J=6.2 Hz), 3.23(2H, t, J=6.2Hz), 4.28(2H, q, J=7.0Hz), 6.82(2H, d, J=9.0Hz), 7.09(2H, d, J=9.0Hz).

### REFERENCE EXAMPLE 57

5-Diethoxymethyl-4,5,6,7-tetrahydro-4-oxo-3-phenoxybenzo[c]thiophene-1-carboxylic Acid Ethyl Ester:

According to the same manner as that in Reference Example 19, the title compound was obtained as colorless prisms from the compound obtained in Reference Examples 37. Melting point: 73-74° C. (recrystallization solvent: AcOEt-hexane).

According to the same manner as that in Reference Example 57, compounds in Reference Examples 58 to 76 were synthesized.

TABLE 4

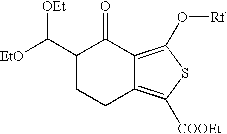

| Reference | | Melting |
|---|---|---|
| Example No. | —Rf | point (° C.) |
| 58 | 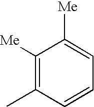 | 110-112 |
| 59 | 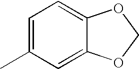 | 87-89 |
| 60 | 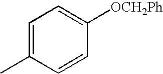 | 122-124 |
| 61 | 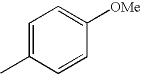 | Oily substance³⁾ |
| 62 | 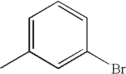 | 95-96 |
| 63 | 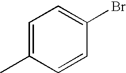 | Oily substance⁴⁾ |
| 64 | 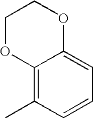 | Oily substance⁵⁾ |
| 65 | 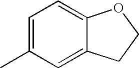 | 108-109 |
| 66 | 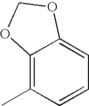 | Oily substance⁶⁾ |
| 67 | 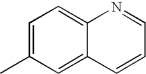 | 100-102 |

³⁾¹H NMR(δ ppm in CDCl₃): 1.26(3H, t, J=7.0Hz), 1.29(6H, t, J=7.0Hz), 2.18-2.34(2H, m), 2.66-2.80(1H, m), 2.80-2.99(1H, m), 3.51-3.88(5H, m), 3.83(3H, s), 4.25(2H, q, J=7.0Hz), 5.15(1H, d, J=3.2Hz), 6.91-6.96(2H, m), 7.17-7.22(2H, m).
⁴⁾¹H NMR(δ ppm in CDCl₃): 1.23(3H, t, J=7.0Hz), 1.25(3H, t, J=7.0Hz), 1.26(3H, t, J=7.0Hz), 2.15-2.39(2H, m), 2.65(1H, m), 2.81-2.99(1H, m), 3.53-3.78(4H, m), 4.27(2H, q, J=7.0Hz), 5.12(1H, d, J=3.4Hz), 7.12(2H, dt, J=9.2, 2.2Hz), 7.55(2H, dt, J=9.2, 2.2Hz).
⁵⁾¹H NMR(δ ppm in CDCl₃): 1.17(3H, t, J=7.0Hz), 2.08-2.30(2H, m), 2.66-2.96(2H, m), 3.54-3.84(5H, m), 4.25(2H, q, J=7.0Hz), 4.27(4H, t, J=4.8 Hz), 5.16(1H, d, J=3.2Hz), 6.81-6.88(3H, m).
⁶⁾¹H NMR(δ ppm in CDCl₃): 1.31(3H, t, J=7.0Hz), 1.25(6H, t, J=7.0Hz), 2.16-2.33(2H, m), 2.66-2.33(1H, m), 2.75-2.98(1H, m), 3.54-3.83(5H, m), 4.27(2H, q, J=7.0Hz), 5.14(1H, d, J=3.4 Hz), 6.01(2H, s), 6.76-7.25(3H, m).

TABLE 5

| | | |
|---|---|---|
| 68 | 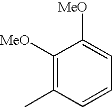 | Oily substance⁷⁾ |
| 69 | 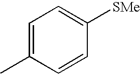 | 75-77 |
| 70 | 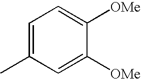 | 83-84 |
| 71 | 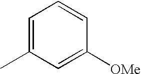 | Oily substance⁸⁾ |
| 72 | 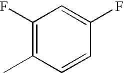 | Oily substance⁹⁾ |
| 73 | 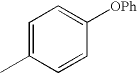 | Oily substance¹⁰⁾ |
| 74 | 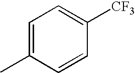 | 111-112 |
| 75 | 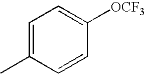 | 68-69 |
| 76 | 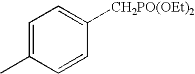 | Oily substance¹¹⁾ |

⁷⁾¹H NMR(δ ppm in CDCl₃): 1.22(3H, t, J=7.0Hz), 1.26(3H, t, J=7.0Hz), 1.28(3H, t, J=7.0Hz), 2.00-2.40(2H, m), 2.60-3.00(2H, m), 3.40-3.90(5H, m), 3.87(3H, s), 3.90(3H, s), 4.23(2H, q, J=7.0Hz), 5.16(1H, d, J=3.6Hz), 6.80-7.20(3H, m).
⁸⁾¹H NMR(δ ppm in CDCl₃): 1.67(3H, t, J=7.2Hz), 1.26(3H, t, J=7.2Hz), 1.30(3H, t, J=7.2Hz), 2.00-2.40(2H, m), 2.71(1H, ddd, J=10.6, 4.8, 3.4Hz), 2.90(1H, ddd, J=18.0, 10.6, 5.2Hz), 3.50-3.90(5H, m), 3.81(3H, s), 4.26(2H, q, J=7.2Hz), 5.14(1H, d, J=3.4Hz), 6.70-6.90(3H, m), 7.33(1H, t, J=8.0Hz).
⁹⁾¹H NMR(δ ppm in CDCl₃): 1.16(3H, t, J=7.2Hz), 1.25(3H, t, J=7.2Hz), 1.31(3H, t, J=7.2Hz), 2.10-2.40(2H, m), 2.60-2.80(1H, m), 2.90(1H, ddd, J=17.4, 10.0, 5.6Hz), 3.50-3.90(5H, m), 4.27(2H, q, J=7.2 Hz), 5.13(1H, d, J=3.2Hz), 6.90-7.10(2H, m), 7.20-7.40(1H m).
¹⁰⁾¹H NMR(δ ppm in CDCl₃): 1.17(3H, t, J=7.0Hz), 1.26(3H, t, J=7.0Hz), 1.32(3H, t, J=7.0Hz), 2.00-2.40(2H, m), 2.60-2.80(1H, m), 2.90(1H, ddd, J=18.2, 10.8, 5.2Hz), 3.50-3.90(5H, m), 4.27(2H, q, J=7.0Hz), 5.15(1H, d, J=3.4Hz), 7.00-7.50(9H, m).
¹¹⁾¹H NMR(δ ppm in CDCl₃): 1.17(3H, t, J=7.0Hz), 1.25(6H, t, J=7.0Hz), 1.26(6H, t, J=7.0Hz), 2.00-2.40(2H, m), 3.50-4.10(9H, m), 4.25(2H, q, J=7.0Hz), 5.14(1H, d, J=3.4Hz), 7.21(2H, d, J=8.4Hz), 7.39(2H, dd, J=8.4, 2.4Hz).

### REFERENCE EXAMPLE 77

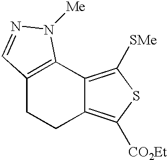

4,5,-Dihydro-1-methyl-8-methylsulfanyl-1H-thieno[3,4-g]indazole-6-carboxylic Acid Ethyl Ester:

A solution of 5-diethoxymethyl-3-methylsulfanyl-4-oxo-4,5,6,7-tetrahydrobenzo[c]thiophene-1-carboxylic acid ethyl ester (21.5 g), methylhydrazine monohydrate (6.87 g), 2 N hydrochloric acid (100 ml), and ethanol (200 ml) was heated at reflux for 2 hours. The reaction solution was concentrated under reduced pressure and the residual oily substance was poured into water and extracted with ethyl acetate. The organic layer was dried (MgSO₄) and then the solvent was evaporated under reduced pressure. The thus-obtained, oily substance was subjected to column chromatography on silica gel. From the fractions eluted with ethyl acetate-hexane (1:4), the title compound (15.25 g, 89%) was obtained as yellow needles. Melting point: 113-114° C.

### REFERENCE EXAMPLE 78

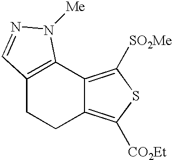

4,5,-Dihydro-1-methyl-8-methylsulfonyl-1H-thieno[3,4-g]indazole-6-carboxylic Acid Ethyl Ester:

A solution of 4,5,-dihydro-1-methyl-8-methylsulfanyl-1H-thieno[3,4-g]indazole-6-carboxylic acid ethyl ester (5.4 g) and m-chloroperbenzoic acid (15 g) in dichloromethane (120 ml) was stirred at room temperature for 2 days. The reaction solution was concentrated under reduced pressure, the precipitated crystals were collected by filtration, and then the thus-obtained crystals were washed with an aqueous solution of sodium hydrogen carbonate and ethyl acetate-hexane to obtain the title compound (1.3 g, 22%) as yellow needles Melting point: 140-141° C.

### REFERENCE EXAMPLE 79

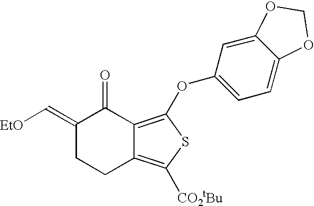

5-[(E)-ethoxymethylidene]-4,5,6,7-tetrahydro-3-(3,4-methylenedioxyphenoxy)-4-oxobenzo[c]thiophene-1-carboxylic acid t-butyl Ester:

To a solution of 5-diethoxymethyl-4,5,6,7-tetrahydro-3-methylsulfonyl-4-oxobenzo[c]thiophene-1-carboxylic acid t-butyl ester (5.55 g) in THF (100 ml) was added potassium t-butoxide (1.58 g), the resulting mixture was stirred at room temperature for 1 hour, and then the reaction solution was poured into water and extracted with ethyl acetate. The extract was washed with water, dried (MgSO₄), and then concentrated under reduced pressure. Then, the thus-obtained crystals were added to a solution of sesamol (0.95 g) and potassium t-butoxide (0.77 g) in THF (100 ml), the resulting mixture was stirred at room temperature for 1 hour, then poured into water, and extracted with ethyl acetate. The organic layer was washed with water and an aqueous, saturated solution of sodium chloride, dried (MgSO₄) and then concentrated under reduced pressure. The thus-obtained crystals were recrystallized from ethyl acetate-isopropyl ether to give the title compound. ¹H NMR (δ ppm in CDCl₃): 1.36 (3H, t, J=7.0 Hz), 1.52 (9H, s), 2.67 (2H, t, J=7.0 Hz), 3.17 (2H, t, J=7.0 Hz), 4.13 (2H, q, J=7.0 Hz), 6.03 (2H, s), 6.70-6.83 (3H, m), 7.51 (1H, s).

### REFERENCE EXAMPLE 80

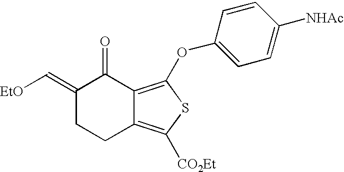

3-[4-(Acetylamino)phenoxy]-5-[(E)-ethoxymethylidene]-4,5,6,7-tetrahydro-4-oxobenzo[c]thiophene-1-carboxylic Acid Ethyl Ester:

After a solution of N-acetyl-4-hydroxyaniline (1.5 g) and potassium t-butoxide (1.17 g) in THF (100 ml) was stirred at room temperature for 1 hour, thereto was added 5-[(E)-diethoxymethylidene]-4,5,6,7-tetrahydro-8-methanesulfonyl-4-oxobenzo[c]thiophene-1-carboxylic acid ethyl ester (4.04 g). The reaction mixture was stirred at room temperature for 15 hour, then poured into water, and extracted with a THF-ethyl acetate mixed solution. The organic layer was washed with water, sodium hydrogen carbonate, and an aqueous, saturated solution of sodium chloride, dried (MgSO₄), and then concentrated under reduced pressure. The precipitated crystals were washed with ethanol to give the title compound (2.48 g, 58%) as colorless prisms. Melting point: 230° C. (decomposition).

According to the same manner as that in Reference Example 80, compounds in Reference Examples 81 to 83 were obtained.

### REFERENCE EXAMPLE 81

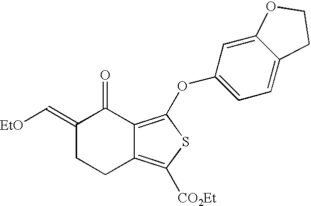

3-(2,3-Dihydrobenzofuran-6-yloxy)-5-[(E)-ethoxy-methylidene]-4,5,6,7-tetrahydro-4-oxobenzo[c]thiophene-1-carboxylic Acid Ethyl Ester:

Yellow prisms (yield: 78%). Melting point: 166-167° C. (recrystallization solvent: ethanol).

### REFERENCE EXAMPLE 82

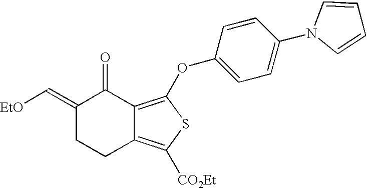

5-[(E)-Ethoxymethylidene]-4,5,6,7-tetrahydro-4-oxo-3-[4-(1H-pyrrol-6-yl)phenoxy]benzo[c]thiophene-1-carboxylic Acid Ethyl Ester:

Yellow prisms (yield: 85%). Melting point: 192-193° C. (recrystallization solvent: ethanol).

According to the same manner as that in Reference Example 80, compounds in Reference Examples 83 to 84 were synthesized.

### REFERENCE EXAMPLE 83

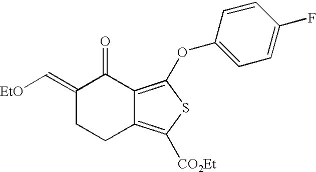

5-[(E)-Ethoxymethylidene]-3-(4-fluorophenoxy)-4,5,6,7-tetrahydro-4-oxobenzo[c]thiophene-1-carboxylic Acid Ethyl Ester:

Yellow prisms (yield: 65%). Melting point: 144-146° C. (recrystallization solvent: AcOEt-hexane).

### REFERENCE EXAMPLE 84

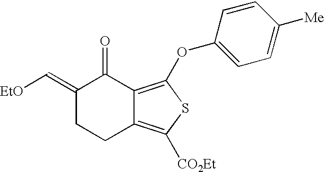

5-[(E)-Ethoxymethylidene]-4,5,6,7-tetrahydro-3-(4-methylphenoxy)-4-oxobenzo[c]thiophene-1-carboxylic Acid Ethyl Ester:

Yellow prisms (yield: 72%). Melting point: 190-191° C. (recrystallization solvent: AcOEt-hexane).

### REFERENCE EXAMPLE 85

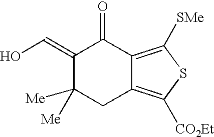

6,6-Dimethyl-4,5,6,7-tetrahydro-5-[(E)-hydroxymethylidene]-3-(methylsulfanyl)-4-oxobenzo[c]thiophene-1-carboxylic Acid Ethyl Ester:

To a suspension of sodium hydride (0.27 g) in THF (30 ml) was added ethyl formate (1.24 g) under ice cooling. The resulting mixture was stirred for 10 minutes, and then a solution of 4,5,6,7-tetrahydro-6,6-dimethyl-3-methylsulfanyl)-4-oxobenzo[c]thiophene (1.0 g) in THF (10 ml) was added dropwise over a period of 10 minutes. The temperature was returned to the room temperature and the resulting solution was stirred for 18 hours. The reaction solution was poured into an aqueous solution of citric acid and extracted with ethyl acetate. The organic layer was washed with water and an aqueous, saturated solution of sodium chloride, dried (MgSO₄) and then evaporated to remove the solvent. The thus-obtained, crude crystals were recrystallized from ethyl acetate-hexane to give the title compound (0.65 g, 60%). ¹H NMR (δ ppm in CDCl₃): 1.20 (6H, S), 1.39 (3H, t, J=7.0 Hz), 2.66 (3H, s), 3.10 (2H, s), 4.34 (2H, q, J=7.0 Hz), 7.56 (1H, d, J=10.6 Hz).

### REFERENCE EXAMPLE 86

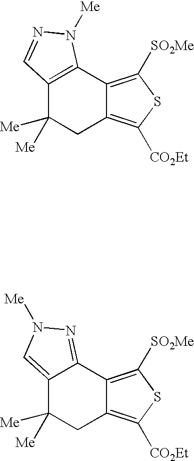

4,5-Dihydro-(8-methylsulfonyl)-1,4,4-trimethyl-1H-thieno[3,4-g]indazole-6-carboxylic Acid Ethyl Ester and 4,5-dihydro-(8-methylsulfonyl)-2,4,4-trimethyl-2H-thieno[3,4-g]indazole-6-carboxylic Acid Ethyl Ester:

A mixture of 6,6-dimetyl-4,5,6,7-tetrahydro-5-[(E)-hydroxymethylidene]-3-methanesulfanyl)-4-oxobenzo[c]thiophene-1-carboxylic acid ethyl ester (7.50 g), methylhydrazine sulfate (4.97 g), ethanol (100 ml), and water (20 ml) was stirred at 70° C. for 2 hours. The reaction solution was concentrated and then extracted with ethyl acetate. The organic layer was washed with sodium hydrogen carbonate and an aqueous, saturated solution of sodium chloride, dried (MgSO₄), and then evaporated to remove the solvent to obtain a mixture (7.25 g, 94%) of 4,5-dihydro-(8-methylsulfanyl)-1,4,4-trimethyl-1H-thieno[3,4-g]indazole-6-carboxylic acid ethyl ester and 4,5-dihydro-(8-methylsulfanyl)-2,4,4-trimethyl-2H-thieno[3,4-g]indazole-6-carboxylic acid ethyl ester. This mixture (7.13 g) was dissolved in THF (200 ml), treated with m-chloroperbenzoic acid (12 g), and stirred at room temperature for 12 hours. The reaction solution was concentrated and poured into an aqueous solution of sodium hydrogen carbonate. After extraction with ethyl acetate, the organic layer was washed with an aqueous, saturated solution of sodium chloride, dried (MgSO₄), then evaporated to remove the solvent, and the residual crude crystals were subjected to chromatography on silica gel. From the fractions eluted with ethyl acetate-hexane (AcOEt:hexane=1:2), the title compounds were obtained.

4,5-Dihydro-(8-methylsulfonyl)-1,4,4-trimethyl-1H-thieno[3,4-g]indazole-6-carboxylic acid ethyl ester: colorless prisms (5.77 g, 74%), melting point: 129-131° C.

4,5-Dihydro-(8-methylsulfonyl)-2,4,4-trimethyl-2H-thieno[3,4-g]indazole-6-carboxylic acid ethyl ester: light yellow needles (1.82 g, 23%), melting point: 208-210° C. (recrystallization solvent: AcOEt).

Accordieng to the same manner as that in

Reference Example 37, compounds in Reference Examples 87 to 88 were synthesized.

TABLE 6

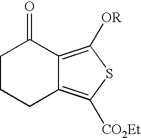

| Reference | | |
|---|---|---|
| Example No. | R | Melting point (° C.) |
| 87 | 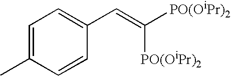 | 125-126 |
| 88 | 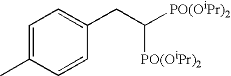 | Oily substance¹⁾ |

¹⁾¹H NMR(δ ppm in CDCl₃): 1.2-1.4(27H, m), 2.0-2.2(2H, m), 2.4-2.7(3H, m), 3.1-3.4(4H, m), 4.25(2H, q, J=7.0Hz), 4.7-4.9(4H, m), 7.17(2H, d, J=8.8Hz), 7.37(2H, d, J=8.8).

According to the same manner as that in Reference Example 19, compounds in Reference Examples 89 to 90 were

TABLE 7

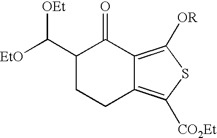

| Reference | | |
|---|---|---|
| Example No. | R | Melting point (° C.) |
| 89 | 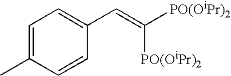 | Oily substance¹⁾ |
| 90 | 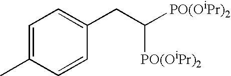 | Oily substance²⁾ |

¹⁾¹H NMR(δ ppm in CDCl₃): 1.22(6H, d, J=6.0Hz), 1.25(6H, d, J=6.0Hz), 1.37(6H, d, J=6.0Hz), 1.40(6H, d, J=6.0Hz), 1.2-1.4(9H, m), 2.0-2.4(2H, m), 2.6-3.0(2H, m), 3.5-3.9(5H, m), 4.26(2H, q, J=7.0Hz), 4.6-4.9(4H, m), 5.11(1H, d, J=3.8Hz), 7.23(2H, d, J=8.0Hz), 7.95(2H, d, J=8.0 Hz), 8.25(1H, dd, J=10.0, 47.2Hz).
²⁾¹H NMR(δ ppm in CDCl₃): 1.2-1.5(33H, m), 2.1-3.9(12H, m), 4.24(2H, q, J=7.0Hz), 4.6-4.9(4H, m), 5.15(1H, d, J=3.4 Hz), 7.16(2H, d, J=8.4Hz), 7.36(2H, d, J=8.4Hz).

According to the same manner as that in Reference Example 79, compounds in Reference Examples 91 to 92 were obtained.

TABLE 8

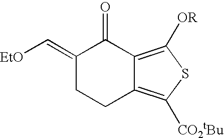

| Reference | | Melting |
|---|---|---|
| Example No. | R | point (° C.) |
| 91 | 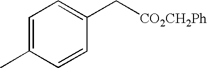 | 127-128 |
| 92 | 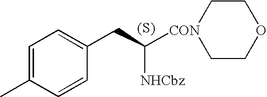 | 95-96 |

### REFERENCE EXAMPLE 93

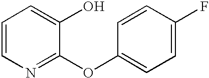

2-(4-Fluorophenoxy)-3-pyridinol

To a solution of 2-(4-fluorophenoxy)-3-aminopyridine (20 g) dissolved in 6N hydrochloric acid (80 ml) was added a solution of sodium nitrate (7.59 g) dissolved in water (28 ml) under ice-cooling. After stirring for 30 minutes, 60% hexafluorophosphin (16 ml) was added dropwise and the mixture was stirred for additional 15 minutes. Water (15 ml) was added and the precipitated crystals were filtered off. These crystals were added little by little to acetic anhydride (100 ml) at 100° C. and the mixture was stirred for 1 hour. The reaction mixture was concentrated under reduced pressure. The residue was neutralized with saturaded sodium bicarbonate and extracted with ethyl acetate. The organic layer was washed with saturated saline and dried (MgSO₄). The solvent was distilled off and the residue was subjected to silica gel chromatography. From the fraction eluted with ethyl acetate-hexane (1:5), 3-acetoxy-2-(4-fluorophenoxy)pyridine (17.4 g) was obtained as a coloress oily substance. This was dissolved in methanol (100 ml) and 1N aqueous sodium hydroxide solution (100 ml) was added dropwise thereto. The mixture was stirred at room temperature for 30 minutes and the reaction mixture was concentrated under redueced pressure. After neutralizing with 6N hydrochloric acid, the mixture was extracted with ethyl acetate and the organic layer was washed with saturated saline and dried (MgSO₄). The solvent was removed under reduced pressure to obtain the title compound (15.4 g, 75%) as colorless crystals. ¹H-NMR (CDCl₃) δ: 5.62 (1H, s), 6.95 (1H, dd, J=7.8, 5.0 Hz), 7.04-7.18 (4H, m), 7.28 (1H, dd, J=7.8, 1.4 Hz).

According to the same manner as that in Reference Example 93, the following title compounds were obtained.

### REFERENCE EXAMPLE 94

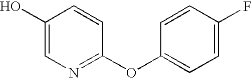

2-(4-Fluorophenoxy)-5-pyridinol

¹H-NMR (CDCl₃) δ: 6.34 '1H, brs.), 6.80 (1H, d, J=8.6 Hz), 7.02-7.06 (4H, m), 7.25 (1H, dd, J=8.8, 3.0 Hz), 7.78 (1H, d, J=2.8 Hz).

### REFERENCE EXAMPLE 95

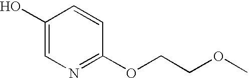

6-(2-Methoxyethoxy)-3-pyridinol

¹H-NMR (CDCl₃) δ: 3.45 (3H, s), 3.72-3.77 (2H, m), 4.36-4.41 (2H, m), 6.68 (1H, d, J=8.8 Hz), 7.16 (1H, dd, J=8.8, 3.0 Hz), 7.72 (1H, d, J=2.8 Hz).

### REFERENCE EXAMPLE 96

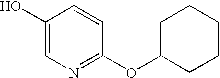

6-(Cyclohexyl)-3-pyridinol

¹H-NMR (CDCl₃) δ: 1.20-2.15 (10H, m), 4,75-4,85 (1H, m), 6.62 (1H, d, J=8.8 Hz), 7.18 (1H, dd, J=8.8, 3.2 hz), 7.76 (1H, d, J=3.0 Hz).

### REFERENCE EXAMPLE 97

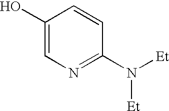

2-Dietheylamino-5-hydroxypyridine

¹H-NMR (CDCl₃) δ: 1.16 (6H, t, J=7.2 Hz), 3.46 (2H, q, J=7.2 Hz), 6.45 (1H, d, J=9.2 Hz), 7.15 (1H, dd, J=3.0 Hz), 7.85 (1H, d, J=2.8 Hz).

### REFERENCE EXAMPLE 98

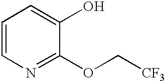

3-Hydroxy-2-(2,2,2-trifluoroethyl)pyridine

To a solution of 3-benzyloxy-2-chloropyridine (4.55 g) and 2,2,2-trifluoroethanol (2.69 g) in DMF (30 ml) was added sodium hydride (0.99 g) under ice-cooling and the mixture was stirred at 90° C. for 14 hours. The reaction mixture was concentrated, poured in water and extracted with ethyl acetate. The organic layer was washed with saturated aqueous sodium bicarbonate solution and saturated saline successively and dried (MgSO₄). The solvent was removed to obtain 3-benzyloxy-2-(2,2,2-trifluoroethyl)pyridine. This was dissolved in ethanol (200 ml) and 10% palladium-carbon (0.2 g) was added thereto. The mixture was stirred under a hydrogen atmosphere for 2 hours. Insoluble materials were filtered off and the filtrate was concentrated under reduced pressure to obtain the title compound (2.57 g, 64%) as a colorless oily substance. ¹H-NMR (CDCl₃) δ: 4.83 (2H, g, J=8.4 Hz), 5.60 (1H, brs), 6.92 (1H, dd, J=7.2, 4.8 Hz), 7.21 (1H, dd, J=7.2, 1.6 Hz), 7.69 (1H, dd, J=4.8, 1.6 Hz).

According to the same manner as that in Reference Example 98, the following title compounes were obtained.

### REFERENCE EXAMPLE 99

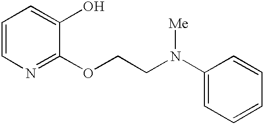

2-{2-[Methyl(phenyl)amino]ethoxy}pyridin-3-ol

¹H-NMR (CDCl₃) δ: 2.99 (3H, s), 3.77 (2H, t, J=5.6 Hz), 4.56 (2H, t, J=5.6 Hz), 6.17-6.89 (4H, m), 7.08 (1H, dd, J=7.8, 1.4 Hz), 7.21-7.30 (2H, m), 7.65 (1H, dd, J=4.8, 1.4 Hz).

### REFERENCE EXAMPLE 100

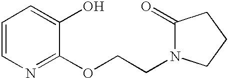

1-{2-[(3-Hydroxypyridin-2-yl)oxy]ethyl}pyrrolidine-2-one

¹H-NMR (CDCl₃) δ: 2.05 (2H, tt, J=8.0, 7.0 Hz), 2.41 (2H, t, J=8.0 Hz), 3.50 (2H, t, J=7.0 Hz), 3.71 (2H, t, J=5.2 Hz), 4.51 (2H, t, J=5.2 Hz), 6.82 (1H, dd, J=7.8, 5.2 Hz), 7.14 (1H, dd, J=7.8, 1.4 Hz), 7.65 (1H, dd, J=5.21, 1.4 Hz).

### EXAMPLE 1

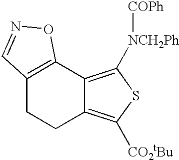

8-[(N-Benzoyl-N-benzyl)amino]-4,5-dihydrothieno[3,4-g]-1,2-benzisoxazole-6-carboxylic Acid t-butyl Ester:

A solution of 3-[(N-benzoyl-N-benzyl)amino]-5-diethoxymethyl-4,5,6,7-tetrahydro-4-oxobenzo[c]thiophene-1-carboxylic acid t-butyl ester (2.8 g), hydroxylamine hydrochloride (1.8 g), and sodium acetate (2.1 g) dissolved in ethanol (60 ml) was stirred at 80° C. for 15 hours. The reaction solution was concentrated under reduced pressure and the residue was diluted with ethyl acetate. The organic layer was washed with an aqueous, saturated solution of sodium chloride, dried (MgSO₄), and then concentrated under reduced pressure. The thus-obtained residual oily substance was dissolved in tetrahydrofuran (50 ml) and 4 N hydrochloric acid-ethyl acetate (5 ml) was added. After stirring at 80° C. for 2 hours, the reaction solution was poured into an aqueous solution of sodium hydrogen carbonate and extracted with ethyl acetate. The organic layer was washed with an aqueous, saturated solution of sodium chloride, then dried (MgSO₄), and concentrated under reduced pressure. The residual oily substance was subjected to chromatography on silica gel. From the fractions eluted with ethyl acetate-hexane (1:2), the title compound (0.60 g, 25%) was obtained as a yellow oily substance.

¹H NMR (δ ppm in CDCl₃): 1.54 (9H, s), 2.4-2.6 (2H, m), 3.0-3.2 (2H, m), 5.1-5.3 (2H, m), 7.1-7.5 (10H, m), 8.05 (1H, s).

According to the same manner as that in Example 1, compounds in Examples 2 to 4 were obtained.

### EXAMPLE 2

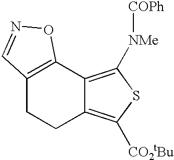

8-[(N-Benzoyl-N-methyl)amino]-4,5-dihydrothieno[3,4-g]-1,2-benzisoxazole-6-carboxylic Acid t-butyl Ester:

A yellow oily substance (yield: 57%), ¹H NMR (δ ppm in CDCl₃): 1.56 (9H, s), 2.64 (2H, t, J=7.6 Hz), 3.24 (2H, t, J=7.6 Hz), 3.53 (3H, s), 7.1-7.5 (5H, m), 8.15 (1H, s).

### EXAMPLE 3

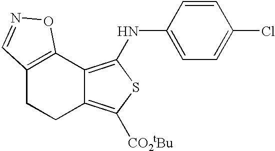

8-(4-Chlorophenylamino)-4,5-dihydrothieno[3,4-g]-1,2-benzisoxazole-6-carboxylic Acid t-butyl Ester:

Light brown crystals (yield: 65%), melting point: 197-198° C. (recrystallization solvent: AcOEt-hexane)

### EXAMPLE 4

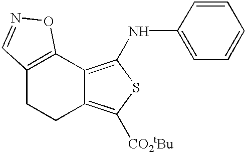

4,5-Dihydro-8-phenylaminothieno[3,4-g]-1,2-benzisoxazole-6-carboxylic Acid t-butyl Ester:

Colorless needles (yield: 62%), melting point: 148-149° C. (recrystallization solvent: AcOEt-hexane)

### EXAMPLE 5

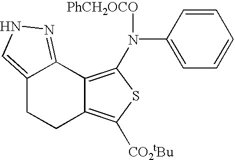

8-[(N-Benzyloxycarbonyl-N-phenyl)amino]-4,5-dihydro-1H-thieno[3,4-g]indazole-6-carboxylic Acid t-butyl Ester:

A solution of 3-[(N-benzyloxycarbonyl-N-phenyl)amino]-5-diethoxymethyl-4,5,6,7-tetrahydro-4-oxobenzo[c]thiophene-1-carboxylic acid t-butyl ester (3.0 g), hydrazine monohydrate (1.5 ml), and 1 N hydrochloric acid (8.0 ml) dissolved in ethanol (50 ml) was stirred under reflux for 15 hours. The reaction solution was concentrated under reduced pressure and the residue was dissolved in ethyl acetate. The organic layer was washed with water and an aqueous, saturated solution of sodium chloride, and dried (MgSO₄). The solvent was evaporated under reduced pressure and the thus-obtained, yellow oily substance was subjected to chromatography on silica gel. From the fractions eluted with ethyl acetate-hexane (1:2), the title compound (2.5 g, 97%) was obtained as light yellow crystals. Recrystallization from ether-hexane gave colorless prisms. Melting point: 183-184° C.

### EXAMPLE 6

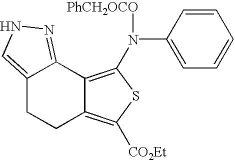

8-[(N-Benzyloxycarbonyl-N-phenyl)amino]-4,5-dihydro-1H-thieno[3,4-g]indazole-6-carboxylic Acid Ethyl Ester:

According to the same manner as that in Example 5, the title compound was obtained from the compound obtained from Reference Example 24. Light pink prisms (yield: 92%), melting point: 181-182° C. (recrystallization solvent: AcOEt-hexane).

### EXAMPLE 7

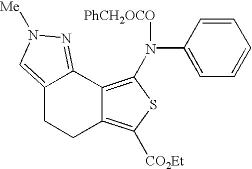

8-[(N-Benzyloxycarbonyl-N-phenyl)amino]-4,5-dihydro-2-methyl-2H-thieno[3,4-g]indazole-6-carboxylic Acid Ethyl Ester:

To a solution of 8-[(N-benzyloxycarbonyl-N-phenyl)amino]-4,5-dihydro-1H-thieno[3,4-g]indazole-6-carboxylic acid ethyl ester (1.2 g) in N,N-dimethylformamide (DMF) (15 ml) was added 60% sodium hydride (0.12 g) under ice cooling and the resulting solution was stirred for 20 minutes. To this solution was added methyl iodide (0.20 ml) and the resulting solution was stirred under ice cooling for 2 hours. The reaction solution was poured into an aqueous solution of citric acid and extracted with ethyl acetate. The extract was washed with water and an aqueous, saturated solution of sodium chloride, then dried (MgSO₄). The solvent was evaporated under reduced pressure and the thus-obtained, yellow oily substance was subjected to column chromatography on silica gel. From the fractions eluted with ethyl acetate-hexane (1:2), the title compound (1.0 g, 81%) was obtained as a light yellow, oily substance. ¹H NMR (δ ppm in CDCl₃): 1.33 (3H, t, J=7.2 Hz), 2.75 (2H, t, J=7.2 Hz), 3.32 (2H, t, J=7.2 Hz), 3.77 (3H, s), 4.29 (2H, q, J=7.2 Hz), 5.15 (2H, S), 7.0-7.6 (11H, m).

### EXAMPLE 8

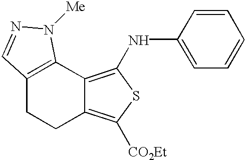

4,5-Dihydro-8-(N-phenylamino)-1H-thieno[3,4-g]indazole-6-carboxylic Acid Ethyl Ester:

A solution of 3-[(N-benzyloxycarbonyl-N-phenyl)amino]-5-diethoymethyl-4,5,6,7-tetrahydro-4-oxobenzo[c]thiophene-1-carboxylic acid ethyl ester (5.5 g), methylhydrazine (1.0 g), and 1 N hydrochloric acid (10 ml) in ethanol (50 ml) was heated at reflux for 12 hours. The reaction solution was concentrated under reduced pressure and the residue was poured into an aqueous solution of citric acid and extracted with ethyl acetate. The extract was washed with water and an aqueous, saturated solution of sodium chloride and then dried (MgSO₄). The solvent was evaporated under reduced pressure, the thus-obtained yellow oily substance and 5% Pd-C (2.2 g) were added into ethanol (50 ml)-tetrahydrofuran (THF) (25 ml), and the resulting mixture was subjected to hydrogenation at atmospheric pressure for 2 hours at room temperature. The catalyst was filtered off and the filtrate was evaporated under reduced pressure. The thus-obtained crystals were recrystallized from THF-ethyl acetate to obtain the title compound (2.8 g, 80%) as light yellow needles. Melting point: 202-203° C.

### EXAMPLE 9

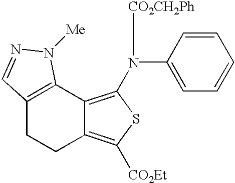

8-[(N-benzyloxycarbonyl-N-phenyl)amino]-4,5-dihydro-1-methyl-1H-thieno[3,4-g]indazole-6-carboxylic Acid Ethyl Ester:

To a solution of 4,5-dihydro-1-methyl-8-phenylamino-1H-thieno[3,4-g]indazole-6-carboxylic acid ethyl ester (150 mg) in DMF (8 ml) was added 60% sodium hydride (20 mg) under ice cooling and the resulting solution was stirred for 10 minutes. To this solution was added benzyl chloroformate (73 μl) and the resulting solution was stirred under ice cooling for 4 hours. The reaction solution was poured into an aqueous solution of citric acid and extracted with ethyl acetate. The extract was washed with water and an aqueous, saturated solution of sodium chloride and then dried (MgSO₄). The solvent was evaporated under reduced pressure and the thus-obtained oily substance was subjected to column chromatography on silica gel. From the fractions eluted with ethyl acetate-hexane (1:2), the title compound (130 mg, 63%) was obtained as a colorless oily substance. ¹H NMR (δ ppm in CDCl₃): 1.33 (3H, t, J=7.2 Hz), 2.5-2.7 (2H, m), 3.0-3.5 (2H, m), 3.81 (3H, s), 4.30 (2H, q, J=7.2 Hz), 5.14 (2H, s), 7.0-7.4 (10H, m).

### EXAMPLE 10

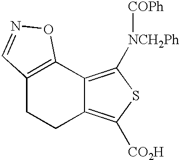

8-[(N-Benzoyl-N-benzyl)amino]-4,5-dihydrothieno[3,4-g]-1,2-benzisoxazole-6-carboxylic Acid:

A mixture of 8-[(N-benzoyl-N-benzyl)amino]-4,5-dihydrothieno[3,4-g]-1,2-benzisoxazole-6-carboxylic acid t-butyl ester (0.58 g), anisole (1 drop), and trifluoroacetic acid (10 ml) was stirred under ice cooling for 3 hours. The reaction solution was concentrated under reduced pressure and the residue was washed with isopropyl ether to obtain the title compound (0.35 g, 68%) as light yellow crystals. Melting point: 217-218° C. (decomposition).

According to the same manner as that in Example 10, compounds in Examples 11 to 14 were obtained.

### EXAMPLE 11

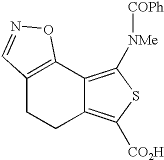

8-[(N-Benzoyl-N-methyl)amino]-4,5-dihydrothieno[3,4-g]-1,2-benzisoxazole-6-carboxylic Acid:

Light brown crystals (yield: 90%), melting point: 232-233° C. (decomposition)

### EXAMPLE 12

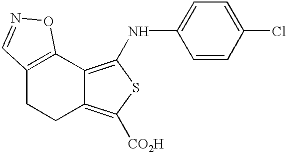

8-(4-Chlorophenylamino)-4,5-dihydrothieno[3,4-g]-1,2-benzisoxazole-6-carboxylic Acid:

Light brown crystals (yield: 75%), melting point: 232-233° C. (decomposition) (recrystallization solvent: THF-AcOEt).

### EXAMPLE 13

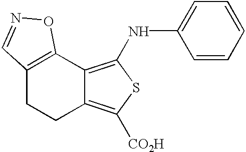

4,5-Dihydro-8-phenylaminothieno[3,4-g]-1,2-benzisoxazole-6-carboxylic Acid:

Light brown crystals (yield: 78%), melting point: 211-212° C. (decomposition)

### EXAMPLE 14

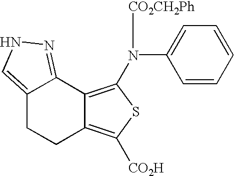

8-[(N-Benzyloxycarbonyl-N-phenyl)amino]-4,5-dihydro-1H-thieno[3,4-g]indazole-6-carboxylic Acid:

Light pink crystals (yield: 86%), melting point: 278-279° C.

### EXAMPLE 15

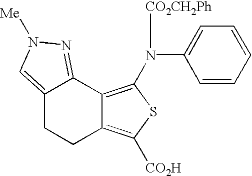

8-[(N-Benzyloxycarbonyl-N-phenyl)amino]-4,5-dihydro-2-methyl-2H-thieno[3,4-g]indazole-6-carboxylic Acid:

A solution of 8-[(N-benzyloxycarbonyl-N-phenyl)amino]-4,5-dihydro-2-methyl-2H-thieno[3,4-g]indazole-6-carboxylic acid ethyl ester (1.5 g) dissolved in a mixed solvent of THF (10 ml) and methanol (10 ml) was cooled with ice. To this solution was added an aqueous solution of 85% potassium hydroxide (0.30 g) dissolved in water (5 ml). The resulting solution was stirred at room temperature for 15 hours, then poured into an aqueous solution of citric acid, and extracted with ethyl acetate. The extract was washed with an aqueous, saturated solution of sodium chloride and then dried (MgSO₄). The solvent was evaporated under reduced pressure and the thus-obtained, yellow oily substance was crystallized from ethyl acetate-hexane to obtain the title compound (720 mg, 76%) as colorless prisms. Melting point: 200-201° C. (recrystallization solvent: AcOEt-hexane).

### EXAMPLE 16

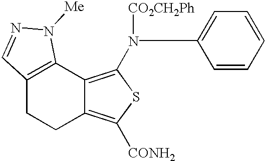

8-[(N-Benzyloxycarbonyl-N-phenyl)amino]-4,5-dihydro-1-methyl-1H-thieno[3,4-g]indazole-6-carboamide:

A solution of 8-[(N-benzyloxycarbonyl-N-phenyl)amino]-4,5-dihydro-1-methyl-1H-thieno[3,4-g]indazole-6-carboxylic acid ethyl ester (0.85 g) dissolved in a mixed solvent of THF (15 ml) and methanol (15 ml) was cooled with ice. To this solution was added an aqueous solution of 85% potassium hydroxide (0.30 g) dissolved in water (5 ml). The resulting solution was stirred at room temperature for 7 hours, then poured into an aqueous solution of citric acid, and extracted with ethyl acetate. The extract was washed with an aqueous, saturated solution of sodium chloride and then dried (MgSO₄). The solvent was evaporated under reduced pressure and the thus-obtained yellow oily substance was dissolved in DMF (15 ml). After cooling with ice, to this solution were added 1-hydroxybenzotriazole-ammonia complex (HOBt-NH₃) (0.35 g) and 1-ethyl-3-(3-dimetyhylaminopropyl)carbodiimide hydrochloride (WSC) (0.44 g) and the resulting solution was stirred under ice cooling for 15 hours. The reaction solution was poured into an aqueous solution of citric acid and extracted with ethyl acetate. The extract was washed with an aqueous solution of sodium hydrogen carbonate and an aqueous, saturated solution of sodium chloride and then dried (MgSO₄). The solvent was evaporated under reduced pressure and the thus-obtained, brown oily substance was subjected to column chromatography on silica gel. From the fractions eluted with chloroform-methanol (50:1), the title compound (210 mg, 26%) was obtained as a light brown, oily substance. ¹H NMR (δ ppm in CDCl₃): 2.5-2.7 (2H, m), 3.0-3.3 (2H, m), 3.81 (3H, s), 5.15 (2H, s), 5.68 (2H, br s), 7.1-7.4 (10H, m).

### EXAMPLE 17

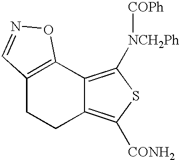

8-[(N-Benzoyl-N-benzyl)amino]-4,5-dihydrothieno[3,4-g]-1,2-benzisoxazole-6-carboxamide:

A solution of 8-[(N-benzyl-N-benzoyl)amino]-4,5-dihydrothieno[3,4-g]-1,2-benzisoxazole-6-carboxylic acid (0.25 g) dissolved in DMF (10 ml) was cooled with ice. To this solution were added HOBt-NH₃ (0.14 g) and WSC (0.17 g) and the resulting solution was stirred at room temperature for 15 hours. The reaction solution was poured into an aqueous solution of citric acid and extracted with ethyl acetate. The extract was washed with an aqueous solution of sodium hydrogen carbonate and an aqueous, saturated solution of sodium chloride and then dried (MgSO₄). The solvent was evaporated under reduced pressure and the thus-obtained light brown oily substance was crystallized from ethyl acetate-hexane to give the title compound (214 mg, 86%) as light brown prisms. Melting point: 235-236° C. (recrystallization solvent: AcOEt-hexane).

According to the same manner as that in Example 17, compounds in Examples 18 to 22 were obtained.

### EXAMPLE 18

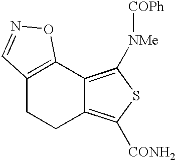

8-[(N-Benzoyl-N-methyl)amino]-4,5-dihydrothieno[3,4-g]-1,2-benzisoxazole-6-carboxamide:

Light yellow prisms (yield: 60%), melting point: 197-198° C. (recrystallization solvent: AcOEt-hexane).

### EXAMPLE 19

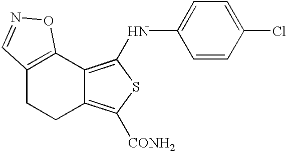

8-(4-Chlorophenylamino)-4,5-dihydrothieno[3,4-g]-1,2-benzisoxazole-6-carboxamide:

Light brown prisms (yield: 63%), melting point: 224-225° C. (recrystallization solvent: THF).

### EXAMPLE 20

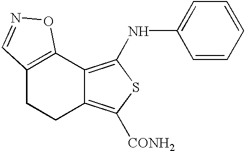

4,5-Dihydro-8-phenylaminothieno[3,4-g]-1,2-benzisoxazole-6-carboxamide:

Light yellow crystals (yield: 40%), melting point: 235-236° C. (decomposition).

### EXAMPLE 21

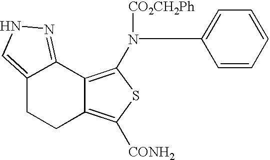

8-[(N-Benzyloxycarbonyl-N-phenyl)amino]-4,5-dihydro-1H-thieno[3,4-g]indazole-6-carboxamide:

Colorless prisms (yield: 77%), melting point: 240-241° C. (recrystallization solvent: THF-AcOEt)

### EXAMPLE 22

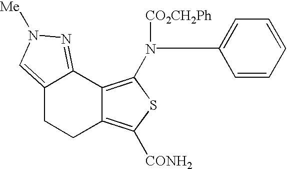

8-[(N-Benzyloxycarbonyl-N-phenyl)amino]-4,5-dihydro-2-methyl-2H-thieno[3,4-g]indazole-6-carboxamide:

Light pink prisms (yield: 94%), melting point: 220-221° C. (recrystallization solvent: AcOEt-hexane).

### EXAMPLE 23

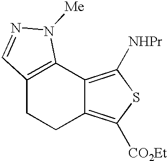

4,5-Dihydro-1-methyl-8-n-propylamino-1H-thieno[3,4-g]indazole-6-carboxylic Acid Ethyl Ester:

According to the same manner as that in Example 8, the title compound was obtained from the compound obtained in Reference Example 25. Colorless needles (yield: 73%), melting point: 161-162° C. (recrystallization solvent: AcOEt-hexane).

### EXAMPLE 24

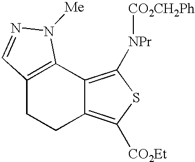

8-[(N-Benzyloxycarbonyl-N-n-propyl)amino]-4,5-dihydro-1-methyl-1H-thieno[3,4-g]indazole-6-carboxylic Acid Ethyl Ester:

According to the same manner as that in Example 9, the title compound was obtained from the compound obtained in Example 23. Light yellow, oily substance (yield: 92%),

¹H NMR (δ ppm in CDCl₃): 0.86 (3H, t, J=7.4 Hz), 1.58 (3H, t, J=7.2 Hz), 1.5-1.8 (2H, m), 2.4-3.1 (4H, m), 3.69 (3H, s), 3.5-4.0 (2H, m), 4.35 (2H, q, J=7.2 Hz), 5.18 (2H, s), 7.2-7.4 (5H, m), 7.35 (1H, s).

### EXAMPLE 25

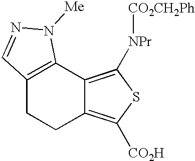

8-[(N-Benzyloxycarbonyl-N-n-propyl)amino]-4,5-dihydro-1-methyl-1H-thieno[3,4-g]indazole-6-carboxylic Acid:

According to the same manner as that in Example 15, the title compound was obtained from the compound obtained in Example 24. Colorless prisms (yield: 73%), melting point: 231-232° C. (recrystallization solvent: THF-AcOEt).

### EXAMPLE 26

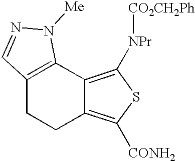

8-[(N-Benzyloxycarbonyl-N-n-propyl)amino]-4,5-dihydro-1-methyl-1H-thieno[3,4-g]indazole-6-carboxamide:

According to the same manner as that in Example 17, the title compound was obtained from the compound obtained in Example 25. Colorless oily substance (yield: 89%), ¹H NMR (δ ppm in CDCl₃): 0.86 (3H, t, J=7.4 Hz), 1.5-1.8 (2H, m), 2.5-3.2 (4H, m), 3.4-3.6 (1H, m), 3.69 (3H, s), 3.8-4.0 (1H, m), 5.18 (2H, s), 5.65 (2H, br s), 7.2-7.4 (5H, m), 7.35 (1H, s).

### EXAMPLE 27

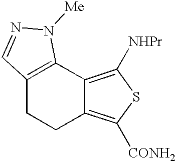

4,5-Dihydro-1-methyl-8-n-propylamino-1H-thieno[3,4-g]indazole-6-carboxamide:

8-[(N-Benzyloxycarbonyl-N-n-propyl)amino]-4,5-dihydro-1-methyl-1H-thieno[3,4-g]indazole-6-carboxamide (0.55 g) and 5% Pd-C (0.25 g) were added into methanol (15 ml)-tetrahydrofuran (THF) (15 ml) and the resulting mixture was subjected to hydrogenation at atmospheric pressure for 80 minutes at room temperature. The catalysts were filtered off and the filtrate was evaporated under reduced pressure. The thus-obtained crystals were recrystallized from methanol-ethyl acetate to obtain the title compound (295 mg, 78%) as colorless prisms. Melting point: 207-208° C.

According to the same manner as that in Example 27, compounds in Examples 28 to 30 were obtained.

### EXAMPLE 28

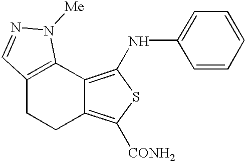

4,5-Dihydro-1-methyl-8-phenylamino-1H-thieno[3,4-g]indazole-6-carboxamide:

Light yellow prisms (yield: 57%), melting point: 140-141° C. (recrystallization solvent: AcOEt-hexane).

### EXAMPLE 29

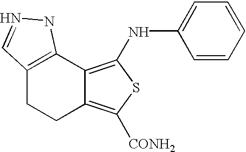

4,5-Dihydro-8-phenylamino-1H-thieno[3,4-g]indazole-6-carboxamide:

Light yellow needles (yield: 57%), melting point: 233-234° C. (recrystallization solvent: AcOEt-MeOH)

### EXAMPLE 30

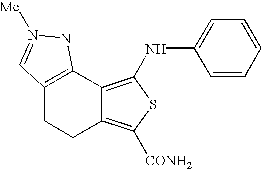

4,5-Dihydro-2-methyl-8-phenylamino]-2H-thieno[3,4-g]indazole-6-carboxamide:

Colorless prisms (yield: 65%), melting point: 245-246° C. (recrystallization solvent: THF-AcOEt)

### EXAMPLE 31

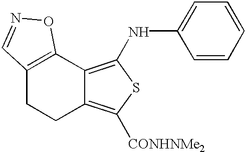

N′,N′-Dimethyl-4,5-dihydro-8-phenylaminothieno[3,4-g]-1,2-benzisoxazole-6-carbohydrazide:

A solution of 4,5-dihydro-8-phenylaminothieno[3,4-g]-1,2-benzisoxazole-6-carboxylic acid (0.30 g) and 1,1-dimethylhydrazine (88 ml) dissolved in DMF (15 ml) was cooled with ice. To this solution were added 1-hydroxybenzotriazole (HOBt) (0.18 g) and 1-ethyl-3-(3-dimetyhylaminopropyl)carbodiimide hydrochloride (WSC) (0.22 g) and the resulting solution was stirred under ice cooling for 7 hours. The reaction solution was poured into an aqueous solution of citric acid and extracted with ethyl acetate. The extract was washed with an aqueous solution of sodium hydrogen carbonate and an aqueous, saturated solution of sodium chloride and then dried (MgSO₄). The solvent was evaporated under reduced pressure and the thus-obtained brown crystals were subjected to column chromatography on silica gel. From the fractions eluted with ethyl acetate-methanol (20:1), the title compound (230 mg, 68%) was obtained as light yellow crystals. Melting point: 179-180° C.

### EXAMPLE 32

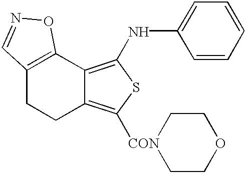

4,5-Dihydro-6-morpholinocarbonyl-8-(phenylamino)thieno[3,4-g]-1,2-benzisoxazole:

To a solution of 4,5-dihydro-8-(phenylamino)thieno[3,4-g]-1,2-benzisoxazole-6-carboxylic acid (0.20 g) in anhydrous THF (10 ml) were added oxalyl chloride (84 ml) and DMF (1 drop) under ice-cooling and the resulting solution was stirred at room temperature for 2 hours. The reaction solution was cooled and morpholine (0.20 ml) was added. After stirring under ice cooling for 1 hour, the reaction solution was poured into an aqueous solution of citric acid and extracted with ethyl acetate. The extract was washed with water and an aqueous, saturated solution of sodium chloride and then dried (MgSO₄). The solvent was evaporated under reduced pressure and the thus-obtained, yellow prisms were washed with ethyl acetate-hexane to obtain the title compound (167 mg, 68%) as light yellow prisms. Melting point: 177-178° C.

According to the same manner as in Example 32, the compound in Example 33 was obtained.

### EXAMPLE 33

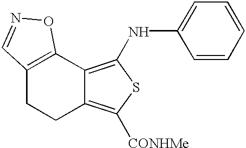

N-Methyl-4,5-dihydro-8-(phenylamino)thieno[3,4-g]-1,2-benzisoxazole-6-carboxamide:

Light yellow prisms (yield: 58%), melting point: 193-194° C.

### EXAMPLE 34

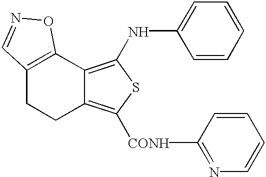

N-(2-Pyridyl)-4,5-dihydro-8-(phenylamino)thieno[3,4-g]-1,2-benzisoxazole-6-carboxamide:

To a solution of 4,5-dihydro-8-(phenylamino)thieno[3,4-g]-1,2-benzisoxazole-6-carboxylic acid (0.20 g) in anhydrous THF (10 ml) were added oxalyl chloride (84 ml) and DMF (1 drop) under ice-cooling and the resulting solution was stirred at room temperature for 2 hours. The reaction solution was cooled and 2-aminopyridine (72 mg) and triethylamine (0.17 ml) were added. After stirring under ice cooling for 2 hours, the reaction solution was poured into an aqueous solution of citric acid and extracted with ethyl acetate. The extract was washed with water and an aqueous, saturated solution of sodium chloride and then dried (MgSO₄). The solvent was evaporated under reduced pressure and the thus-obtained residue was subjected to column chromatography on silica gel. From the fractions eluted with ethyl acetate-hexane (2:1), the title compound (90 mg, 36%) was obtained as light yellow prisms. Melting point: 196-197° C. (recrystallization solvent: THF-AcOEt).

### EXAMPLE 35

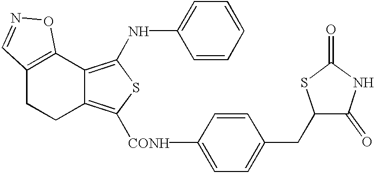

N-[4-(2,4-Dioxothiazolidin-5-ylmethyl)phenyl]-4,5-dihydro-8-phenylaminothieno[3,4-g]-1,2-benzisoxazole-6-carboxamide:

To a solution of 4,5-dihydro-8-(phenylamino)thieno[3,4-g]-1,2-benzisoxazole-6-carboxylic acid (0.30 g) in anhydrous THF (20 ml) were added oxalyl chloride (0.13 ml) and DMF (1 drop) under ice-cooling and the resulting solution was stirred at room temperature for 2 hours. The reaction solution was cooled and 4-(2,4-dioxothiazolidin-5-ylmethyl)aniline (0.26 g) and 4-(N,N-dimetylamino)pyridine (0.39 g) were added. After stirring under ice cooling for 3 hours, the reaction solution was poured into an aqueous solution of citric acid and extracted with ethyl acetate. The extract was washed with water and an aqueous, saturated solution of sodium chloride and then dried (MgSO₄). The solvent was evaporated under reduced pressure and the thus-obtained residue was subjected to column chromatography on silica gel. From the fractions eluted with ethyl acetate-hexane (2:1), the title compound (80 mg, 16%) was obtained as light yellow prisms. Melting point: 151-152° C.

### EXAMPLE 36

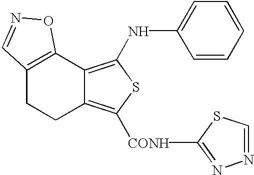

N-(1,3,4-Thiadiazol-2-yl)-4,5-dihydro-8-(phenylamino)thieno[3,4-g]-1,2-benzisoxazole-6-carboxamide:

According to the same manner as that in Example 35, the title compound (yield: 29%) was obtained as light yellow crystals. Melting point: 227-229° C. (recrystallization solvent: AcOEt-MeOH).

### EXAMPLE 37

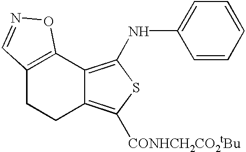

N-(4,5-Dihydro-8-phenylaminothieno[3,4-g]-1,2-benzisoxazole-6-ylcarbonyl)glycine t-butyl Ester:

According to the same manner as that in Example 31, the title compound (yield: 69%) was obtained as light yellow crystals. Melting point: 115-116° C. (recrystallization solvent: ether-i-Pr₂O).

### EXAMPLE 38

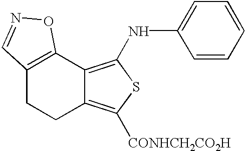

N-(4,5-Dihydro-8-(phenylamino)thieno[3,4-g]-1,2-benzisoxazole-6-ylcarbonyl)glycine:

According to the same manner as that in Example 10, the title compound (yield: 98%) was obtained as light yellow crystals from the compound obtained in Example 37. Melting point: 193-194° C.

### EXAMPLE 39

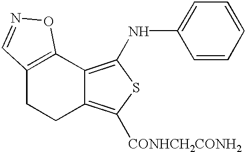

N-Carbamoylmethyl-4,5-dihydro-8-(phenylamino)thieno[3,4-g]-1,2-benzisoxazole-6-carboxamide:

According to the same manner as that in Example 17, the title compound (yield: 71%) was obtained as light yellow prisms from the compound obtained in Example 38. Melting point: 213-214° C. (recrystallization solvent: AcOEt)

### EXAMPLE 40

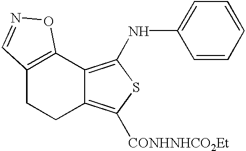

N-(4,5-Dihydro-8-(phenylamino)thieno[3,4-g]-1,2-benzisoxazole-6-ylcarbonyl)carbazinic Acid Ethyl Ester:

Accordng to the same manner as that in Example 31, the title compound (yield: 71%) was obtained as light yellow crystals. Melting point: 216-217° C. (recrystallization solvent: ethyl acetate-hexane).

### EXAMPLE 41

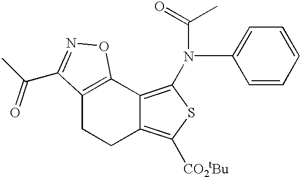

3-Acetyl-4,5-dihydro-8-[(N-acetyl-N-phenylamino)thieno[3,4-g]-1,2-benzisoxazole-6-carboxylic Acid t-butyl Ester:

A mixture of 4,5-dihydro-8-phenylaminothieno[3,4-g]-1,2-benzisoxazole-6-carboxylic acid t-butyl ester (2.5 g), 4-dimetylaminopyridine (0.25 g), and acetic anhydride (80 ml) was stirred at 70° C. for 20 hours. The reaction solution was concentrated under reduced pressure and the residue was poured into an aqueous solution of citric acid and extracted with ethyl acetate. The extract was washed with an aqueous solution of sodium hydrogen carbonate, water, and an aqueous, saturated solution of sodium chloride and then dried (MgSO₄). The solvent was evaporated under reduced pressure and the thus-obtained residue was subjected to column chromatography on silica gel. From the fractions eluted with ethyl acetate-hexane (1:3), the title compound (1.2 g, 43%) was obtained as a light yellow, oily substance. ¹H NMR (δ ppm in CDCl₃): 1.52 (9H, s), 2.10 (3H, s), 2.20 (3H, s), 2.72 (2H, t, J=7.4 Hz), 3.2-3.5 (2H, m), 7.2-7.5 (5H, m).

### EXAMPLE 42

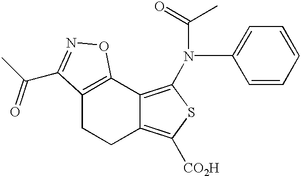

3-Acetyl-8-(N-acetyl-N-phenyl)amino-4,5-dihydrothieno[3,4-g]-1,2-benzisoxazole-6-carboxylic Acid:

According to the same manner as that in Example 10, the title compound (yield: 100%) was obtained as light yellow, oily substance from the compound obtained in Example 41.

¹H NMR (δ ppm in CDCl₃): 2.16 (3H, s), 2.23 (3H, s), 2.74 (2H, t, J=7.6 Hz), 3.2-3.5 (2H, m), 7.2-7.6 (5H, m).

### EXAMPLE 43

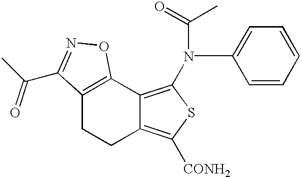

3-Acetyl-8-(N-acetyl-N-phenyl)amino-4,5-dihydro4,5-dihydrothieno[3,4-g]-1,2-benzisoxazole-6-carboxamide:

According to the same manner as that in Example 17, the title compound (yield: 51%) was obtained as light yellow crystals from the compound obtained in Example 42. Melting point: 236-237° C.

### EXAMPLE 44

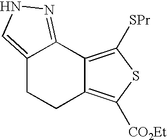

4,5-Dihydro-8-n-propylsulfanyl-1H-thieno[3,4-g]indazole-6-carboxylic Acid Ethyl Ester:

According to the same manner as that in Example 5, the title compound (yield: 68%) was obtained as light pink prisms from the compound obtained in Reference Example 31. Melting point: 161-162° C. (recrystallization solvent: AcOEt-hexane).

### EXAMPLE 45

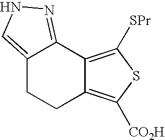

4,5-Dihydro-8-n-propylsulfanyl-1H-thieno[3,4-g]indazole-6-carboxylic Acid:

According to the same manner as that in Example 15, the title compound (yield: 98%) was obtained as light yellow crystals from the compound obtained in Example 44. Melting point: 240-241° C. (decomposition).

### EXAMPLE 46

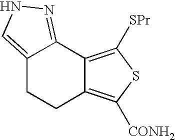

4,5-Dihydro-8-n-propylsulfanyl-1H-thieno[3,4-g]indazole-6-carboxamide:

According to the same manner as that in Example 17, the title compound (yield: 87%) was obtained as light yellow prisms from the compound obtained in Example 45. Melting point: 163-164° C. (recrystallization solvent: THF-AcOEt).

### EXAMPLE 47

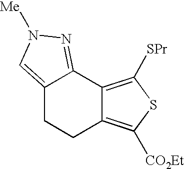

4,5-Dihydro-2-methyl-8-n-propylsulfanyl-2H-thieno[3,4-g]indazole-6-carboxylic Acid Ethyl Ester:

According to the same manner as that in Example 7, the title compound (yield: 25%) was obtained as colorless prisms from the compound obtained in Example 46. Melting point: 90-91° C. (recrystallization solvent: AcOEt-hexane)

### EXAMPLE 48

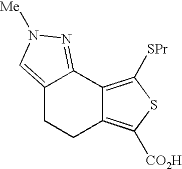

4,5-Dihydro-2-methyl-8-n-propylsulfanyl-2H-thieno[3,4-g]indazole-6-carboxylic Acid:

According to the same manner as that in Example 15, the title compound (yield: 47%) was obtained as colorless prisms from the compound obtained in Example 47. Melting point: 227-228° C. (decomposition) (recrystallization solvent: THF-hexane).

### EXAMPLE 49

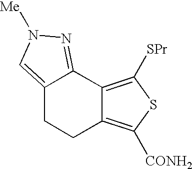

4,5-Dihydro-2-methyl-8-n-propylsulfanyl-2H-thieno[3,4-g]indazole-6-carboxamide:

According to the same manner as that in Example 17, the title compound (yield: 49%) was obtained as colorless prisms from the compound obtained in Example 48. Melting point: 208-209° C. (recrystallization solvent: THF-AcOEt).

### EXAMPLE 50

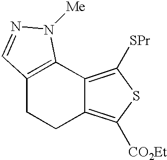

4,5-Dihydro-1-methyl-8-n-propylsulfanyl-1H-thieno[3,4-g]indazole-6-carboxylic Acid Ethyl Ester:

A solution of 5-diethoxymethyl-4,5,6,7-tetrahydro-4-oxo-3-n-propylsulfanylbenzo[c]thiophene-1-carboxylic acid ethyl ester (1.0 g), methylhydrazine (0.35 ml), and 1 N hydrochloric acid (8.7 ml) in ethanol (20 ml) was heated under reflux for 30 hours. The reaction solution was concentrated under reduced pressure and the thus-obtained residue was poured into an aqueous solution of citric acid and extracted with ethyl acetate. The organic layer was washed successively with water and an aqueous, saturated solution of sodium chloride, and then dried (MgSO₄). The solvent was evaporated under reduced pressure and the thus-obtained, oily residue was subjected to column chromatography on silica gel. From the fractions eluted with ethyl acetate-hexane (1:4), the title compound (0.67 g, 80%) was obtained as a light yellow, oily substance. ¹H NMR (δ ppm in CDCl₃): 0.98 (3H, t, J=7.4 Hz), 1.39 (3H, t, J=7.4 Hz), 1.5-1.8 (2H, m), 2.61 (2H, t, J=6.8 Hz), 2.92 (2H, t, J=7.4 Hz), 3.17 (2H, t, J=6.8 Hz), 4.19 (3H, s), 4.35 (2H, q, J=7.4 Hz), 7.41 (1H, s)

### EXAMPLE 51

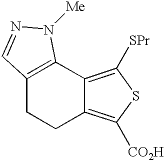

4,5-Dihydro-1-methyl-8-n-propylsulfanyl-1H-thieno[3,4-g]indazole-6-carboxylic Acid:

According to the same manner as that in Example 15, the title compound (yield: 93%) was obtained as colorless prisms from the compound obtained in Example 50. Melting point: 216-217° C. (recrystallization solvent: AcOEt)

### EXAMPLE 52

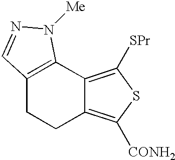

4,5-Dihydro-1-methyl-8-n-propylsulfanyl-1H-thieno[3,4-g]indazole-6-carboxamide:

According to the same manner as that in Example 17, the title compound (yield: 79%) was obtained as colorless prisms from the compound obtained in Example 51. Melting point: 112-113° C. (recrystallization solvent: MeOH-AcOEt).

### EXAMPLE 53

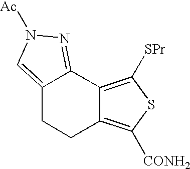

2-Acetyl-4,5-dihydro-8-n-propylsulfanyl-2H-thieno[3,4-g]indazole-6-carboxamide:

To a solution of 4,5-dihydro-8-n-propylsulfanyl-1H-thieno[3,4-g]indazole-6-carboxylic acid (0.70 g) in DMF (15 ml) was added 60% sodium hydride (0.21 g) under ice cooling and the resulting mixture was stirred for 20 minutes. To this mixture was added dropwise acetyl chloride (0.25 ml) and the resulting mixture was stirred under ice cooling for 3 hours. Then, the reaction solution was mixed with ammonia water and stirred for 30 minutes. The reaction solution was poured into an aqueous solution of citric acid and extracted with ethyl acetate. The organic layer was washed successively with water and an aqueous, saturated solution of sodium chloride and then dried (MgSO₄). The solvent was evaporated under reduced pressure and the thus-obtained residue was dissolved in DMF (20 ml) and the resulting solution was cooled with ice. To this solution were added HOBt-NH₃ (0.47 g) and WSC (0.60 g) and the resulting solution was stirred at room temperature for 16 hours. The reaction solution was poured into an aqueous solution of citric acid and extracted with ethyl acetate-THF. The organic layer was washed successively with water, an aqueous solution of sodium hydrogen carbonate, and an aqueous, saturated solution of sodium chloride and then dried (MgSO₄). The solvent was evaporated under reduced pressure and the thus-obtained residue was subjected to column chromatography on silica gel. From the fractions eluted with chloroform-methanol (30:1), the title compound (0.34 g, 43%) was obtained as light yellow prisms. Melting point: 219-220° C. (recrystallization solvent: THF-AcOEt).

### EXAMPLE 54

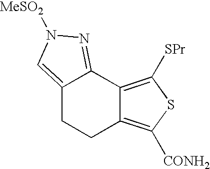

4,5-Dihydro-2-methylsulfonyl-8-n-propylsulfanyl-2H-thieno[3,4-g]indazole-6-carboxamide:

To a solution of 4,5-dihydro-8-n-propylsulfanyl-1H-thieno[3,4-g]indazole-6-carboxamide (0.30 g) in DMF (15 ml) was added 60% sodium hydride (50 mg) under ice cooling and the resulting mixture was stirred for 20 minutes. To this mixture was added dropwise methanesulfonyl chloride (95 μl) and the resulting mixture was stirred under ice cooling for 2 hours. The reaction solution was poured into an aqueous solution of citric acid and extracted with ethyl acetate. The organic layer was washed successively with water, an aqueous solution of sodium hydrogen carbonate, and an aqueous, saturated solution of sodium chloride and then dried (MgSO₄). The solvent was evaporated under reduced pressure and the thus-obtained residue was subjected to column chromatography on silica gel. From the fractions eluted with chloroform-methanol (50:1), the title compound (0.16 g, 41%) was obtained as light yellow prisms. Melting point: 191-192° C. (recrystallization solvent: AcOEt-MeOH).

Compounds in Example 55 to Example 58 were obtained according to the same manner as that in Example 54 and the compound in Example 59 was obtained according to the same manner as that in Example 10 from the compound obtained in Example 58.

TABLE 9

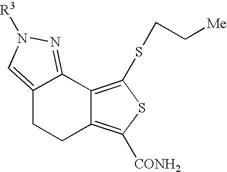

| Example | | |
|---|---|---|
| No. | R³ | Melting point (recrystallization solvent) |
| 55 | CO₂Me | 197-198° C. (MeOH—AcOEt) |
| 56 | COPh | 163-164° C. (AcOEt—hexane) |
| 57 | CONHMe | 194-195° C. (THF—AcOEt) |
| 58 | CH₂^{t}Bu | 120-121° C. (AcOEt—hexane) |
| 59 | CH₂CO₂H | 221-222° C. (THF—AcOEt) |

### EXAMPLE 60

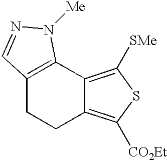

4,5-Dihydro-1-methyl-8-n-propylsulfanyl-1H-thieno[3,4g]indazole-6-carboxylic Acid Ethyl Ester:

According to the same manner as that in Example 50, the title compound (yield: 77%) was obtained as light yellow prisms from the compound obtained in Reference Example 32. Melting point: 113-114° C. (recrystallization solvent: AcOEt-hexane).

### EXAMPLE 61

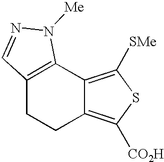

4,5-Dihydro-1-methyl-8-n-propylsulfanyl-1H-thieno[3,4-g]indazole-6-carboxylic Acid:

According to the same manner as that in Example 51, the title compound (yield: 75%) was obtained as light yellow crystals from the compound obtained in Example 60. Melting point: 281-282° C.

### EXAMPLE 62

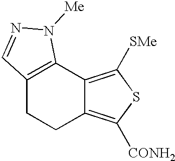

4,5-Dihydro-1-methyl-8-n-propylsulfanyl-1H-thieno[3,4-g]indazole-6-carboxamide:

According to the same manner as that in Example 52, the title compound (yield: 77%) was obtained as light yellow crystals from the compound obtained in Example 61. Melting point: 177-178° C. (recrystallization solvent: AcOEt).

### EXAMPLE 63

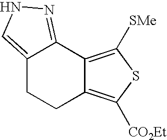

4,5-Dihydro-8-methylsulfanyl-1H-thieno[3,4-g]indazole-6-carboxylic Acid Ethyl Ester:

According to the same manner as that in Example 44, the title compound (yield: 96%) was obtained as light yellow needles from the compound obtained in Reference Example 32. Melting point: 207-208° C. (recrystallization solvent: AcOEt-hexane).

### EXAMPLE 64

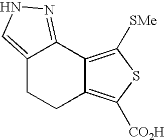

4,5-dihydro-8-methylsulfanyl-1H-thieno[3,4-g]indazole-6-carboxylic Acid:

According to the same manner as that in Example 45, the title compound (yield: 84%) was obtained as light yellow prisms from the compound obtained in Example 63. Melting point: 262-263° C. (decomposition) (recrystallization solvent: AcOEt-hexane).

### EXAMPLE 65

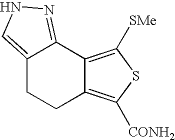

4,5-Dihydro-8-methylsulfanyl-1H-thieno[3,4-g]indazole-6-carboxamide:

According to the same manner as that in Example 46, the title compound (yield: 40%) was obtained as light yellow prisms from the compound obtained in Example 64. Melting point: 210-211° C. (recrystallization solvent: AcOEt-THF).

### EXAMPLE 66

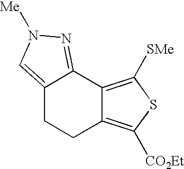

4,5-Dihydro-2-methyl-8-methylsulfanyl-2H-thieno[3,4-g]indazole-6-carboxylic Acid Ethyl Ester:

According to the same manner as that in Example 47, the title compound (yield: 78%) was obtained as light yellow crystals from the compound obtained in Example 63. Melting point: 193-194° C.

### EXAMPLE 67

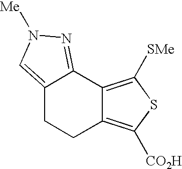

4,5-Dihydro-2-methyl-8-methylsulfanyl-2H-thieno[3,4-g]indazole-6-carboxylic Acid:

According to the same manner as that in Example 48, the title compound (yield: 87%) was obtained as light yellow prisms from the compound obtained in Example 66. Melting point: 290-291° C. (recrystallization solvent: THF-AcOEt).

### EXAMPLE 68

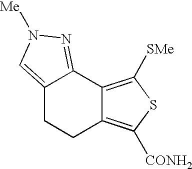

4,5-Dihydro-2-methyl-8-methylsulfanyl-2H-thieno[3,4-g]indazole-6-carboxamide:

According to the same manner as that in Example 49, the title compound (yield: 73%) was obtained as light yellow crystals from the compound obtained in Example 67. Melting point: 285-286° C.

### EXAMPLE 69

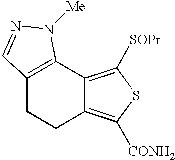

4,5-Dihydro-1-methyl-8-n-propylsulfinyl-1H-thieno[3,4-g]indazole-6-carboxamide:

To a solution of 4,5-dihydro-1-methyl-8-n-propylsulfanyl-1H-thieno[3,4-g]indazole-6-carboxamide (0.30 g) in dichloromethane (12 ml) was added m-chloroperbenzoic acid (0.25 g) under ice cooling. After stirring at room temperature for 2 hours, the reaction solution was concentrated under reduced pressure and the thus-obtained residue was diluted with ethyl acetate. The solution was washed successively with an aqueous solution of sodium hydrogen carbonate and an aqueous, saturated solution of sodium chloride and then dried (MgSO₄). The solvent was evaporated under reduced pressure and the thus-obtained residue was crystallized from ethyl acetate-hexane to obtain the title compound (0.14 g, 43%) as light yellow prisms. Melting point: 194-195° C. (recrystallization solvent: MeOH-AcOEt).

### EXAMPLE 70

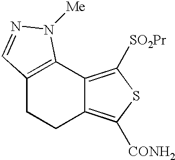

4,5-Dihydro-1-methyl-8-n-propylsulfonyl-1H-thieno[3,4-g]indazole-6-carboxamide:

To a solution of 4,5-dihydro-1-methyl-8-n-propylsulfanyl-1H-thieno[3,4-g]indazole-6-carboxamide (0.69 g) in dichloromethane (20 ml) was added m-chloroperbenzoic acid (1.3 g) under ice cooling. After stirring at room temperature for 16 hours, the reaction solution was concentrated under reduced pressure and the thus-obtained residue was diluted with ethyl acetate. The solution was washed successively with an aqueous solution of sodium hydrogen carbonate and an aqueous, saturated solution of sodium chloride and then dried (MgSO₄). The solvent was evaporated under reduced pressure and the thus-obtained residue was subjected to column chromatography on silica gel. From the fractions eluted with chloroform-methanol (30:1), the title compound (0.32 g, 42%) was obtained as light yellow crystals. Melting point: 142-143° C. (recrystallization solvent: AcOEt-hexane).

### EXAMPLE 71

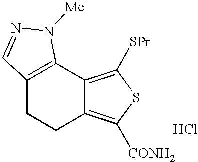

4,5-Dihydro-1-methyl-8-n-propylsulfanyl-1H-thieno[3,4-g]indazole-6-carboxamide hydrochloride:

To a solution of 4,5-dihydro-1-methyl-8-n-propylsulfanyl-1H-thieno[3,4-g]indazole-6-carboxamide (0.30 g) in THF (5 ml) was added 4 N hydrochloric acid-ethyl acetate (0.5 ml). The solvent was evaporated under reduced pressure and the thus-obtained residue was washed with ethyl acetate to obtain the title compound (0.31 g, 91%) as light yellow crystals. Melting point: 142-143° C.

### EXAMPLE 72

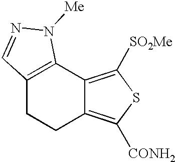

4,5-Dihydro-1-methyl-8-methylsulfonyl-1H-thieno[3,4-g]indazole-6-carboxylic Acid Ethyl Ester:

According to the same manner as that in Example 70, the title compound (yield: 22%) was obtained as light yellow prisms from the compound that was obtained in Example 60. Melting point: 140-141° C. (recrystallization solvent: AcOEt-hexane).

### EXAMPLE 73

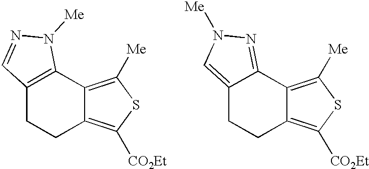

4,5-Dihydro-1,8-dimethyl-1H-thieno[3,4-g]indazole-6-carboxylic Acid Ethyl Ester and 4,5-dihydro-2,8-dimethyl-2H-thieno[3,4-g]indazole-6-carboxylic Acid Ethyl Ester:

According to the same manner as that in Example 60, there were obtained from the compound, which was obtained in Reference Example 33, 4,5-dihydro-1,8-dimethyl-1H-thieno[3,4-g]indazole-6-carboxylic acid ethyl ester (yield: 68%) as light yellow prisms (melting point: 118-119° C., AcOEt-hexane) and 4,5-dihydro-2,8-dimethyl-2H-thieno[3,4-g]indazole-6-carboxylic acid ethyl ester (yield: 23%) as colorless prisms (melting point: 149-150° C., recrystallization solvent: AcOEt-hexane).

### EXAMPLE 74

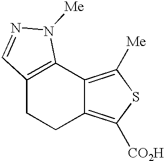

4,5-Dihydro-1,8-dimethyl-1H-thieno[3,4-g]indazole-6-carboxylic Acid:

According to the same manner as that in Example 61, the title compound (yield: 97%) was obtained as colorless crystals from 4,5-dihydro-1,8-dimethyl-1H-thieno[3,4-g]indazole-6-carboxylic acid ethyl ester. Melting point: 303° C. (decomposition)

### EXAMPLE 75

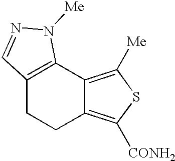

4,5-Dihydro-1,8-dimethyl-1H-thieno[3,4-g]indazole-6-carboxamide:

According to the same manner as that in Example 62, the title compound (yield: 43%) was obtained as colorless prisms from the compound obtained in Example 74. Melting point: 184-185° C. (recrystallization solvent: AcOEt).

### EXAMPLE 76

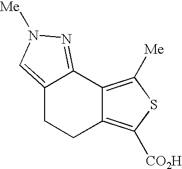

4,5-Dihydro-2,8-dimethyl-2H-thieno[3,4-g]indazole-6-carboxylic Acid:

According to the same manner as that in Example 61, the title compound (yield: 100%) was obtained as colorless crystals from 4,5-dihydro-2,8-dimethyl-2H-thieno[3,4-g]indazole-6-carboxylic acid ethyl ester. Melting point: 304-305° C. (decomposition)

### EXAMPLE 77

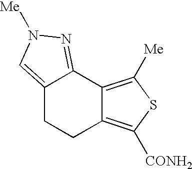

4,5-Dihydro-2,8-dimethyl-2H-thieno[3,4-g]indazole-6-carboxamide:

According to the same manner as that in Example 62, the title compound (yield: 84%) was obtained as colorless needles from the compound obtained in Example 76. Melting point: 276-277° C. (recrystallization solvent: AcOEt-THF).

### EXAMPLE 78

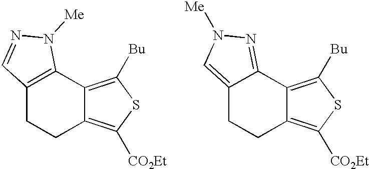

8-n-Butyl-4,5-dihydro-1-methyl-1H-thieno[3,4-g]indazole-6-carboxylic Acid Ethyl Ester and 8-n-butyl-4,5-dihydro-2-methyl-2H-thieno[3,4-g]indazole-6-carboxylic Acid Ethyl Ester:

According to the same manner as that in Example 60, there were obtained from the compound, which was obtained in Reference Example 34, 8-n-butyl-4,5-dihydro-1-methyl-1H-thieno[3,4-g]indazole-6-carboxylic acid ethyl ester (yield: 78%) as light yellow needles (melting point: 80-81° C., AcOEt-hexane) and 8-n-butyl-4,5-dihydro-2-methyl-2H-thieno[3,4-g]indazole-6-carboxylic acid ethyl ester (yield: 19%) as light yellow prisms (melting point: 69-70° C., recrystallization solvent: AcOEt-hexane).

### EXAMPLE 79

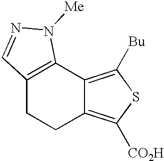

8-n-Butyl-4,5-dihydro-1-methyl-1H-thieno[3,4-g]indazole-6-carboxylic Acid:

According to the same manner as that in Example 61, the title compound (yield: 97%) was obtained as colorless crystals from 8-n-butyl-4,5-dihydro-1-methyl-1H-thieno[3,4-g]indazole-6-carboxylic acid ethyl ester. Melting point: 286-287° C.

### EXAMPLE 80

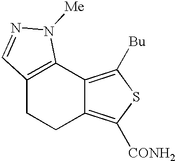

8-n-Butyl-4,5-Dihydro-1-methyl-1H-thieno[3,4-g]indazole-6-carboxamide:

According to the same manner as that in Example 62, the title compound (yield: 80%) was obtained as colorless prisms from the compound obtained in Example 79. Melting point: 119-121° C. (recrystallization solvent: AcOEt-hexane).

### EXAMPLE 81

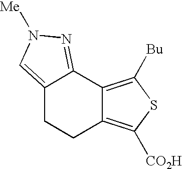

8-n-Butyl-4,5-dihydro-2-methyl-2H-thieno[3,4-g]indazole-6-carboxylic Acid:

According to the same manner as that in Example 61, the title compound (yield: 87%) was obtained as colorless prisms from 8-n-butyl-4,5-dihydro-2-methyl-2H-thieno[3,4-g]indazole-6-carboxylic acid ethyl ester. Melting point: 244-245° C.

### EXAMPLE 82

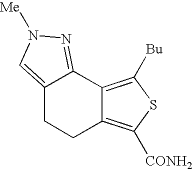

8-n-Butyl-4,5-dihydro-2-methyl-2H-thieno[3,4-g]indazole-6-carboxamide:

According to the same manner as that in Example 62, the title compound (yield: 94%) was obtained as colorless needles from the compound obtained in Example 81. Melting point: 227-228° C. (recrystallization solvent: AcOEt)

### EXAMPLE 83

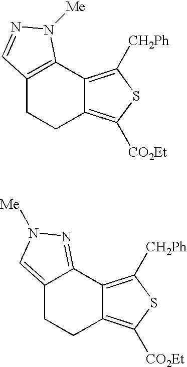

8-Benzyl-4,5-dihydro-1-methyl-1H-thieno[3,4-g]indazole-6-carboxylic Acid Ethyl Ester and 8-benzyl-4,5-dihydro-2-methyl-2H-thieno[3,4-g]indazole-6-carboxylic Acid Ethyl Ester:

According to the same manner as that in Example 60, there were obtained, from the compound obtained in Reference Example 35, 8-benzyl-4,5-dihydro-1-methyl-1H-thieno[3,4-g]indazole-6-carboxylic acid ethyl ester (yield: 79%) as a light yellow, oily substance and 8-benzyl-4,5-dihydro-2-methyl-2H-thieno[3,4-g]indazole-6-carboxylic acid ethyl ester (yield: 20%) as a light yellow, oily substance.

8-Benzyl-4,5-dihydro-1-methyl-1H-thieno[3,4-g]indazole-6-carboxylic Acid Ethyl Ester:

¹H NMR (δ ppm in CDCl₃): 1.33 (3H, t, J=7.2 Hz), 2.64 (2H, t, J=6.8 Hz), 3.20 (2H, t, J=6.8 Hz), 3.97 (3H, s), 4.30 (2H, q, J=7.2 Hz), 4.38 (2H, s), 7.2-7.4 (5H, m), 7.44 (1H, s).

8-Benzyl-4,5-dihydro-2-methyl-2H-thieno[3,4-g]indazole-6-carboxylic Acid Ethyl Ester:

¹H NMR (δ ppm in CDCl₃): 1.32 (3H, t, J=7.0 Hz), 2.75 (2H, t, J=7.2 Hz), 3.30 (2H, t, J=7.2 Hz), 3.92 (3H, s), 4.27 (2H, q, J=7.0 Hz), 4.62 (2H, s), 7.18 (1H, s), 7.2-7.5 (5H, m).

### EXAMPLE 84

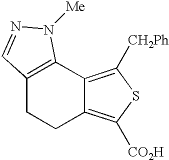

8-Benzyl-4,5-dihydro-1-methyl-1H-thieno[3,4-g]indazole-6-carboxylic Acid:

According to the same manner as that in Example 61, the title compound (yield: 84%) was obtained as light yellow crystals from 8-benzyl-4,5-dihydro-1-methyl-1H-thieno[3,4-g]indazole-6-carboxylic acid ethyl ester. Melting point: above 300° C. (recrystallization solvent: THF-MeOH).

### EXAMPLE 85

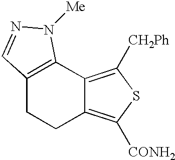

8-Benzyl-4,5-dihydro-1-methyl-1H-thieno[3,4-g]indazole-6-carboxamide:

According to the same manner as that in Example 62, the title compound (yield: 94%) was obtained as light yellow prisms from the compound obtained in Example 84. Melting point: 126-127° C. (recrystallization solvent: AcOEt).

### EXAMPLE 86

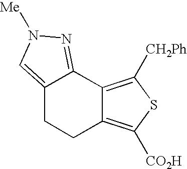

8-Benzyl-4,5-dihydro-2-methyl-2H-thieno[3,4-g]indazole-6-carboxylic Acid:

According to the same manner as that in Example 61, the title compound (yield: 98%) was obtained as colorless prisms from 8-benzyl-4,5-dihydro-2-methyl-2H-thieno[3,4-g]indazole-6-carboxylic acid ethyl ester. Melting point: 279-280° C. (decomposition) (recrystallization solvent: AcOEt).

### EXAMPLE 87

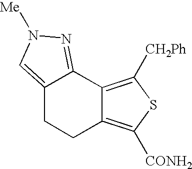

8-Benzyl-4,5-dihydro-2-methyl-2H-thieno[3,4-g]indazole-6-carboxamide:

According to the same manner as that in Example 62, the title compound (yield: 88%) was obtained as colorless needles from the compound obtained in Example 86. Melting point: 212-213° C. (recrystallization solvent: AcOEt).

### EXAMPLE 88

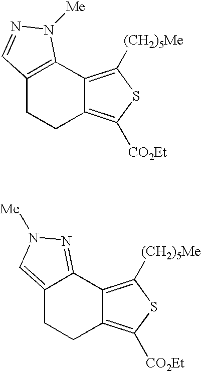

8-n-Hexyl-4,5-dihydro-1-methyl-1H-thieno[3,4-g]indazole-6-carboxylic Acid Ethyl Ester and 8-n-hexyl-4,5-dihydro-2-methyl-2H-thieno[3,4-g]indazole-6-carboxylic Acid Ethyl Ester:

According to the same manner as that in Example 60, there were obtained, from the compound obtained in Reference Example 36, 8-n-hexyl-4,5-dihydro-1-methyl-1H-thieno[3,4-g]indazole-6-carboxylic acid ethyl ester (yield: 78%) as a light yellow, oily substance and 8-n-hexyl-4,5-dihydro-2-methyl-2H-thieno[3,4-g]indazole-6-carboxylic acid ethyl ester (yield: 16%) as a light yellow, oily substance.

8-n-Hexyl-4,5-dihydro-1-methyl-1H-thieno[3,4-g]indazole-6-carboxylic Acid Ethyl Ester:

¹H NMR (δ ppm in CDCl₃): 1.88 (3H, t, J=7.0 Hz), 1.2-1.5 (6H, m), 1.39 (3H, t, J=7.2 Hz), 1.6-1.9 (2H, m), 2.60 (2H, t, J=6.8 Hz), 3.01 (2H, t, J=8.2 Hz), 3.16 (2H, t, J=6.8 Hz), 3.99 (3H, s), 4.35 (2H, q, J=7.2 Hz), 7.42 (1H, s).

8-n-Hexyl-4,5-dihydro-2-methyl-2H-thieno[3,4-g]indazole-6-carboxylic Acid Ethyl Ester:

¹H NMR (δ ppm in CDCl₃): 0.89 (3H, t, J=7.0 Hz), 1.2-1.5 (6H, m), 1.37 (3H, t, J=7.0 Hz), 1.6-1.9 (2H, m), 2.72 (2H, t, J=7.2 Hz), 3.25 (2H, t, J=6.4 Hz), 3.28 (2H, t, J=7.2 Hz), 3.91 (3H, s), 4.32 (2H, q, J=7.2 Hz), 7.15 (1H, s).

### EXAMPLE 89

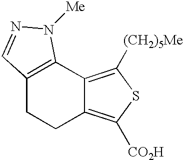

8-n-Hexyl-4,5-dihydro-1-methyl-1H-thieno[3,4-g]indazole-6-carboxylic Acid:

According to the same manner as that in Example 61, the title compound (yield: 95%) was obtained as colorless prisms from 8-n-hexyl-4,5-dihydro-1-methyl-1H-thieno[3,4-g]indazole-6-carboxylic acid ethyl ester. Melting point: 242-243° C. (recrystallization solvent: THF-AcOEt).

### EXAMPLE 90

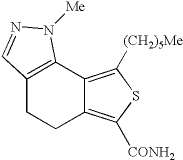

8-n-Hexyl-4,5-dihydro-1-methyl-1H-thieno[3,4-g]indazole-6-carboxamide:

According to the same manner as that in Example 62, the title compound (yield: 81%) was obtained as colorless prisms from the compound obtained in Example 89. Melting point: 100-101° C. (recrystallization solvent: THF-AcOEt).

### EXAMPLE 91

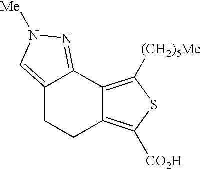

8-n-Hexyl-4,5-dihydro-2-methyl-2H-thieno[3,4-g]indazole-6-carboxylic Acid:

According to the same manner as that in Example 61, the title compound (yield: 82%) was obtained as light yellow prisms from 8-n-hexyl-4,5-dihydro-2-methyl-2H-thieno[3,4-g]indazole-6-carboxylic acid ethyl ester. Melting point: 201-202° C. (decomposition) (recrystallization solvent: AcOEt-hexane).

### EXAMPLE 92

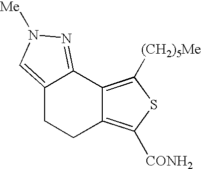

8-n-Hexyl-4,5-dihydro-2-methyl-2H-thieno[3,4-g]indazole-6-carboxamide:

According to the same manner as that in Example 62, the title compound (yield: 92%) was obtained as light yellow prisms from the compound obtained in Example 91. Melting point: 194-195° C. (recrystallization solvent: THF-AcOEt).

According to the same manner as that in Example 32, compounds in Examples 93 to 97 were obtained.

TABLE 10

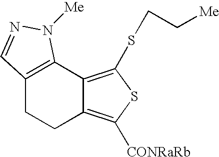

| Example | | | Melting point |
|---|---|---|---|
| No. | Ra | Rb | (recrystallization solvent) |
| 93 | n—Bu | H | 111-112° C. (AcOEt) |
| 94 | Me | H | 125-127° C. (EtOH—ether) |
| 95 | H | NMe₂ | 149-150° C. (AcOEt—hexane) |
| 96 | Me | Me | Oily substance¹⁾ |
| 97 | Et | H | 79-80° C. (ether) |

¹⁾¹H NMR(δ ppm in CDCl₃): 0.93(3H, t, J=7.4Hz), 1.5-1.7(2H, m), 2.5-2.8(4H, m), 2.81(2H, t, J=7.2Hz), 3.08(6H, s), 4.23(3H, s), 7.40(1H, s).

According to the same manner as that in Example 50, compounds in Example 98 to Example 100 were synthesized.

### EXAMPLE 98

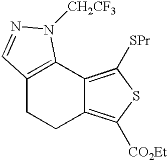

4,5-Dihydro-8-n-propylsulfanyl-1-(2,2,2-trifluoroethyl)-1H-thieno[3,4-g]indazole-6-carboxylic Acid Ethyl Ester:

Light yellow, oily substance (yield: 67%), ¹H NMR (δ ppm in CDCl₃): 1.04 (3H, t, J=7.4 Hz), 1.39 (3H, t, J=7.4 Hz), 1.6-1.9 (2H, m), 2.81 (2H, t, J=7.0 Hz), 2.99 (2H, t, J=7.0 Hz), 3.33 (2H, t, J=7.0 Hz), 4.35 (2H, q, J=7.4 Hz), 4.7-5.2 (2H, m).

### EXAMPLE 99

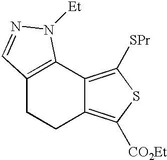

1-Ethyl-4,5-dihydro-8-n-propylsulfanyl-1H-thieno[3,4-g]indazole-6-carboxylic Acid Ethyl Ester:

Light yellow, oily substance (yield: 89%), ¹H NMR (δ ppm in CDCl₃): 0.98 (3H, t, J=7.2 Hz), 1.39 (3H, t, J=7.2 Hz), 1.49 (3H, t, J=7.2 Hz), 2.61 (2H, t, J=6.8 Hz), 2.93 (2H, t, J=7.2 Hz), 3.18 (2H, t, J=6.8 Hz), 4.35 (2H, q, J=7.2 Hz), 4.58 (2H, q, J=7.2 Hz), 7.46 (1H, s).

### EXAMPLE 100

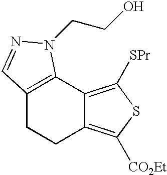

4,5-Dihydro-1-(2-hydroxyethyl)-8-n-propylsulfanyl-1H-thieno[3,4-g]indazole-6-carboxylic Acid Ethyl Ester:

Light yellow, oily substance (yield: 95%), ¹H NMR (δ ppm in CDCl₃): 0.98 (3H, t, J=7.4 Hz), 1.39 (3H, t, J=7.4 Hz), 1.5-1.8 (2H, m), 2.62 (2H, t, J=6.8 Hz), 2.92 (2H, t, J=7.2 Hz), 3.19 (2H, t, J=6.8 Hz), 4.01 (2H, t, J=4.8 Hz), 4.35 (2H, q, J=7.4 Hz), 4.66 (2H, t, J=4.8 Hz), 7.47 (1H, s).

According to the same manner as that in Example 51, compounds in Example 101 to Example 103 were synthesized.

TABLE 11

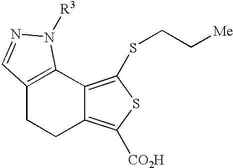

| Example | | Melting point |
|---|---|---|
| No. | R³ | (recrystallization solvent) |
| 101 | CH₂CF₃ | 212-213° C. (AcOEt) |
| 102 | CH₂CH₃ | 169-170° C. (THF—AcOEt) |
| 103 | CH₂CH₂OH | 174-175° C. (AcOEt) |

According to the same manner as that in Example 52, compounds in Example 104 to Example 106 were synthesized.

TABLE 12

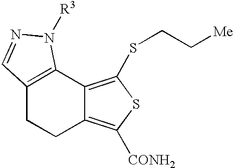

| Example | | Melting point |
|---|---|---|
| No. | R³ | (recrystallization solvent) |
| 104 | CH₂CF₃ | 107-108° C. (i-Pr₂) |
| 105 | CH₂CH₃ | 149-150° C. |
| 106 | CH₂CH₂OH | 112-113° C. (AcOEt-hexane) |

### EXAMPLE 107

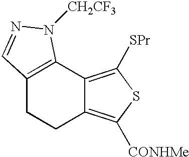

N-Methyl-4,5-dihydro-8-n-propylsulfanyl-1-(2,2,2-trifluoroethyl)-1H-thieno[3,4-g]indazole-6-carboxamide:

According to the same manner as that in Example 33, the title compound (yield: 91%) was obtained as light yellow needles from the compound obtained in Example 101. Melting point: 75-76° C. (recrystallization solvent: i-Pr₂O-hexane).

### EXAMPLE 108

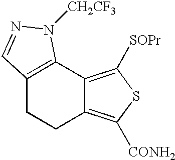

4,5-Dihydro-8-propylsulfinyl-1-(2,2,2-trifluoroethyl)-1H-thieno[3,4-g]indazole-6-carboxamide:

According to the same manner as that in Example 69, the title compound (yield: 96%) was obtained as colorless prisms from the compound obtained in Example 104. Melting point: 165-166° C. (recrystallization solvent: AcOEt-hexane).

### EXAMPLE 109

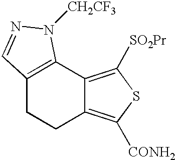

4,5-Dihydro-8-n-propylsulfonyl-1-(2,2,2-trifluoroethyl)-1H-thieno[3,4-g]indazole-6-carboxamide:

According to the same manner as that in Example 70, the title compound (yield: 83%) was obtained as light yellow prisms from the compound obtained in Example 104. Melting point: 141-142° C. (recrystallization solvent: ether-hexane).

### EXAMPLE 110

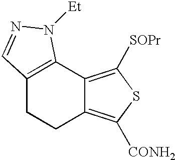

1-Ethyl-4,5-dihydro-8-n-propylsulfinyl-1H-thieno[3,4-g]indazole-6-carboxamide:

According to the same manner as that in Example 69, the title compound (yield: 89%) was obtained as light yellow prisms from the compound obtained in Example 105. Melting point: 151-152° C. (recrystallization solvent: AcOEt-hexane).

### EXAMPLE 111

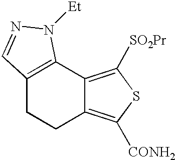

1-Ethyl-4,5-dihydro-8-n-propylsulfonyl-1H-thieno[3,4-g]indazole-6-carboxamide:

According to the same manner as that in Example 70, the title compound (yield: 31%) was obtained as colorless prisms from the compound obtained in Example 105. Melting point: 143-144° C. (recrystallization solvent: AcOEt-hexane).

According to the same manner as those in

Reference example 32 and Example 60, compounds in Example 112 to Example 114 were obtained.

### EXAMPLE 112

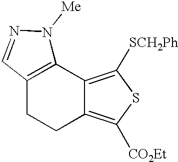

8-Benzylsulfanyl-4,5-dihydro-1-methyl-1H-thieno[3,4-g]indazole-6-carboxylic Acid Ethyl Ester:

Light yellow, oily substance (yield: 67%), ¹H NMR (δ ppm in CDCl₃): 1.39 (3H, t, J=7.2 Hz), 2.50 (2H, t, J=6.8 Hz), 3.11 (2H, t, J=6.8 Hz), 4.01 (2H, s), 4.08 (3H, s), 4.35 (2H, q, J=7.2 Hz), 7.0-7.2 (5H, m), 7.32 (1H, s).

### EXAMPLE 113

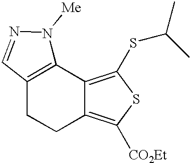

4,5-Dihydro-8-isopropylsulfanyl-1-methyl-1H-thieno[3,4-g]indazole-6-carboxylic Acid Ethyl Ester:

Light yellow, oily substance (yield: 84%), ¹H NMR (δ ppm in CDCl₃): 1.24 (6H, d, J=6.8 Hz), 1.39 (3H, t, J=6.8 Hz), 2.62 (2H, t, J=6.8 Hz), 3.19 (2H, t, J=6.8 Hz), 3.2-3.4 (1H, m), 4.19 (3H, s), 4.35 (2H, q, J=6.8 Hz), 7.41 (1H, s).

### EXAMPLE 114

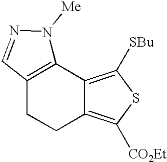

8-n-Butylsulfanyl-4,5-dihydro-1-methyl-1H-thieno[3,4-g]indazole-6-carboxylic Acid Ethyl Ester:

Light yellow, oily substance (yield: 97%), ¹H NMR (δ ppm in CDCl₃): 0.87 (3H, t, J=7.2 Hz), 1.39 (3H, t, J=7.2 Hz), 1.3-1.7 (4H, m), 2.61 (2H, t, J=6.8 Hz), 2.94 (2H, t, J=6.8 Hz), 3.17 (2H, t, J=6.8 Hz), 4.17 (3H, s), 4.35 (2H, q, J=7.2 Hz), 7.40 (1H, s)

According to the same manner as that in Example 51, compounds in Example 115 to Example 117 were obtained.

TABLE 13

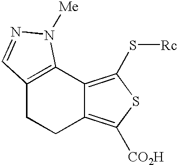

| Example | | |
|---|---|---|
| No. | Rc | Melting point |
| 115 | CH₂Ph | 228-229° C. |
| 116 | i-Pr | 246-247° C. |
| 117 | n-Bu | 225-226° C. |

According to the same manner as that in Example 52, compounds in Example 118 to Example 120 were obtained.

TABLE 14

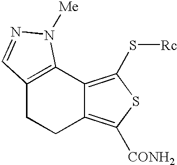

| Example | | Melting point |
|---|---|---|
| No. | Rc | (recrystallization solvent) |
| 118 | CH₂Ph | 167-168° C. (THF—AcOEt) |
| 119 | i-Pr | 150-151° C. (AcOEt) |
| 120 | n-Bu | 133-134° C. (AcOEt-hexane) |

According to the same manner as that in Example 69, compounds in Example 121 to Example 123 were obtained.

TABLE 15

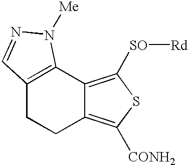

| Example | | |
|---|---|---|
| No. | Rd | Melting point |
| 121 | CH₂Ph | 224-225° C. |
| 122 | i-Pr | 195-196° C. (decomposition) |
| 123 | n-Bu | 170-171° C. |

According to the same manner as that in Example 70, compounds in Example 124 to Example 126 were obtained.

TABLE 16

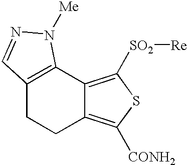

| Example | | Melting point |
|---|---|---|
| No. | Rc | (recrystallization solvent) |
| 124 | CH₂Ph | Amorphous solid¹⁾ |
| 125 | i-Pr | 193-194° C. (AcOEt-hexane) |
| 126 | n-Bu | 135-136° C. (AcOEt-hexane) |

¹⁾¹H NMR (δ ppm in CDCl₃): 2.51 (2H, t, J=6.8Hz), 3.00 (2H, t, J=6.8Hz), 4.01 (3H, s), 4.39 (2H, s), 5.81 (2H, br s), 7.0-7.3 (5H, m), 7.43 (1H, s).

### EXAMPLE 127

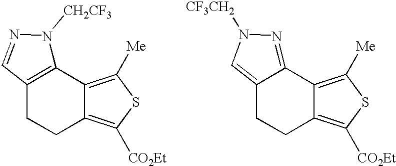

4,5-Dihydro-8-methyl-1-(2,2,2-trifluoroethyl)-1H-thieno[3,4-g]indazole-6-carboxylic Acid Ethyl Ester and 4,5-dihydro-8-methyl-2-(2,2,2-trifluoroethyl)-2H-thieno[3,4-g]indazole-6-carboxylic Acid Ethyl Ester:

According to the same manner as that in Example 73, there were obtained, from the compound obtained in Reference Example 33, 4,5-dihydro-8-methyl-1-(2,2,2-trifluoroethyl)-1H-thieno[3,4-g]indazole-6-carboxylic acid ethyl ester (yield: 69%) as colorless prisms (melting point: 120-121° C., recrystallization solvent: AcOEt-hexane) and 4,5-dihydro-8-methyl-2-(2,2,2-trifluoroethyl)-2H-thieno[3,4-g]indazole-6-carboxylic acid ethyl ester (yield: 10%) as colorless prisms (melting point: 147-148° C., recrystallization solvent: i-Pr₂O-hexane).

### EXAMPLE 128

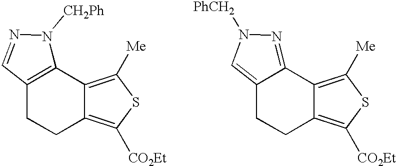

1-Benzyl-4,5-dihydro-8-methyl-1H-thieno[3,4-g]indazole-6-carboxylic Acid Ethyl Ester and 2-Benzyl-4,5-dihydro-8-methyl-2H-thieno[3,4-g]indazole-6-carboxylic Acid Ethyl Ester:

According to the same manner as that in Example 73, there were obtained, from the compound obtained in Reference Example 33, 1-benzyl-4,5-dihydro-8-methyl-1H-thieno[3,4-g]indazole-6-carboxylic acid ethyl ester (yield: 64%) as colorless prisms (melting point: 131-132° C., recrystallization solvent: AcOEt-hexane) and 2-benzyl-4,5-dihydro-8-methyl-2H-thieno[3,4-g]indazole-6-carboxylic acid ethyl ester (yield: 18%) as colorless prisms (melting point: 102-103° C., recrystallization solvent: AcOEt-hexane)

### EXAMPLE 129

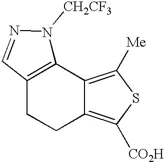

4,5-Dihydro-8-methyl-1-(2,2,2-trifluoroethyl)-1H-thieno[3,4-g]indazole-6-carboxylic Acid:

According to the same manner as that in Example 74, the title compound (yield: 96%) was obtained as colorless needles from 4,5-dihydro-8-methyl-1-(2,2,2-trifluoroethyl)-1H-thieno[3,4-g]indazole-6-carboxylic acid ethyl ester. Melting point: 250-251° C. (recrystallization solvent: AcOEt-hexane).

### EXAMPLE 130

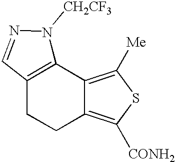

4,5-Dihydro-8-methyl-1-(2,2,2-trifluoroethyl)-1H-thieno[3,4-g]indazole-6-carboxamide:

According to the same manner as that in Example 75, the title compound (yield: 99%) was obtained as colorless prisms from the compound obtained in Example 129. Melting point: 144-145° C. (recrystallization solvent: AcOEt-hexane).

### EXAMPLE 131

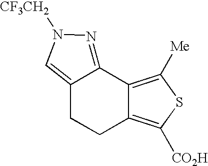

4,5-Dihydro-8-methyl-2-(2,2,2-trifluoroethyl)-2H-thieno[3,4-g]indazole-6-carboxylic Acid:

According to the same manner as that in Example 76, the title compound (yield: 80%) was obtained as colorless prisms from 4,5-dihydro-8-methyl-(2,2,2-trifluoroethyl)-2H-thieno[3,4-g]indazole-6-carboxylic acid ethyl ester. Melting point: 270° C. (decomposition)

### EXAMPLE 132

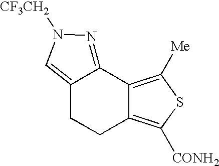

4,5-Dihydro-8-methyl-2-(2,2,2-trifluoroethyl)-2H-thieno[3,4-g]indazole-6-carboxamide:

According to the same manner as that in Example 77, the title compound (yield: 96%) was obtained as colorless prisms from the compound obtained in Example 131. Melting point: 227-228° C.

### EXAMPLE 133

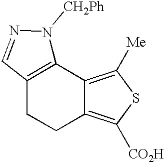

1-Benzyl-4,5-dihydro-8-methyl-1H-thieno[3,4-g]indazole-6-carboxylic Acid:

According to the same manner as that in Example 74, the title compound (yield: 96%) was obtained as colorless crystals from 1-benzyl-4,5-dihydro-8-methyl-1H-thieno[3,4-g]indazole-6-carboxylic acid ethyl ester. Melting point: 231-232° C.

### EXAMPLE 134

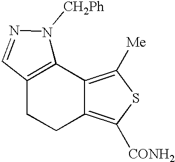

1-Benzyl-4,5-dihydro-8-methyl-1H-thieno[3,4-g]indazole-6-carboxamide:

According to the same manner as that in Example 75, the title compound (yield: 93%) was obtained as colorless needles from the compound obtained in Example 133. Melting point: 205-206° C. (recrystallization solvent: MeOH-AcOEt).

### EXAMPLE 135

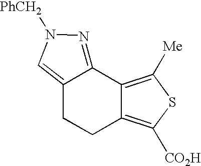

2-Benzyl-4,5-dihydro-8-methyl-2H-thieno[3,4-g]indazole-6-carboxylic Acid:

According to the same manner as that in Example 76, the title compound (yield: 100%) was obtained as colorless prisms from 2-benzyl-4,5-dihydro-8-methyl-2H-thieno[3,4-g]indazole-6-carboxylic acid ethyl ester. Melting point: 231-232° C. (recrystallization solvent: THF-AcOEt)

### EXAMPLE 136

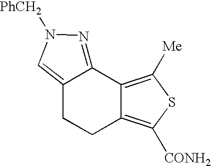

2-Benzyl-4,5-dihydro-8-methyl-2H-thieno[3,4-g]indazole-6-carboxamide:

According to the same manner as that in Example 77, the title compound (yield: 89%) was obtained as colorless needles from the compound obtained in Example 135. Melting point: 182-183° C. (recrystallization solvent: MeOH-AcOEt).

### EXAMPLE 137

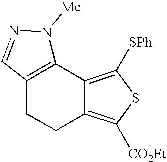

4,5-Dihydro-1-methyl-8-phenylsulfanyl-1H-thieno[3,4-g]indazole-6-carboxylic Acid Ethyl Ester:

To a solution of 4,5-dihydro-1-methyl-8-methylsulfonyl-1H-thieno[3,4-g]indazole-6-carboxylic acid ethyl ester (2.0 g) in THF (70 ml) was added 60% sodium hydride (0.26 g) under ice cooling. The resulting mixture was stirred at the same temperature for 30 minutes and further at room temperature for 5 hours. The reaction solution was poured into an aqueous solution of citric acid and extracted with ethyl acetate. The organic layer was washed successively with water and an aqueous, saturated solution of sodium chloride and then dried (MgSO₄). The solvent was evaporated under reduced pressure and the thus-obtained residue was subjected to column chromatography on silica gel. From the fractions eluted with ethyl acetate-hexane (1:2), the title compound (0.90 g, 41%) was obtained as a light yellow, oily substance. ¹H NMR (δ ppm in CDCl₃): 1.35 (3H, t, J=7.4 Hz), 2.65 (2H, t, J=6.8 Hz), 3.26 (2H, t, J=6.8 Hz), 4.16 (3H, s), 4.32 (2H, q, J=7.4 Hz), 7.1-7.3 (5H, m).

### EXAMPLE 138

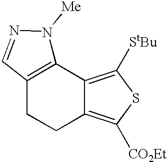

thieno[3,4-g]indazole-6-carboxylic Acid Ethyl Ester:

According to the same manner as that in Example 137, the title compound (yield: 46%) was obtained as light yellow prisms from 4,5-dihydro-1-methyl-8-methylsulfonyl-1H-thieno(3,4-g]indazole-6-carboxylic acid ethyl ester. Melting point: 124-125° C. (recrystallization solvent: ether-hexane).

### EXAMPLE 139

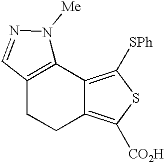

4,5-Dihydro-1-methyl-8-phenylsulfanyl-1H-thieno[3,4-g]indazole-6-carboxylic Acid:

According to the same manner as that in Example 61, the title compound (yield: 92%) was obtained as colorless prisms from the compound that was obtained in Example 137. Melting point: 286-287° C.

### EXAMPLE 140

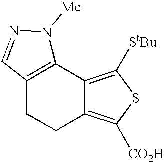

8-t-Butylsulfanyl-4,5-dihydro-1-methyl-1H-thieno[3,4-g]indazole-6-carboxylic Acid:

According to the same manner as that in Example 61, the title compound (yield: 100%) was obtained as colorless crystals from the compound obtained in Example 138. Melting point: 277-278° C.

### EXAMPLE 141

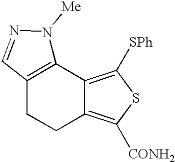

4,5-Dihydro-1-methyl-8-phenylsulfanyl-1H-thieno[3,4-g]indazole-6-carboxamide:

According to the same manner as that in Example 62, the title compound (yield: 95%) was obtained as colorless prisms from the compound obtained in Example 139. Melting point: 205-206° C. (recrystallization solvent: AcOEt-hexane).

### EXAMPLE 142

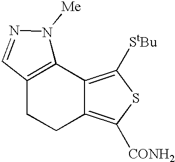

8-t-Butylsulfanyl-4,5-dihydro-1-methyl-1H-thieno[3,4-g]indazole-6-carboxamide:

According to the same menner as that in Example 62, the title compound (yield: 95%) was obtained as colorless prisms from the compound obtained in Example 140. Melting point: 146-147° C. (recrystallization solvent: AcOEt-hexane).

### EXAMPLE 143

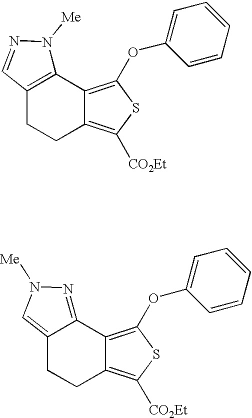

4,5-Dihydro-1-methyl-8-phenoxy-1H-thieno[3,4-g]indazole-1-carboxylic Acid Ethyl Ester and 4,5-dihydro-2-methyl-8-phenoxy-2H-thieno[3,4-g]indazole-1-carboxylic Acid Ethyl Ester:

A mixture of 5-diethoxymethyl-4,5,6,7-tetrahydro-4-oxo-3-phenoxybenzo[c]thiophene-1-carboxylic acid ethyl ester (6.32 g), methylhydrazine monohydrate (0.7 g), and 2 N hydrochloric acid (23 ml) and ethanol (50 ml) was stirred at reflux for 3 hours. The reaction solution was concentrated under reduced pressure and the residue was diluted with ethyl acetate. The organic layer was washed successively with water, an aqueous solution of sodium hydrogen carbonate, and an aqueous, saturated solution of sodium chloride, dried (MgSO₄), and then concentrated under reduced pressure. The thus-obtained residue was subjected to chromatography on silica gel. From the fractions eluted with ethyl acetate-hexane (1:2), each of the title compounds was obtained as colorless prisms. 4,5-Dihydro-1-methyl-8-phenoxy-1H-thieno[3,4-g]indazole-1-carboxylic acid ethyl ester (2.38 g, 44%), melting point: 91-93° C. (recrystallization solvent: AcOEt-hexane). 4,5-Dihydro-2-methyl-8-phenoxy-2H-thieno[3,4-g]indazole-1-carboxylic acid ethyl ester (0.87 g, 16%), melting point: 89-90° C. (recrystallization solvent: AcOEt-hexane).

According to the same manner as that in Example 143, compounds in Examples 144-166 were synthesized.

TABLE 17

| | |
|---|---|
| 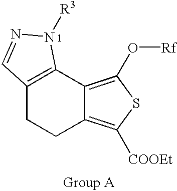 | 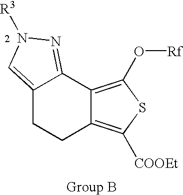 |

| | | | Melting | Melting |
|---|---|---|---|---|
| | | | point (° C.) | point (° C.) |
| | | | in Group A | in Group B |
| | | | where R³ is | where R³ is |
| Example | | | bound at | bound at |
| No. | —Rf | R³ | poison 1 | poison 2 |
| 144 | 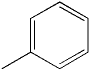 | H | 161-163 | |
| 145 | 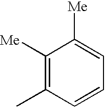 | Me | 144-145 | 108-109 |
| 146 | 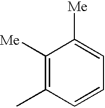 | Et | 101-102 | |
| 147 | 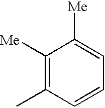 | CH₂CF₃ | 116-117 | |
| 148 | 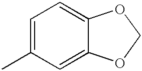 | Me | 109-111 | 168-109 |
| 149 | 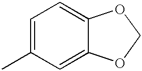 | CH₂CF₃ | 149-150 | |

TABLE 18

| | | | | |
|---|---|---|---|---|
| 150 | 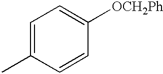 | Me | 139-140 | 155-157 |
| 151 | 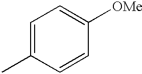 | Me | 105-106 | |
| 152 | 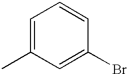 | Me | 99-100 | |
| 153 | 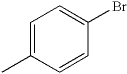 | Me | 140-142 | 159-160 |
| 154 | 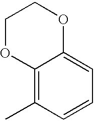 | Me | 157-158 | 155-157 |
| 155 | 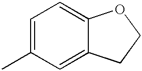 | Me | 185-187 | 166-167 |
| 156 | 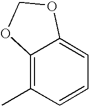 | Me | 162-163 | |
| 157 | 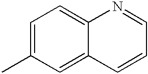 | Me | 125-126 | |
| 158 | 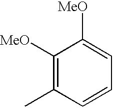 | Me | 128-129 | Oily substance¹⁶⁾ |
| 159 | 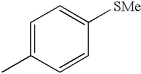 | Me | 120-121 | 155-156 |

¹⁶⁾¹H NMR (δ ppm in CDCl₃): 1.30 (3H, t, J=7.2Hz), 2.76 (2H, t, J=7.6Hz), 3.32 (2H, t, J=7.0Hz), 3.93 (3H, s), 4.25 (2H, q, J=7.2Hz), 6.81 (1H, dd, J=8.4, 1.8Hz), 6.90 (1H, dd, J=8.4, 1.8Hz), 7.02 (1H, t, J=8.4Hz), 7.15 (1H, s).

TABLE 19

| | | | | |
|---|---|---|---|---|
| 160 | 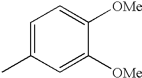 | Me | 134-135 | 169-170 |
| 161 | 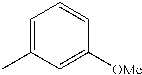 | Me | 85-86 | 107-108 |
| 162 | 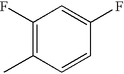 | Me | 104-106 | 159-160 |
| 163 | 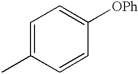 | Me | Oily substance¹²⁾ | 119-120 |
| 164 | 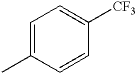 | Me | 122-123 | 148-149 |
| 165 | 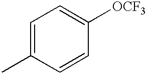 | Me | 109-108 | 101-102 |
| 166 | 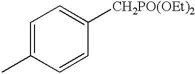 | Me | 76-77 | 112-113 |

¹²⁾¹H NMR (δ ppm in CDCl₃): 1.34 (3H, t, J=7.2Hz), 2.70 (2H, t, J=6.8Hz), 3.26 (2H, t, J=6.8Hz), 4.10 (3H, s), 4.30 (2H, q, J=7.2Hz), 7.00-7.40 (10H, m).

### EXAMPLE 167

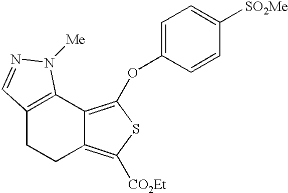

4,5,-Dihydro-8-(4-methylsulfonylphenoxy)-1-methyl-1H-thieno[3,4-g]indazole-6-carboxylic Acid Ethyl Ester:

To a solution of 4,5,-dihydro-8-(4-methylsulfanylphenoxy)-1-methyl-1H-thieno[3,4-g]indazole-6-carboxylic acid ethyl ester (1.95 g) in dichloromethane (20 ml) was added m-chloroperbenzoic acid (3.0 g) and the resulting solution was stirred at room temperature for 12 hours and then washed successively with an aqueous solution of sodium hydrogen carbonate and an aqueous, saturated solution of sodium chloride. The organic layer was dried (MgSO₄) and the solvent was evaporated under reduced pressure to obtain crude crystals. Recrystallization from ethyl acetate-diisopropyl ether gave the title compound (1.85 g, 88%) as light yellow prisms. Melting point: 190-191° C.

### EXAMPLE 168

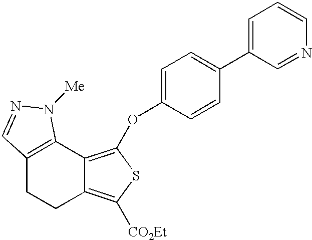

4,5,-Dihydro-1-methyl-8-[4-(3-pyridinyl)phenoxy)-1H-thieno[3,4-g]indazole-6-carboxylic Acid Ethyl Ester:

A solution of 8-(4-bromophenoxy)-4,5,-dihydro-1-methyl-1H-thieno[3,4-g]indazole-6-carboxylic acid ethyl ester (2.00 g), diethyl(3-pyridyl)borane (0.76 g), tetrakis(triphenylphosphine)palladium (0.26 g), and an aqueous 2 N solution of sodium carbonate (6.6 ml) in dimethoxyethane (60 ml) was heated at reflux for 12 hours under argon atmosphere. The reaction solution was poured into water and extracted with ethyl acetate. The organic layer was washed successively with water and an aqueous, saturated solution of sodium chloride, dried (MgSO₄), and then evaporated under reduced pressure to remove the solvent. The thus-obtained, residual oily substance was subjected to chromatography on silica gel. From the fractions eluted with ethyl acetate-hexane (2:1), the title compound (1.61 g, 81%) was obtained as colorless prisms. Melting point: 160-161° C.

According to the same manner as that in Example 168, compounds in Examples 169 to 171 were synthesized.

TABLE 20

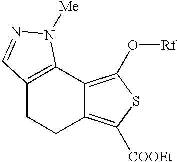

| Example | | |
|---|---|---|
| No. | —Rf | Melting point (° C.) |
| 169 | 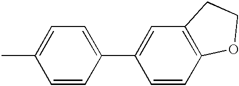 | 114-115 |
| 170 | 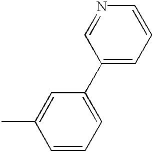 | Oily substance¹³⁾ |
| 171 | 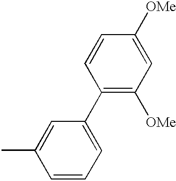 | Oily substance¹⁴⁾ |

¹³⁾¹H NMR (δ ppm in CDCl₃): 1.34 (3H, t, J=7.0Hz), 2.72 (2H, t, J=6.6Hz), 3.28 (2H, t, J=6.6Hz), 4.09 (3H, s), 4.31 (2H, q, J=7.0Hz), 7.20-7.25 (1H, m), 7.35-7.33 (4H, m), 7.37 (1H, s), 7.83-7.89 (1H, m), 8.63 (1H, dd, J=4.8, 2.0Hz), 8.83 (1H, dd, J=1.4, 0.8Hz).
¹⁴⁾¹H NMR (δ ppm in CDCl₃): 1.33 (3H, t, J=7.0Hz), 2.70 (2H, t, J=6.6Hz), 3.27 (2H, t, J=6.6Hz), 3.80 (3H, s), 3.85 (3H, s), 4.10 (3H, s), 4.30 (2H, q, J=7.0Hz), 6.55 (1H, s), 6.57 (1H, dd, J=7.2, 2.2Hz), 7.10 (1H, ddd, J=7.4, 2.6, 1.8Hz).

### EXAMPLE 172

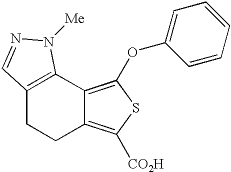

4,5-Dihydro-1-methyl-8-phenoxy-1H-thieno[3,4-g]indazole-6-carboxylic Acid:

A mixture of 4,5-dihydro-1-methyl-8-phenoxy-1H-thieno[3,4-g]indazole-6-carboxylic acid ethyl ester (2.03 g), ethanol (50 ml), and an aqueous 0.6 N solution of potassium hydroxide (20 ml) was stirred at room temperature for 14 hours. The reaction solution was concentrated under reduced pressure, made acid with 2 N hydrochloric acid, and extracted with a mixed solution of ethyl acetate-THF. The organic layer was washed successively with water and an aqueous, saturated solution of sodium chloride, dried (MgSO₄), and concentrated under reduced pressure. Recrystallization of the thus-obtained crude crystals from tetrahydrofuran gave the title compound (1.87 g, 100%) as colorless needles. Melting point: 259-261° C.

According to the same manner similar as that in Example 172, compounds in Examples 173 to 210 were synthesized.

TABLE 21

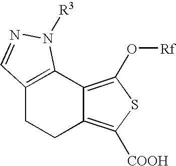

| Example | | | Melting point |
|---|---|---|---|
| No. | —Rf | R³ | (° C.) |
| 173 | 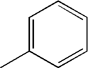 | H | 259-261 |
| 174 | 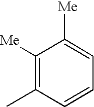 | Me | 254-255 |
| 175 | 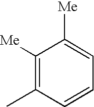 | Et | 240-241 |
| 176 | 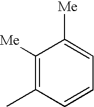 | CH₂CF₃ | 194-195 |
| 177 | 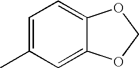 | Me | 262-264 |

TABLE 22

| | | | |
|---|---|---|---|
| 178 | 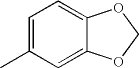 | CH₂CF₃ | 232-234 |
| 179 | 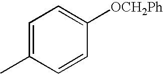 | Me | 250-252 |
| 180 | 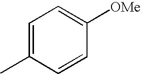 | Me | 267-268 |
| 181 | 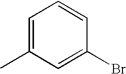 | Me | 274-276 |
| 182 | 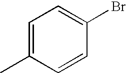 | Me | 232-234 |
| 183 | 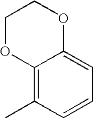 | Me | 266-268 |
| 184 | 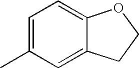 | Me | 267-269 |
| 185 | 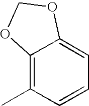 | Me | 268-270 |
| 186 | 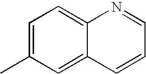 | Me | 258-260 |
| 187 | 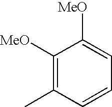 | Me | 248-249 |
| 188 | 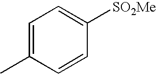 | Me | 274-276 |
| 189 | 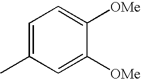 | Me | 251-252 |

TABLE 23

| | | | |
|---|---|---|---|
| 190 | 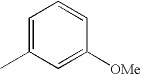 | Me | 201-202 |
| 191 | 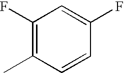 | Me | 268-269 |
| 192 | 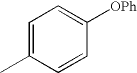 | Me | 259-260 |
| 193 | 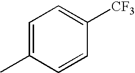 | Me | 290-291 |
| 194 | 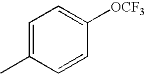 | Me | 266-267 |
| 195 | 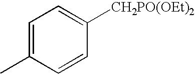 | Me | 201-202 |
| 196 | 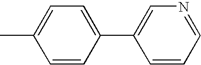 | Me | 264-266 |
| 197 | 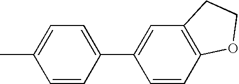 | Me | 232-234 |
| 198 | 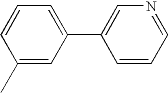 | Me | 232-234 |
| 199 | 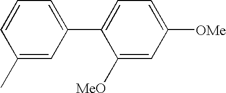 | Me | 214-216 |
| 200 | 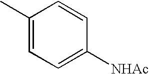 | Me | 280 (decomposition) |
| 201 | 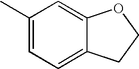 | Me | 260-262 |
| 202 | 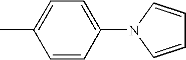 | Me | 273-274 |

TABLE 24

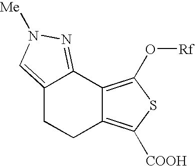

| Example | | Melting point |
|---|---|---|
| No. | —Rf | (° C.) |
| 203 | 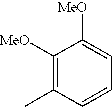 | 223-224 |
| 204 | 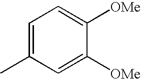 | 227-228 |
| 205 | 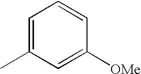 | 227-228 |
| 206 | 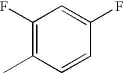 | 279-280 |
| 207 | 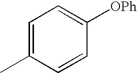 | 221-222 |
| 208 | 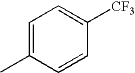 | 260-261 |
| 209 | 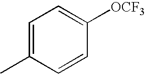 | 248-249 |
| 210 | 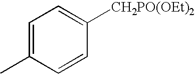 | 230-231 |

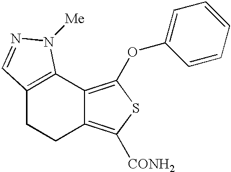

4,5-Dihydro-1-methyl-8-phenoxy-1H-thieno[3,4-g]indazole-1-carboxamide:

A solution of 4,5-dihydro-1-methyl-8-phenoxy-1H-thieno[3,4-g]indazole-1-carboxylic acid (0.50 g), HOBt-NH₃ (0.26 g), WSC (0.36 g), and DMF (10 ml) was stirred at room temperature for 14 hours. The reaction solution was concentrated under reduced pressure and the residual oily substance was mixed with water, and extracted with a mixed solution of ethyl acetate-THF. The organic layer was washed successively with water and an aqueous, saturated solution of sodium chloride, then dried (MgSO₄), and concentrated under reduced pressure. The thus-obtained crude crystals were recrystallized from ethyl acetate to obtain the title compound (0.49 g, 99%) as colorless prisms. Melting point: 200-202° C.

According to the same manner as that in Example 211, compounds in Examples 212 to 249 were synthesized.

TABLE 25

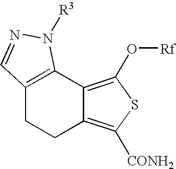

| | | | Melting point |
|---|---|---|---|
| Example No. | —Rf | R³ | (° C.) |
| 212 | 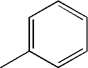 | H | 218-219 |
| 213 | 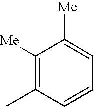 | Me | 191-192 |
| 214 | 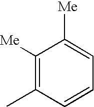 | Et | 152-154 |
| 215 | 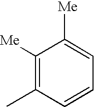 | CH₂CF₃ | 190-191 |
| 216 | 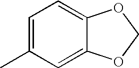 | Me | 214-205 |
| 217 | 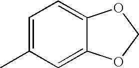 | CH₂CF₃ | 216-218 |
| 218 | 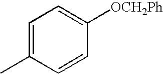 | Me | 200-201 |
| 219 | 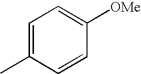 | Me | 203-205 |

TABLE 26

| | | | |
|---|---|---|---|
| 220 | 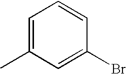 | Me | 250-251 |
| 221 | 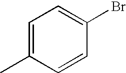 | Me | 191-192 |
| 222 | 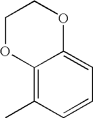 | Me | 206-207 |
| 223 | 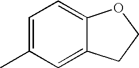 | Me | 241-243 |
| 224 | 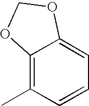 | Me | 210-212 |
| 225 | 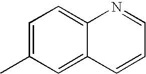 | Me | 190-191 |
| 226 | 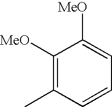 | Me | 119-120 |
| 227 | 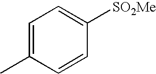 | Me | 246-248 |
| 228 | 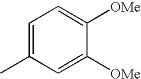 | Me | 199-200 |
| 229 | 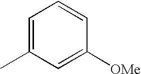 | Me | 128-129 |

TABLE 27

| | | | |
|---|---|---|---|
| 230 | 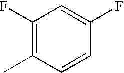 | Me | 128-129 |
| 231 | 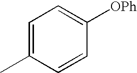 | Me | 178-179 |
| 232 | 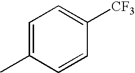 | Me | 209-210 |
| 233 | 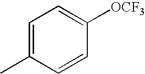 | Me | 212-213 |
| 234 | 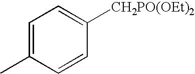 | Me | 92-93 |
| 235 | 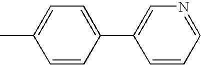 | Me | 210-212 |
| 236 | 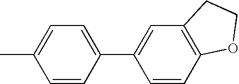 | Me | 241-243 |
| 237 | 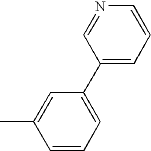 | Me | 191-192 |
| 238 | 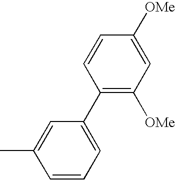 | Me | 104-106 |
| 239 | 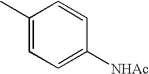 | Me | 264-266 |
| 240 | 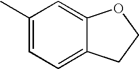 | Me | 185-186 |
| 241 | 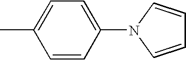 | Me | 209-210 |

TABLE 28

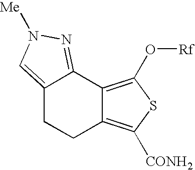

| Example | | Melting point |
|---|---|---|
| No. | —Rf | (° C.) |
| 242 | 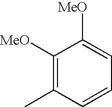 | 183-184 |
| 243 | 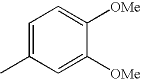 | 201-202 |
| 244 | 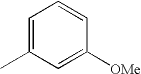 | 166-167 |
| 245 | 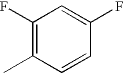 | 185-186 |
| 246 | 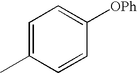 | 195-196 |
| 247 | 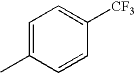 | 266-267 |
| 248 | 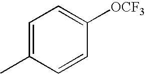 | 186-187 |
| 249 | 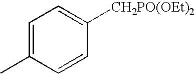 | 180-181 |

### EXAMPLE 250

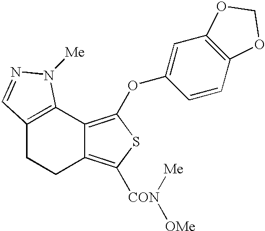

N-Methoxy-N,1-dimethyl-4,5-dihydro-8-(3,4-methylenedioxyphenoxy)-1H-thieno[3,4-g]indazole-1-carboxamide:

A solution of 4,5-dihydro-1-methyl-8-(3,4-methylenedioxyphenoxy)-1H-thieno[3,4-g]indazole-1-carboxylic acid (3.50 g), N,O-dimethylhydroxylamine hydrochloride (1.11 g), WSC (2.17 g), and triethylamine (1.6 ml) in DMF (50 ml) was stirred at room temperature overnight. The reaction solution was concentrated under reduced pressure and the residue was diluted with ethyl acetate. The organic layer was washed successively with water, an aqueous solution of sodium hydrogen carbonate, and an aqueous, saturated solution of sodium chloride, then dried (MgSO₄), and concentrated under reduced pressure. The thus-obtained crude crystals were recrystallized from ethyl acetate-hexane to obtain the title compound (1.59 g, 41%) as colorless prisms. Melting point: 130-131° C.

### EXAMPLE 251

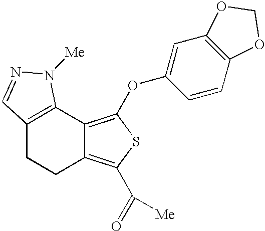

1-[4,5-Dihydro-8-(3,4-methylenedioxyphenoxy)-1-methyl-1H-thieno[3,4-g]indazol-6-yl]-1-ethanone:

To a solution of N-methoxy-N,1-dimethyl-4,5-dihydro-8-(3,4-methylenedioxyphenoxy)-1H-thieno[3,4-g]indazole-1-carboxamide (0.33 g) in THF (25 ml) was added a 1.5 M methyllithium-ether solution (0.7 ml) under cooling at −50° C. After stirring at −40 to −20° C. for 3 hours, the reaction solution was poured into an aqueous solution of citric acid and extracted with ethyl acetate. The organic layer was washed successively with water, an aqueous solution of sodium hydrogen carbonate, and an aqueous, saturated solution of sodium chloride, then dried (MgSO₄), and concentrated under reduced pressure. The thus-obtained crude crystals were recrystallized from ethyl acetate-isopropyl ether to obtain the title compound (0.24 g, 83%) as colorless needles. Melting point: 102-103° C.

### EXAMPLE 252

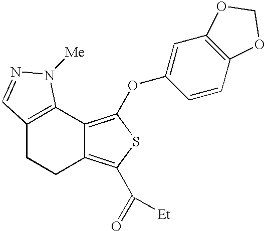

1-[4,5-Dihydro-8-(3,4-methylenedioxyphenoxy)-1-methyl-1H-thieno[3,4-g]indazol-6-yl]-1-propanone:

According to the same manner as that in Example 251, the title compound (2.24 g, 81%) was obtained as colorless needles. Melting point: 99-100° C. (recrystallization solvent: AcOEt-i-Pr₂O).

### EXAMPLE 253

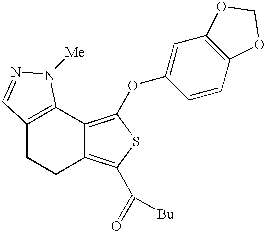

1-[1-Methyl-8-(3,4-methylenedioxyphenoxy)-4,5-dihydro-1H-thieno[3,4-g]indazol-6-yl]-1-pentanone:

According to the same manner as that in Example 251, the title compound (yield: 73%) was synthesized. Melting point: 101-102° C.

### EXAMPLE 254

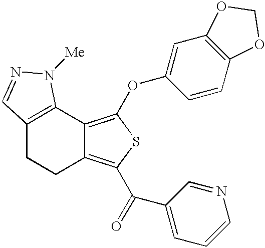

[4,5-Dihydro-8-(3,4-methylenedioxyphenoxy)-1-methyl-1H-thieno[3,4-g]indazol-6-yl](3-pyridinyl)methanone:

To a solution of 3-bromopyridine (0.25 g) in ether (20 ml) was added a 1.6 M n-butyllithium-hexane solution (0.8 ml) under cooling at −70° C. After stirring for 30 minutes, thereto was added a solution of N-methoxy-N,1-dimethyl-4,5-dihydro-8-(3,4-methylenedioxyphenoxy)-1H-thieno[3,4-g]indazole-1-carboxamide (0.6 g) in THF (20 ml), the resulting solution was stirred for 12 hours while raising the temperature gradually. Then, the reaction solution was poured into an aqueous solution of citric acid and extracted with ethyl acetate. The organic layer was washed successively with water, an aqueous solution of sodium hydrogen carbonate, and an aqueous, saturated solution of sodium chloride, then dried (MgSO₄), and concentrated under reduced pressure. The residual oily substance was subjected to column chromatography. From the fractions eluted with ethyl acetate-hexane (3:2), the title compound (0.31 g, 46%) was obtained as yellow prisms. Melting point: 180-182° C.

### EXAMPLE 255

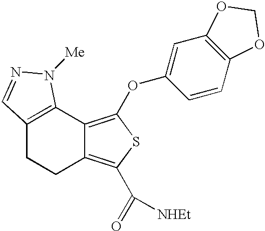

N-ethyl-4,5-dihydro-8-(3,4-methylenedioxyphenoxy)-1-methyl-1H-thieno[3,4-g]indazole-6-carboxamide:

To a suspension of 4,5-dihydro-1-methyl-8-(3,4-methylenedioxyphenoxy)-1H-thieno[3,4-g]indazole-6-carboxylic acid (1.50 g) in THF (50 ml) were added oxalyl chloride (0.7 ml) and N,N-dimethylformamide (3 drops) under ice-cooling and the resulting solution was stirred at room temperature for 30 minutes and then concentrated under reduced pressure. To a solution of the residue dissolved in THF (50 ml) was added a 70% ethylamine solution (2 ml) under ice cooling. After stirring for 30 minutes, the reaction solution was poured into water and extracted with ethyl acetate. The organic layer was washed with water, an aqueous solution of sodium hydrogen carbonate, and an aqueous, saturated solution of sodium chloride, dried (MgSO₄), and then evaporated under reduced pressure to remove the solvent. The residual oily substance was subjected to column chromatography. From the fractions eluted with ethyl acetate-hexane (3:2), the title compound (1.36 g, 84%) was obtained as colorless needles. Melting point: 138-139° C.

### EXAMPLE 256

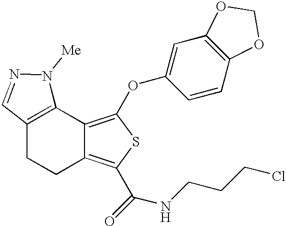

N-(3-Chloropropyl-4,5-dihydro-8-(3,4-methylenedioxyphenoxy)-1-methyl-1H-thieno[3,4-g]indazole-6-carboxamide:

According to the same manner as that in Example 255, the title compound (yield: 37%) was synthesized. Melting point: 145-147° C.

### EXAMPLE 257

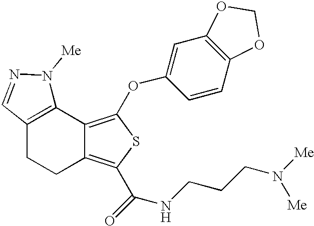

N-(3-Dimethyaminopropyl-4,5-dihydro-1-methyl-8-(3,4-methylenedioxyphenoxy)-1H-thieno[3,4-g]indazole-6-carboxamide:

According to the same manner as that in Example 255, the title compound (yield: 89%) was synthesized. Melting point: 109-110° C.

### EXAMPLE 258

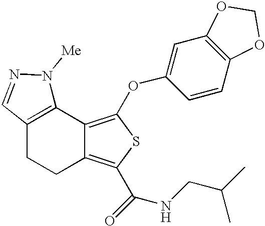

N-(2-Methylpropyl)-4,5-dihydro-1-methyl-8-(3,4-methylenedioxyphenoxy)-1H-thieno[3,4-g]indazole-6-carboxamide:

A solution of 4,5-dihydro-1-methyl-8-(3,4-methylenedioxyphenoxy)-1H-thieno[3,4-g]indazole-6-carboxylic acid (1.00 g), isobutylamine (0.22 g), WSC (0.57 g), HOBt (0.45 g), and DMF (20 ml) was stirred at room temperature for 14 hours. The reaction solution was concentrated under reduced pressure and the residual oily substance was mixed with water and extracted with a mixed solution of ethyl acetate-THF. The organic layer was washed successively with water and an aqueous, saturated solution of sodium chloride, then dried (MgSO₄), and concentrated under reduced pressure. The thus-obtained crude crystals were recrystallized from ethyl acetate to obtain the title compound (1.13 g, 98%) as colorless prisms. Melting point: 155-157° C.

### EXAMPLE 259

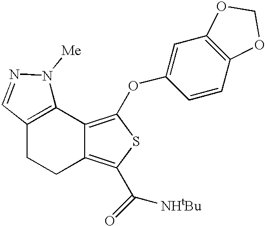

N-(1,1-Dimethylethyl)-4,5-dihydro-1-methyl-8-(3,4-methylenedioxyphenoxy)-1H-thieno[3,4-g]indazole-6-carboxamide:

According to the same manner as that in Example 258, the title compound (yield: 98%) was synthesized. Melting point: 171-179° C.

### EXAMPLE 260

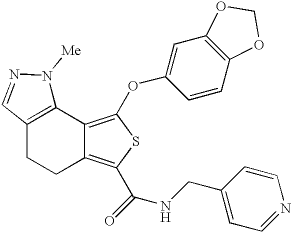

4,5-Dihydro-8-(3,4-methylenedioxyphenoxy)-1-methyl-N-(4-pyridylmethyl)-1H-thieno[3,4-g]indazole-6-carboxamide:

According to the same manner as that in Example 258, the title compound (yield: 90%) was synthesized. Melting point: 202-204° C.

### EXAMPLE 261

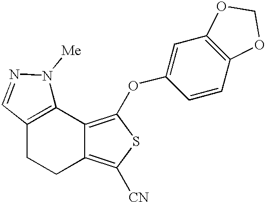

6-Cyano-4,5-dihydro-1-methyl-8-(3,4-methylenedioxyphenoxy)-1H-thieno[3,4-g]indazole:

A solution of 4,5-dihydro-1-methyl-8-(3,4-methylenedioxyphenoxy)-1H-thieno[3,4-g]indazole-6-carboxamide (1.91 g) dissolved in anhydrous trifluoroacetic acid (60 ml) was stirred at room temperature for 3 hours. The reaction solution was concentrated under reduced pressure, mixed with water, and extracted with ethyl acetate. The organic layer was washed successively with water, an aqueous solution of sodium hydrogen carbonate, and an aqueous, saturated solution of sodium chloride, dried (MgSO₄), and then evaporated to remove the solvent. The residual oily substance was subjected to column chromatography. From the fractions eluted with ethyl acetate-hexane (1:1), the title compound (1.45 g, 80%) was obtained as colorless needles. Melting point: 132-134° C.

### EXAMPLE 262

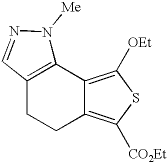

8-Ethoxy-4,5-dihydro-1-methyl-1H-thieno[3,4-g]indazole-6-carboxylic Acid Ethyl Ester:

To a solution of 4,5-dihydro-1-methyl-8-methylsulfonyl-1H-thieno[3,4-g]indazole-6-carboxylic acid ethyl ester (0.32 g) and ethanol (0.1 g) in THF (6 ml) was added sodium hydride (0.05 g) under ice cooling and the resulting mixture was stirred at room temperature for 14 hours. The reaction solution was poured into an aqueous solution of citric acid and extracted with ethyl acetate. The organic layer was washed successively with water and an aqueous, saturated solution of sodium chloride, then dried (MgSO₄), and concentrated under reduced pressure. The residual oily substance was subjected to column chromatography on silica gel. From the fractions eluted with ethyl acetate-hexane (2:1), the title compound (0.09, 30%) was obtained as colorless prisms. Melting point: 109-110° C.

According to the same manner as that in Example 262, compounds in Examples 263 to 269 were obtained.

TABLE 29

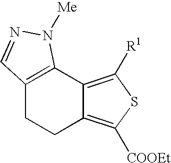

| Example | | Melting point |
|---|---|---|
| No. | R¹ | (° C.) |
| 263 | OMe | 82-84 |
| 264 | 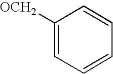 | 95-97 |
| 265 | 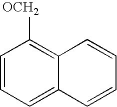 | 141-142 |
| 266 | 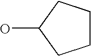 | 61-62 |
| 267 | 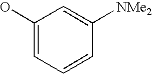 | 118-119 |
| 268 | 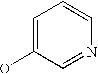 | 74-75 |
| 269 | 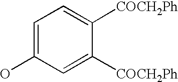 | Oily sustance¹⁵⁾ |

¹⁵⁾¹H NMR(δ ppm in CDCl₃): 1.34(3H, t, J=7.0Hz), 2.69(2H, t, J=6.6Hz), 3.25(2H, t, J=6.6Hz), 4.02(3H, s), 4.29(2H, q, J=7.0Hz), 5.14(2H, s), 5.15(2H, s), 6.70(1H, dd, J=8.8, 3.0Hz), 6.80(1H, d, J=3.0Hz), 6.92(1H, d, J=8.8Hz), 7.30-7.46(10H, m).

### EXAMPLE 270

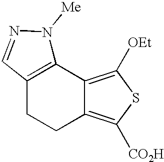

8-Ethoxy-4,5-dihydro-1-methyl-1H-thieno[3,4-g]indazole-6-carboxylic Acid:

A mixture of 8-ethoxy-4,5-dihydro-1-methyl-1H-thieno[3,4-g]indazole-6-carboxylic acid ethyl ester (0.43 g), 0.4 N KOH (20 ml), THF (20 ml), and ethanol (20 ml) was heated at reflux for 4 hours. The reaction solution was concentrated under reduced pressure, the residual oily substance was made acid with 2 N hydrochloric acid, and the precipitated crystals were collected by filtration. Recrystallization from THF gave the title compound (0.39, 99%). Melting point: 258-260° C.

According to the same manner as in Example 270, compounds in Examples 271 to 277 were obtained.

TABLE 30

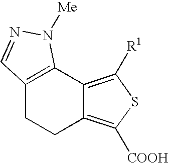

| Example | | Melting point |
|---|---|---|
| No. | R¹ | (° C.) |
| 271 | OMe | 250-252 |
| 272 | 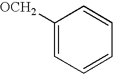 | 222-224 |
| 273 | 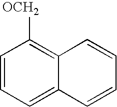 | 193-194 |
| 274 | 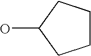 | 252-253 |
| 275 | 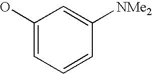 | 248-249 |
| 276 | 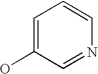 | 247-248 |
| 277 | 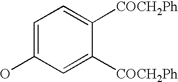 | 255-256 |

### EXAMPLE 278

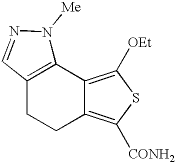

8-Ethoxy-4,5-dihydro-1-methyl-1H-thieno[3,4-g]indazole-6-carboxamide:

A solution of 8-ethoxy-4,5-dihydro-1-methyl-1H-thieno[3,4-g]indazole-6-carboxylic acid (0.30 g), HOBt-NH₃ (0.18 g), WSC (0.23 g), and DMF (8 ml) was stirred at room temperature for 15 hours. The reaction solution was concentrated under reduced pressure and the residual oily substance was subjected to addition of water and extraction with a mixed solution of ethyl acetate-THF. The organic layer was washed successively with water and an aqueous, saturated solution of sodium chloride, then dried (MgSO₄), and concentrated under reduced pressure. The thus-obtained crude crystals were recrystallized from ethyl acetate to obtain the title compound (0.25 g, 83%) as colorless prisms. Melting point: 200-202° C.

According to the same manner as that in Example 278, Examples 279 to 285 were obtained.

TABLE 31

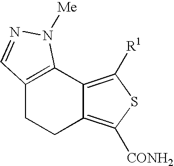

| Example | | Melting point |
|---|---|---|
| No. | R¹ | (° C.) |
| 279 | OMe | 154-156 |
| 280 | 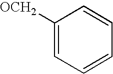 | 173-174 |
| 281 | 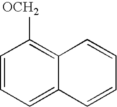 | 165-167 |
| 282 | 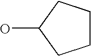 | 191-193 |
| 283 | 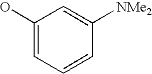 | 109-110 |
| 284 | 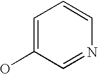 | 124-125 |
| 285 | 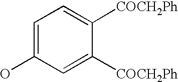 | 168-169 |

### EXAMPLE 286

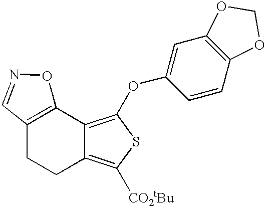

4,5-Dihydro-8-(3,4-methylenedioxyphenoxy)thieno[3,4-g]-1,2-benzisoxazole-6-carboxylic Acid t-butyl Ester:

A solution of 5-(ethoxymethylidene)-4,5,6,7-tetrahydro-8-(3,4-methylenedioxyphenoxy)-4-oxobenzo[c]thiophene-1-carboxylic acid t-butyl ester (1.95 g) and hydroxyammonium chloride (0.92 g) in ethanol (50 ml) was stirred at 70° C. for 5 hours, concentrated under reduced pressure, and then extracted with ethyl acetate. The organic layer was washed with water and an aqueous, saturated solution of sodium chloride, dried (MgSO₄), and then concentrated under reduced pressure and the residual oily substance was subjected to column chromatography. From the fractions eluted with ethyl acetate-hexane (1:4), the title compound (1.18 g, 64%) was obtained as colorless needles. Melting point: 165-166° C.

### EXAMPLE 287

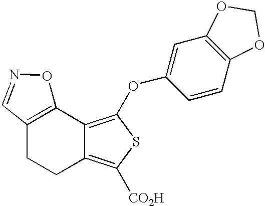

4,5-Dihydro-8-(3,4-methylenedioxyphenoxy)thieno[3,4-g]-1,2-benzisoxazole-6-carboxylic Acid:

According to the same manner as that in Example 10, the title compound was obtained. Melting point: 226-228° C. (recrystallization solvent: AcOEt).

### EXAMPLE 288

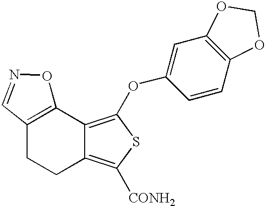

4,5-Dihydro-8-(3,4-methylenedioxyphenoxy) thieno [3,4-g]-1,2-benzisoxazole-6-carboxamide:

According to the same manner as that in Example 211, the title compound was obtained. Melting point: 167-169° C. (recrystallization solvent: AcOEt-i-Pr₂O).

### EXAMPLE 289

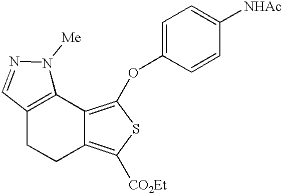

3-[4-(Acetylamino)phenoxy]-4,5-dihydro-8-methyl-1H-thieno[3,4-g]indazole-6-carboxylic Acid Ethyl Ester:

A solution of 3-[4-(acetylamino)phenoxy]-5-[(E)-ethoxymethylidene]-6,7-dihydro-4-oxobenzo [c] thiophene-1-carboxylic acid ethyl ester (2.35 g) and methylhydrazine sulfate (0.95 g) in ethanol (100 ml) was subjected to stirring at 60° C. for 3 hours, cooling, addition of water, and then extraction with ethyl acetate. The organic layer was washed with water and an aqueous, saturated solution of sodium chloride, dried (MgSO₄), and then concentrated under reduced pressure. The residual oily substance was subjected to column chromatography. From the fractions eluted with ethyl acetate-hexane (1:2), the title compound (1.13 g, 50%) was obtained as colorless needles. Melting point: 181-182° C.

### EXAMPLE 290

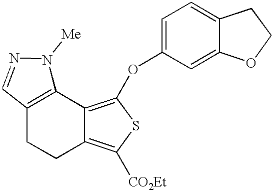

4,5-Dihydro-8-(2,3-dihydrobenzofuran-6-yloxy)-1-methyl-1H-thieno[3,4-g]indazole-6-carboxylic Acid Ethyl Ester:

To a solution of 6,7-dihydro-3-(2,3-dihydrobenzofuran-6-yloxy)-5-((E)-ethoxymethylidene)-4-oxobenzo[c]thiophene-1-carboxylic acid ethyl ester (3.28 g) and methanesulfonic acid (2.7 ml) in ethanol (50 ml) was added methylhydrazine monohydrate (0.55 g) and the resulting solution was stirred at 60° C. for 3 hours. The reaction solution was concentrated, then poured into an aqueous, saturated solution of sodium hydrogen carbonate, and extracted with ethyl acetate. The organic layer was washed successively with water and an aqueous, saturated solution of sodium chloride, dried (MgSO₄), and then concentrated under reduced pressure. The residual oily substance was subjected to column chromatography. From the fractions eluted with ethyl acetate-hexane (1:4), the title compound (2.10 g, 67%) was obtained as colorless prisms. Melting point: 137-138° C. (recrystallization solvent: AcOEt-i-Pr₂O).

### EXAMPLE 291

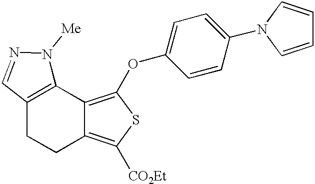

4,5-Dihydro-1-methyl-8-[4-(1H-pyrol-1-yl)phenoxy]-1H-thieno[3,4-g]indazole-6-carboxylic Acid Ethyl Ester:

According to the same manner as that in Example 289, the title compound (yield: 39%) was obtained as colorless needles from the compound obtained in Reference Example 82. Melting point: 139-140° C. (recrystallization solvent: AcOEt).

### EXAMPLE 292

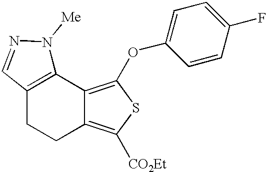

8-(4-Fluorophenoxy)-4,5-dihydro-1-methyl-1H-thieno[3,4-g]indazole-6-carboxylic Acid Ethyl Ester:

According to the same manner as that in Example 290, the title compound (yield: 72%) was obtained as colorless needles from the compound obtained in Reference Example 83. Melting point: 94-95° C. (recrystallization solvent: AcOEt-i-Pr₂O).

### EXAMPLE 293

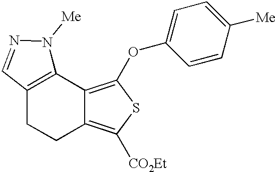

4,5-Dihydro-1-methyl-8-(4-methylphenoxy)-1H-thieno[3,4-g]indazole-6-carboxylic Acid Ethyl Ester:

According to the same manner as that in Example 290, the title compound (yield: 60%) was obtained as colorless needles from the compound obtained in Reference

### EXAMPLE 84

### Melting point: 147-148° C. (recrystallization solvent: AcOEt-hexane).

### EXAMPLE 294

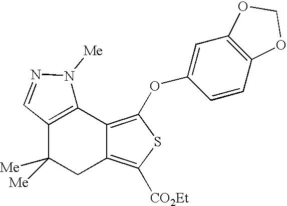

4,5-Dihydro-1,4,4,-trimethyl-8-(3,4-methylenedioxyphenoxy)-1H-thieno[3,4-g]indazole-6-carboxylic Acid Ethyl Ester:

A solution of 6,7-dihydro-1,4,4,-trimethyl-8-methylsulfonyl-1H-thieno[3,4-g]indazole-6-carboxylic acid ethyl ester (2.22 g), sesamol (0.88 g), and potassium t-butoxide (0.77 g) in 1-methyl-2-pyrrolidone (30 ml) was stirred at 90° C. for 12.5 hours, then poured into water, and extracted with ethyl acetate. The extract was washed with an aqueous, saturated solution of sodium hydrogen carbonate, water, and an aqueous, saturated solution of sodium chloride, dried (MgSO₄), and then evaporated under reduced pressure to remove the solvent. The crude crystals were recrystallized from ethyl acetate-hexane to obtain the title compound (1.07 g, 42%) as colorless needles. Melting point: 128-130° C. (recrystallization solvent: AcOEt-hexane)

### EXAMPLE 295

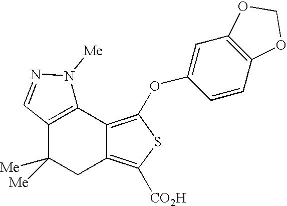

4,5-Dihydro-1,4,4,-trimethyl-8-(3,4-methylenedioxyphenoxy)-1H-thieno[3,4-g]indazole-6-carboxylic Acid:

According to the same manner as that in Example 172, the title compound (yield: 85%) was obtained. Melting point: 291-293° C. (recrystallization solvent: THF).

### EXAMPLE 296

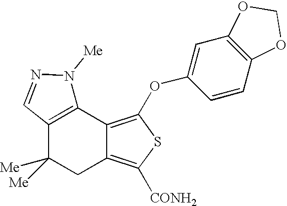

4,5-Dihydro-1,4,4,-trimethyl-8-(3,4-methylenedioxyphenoxy)-1H-thieno[3,4-g]indazole-6-carboxamide:

According to the same manner as that in Example 211, the title compound (yield: 89%) was obtained. Melting point: 219-220° C. (recrystallization solvent: THF).

### EXAMPLE 297

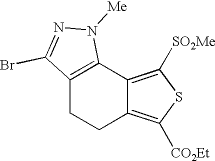

3-Bromo-4,5-dihydro-1-methyl-8-methylsulfonyl-1H-thieno[3,4-g]indazole-6-carboxylic Acid Ethyl Ester:

A solution of the compound that was obtained in Example 12a hereinafter (0.50 g), bromine (0.70 g), and sodium acetate (0.36 g) in acetic acid (10 ml) was stirred at room temperature for 18 hours. The reaction solution was concentrated under reduced pressure and the residue was diluted with ethyl acetate. This solution was washed with an aqueous solution of sodium hydrogen carbonate and an aqueous solution of sodium chloride, then dried (MgSO₄), and concentrated under reduced pressure. The thus-obtained, oily substance was crystallized from ethyl acetate-hexane to obtain the title compound (0.33 g, 54% yield) as light yellow prisms. Melting point: 195-196° C.

### EXAMPLE 298

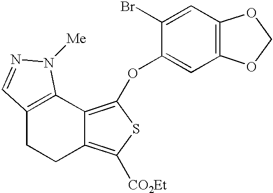

8-(6-Bromo-3,4-methylenedioxyphenoxy)-4,5-dihydro-1-methyl-1H-thieno[3,4-g]indazole-6-carboxylic Acid Ethyl Ester:

According to the same manner as that in Example 297, the title compound (1.71 g, 84% yield) was obtained from the compound (1.7 g) that was obtained in Example 14a hereinafter. Melting point: 175-176° C.

### EXAMPLE 299

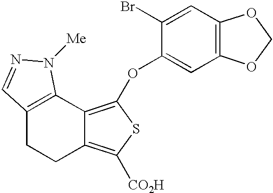

8-(6-Bromo-3,4-methylenedioxyphenoxy)-4,5-dihydro-1-methyl-1H-thieno[3,4-g]indazole-6-carboxylic Acid:

According to the same manner as that in Example 172, the title compound (1.44 g, 97% yield) was obtained as light yellow crystals from the compound (1.58 g) that was obtained in Example 298. Melting point: 285-286° C.

### EXAMPLE 300

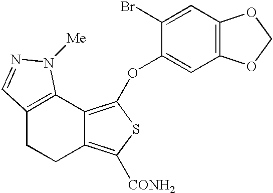

8-(6-Bromo-3,4-methylenedioxyphenoxy)-4,5-dihydro-1-methyl-1H-thieno[3,4-g]indazole-6-carboxamide:

According to the same manner as that in Example 211, the title compound (1.12 g, 94% yield) was obtained as light yellow prisms from the compound (1.2 g) obtained in

### EXAMPLE 299

### Melting point: 196-197° C

### EXAMPLE 301

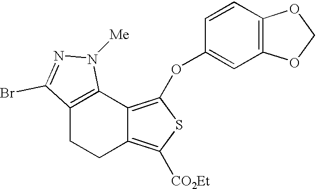

3-Bromo-4,5-dihydro-8-(3,4-methylenedioxyphenoxy)-1-methyl-1H-thieno[3,4-g]indazole-6-carboxylic Acid Ethyl Ester:

According to the same manner as that in Example 13a hereinafter, the title compound (5.78 g, 81% yield) was obtained as light yellow needles from the compound (6.25 g) obtained in Example 297. Melting point: 170-171° C.

### EXAMPLE 302

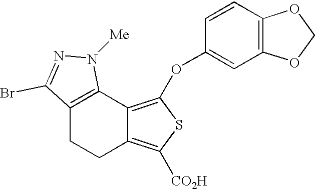

3-Bromo-4,5-dihydro-8-(3,4-methylenedioxyphenoxy)-1-methyl-1H-thieno[3,4-g]indazole-6-carboxylic Acid:

According to the same manner as that in Example 172, the title compound (5.3 g, 100% yield) was obtained as colorless prisms from the compound (5.6 g) that was obtained in Example 301. Melting point: 242-243° C.

### EXAMPLE 303

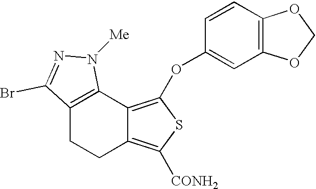

3-Bromo-4,5-dihydro-8-(3,4-methylenedioxyphenoxy)-1-methyl-1H-thieno[3,4-g]indazole-6-carboxamide:

According to the same manner as that in Example 211, the title compound (3.25 g, 93% yield) was obtained as light yellow prisms from the compound (3.5 g) that was obtained in Example 302. Melting point: 196-197° C.

According to the same manner as that in Example 168, compounds in Examples 304 to 313 were synthesized.

TABLE 32

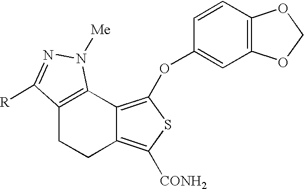

| Example | | Melting point |
|---|---|---|
| No. | R¹ | (° C.) |
| 304 | 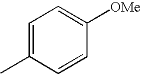 | 241-242 |
| 305 | 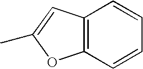 | 255-256 |
| 306 | 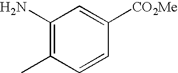 | 219-220 |
| 307 | 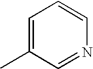 | 238-239 |
| 308 | 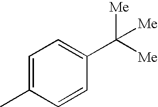 | 147-148 |
| 309 | 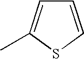 | 222-223 |
| 310 | 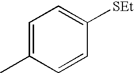 | 191-192 |
| 311 | 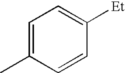 | 218-219 |
| 312 | 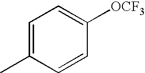 | 213-214 |
| 313 | 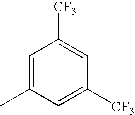 | 246-247 |

### EXAMPLE 314

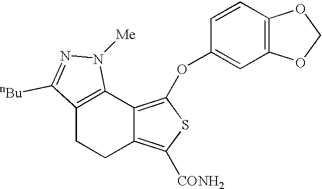

3-n-Butyl-4,5-dihydro-1-methyl-8-(3,4-methylenedioxy-phenoxy)-1H-thieno[3,4-g]indazole-6-carboxamide:

A solution of 3-bromo-1-methyl-8-(3,4-methylenedioxyphenoxy)-1H-thieno[3,4-g]indazole-6-carboxamide (0.40 g), tetra-n-butyltin (0.93 ml), tetrakis(triphenylphosphine)palladium (31 mg), and lithium chloride (115 mg) in DMF (5 ml) was stirred at 100° C. for 24 hours under argon atmosphere. The reaction solution was diluted with ethyl acetate, washed with water and an aqueous solution of sodium chloride, then dried (MgSO₄), and concentrated under reduced pressure. The thus-obtained residual oily substance was dissolved in a mixed solution of tetrahydrofuran (THF) (10 ml) and methanol (10 ml) and subjected to the addition of 10% palladium carbon (0.10 g), followed by hydrogenation under hydrogen atmosphere at atmospheric pressure at room temperature. The catalyst was filtered off and the filtrate was concentrated under reduced pressure. The thus-obtained yellow oily substance was subjected to chromatography on silica gel. From the fractions eluted with ethyl acetate-hexane (2:1), the title compound (45 mg, 12% yield) was obtained as colorless needles. Melting point: 161-162° C.

### EXAMPLE 315

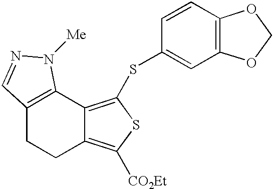

4,5-Dihydro-1-methyl-8-(3,4-methylenedioxyphenylsufanyl)-1H-thieno[3,4-g]indazole-6-carboxylic Acid Ethyl Ester:

According to the same manner as that in Example 13a hereinafter, the title compound (10.6 g, 88% yield) was obtained as light yellow prisms from the compound (10 g) obtained in Example 12a hereinafter and 3,4-methylenedioxythiophenol (4.8 g). Melting point: 101-102° C.

### EXAMPLE 316

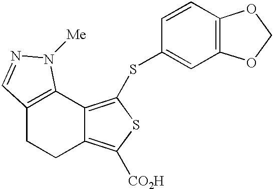

4,5-Dihydro-1-methyl-8-(3,4-methylenedioxyphenylsufanyl)-1H-thieno[3,4-g]indazole-6-carboxylic Acid:

According to the same manner as that in Example 15, the title compound (6.5 g, 100% yield) was obtained as colorless crystals from the compound (7.0 g) that was obtained in Example 315. Melting point: 286-288° C. (decomposition).

### EXAMPLE 317

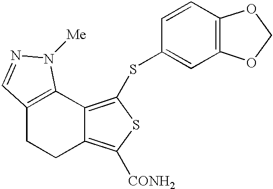

4,5-Dihydro-1-methyl-8-(3,4-methylenedioxyphenylsufanyl)-1H-thieno[3,4-g]indazole-6-carboxamide:

According to the same manner as that in Example 211, the title compound (5.2 g, 100% yield) was obtained as colorless crystals from the compound (5.5 g) that was obtained in Example 316. Melting point: 171-172° C.

### EXAMPLE 318

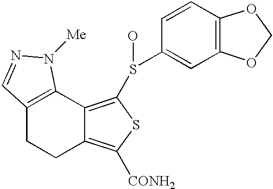

4,5-Dihydro-1-methyl-8-(3,4-methylenedioxyphenylsufinyl)-1H-thieno[3,4-g]indazole-6-carboxamide:

According to the same manner as that in Example 69, the title compound (0.64 g, 62% yield) was obtained as light yellow prisms from the compound (1.0 g) that was obtained in Example 317. Melting point: 182-183° C.

### EXAMPLE 319

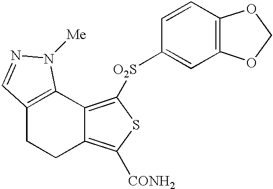

4,5-Dihydro-1-methyl-8-(3,4-methylenedioxyphenylsufonyl)-1H-thieno[3,4-g]indazole-6-carboxamide:

According to the same manner as that in Example 12a hereinafter, the title compound (0.85 g, 78% yield) was obtained as light yellow prisms from the compound (1.0 g) that was obtained in Example 317. Melting point: 234-235° C.

### EXAMPLE 320

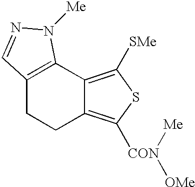

N-Methoxy-N-methyl-4,5-dihydro-1-methyl-8-(methysufanyl)-1H-thieno[3,4-g]indazole-6-carboxamide:

According to the same manner as that in Example 250, the title compound (6.0 g, 39% yield) was obtained as light yellow needles from the compound (13.4 g) obtained in Example 61. Melting point: 87-88° C.

### EXAMPLE 321

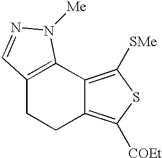

1-[4,5-Dihydro-1-methyl-8-methylsulfanyl-1H-thieno[3,4-g]indazol-6-yl]-1-propanone:

A solution of the compound (5.7 g), which was obtained in Example 320, dissolved in anhydrous THF (150 ml) was cooled at −40° C. To this solution was added a 1 M solution of ethylmagnesium bromide in THF (44 ml) and the resulting solution was stirred at 4° C. for 3 hours. The thus-obtained, reaction solution was poured into an aqueous solution of citric acid and evaporated under reduced pressure to remove THF. The residue was extracted with ethyl acetate, the extract was washed with an aqueous solution of sodium chloride, then dried (MgSO₄), and concentrated under reduced pressure. The thus-obtained crystals were recrystallized from ethyl acetate-hexane to obtain the title compound (5.0 g, 97% yield) as light yellow needles. Melting point: 119-120° C.

### EXAMPLE 322

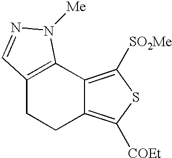

1-[4,5-Dihydro-1-methyl-8-methylsulfonyl-1H-thieno[3,4-g]indazol-6-yl]-1-propanone:

According to the same manner as that in Example 70, the title compound (3.2 g, 69% yield) was obtained as a light yellow, oily substance from the compound (4.2 g) that was obtained in Example 321. ¹H-NMR (δ ppm in CDCl₃): 1.26 (3H, t, J=7.4 Hz), 2.65 (2H, t, J=6.6 Hz), 2.93 (2H, q, J=7.4 Hz), 3.18 (2H, t, J=6.6 Hz), 3.21 (3H, s), 4.09 (3H, s), 7.48 (1H, s).

### EXAMPLE 323

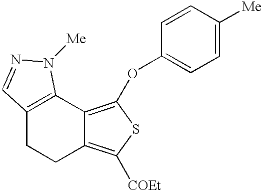

1-[4,5-Dihydro-1-methyl-8-(4-methylphenoxy)-1H-thieno[3,4-g]indazol-6-yl]-1-propanone:

According to the same manner as that in Example 13a hereinafter, the title compound (0.15 g, 14% yield) was obtained as light brown needles from the compound (1.0 g) that was obtained in Example 322. Melting point: 118-119° C.

### EXAMPLE 324

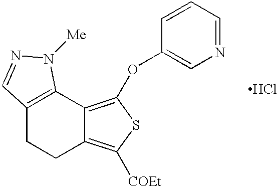

1-[4,5-Dihydro-1-methyl-8-(3-pyridinyloxy)-1H-thieno[3,4-g]indazol-6-yl]-1-propanone hydrochloride:

According to the same manner as that in Example 13a hereinafter, the title compound (0.15 g, 13% yield) was obtained as a light brown, amorphous solid from the compound (1.0 g) that was obtained in Example 322. ¹H-NMR (δ ppm in CDCl₃): 1.05 (3H, t, J=7.2 Hz), 2.68 (2H, t, J=6.8 Hz), 2.84 (2H, q, J=7.2 Hz), 3.19 (2H, t, J=6.8 Hz), 3.98 (3H, s), 7.40 (1H, s), 7.72 (1H, dd, J=4.6, 8.6 Hz), 8.07 (1H, dd, J=1.8, 8.6 Hz), 8.63 (1H, d, J=4.6 Hz), 8.85 (1H, d, J=1.8 Hz).

### EXAMPLE 325

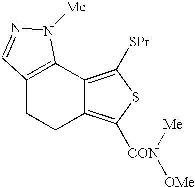

N-Methoxy-N-methyl-4,5-dihydro-1-methyl-8-propylsufanyl-1H-thieno[3,4-g]indazole-6-carboxamide:

According to the same manner as that in Example 250, the title compound (3.7 g, 79% yield) was obtained as a light yellow, oily substance from the compound (4.1 g) obtained in Example 45. ¹H-NMR (δ ppm in CDCl₃): 0.96 (3H, t, J=7.4 Hz), 1.5-1.7 (2H, m), 2.60 (2H, t, J=6.6 Hz), 2.86 (2H, t, J=7.0 Hz), 3.14 (2H, t, J=6.6 Hz), 3.35 (3H, s), 3.74 (3H, s), 4.21 (3H, s), 7.40 (1H, s).

### EXAMPLE 326

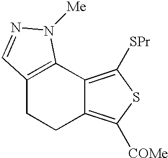

1-[4,5-Dihydro-1-methyl-8-propylsulfanyl-1H-thieno[3,4-g]indazol-6-yl]-1-ethanone:

According to the same manner as that in Example 321, the title compound (0.66 g, 76% yield) was obtained as a light yellow, oily substance from the compound (1.0 g) that was obtained in Example 325. ¹H-NMR (δ ppm in CDCl₃): 1.00 (3H, t, J=7.4 Hz), 1.6-1.8 (2H, m), 2.53 (3H, s), 2.61 (2H, t, J=6.8 Hz), 2.95 (2H, t, J=7.2 Hz), 3.17 (2H, t, J=6.8 Hz), 4.18 (3H, s), 7.41 (1H, s)

### EXAMPLE 327

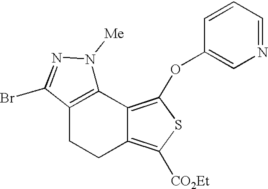

3-Bromo-4,5-dihydro-1-methyl-8-(3-pyridinyloxy)-1H-thieno[3,4-g]indazole-6-carboxylic Acid Ethyl Ester:

According to the same manner as that in Example 297, the title compound (0.16 g, 22% yield) was obtained as light yellow needles from the compound (0.60 g) obtained in Example 268. Melting point: 130-131° C.

### EXAMPLE 328

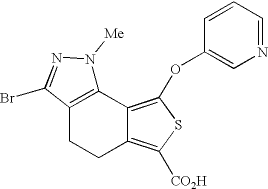

3-Bromo-4,5-dihydro-1-methyl-8-(3-pyridinyloxy)-1H-thieno[3,4-g]indazole-6-carboxylic Acid:

According to the same manner as that in Example 15, the title compound (0.11 g, 86% yield) was obtained as colorless needles from the compound (0.13 g) obtained in Example 327. Melting point: 275-276° C.

### EXAMPLE 329

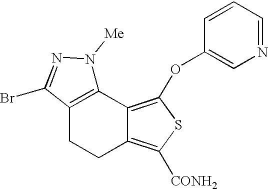

3-Bromo-4,5-dihydro-1-methyl-8-(3-pyridinyloxy)-1H-thieno[3,4-g]indazole-6-carboxamide:

According to the same manner as that in Example 211, the title compound (79 mg, 95% yield) was obtained as colorless prisms from the compound (84 mg) obtained in Example 328. Melting point: 233-234° C.

### EXAMPLE 330

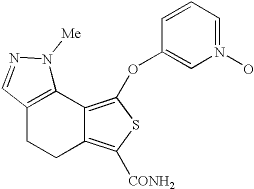

4,5-Dihydro-1-methyl-8-(1-oxo-3-pyridinyloxy)-1H-thieno[3,4-g]indazole-6-carboxamide:

To a solution of the compound (0.17 g), which was obtained in Example 284, dissolved in acetic acid (5 ml) was added an aqueous, 30% solution of hydrogen peroxide (0.17 ml). The resulting solution was stirred at 60° C. for 9 hours and evaporated under reduced pressure to remove the solvent. The thus-obtained oily substance was subjected to column chromatography on silica gel. From the fractions eluted with chloroform-methanol (9:1), the title compound (53 mg, 30% yield) was obtained as light yellow prisms. Melting point: 194-195° C.

### EXAMPLE 331

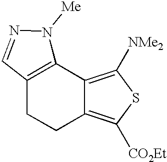

4,5-Dihydro-8-dimethylamino-1-methyl-1H-thieno[3,4-g]indazole-6-carboxylic Acid Ethyl Ester:

To a solution of the compound (11.8 g), which was obtained in Reference Example 39, in N,N-dimethylformamide (50 ml) was added tris(dimethylamino)methane (26 ml) and the resulting solution was stirred at room temperature for 5 hours at 60-70° C. The reaction solution was poured into ice water and extracted with ethyl acetate. The extract was washed with an aqueous solution of sodium chloride and then evaporated under reduced pressure to remove the solvent. To a solution of the residue in ethanol (10 ml) were added hydrazine monohydrate (3.2 ml) and 5 N hydrochloric acid (20 ml). The resulting mixture was stirred at 60-70° C. for 2 hours and then evaporated under reduced pressure. The residue was diluted with ethyl acetate and the resulting solution was washed with an aqueous solution of sodium chloride, then dried (MgSO₄), and concentrated under reduced pressure. The thus-obtained oily substance was subjected to column chromatography on silica gel. From the fractions eluted with ethyl acetate-hexane (3:2), the title compound (7.2 g, 60% yield) was obtained as light pink needles. Melting point: 118-119° C.

### EXAMPLE 332

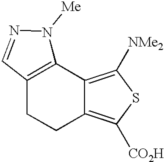

4,5-Dihydro-8-dimethylamino-1-methyl-1H-thieno[3,4-g]indazole-6-carboxylic Acid:

According to the same manner as that in Example 15, the title compound (5.2 g, 87% yield) was obtained as light yellow crystals from the compound (6.6 g) that was obtained in Example 332. Melting point: 208-209° C.

### EXAMPLE 333

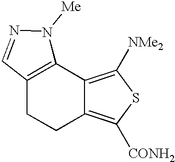

4,5-Dihydro-8-dimethylamino-1-methyl-1H-thieno[3,4-g]indazole-6-carboxamide:

According to the same manner as that in Example 211, the title compound (2.5 g, 64% yield) was obtained as light yellow prisms from the compound (4.0 g) obtained in

### EXAMPLE 332

### Melting point: 161-162° C

### EXAMPLE 334

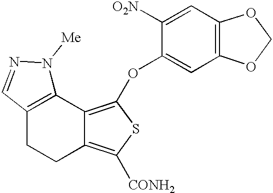

4,5-Dihydro-1-methyl-8-(3,4-metylenedioxy-6-nitrophenoxy)-1H-thieno[3,4-g]indazole-6-carboxamide:

A solution of the compound (0.28 g), which was obtained in Example 216, in nitric acid (1 ml) was stirred under ice cooling for 1 hour. The reaction solution was poured into ice water and extracted with ethyl acetate. This extract was washed with an aqueous solution of sodium chloride, then dried (MgSO₄), and concentrated under reduced pressure. The thus-obtained, yellow oily substance was crystallized from ethyl acetate to obtain the title compound (0.28 g, 89% yield) as light yellow prisms. Melting point: 230-231° C.

### EXAMPLE 335

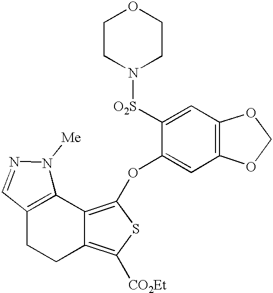

4,5-Dihydro-8-[3,4-methylenedioxy-6-(morpholinosulfonyl)phenoxy]-1-methyl-1H-thieno[3,4-g]indazole-6-carboxylic Acid Ethyl Ester:

The compound (1.5 g) obtained in Example 14a hereinafter was added in small portions to chlorosulfonic acid (3.0 g) and the resulting solution was stirred at room temperature for 30 minutes, then diluted with chloroform (15 ml), and cooled. To this solution was added morpholine (8 ml) and the resulting solution was stirred at room temperature for 2 hours. The reaction solution was poured into an aqueous solution of citric acid and extracted with ethyl acetate. The extract was washed with an aqueous solution of sodium chloride, then dried (MgSO₄), and concentrated under reduced pressure. The thus-obtained oily substance was subjected to column chromatography on silica gel. From the fractions eluted with ethyl acetate-hexane (3:2), the title compound (0.35 g, 17% yield) was obtained as a light yellow, amorphous solid. ¹H NMR (δ ppm in CDCl₃): 1.36 (3H, t, J=7.2 Hz), 2.73 (2H, t, J=6.8 Hz), 3.1-3.3 (4H, m), 3.29 (2H, t, J=6.8 Hz), 3.6-3.8 (4H, m), 4.07 (3H, s), 4.32 (2H, q, J=7.2 Hz), 6.10 (2H, s), 6.67 (1H, s), 7.36 (1H, s), 7.38 (1H, s).

### EXAMPLE 336

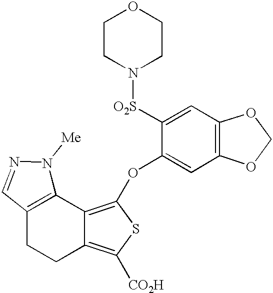

4,5-Dihydro-8-[3,4-methylenedioxy-6-(morpholinosulfonyl)phenoxy]-1-methyl-1H-thieno[3,4-g]indazole-6-carboxylic Acid:

According to the same manner as that in Example 15, the title compound (0.30 g, 100% yield) was obtained as colorless crystals from the compound (0.32 g) obtained in Example 337. Melting point: >300° C.

### EXAMPLE 337

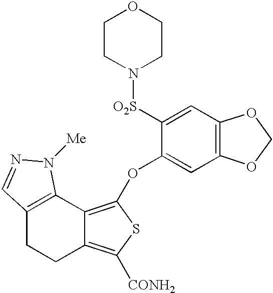

4,5-Dihydro-1-methyl-8-[3,4-methylenedioxy-6-(morpholinosulfonyl)phenoxy]-1H-thieno[3,4-g]indazole-6-carboxamide:

According to the same manner as that in Example 211, the title compound (0.265 g, 99% yield) was obtained as colorless prisms from the compound (0.27 g) obtained in Example 336. Melting point: >300° C.

### EXAMPLE 338

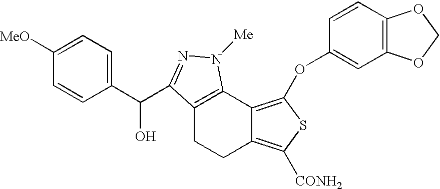

4,5-Dihydro-3-[1-hydroxy-(4-methoxyphenyl)methyl]-1-methyl-8-(3,4-methylenedioxyphenoxy)-1H-thieno[3,4-g]indazole-6-carboxamide:

A solution of the compound (0.50 g), which was obtained in Example 302, dissolved in THF (50 ml) was cooled to −70° C. This solution was subjected to addition of 1.6 M butyllithium (1.5 ml) and stirring at the same temperature for 30 minutes. Further was added p-anisaldehyde (0.20 ml) and the temperature of the resulting solution was raised to −30° C. over a period of 3 hours. The reaction solution was poured into an aqueous solution of citric acid and extracted with ethyl acetate. The extract was washed with an aqueous solution of sodium chloride, then dried (MgSO₄), and concentrated under reduced pressure. The thus-obtained colorless crystals were dissolved in DMF (15 ml) and the resulting solution was cooled with ice. To this solution were added HOBt-NH₃ (0.17 g) and WSC (0.21 g) and the resulting solution was stirred at room temperature for 16 hours. The reaction solution was poured into ice water and extracted with ethyl acetate. The extract was washed with an aqueous solution of sodium hydrogen carbonate and an aqueous solution of sodium chloride, then dried (MgSO₄), and concentrated under reduced pressure. The thus-obtained yellow oily substance was subjected to column chromatography on silica gel. From the fractions eluted with ethyl acetate-hexane-methanol (4:1:0.2), the title compound (69 mg, 13% yield) was obtained as colorless crystals. Melting point: 187-188° C.

### EXAMPLE 339

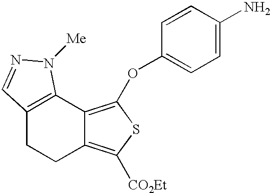

4,5-Dihydro-8-(4-aminophenoxy)-1-methyl-1H-thieno[3,4-g]indazole-6-carboxylic Acid Ethyl Ester:

According to the same manner as that in Example 13a, the title compound (2.5 g, 46% yield) was obtained as light yellow prisms from the compound (5.0 g) obtained in Example 12a hereinafter. Melting point: 203-204° C.

### EXAMPLE 340

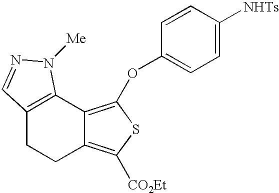

4,5-Dihydro-1-methyl-8-[4-(p-toluenesulfonylamino)phenoxy]-1H-thieno[3,4-g]indazole-6-carboxylic Acid Ethyl Ester:

A solution of the compound (1.0 g) obtained in Example 339, p-toluenesulfonyl chloride (0.62 g), and potassium carbonate (0.45 g) in DMF (20 ml) was stirred at room temperature for 6 hours. The reaction solution was evaporated under reduced pressure and the residue was diluted with ethyl acetate. The resulting mixture was washed successively with citric acid, water, and an aqueous solution of sodium chloride, then dried (MgSO₄), and concentrated under reduced pressure. The thus-obtained yellow oily substance was subjected to column chromatography on silica gel. From the fractions eluted with ethyl acetate-hexane (1:1), the title compound (0.65 g, 46% yield) was obtained as light pink prisms. Melting point: 201-202° C.

### EXAMPLE 341

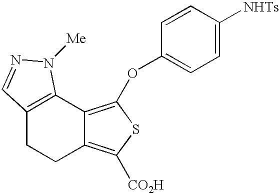

4,5-Dihydro-8-[4-(p-toluenesulfonylamino)phenoxy]-1-methyl-1H-thieno[3,4-g]indazole-6-carboxylic Acid:

According to the same manner as that in Example 15, the title compound (0.69 g, 93% yield) was obtained as colorless crystals from the compound (0.78 g) obtained in Example 340. Melting point: 279-280° C.

### EXAMPLE 342

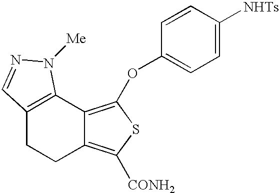

4,5-Dihydro-1-methyl-8-[4-(p-toluenesulfonylamino)phenoxy]-1H-thieno[3,4-g]indazole-6-carboxamide:

According to the same manner as that in Example 211, the title compound (0.586 g, 95% yield) was obtained as colorless prisms from the compound (0.62 g) that was obtained in Example 341. Melting point: 221-222° C.

### EXAMPLE 343

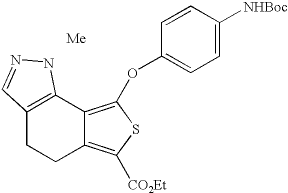

8-[4-(t-Butoxycarbonylamino)phenoxy]-4,5-dihydro-1-methyl-1H-thieno[3,4-g]indazole-6-carboxylic Acid Ethyl Ester:

A solution of the compound (1.34 g) obtained in Example 339 and di-t-butyl dicarbonate (1.2 g) in THF (80 ml) was stirred at 60° C. for 2 days. The reaction solution was concentrated under reduced pressure and the thus-obtained, oily substance was crystallized from ethyl acetate-hexane to obtain the title compound (1.58 g, 93% yield) as light yellow needles. Melting point: 186-187° C.

### EXAMPLE 344

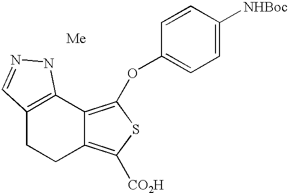

8-[4-(t-Butoxycarbonylamino)phenoxy]-4,5-dihydro-1-methyl-1H-thieno[3,4-g]indazole-6-carboxylic Acid:

According to the same manner as that in Example 15, the title compound (1.38 g, 98% yield) was obtained as light yellow crystals from the compound (1.50 g) obtained in Example 343. Melting point: 260-261° C.

### EXAMPLE 345

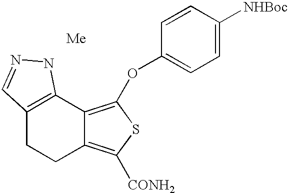

8-[4-(t-Butoxycarbonylamino)phenoxy]-4,5-dihydro-1-methyl-1H-thieno[3,4-g]indazole-6-carboxamide:

According to the same manner as that in Example 211, the title compound (1.2 g, 93% yield) was obtained as light yellow crystals from the compound (1.3 g) obtained in

### EXAMPLE 344

### Melting point: 242-243° C

### EXAMPLE 346

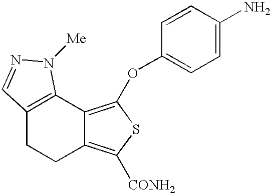

8-(4-Aminophenoxy)-4,5-dihydro-1-methyl-1H-thieno[3,4-g]indazole-6-carboxamide:

A solution of the compound (0.80 g) obtained in Example 345, in a mixed solvent of THF (80 ml) and methanol (20 ml) was subjected to a further addition of 5 N hydrochloric acid (20 ml) and stirring at 80° C. for 6 hours. The reaction solution was concentrated under reduced pressure, the residue was poured into an aqueous solution of sodium hydrogen carbonate and extracted with ethyl acetate, and the extract was dried (MgSO₄) and concentrated under reduced pressure. The thus-obtained, oily substance was crystallized from ethyl acetate-ether to obtain the title compound (0.58 g, 94% yield) as light yellow prisms. Melting point: 214-216° C.

### EXAMPLE 347

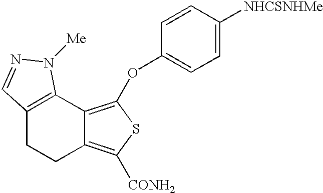

4,5-Dihydro-1-methyl-8-[4-(methylaminothiocarbonylamino)phenoxy]-1H-thieno[3,4-g]indazole-6-carboxamide:

A solution of the compound (0.28 g) obtained in Example 346 and methyl isothiocyanate (0.12 ml) in THF (30 ml) was heated at reflux for 3 days. The reaction solution, the reaction solution was concentrated under reduced pressure, the residue was subjected to column chromatography on silica gel. From the fractions eluted with ethyl acetate-methanol (20:1), the title compound (0.19 g, 56% yield) was obtained as colorless prisms. Melting point: 199-201° C.

### EXAMPLE 348

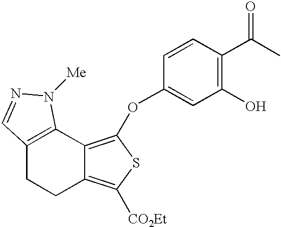

8-(4-Acetyl-3-hydroxyphenoxy)-4,5-dihydro-1-methyl-1H-thieno[3,4-g]indazole-6-carboxylic Acid Ethyl Ester:

To a solution of 4,5-dihydro-8-(3-methoxyphenoxy)-1-methyl-1H-thieno[3,4-g]indazole-6-carboxylic acid ethyl ester (1.5 g) and acetyl chloride (0.62 ml) in 1,2-dichloroethane (30 ml) was added aluminum chloride (2.2 g) under ice cooling and the resulting solution was stirred at room temperature for 25 hours. The reaction solution was poured into dilute hydrochloric acid and extracted with ethyl acetate. The extract was washed with an aqueous solution of sodium chloride, then dried (MgSO₄), and concentrated under reduced pressure. The thus-obtained, yellow oily substance was subjected to column chromatography on silica gel. From the fractions eluted with ethyl acetate-hexane (1:2), the title compound (0.98 g, 61% yield) was obtained as colorless prisms. Melting point: 131-132° C.

### EXAMPLE 349

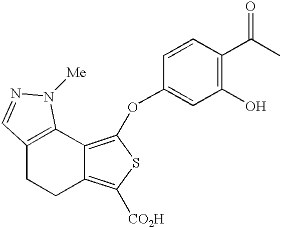

8-(4-Acetyl-3-hydroxyphenoxy)-4,5-dihydro-1-methyl-1H-thieno[3,4-g]indazole-6-carboxylic Acid:

According to the same manner as that in Example 15, the title compound (0.78 g, 98% yield) was obtained as light yellow crystals from the compound (0.85 g) obtained in Example 348. Melting point: >276° C. (decomposition).

### EXAMPLE 350

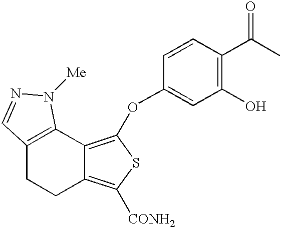

8-(4-Acetyl-3-hydroxyphenoxy)-4,5-dihydro-1-methyl-1H-thieno[3,4-g]indazole-6-carboxamide:

According to the same manner as that in Example 211, the title compound (0.56 g, 92% yield) was obtained as colorless crystals from the compound (0.60 g) obtained in Example 349. Melting point: 275-276° C.

### EXAMPLE 351

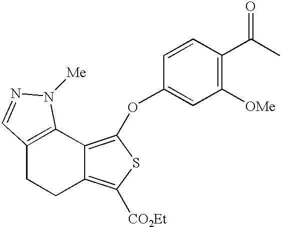

8-(4-Acetyl-3-methoxyphenoxy)-4,5-dihydro-1-methyl-1H-thieno[3,4-g]indazole-6-carboxylic Acid Ethyl Ester:

To a solution of 4,5-dihydro-8-(3-methoxyphenoxy)-1-methyl-1H-thieno[3,4-g]indazole-6-carboxylic acid ethyl ester (1.5 g) and acetyl chloride (0.56 ml) in 1,2-dichloroethane (30 ml) was added tin chloride (1.44 ml) under ice cooling and the resulting solution was stirred at room temperature for 15 hours. The reaction solution was poured into dilute hydrochloric acid and extracted with ethyl acetate. The extract was washed with an aqueous solution of sodium chloride, then dried (MgSO₄), and concentrated under reduced pressure. The thus-obtained yellow oily substance was subjected to column chromatography on silica gel. From the fractions eluted with ethyl acetate-hexane (2:3), the title compound (0.42 g, 25% yield) was obtained as colorless prisms. Melting point: 132-133° C.

### EXAMPLE 352

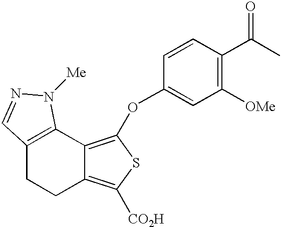

8-(4-Acetyl-3-methoxyphenoxy)-4,5-dihydro-1-methyl-1H-thieno[3,4-g]indazole-6-carboxylic Acid:

According to the same manner as that in Example 15, the title compound (0.33 g, 91% yield) was obtained as colorless crystals from the compound (0.38 g) obtained in Example 351. Melting point: 269-270° C.

### EXAMPLE 353

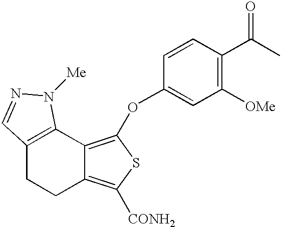

8-(4-Acetyl-3-methoxyphenoxy)-4,5-dihydro-1-methyl-1H-thieno[3,4-g]indazole-6-carboxamide:

According to the same manner as that in Example 211, the title compound (0.25 g, 84% yield) was obtained as colorless prisms from the compound (0.29 g) obtained in Example 352. Melting point: 181-182° C.

### EXAMPLE 354

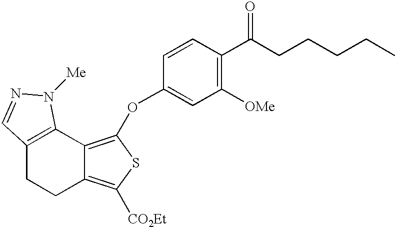

8-(4-n-Hexanoyl-3-methoxyphenoxy)-4,5-dihydro-1-methyl-1H-thieno[3,4-g]indazole-6-carboxylic Acid Ethyl Ester:

To a solution of 4,5-dihydro-8-(3-methoxyphenoxy)-1-methyl-1H-thieno[3,4-g]indazole-6-carboxylic acid ethyl ester (1.25 g) and hexanoyl chloride (0.91 ml) in 1,2-dichloroethane (30 ml) was added aluminum chloride (1.4 g) under ice cooling and the resulting solution was stirred at room temperature for 7 hours. The reaction solution was poured into dilute hydrochloric acid and extracted with ethyl acetate. The extract was washed with an aqueous solution of sodium chloride, then dried (MgSO₄), and concentrated under reduced pressure. The thus-obtained yellow oily substance was subjected to column chromatography on silica gel. From the fractions eluted with ethyl acetate-hexane (1:2), the title compound (0.89 g, 57% yield) was obtained as a light yellow oily substance.

¹H NMR (δ ppm in CDCl₃): 0.94 (3H, t, J=6.6 Hz), 1.2-1.4 (4H, m), 1.35 (3H, t, J=7.0 Hz), 1.6-1.8 (2H, m), 2.72 (2H, t, J=7.0 Hz), 2.94 (2H, t, J=7.0 Hz), 3.28 (2H, t, J=7.0 Hz), 3.88 (3H, s), 4.01 (3H, s), 4.32 (2H, q, J=7.0 Hz), 6.7-6.8 (2H, m), 7.36 (1H, s), 7.75 (1H, d, J=6.0 Hz)

### EXAMPLE 355

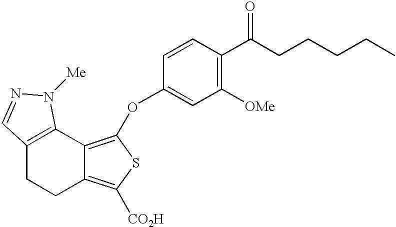

8-(4-n-Hexanoyl-3-methoxyphenoxy)-4,5-dihydro-1-methyl-1H-thieno[3,4-g]indazole-6-carboxylic Acid:

According to the same manner as that in Example 15, the title compound (0.68 g, 85% yield) was obtained as colorless crystals from the compound (0.85 g) obtained in Example 354. Melting point: 265-266° C.

### EXAMPLE 356

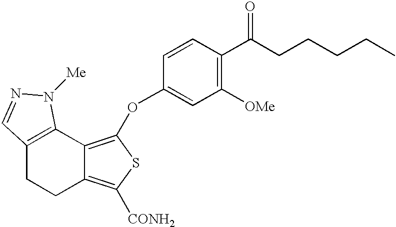

8-(4-n-Hexanoyl-3-methoxyphenoxy)-4,5-dihydro-1-methyl-1H-thieno[3,4-g]indazole-6-carboxamide:

According to the same manner as that in Example 211, the title compound (0.42 g, 73% yield) was obtained as colorless crystals from the compound (0.57 g) obtained in Example 355. Melting point: 164-165° C.

### EXAMPLE 357

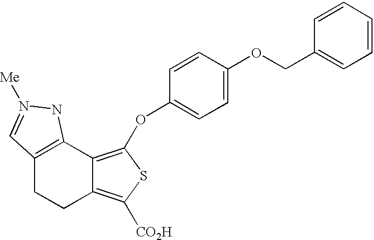

8-(4-Benzyloxyphenoxy)-4,5-dihydro-2-methyl-2H-thieno[3,4-g]indazole-6-carboxylic Acid:

According to the same manner as that in Example 15, the title compound (2.9 g, 91% yield) was obtained as colorless crystals from 8-(4-benzyloxyphenoxy)-4,5-dihydro-2-methyl-2H-thieno[3,4-g]indazole-6-carboxylic acid ethyl ester (3.4 g). Melting point: 222-223° C.

### EXAMPLE 358

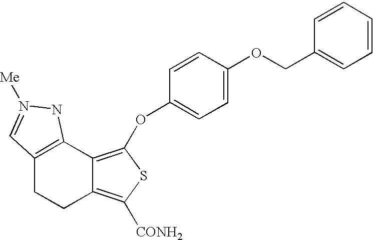

8-(4-Benzyloxyphenoxy)-4,5-dihydro-2-methyl-2H-thieno[3,4-g]indazole-6-carboxamide:

According to the same manner as that in Example 211, the title compound (1.27 g, 85% yield) was obtained as colorless prisms from the compound (1.5 g) obtained in Example 357. Melting point: 216-217° C.

### EXAMPLE 359

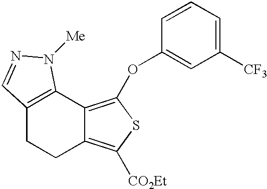

4,5-Dihydro-1-methyl-8-(3-trifluoromethylphenoxy)-1H-thieno[3,4-g]indazole-6-carboxylic Acid Ethyl Ester:

According to the same manner as that in Example 13a hereinafter, the title compound (1.37 g, 37% yield) was obtained as light yellow prisms from the compound (3.0 g) obtained in Example 12a hereinafter. Melting point: 138-139° C.

### EXAMPLE 360

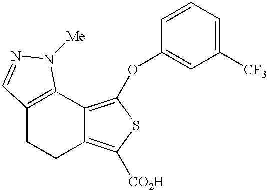

4,5-Dihydro-1-methyl-8-(3-trifluoromethylphenoxy)-1H-thieno[3,4-g]indazole-6-carboxylic Acid:

According to the same manner as that in Example 15, the title compound (1.12 g, 96% yield) was obtained as colorless crystals from the compound (1.26 g) obtained in Example 359. Melting point: 296-298° C.

### EXAMPLE 361

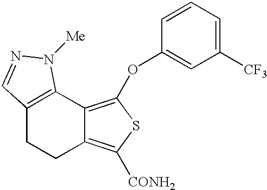

4,5-Dihydro-1-methyl-8-(3-trifluoromethylphenoxy)-1H-thieno[3,4-g]indazole-6-carboxamide:

According to the same manner as that in Example 211, the title compound (0.96 g, 96% yield) was obtained as colorless prisms from the compound (1.0 g) obtained in Example 360. Melting point: 178-179° C.

According to the same manner as that in Example 143, compounds in Examples 362 to 367 were obtained.

TABLE 33

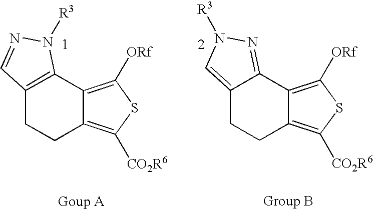

| | | | | Melting | Melting |
|---|---|---|---|---|---|
| | | | | point | point |
| | | | | (° C.) | (° C.) |
| | | | | in | in |
| | | | | Group | Group |
| | | | | A | B |
| | | | | where | where |
| | | | | R³ is | R³ is |
| | | | | bound | bound |
| | | | | at | at |
| Ex. | | | | poison | poison |
| No. | R³ | R⁶ | Rf | 1 | 2 |
| 362 | Me | Et | 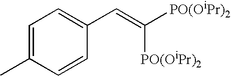 | 129-130 | Oily sub- stance ¹⁾ |
| 363 | H | Et | 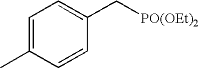 | Oily sub- stance ²⁾ | — |
| 364 | Et | Et | 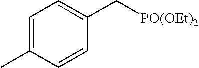 | Oily sub- stance ³⁾ | Oily sub- stance⁴⁾ |
| 365 | Me | Et | 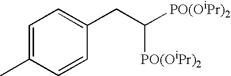 | Oily sub- stance ⁵⁾ | |
| 366 | Me | ^{t}Bu | 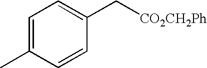 | Oily sub- stance ⁶⁾ | Oily sub- stance ⁷⁾ |
| 367 | Me | ^{t}Bu | 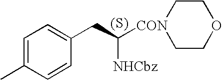 | Amor- phous solid ⁸⁾ | Amor- phous solid ⁹⁾ |

^{1) 1}H NMR (δ ppm in CDCl₃): 1.21 (6H, d, J=6.2Hz), 1.24 (6H, d, J=6.2Hz), 1.33 (3H, t, J=7.0Hz), 1.37 (6H, d, J=6.2Hz), 1.42 (6H, d, J=6.2Hz), 2.77 (2H, t, J=7.0Hz), 3.33 (2H, t, J=7.0Hz), 3.85 (3H, s), 4.29 (2H, q, J = 7.0 Hz), 4.6-4.9 (4H, m), 7.15 (1H, s), 7.23 (2H, d, J = 8.8 Hz), 7.90 (2H, d, J = 8.8 Hz), 8.24 (1H, dd, J = 10.4, 47.6 Hz).
^{2) 1}H NMR (δ ppm in CDCl₃): 1.25 (6H, t, J = 6.8 Hz), 1.32 (3H, t, J = 7.0 Hz), 2.83 (2H, t, J = 7.2 Hz), 3.16 (2H, d, J = 21.6 Hz), 3.35 (2H, t, J = 7.2 Hz), 3.9-4.1 (4H, m), 4.28 (2H, q, J = 7.0 Hz), 7.20 ( 2H, d, J = 8.8 Hz), 7.34 (2H, dd, J = 2.4, 8.8 Hz), 7.42 (1H, s).
^{3) 1}H NMR (δ ppm in CDCl₃): 1.26 (6H, t, J = 7.0 Hz), 1.32 (3H, t, J = 7.0 Hz), 1.39 (3H, t, J = 7.0 Hz), 2.69 (2H, t, J = 6.8 Hz), 3.16 (2H, d, J = 21.6 Hz), 3.26 (2H, t, J = 6.8 Hz), 3.9-4.5 (8H, m), 7.1-7.4 (5H, m).
^{4) 1}H NMR (δ ppm in CDCl₃): 1.25 (6H, t, J = 7.0 Hz), 1.31 (3H, t, J = 7.0 Hz), 1.45 (3H, t, J = 7.0 Hz), 2.77 (2H, t, J = 6.8 Hz), 3.15 (2H, d, J = 21.6 Hz), 3.32 (2H, t, J = 6.8 Hz), 3.9-4.1 (4H, m), 4.17 (2H, q, J = 7.0 Hz), 4.27 (2H, q, J = Hz), 7.1-7.4 (5H, m).
^{5) 1}H NMR (δ ppm in CDCl₃): 1.2-1.4 (15H, m), 2.48 (1H, tt, J = 6.2, 24.0 Hz), 2.70 (2H, t, J = 7.0 Hz), 3.1-3.4 (4H, m), 4.05 (3H, s), 4.28 (2H, q, J = 7.0 Hz), 4.7-4.9 (4H, m), 7.10 (2H, d, J = 8.4 Hz), 7.29 (2H, d, J = 8.4 Hz), 7.35 (1H, s).
^{6) 1}H NMR (δ ppm in CDCl₃): 1.54 (9H, s), 2.70 (2H, t, J = 7.0 Hz), 3.24 (2H, t, J = 7.0 Hz), 3.67 (2H, s), 4.03 (3H, s), 5.15 (2H, s), 7.12 (2H, d, J = 8.8 Hz), 7.2-7.4 (8H, m).
^{7) 1}H NMR (δ ppm in CDCl₃): 1.53 (9H, s), 2.76 (2H, t, J = 7.0 Hz), 3.30 (2H, t, J = 7.0 Hz), 3.67 (2H, s), 3.88 (3H, s), 5.15 (2H, s), 7.15 (1H, s), 7.2-7.4 (9H, m).
^{8) 1}H NMR (δ ppm in CDCl₃): 1.53 (9H, s), 2.69 (2H, t, J = 7.0 Hz), 2.8-3.7 (10H, m), 3.24 (2H, t, J = 7.0 Hz), 4.02 (3H, s), 4.7-4.9 (1H, m), 5.09 (2H, s), 5.65 (1H, d, J = 9.0 Hz), 7.09 (2H, d, J = 8.4 Hz), 7.21 (2H, d, J = 8.4 Hz), 7.34 (6H, (6H, s).
^{9) 1}H NMR (δ ppm in CDCl₃): 1.52 (9H, s), 2.75 (2H, t, J = 7.0 Hz), 2.8-3.7 (10H, m), 3.30 (2H, t, J = 7.0 Hz), 3.88 (3H, s), 4.7-4.9 (1H, m), 5.09 (2H, s), 5.67 (1H, d, J = 9.0 Hz), 7.15 (1H, s), 7.19 (4H, s), 7.35 (5H, s).

According to the same manner as that in Example 15, compounds in Examples 368 to 369 were obtained.

TABLE 34

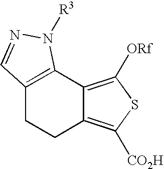

| Example | | | Melting |
|---|---|---|---|
| No. | R³ | Rf | point (° C.) |
| 368 | H | 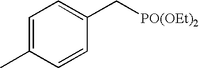 | 212-213 |
| 369 | Me | 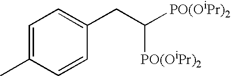 | 163-164 |

According to the same manner as that in Example 211, compounds in Examples 370 to 371 were obtained.

TABLE 35

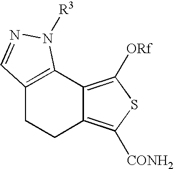

| Example | | | Melting |
|---|---|---|---|
| No. | R³ | Rf | point (° C.) |
| 370 | H | 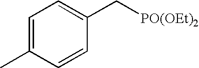 | 186-187 |
| 371 | Me | 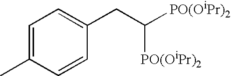 | Oily substance¹⁾ |

### EXAMPLE 372

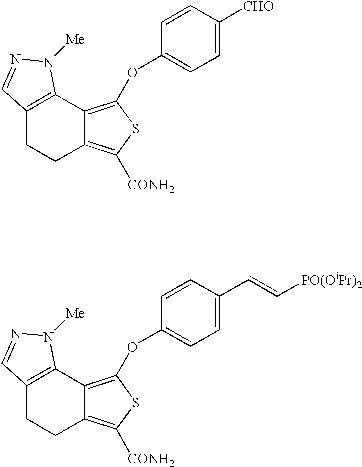

8-(4-Formylphenoxy)-4,5-dihydro-1-methyl-1H-thieno[3,4-g]indazole-6-carboxamide and 8-{4-((E)-2-diisopropoxyphosphorylvinyl)phenoxy}-4,5-dihydro-1-methyl-1H-thieno[3,4-g]indazole-6-carboxamide:

The compound in Group A (2.0 g) obtained in Example 362 was dissolved in a mixture of THF (30 ml) and methanol (30 ml). To this solution was added an aqueous solution (10 ml) of potassium hydroxide (0.47 g) and the resulting mixture was stirred at room temperature for 15 hours. The reaction solution was concentrated under reduced pressure and the residue was made acid with an aqueous solution of citric acid. The resulting mixture was subjected to extraction with ethyl acetate, drying (MgSO₄), and concentration under reduced pressure. A solution of the thus-obtained light yellow crystals (1.7 g) and HOBt-NH₃ (0.46 g) dissolved in DMF (50 ml) was cooled with ice. To this solution was added WSC (0.58 g) and the resulting solution was stirred at room temperature for 15 hours. The reaction solution was concentrated under reduced pressure and poured into ice water. This mixture was subjected to extraction with ethyl acetate, washing with an aqueous solution of sodium hydrogen carbonate and an aqueous solution of sodium chloride, then drying (MgSO₄), and concentration under reduced pressure. The thus-obtained brown oily substance was subjected to column chromatography on silica gel. From the fractions eluted with ethyl acetate, 8-(4-formylphenoxy)-4,5-dihydro-1-methyl-1H-thieno[3,4-g]indazole-6-carboxamide (0.15 g, 15% yield) was obtained as colorless prisms (melting point: 209-210° C.). In addition, from the fractions eluted with ethyl acetate-methanol (30:1), 8-{4-((E)-2-diisopropoxyphosphorylvinyl)phenoxy}-4,5-dihydro-1-methyl-1H-thieno[3,4-g]indazole-6-carboxamide (1.15 g, 79% yield) was obtained as a light yellow amorphous solid. ¹H NMR (δ ppm in CDCl₃): 1.2-1.5 (12H, m), 2.73 (2H, t, J=7.0 Hz), 3.19 (2H, t, J=7.0 Hz), 4.01 (3H, s), 4.6-4.9 (2H, m), 5.63 (2H, brs), 6.20 (1H, t, J=17.2 Hz), 7.14 (2H, d, J=8.8 Hz), 7.35 (1H, s), 7.2-7.4 (1H, m), 7.51 (2H, d, J=8.8 Hz).

### EXAMPLE 373

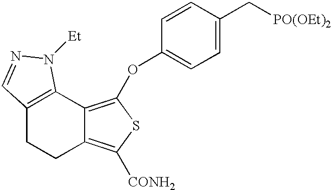

8-(4-Diethoxyphosphorylmethylphenoxy)-4,5-dihydro-1-ethyl-1H-thieno[3,4-g]indazole-6-carboxamide:

The compound in Group A (2.2 g) obtained in Example 364 was dissolved in a mixed solution of THF (20 ml) and methanol (20 ml). To this solution was added an aqueous solution (15 ml) of potassium hydroxide (0.80 g) and the resulting mixture was stirred at room temperature for 15 hours. The reaction solution was concentrated under reduced pressure and the residue was made acid with an aqueous solution of citric acid. The resulting mixture was subjected to extraction with ethyl acetate, drying (MgSO₄), and concentration under reduced pressure. A solution of the thus-obtained light yellow oily substance (2.1 g) and HOBt-NH₃ (0.76 g) dissolved in DMF (40 ml) was cooled with ice. To this solution was added WSC (0.96 g) and the resulting solution was stirred at room temperature for 15 hours. The reaction solution was concentrated under reduced pressure and poured into ice water. This mixed solution was subjected to extraction with ethyl acetate, washing with an aqueous solution of sodium hydrogen carbonate and an aqueous solution of sodium chloride, then drying (MgSO₄), and concentration under reduced pressure. The thus-obtained brown oily substance was subjected to column chromatography on silica gel. From the fractions eluted with ethyl acetate:THF:methylene chloride (1:1:1), the title compound (0.90 g, 45% yield) was obtained as colorless prisms. Melting point: 152-153° C.

According to the same manner as that in Example 10, compounds in Examples 374 to 375 were obtained.

TABLE 36

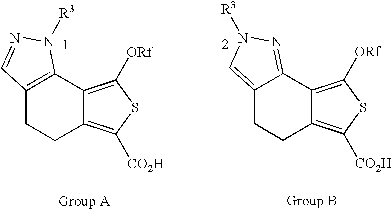

| | | | Melting | Melting |
|---|---|---|---|---|
| | | | point | point |
| | | | (° C.) | (° C.) |
| | | | in Group | in Group |
| | | | A | B |
| | | | where | where |
| | | | R³ is | R³ is |
| Ex. | | | bound at | bound at |
| No. | R³ | Rf | poison 1 | poison 2 |
| 374 | Me | 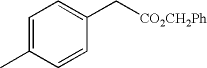 | 227-228 | 196-197 |
| 375 | Me | 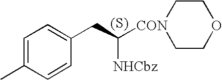 | 209-210 | 144-145 |

According to the same manner as that in Example 211, compounds in Examples 376 to 377 were obtained.

TABLE 37

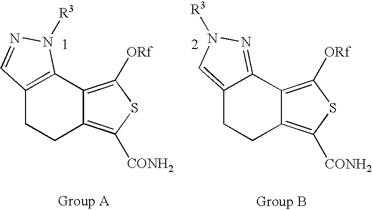

| | | | Melting | Melting |
|---|---|---|---|---|
| | | | point | point |
| | | | (° C.) | (° C.) |
| | | | in Group | in Group |
| | | | A | B |
| | | | where | where |
| | | | R³ is | R³ is |
| Ex. | | | bound at | bound at |
| No. | R³ | Rf | poison 1 | poison 2 |
| 376 | Me | 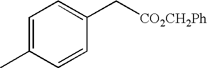 | 1811-182 | 127-128 |
| 377 | Me | 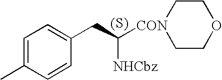 | 141-142 | Amor- phous solid¹⁾ |

^{1) 1}H NMR (δ ppm in CDCl₃): 2.76 (2H, t, J = 7.0 Hz), 2.7-3.7 (10H, m), 3.33 (2H, t, J = 7.0 Hz), 3.91 (3H, s), 4.8-5.0 (1H, m), 5.10 (2H, s), 5.61 (2H, brs), 5.70 (1H, d, J = 8.4 Hz). 7.17 (1H, s), 7.23 (4H, s), 7.35 (5H, s).

### EXAMPLE 378

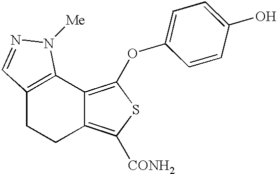

8-(4-Hydroxyphenoxy)-4,5-dihydro-1-methyl-1H-thieno[3,4-g]indazole-6-carboxamide:

To a solution of the compound (2.50 g) as obtained in Example 218 and dissolved in acetic acid (50 ml) was added palladium carbon (0.50 g) and the resulting mixture was stirred under hydrogen atmosphere at atmospheric pressure for 4 hours. The insoluble material was filtered off and the filtrate was concentrated under reduced pressure to obtain crystals. Recrystallization from tetrahydrofuran gave the title compound (1.88 g, 95% yield) as colorless prisms. Melting point: 137-139° C.

### EXAMPLE 379

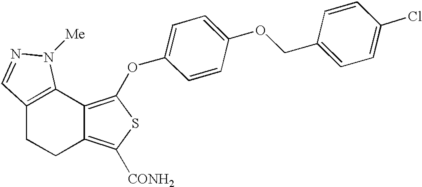

8-(4-(4-Chlorobenzyloxy)phenoxy)-4,5-dihydro-1-methyl-1H-thieno[3,4-g]indazole-6-carboxamide:

A solution of the compound (0.15 g) obtained in Example 378 and 60% sodium hydride (20 mg) in DMF (10 ml) was stirred at room temperature for 10 minutes. To this solution was added 4-chlorobenzyl chloride (64 μl) and the resulting solution was stirred at room temperature for 20 hours. The reaction solution was poured into an aqueous solution of citric acid and extracted with ethyl acetate. This extract was washed with water and an aqueous solution of sodium chloride, then dried (MgSO₄), and concentrated under reduced pressure. The thus-obtained brown solid was subjected to column chromatography on silica gel. From the fractions eluted with ethyl acetate, the title compound (56 mg, 27%) was obtained as light yellow prisms. Melting point: 216-217° C.

### EXAMPLE 380

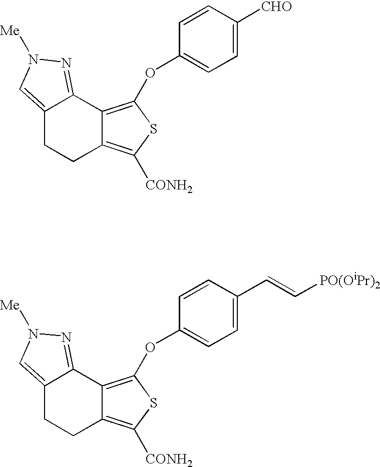

8-(4-Formylphenoxy)-4,5-dihydro-2-methyl-2H-thieno[3,4-g]indazole-6-carboxamide and 8-{4-((E)-2-diisopropoxyphosphorylvinyl)phenoxy}-4,5-dihydro-2-methyl-2H-thieno[3,4-g]indazole-6-carboxamide:

According to the same manner as that in Example 373, the title compounds were obtained from the compound (2.0 g) in Group B obtained in Example 362.

8-(4-Formylphenoxy)-4,5-dihydro-2-methyl-2H-thieno[3,4-g]indazole-6-carboxamide: Light Yellow Prisms (Melting Point: 210-211° C.).

8-{4-((E)-2-Diisopropoxyphosphorylvinyl)phenoxy}-4,5-dihydro-2-methyl-2H-thieno[3,4-g]indazole-6-carboxamide: Yellow Prisms (Melting Point: 216-217° C.).

### EXAMPLE 381

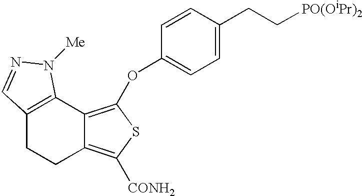

8-{4-(2-Diisopropoxyphosphorylethyl)phenoxy}-4,5-dihydro-1-methyl-1H-thieno[3,4-g]indazole-6-carboxamide:

According to the same manner as that in Example 378, the title compound (0.73 g, 68% yield) was obtained as colorless crystals from 8-{4-((E)-2-diisopropoxyphosphorylvinyl)phenoxy}-4,5-dihydro-1-methyl-1H-thieno[3,4-g]indazole-6-carboxamide (1.0 g) obtained in Example 372. Melting point: 76-77° C.

### EXAMPLE 382

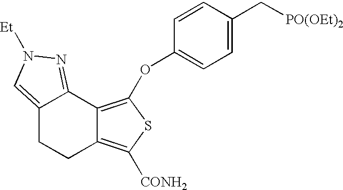

Diehtyl 4-{[6-(aminocarbonyl)-2-ethyl-4,5-dihydro-2H-thieno[3,4-g]indazole-8-yl]oxy}benzylphosphate:

According to the same manner as that in Example 15372, the title compound was obtained as colorless prisms from the compound in Group B obtained in Example 364.

Melting point: 130-131° C.

### EXAMPLE 383

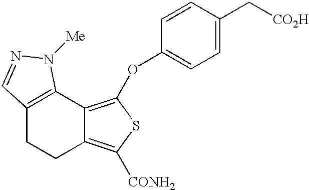

4-[6-(Aminocarbonyl)-4,5-dihydro-1-methyl-1H-thieno[3,4-g]indazol-8-yloxy]phenylacetic Acid:

A mixed solution of the compound in Group A (1.1 g) obtained in Example 376 and 5% palladium carbon (0.40 g) in tetrahydrofuran (25 ml) and methanol (25 ml) was stirred under hydrogen atmosphere at atmospheric pressure for 2 hours. The insoluble material was filtered off and the filtrate was concentrated under reduced pressure to obtain crystals. Recrystallization from tetrahydrofuran-methanol gave the title compound (0.93 g, 100% yield) as colorless prisms. Melting point: 213-214° C.

### EXAMPLE 384

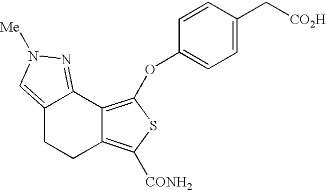

4-(6-Carbamoyl-4,5-dihydro-2-methyl-2H-thieno[3,4-g]indazol-8-yloxy)phenylacetic Acid:

According to the same manner as that in Example 383, the title compound was obtained as light yellow prisms from the compound in Group B obtained in Example 376. Melting point: >290° C. (decomposition).

### EXAMPLE 385

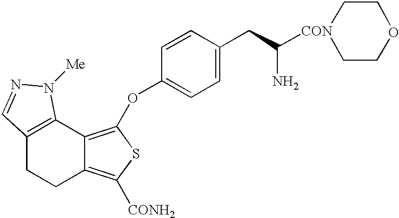

8-{4-[(2S)-2-Amino-3-(4-morpholinyl)-3-oxopropyl]phenoxy}-4,5-dihydro-1-methyl-1H-thieno[3,4-g]indazole-6-carboxamide:

According to the same manner as that in Example 383, the title compound was obtained as a colorless, amorphous solid from the compound in Group A obtained in Example 377. ¹H NMR (δ ppm in CDCl₃): 2.69 (2H, t, J=7.0 Hz), 2.8-3.7 (12H, m), 3.93 (1H, t, J=7.2 Hz), 4.08 (3H, s), 5.66 (2H, brs), 7.14 (2H, d, J=8.4 Hz), 7.24 (2H, d, J=8.4 Hz), 7.36 (1H, s).

### EXAMPLE 386

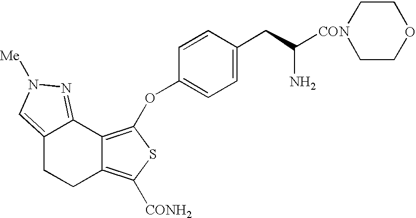

8-{4-[(2S)-2-Amino-3-(4-morpholinyl)-3-oxopropyl]phenoxy}-4,5-dihydro-2-methyl-2H-thieno[3,4-g]indazole-6-carboxamide:

According to the same manner as that in Example 383, the title compound was obtained as a colorless amorphous solid from the compound in Group B obtained in Example 377. ¹H NMR (δ ppm in CDCl₃): 2.76 (2H, t, J=7.0 Hz), 2.8-3.6 (10H, m), 3.31 (2H, t, J=7.0 Hz), 3.91 (3H, s), 3.8-4.0 (1H, m), 5.82 (2H, brs), 7.17 (4H, s), 7.21 (1H, s).

### EXAMPLE 387

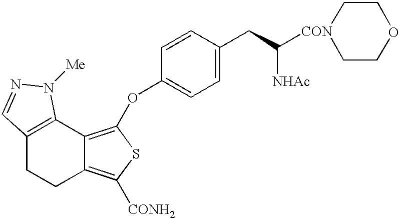

8-{4-[(2S)-2-Acetylamino-3-(4-morpholinyl)-3-oxopropyl]phenoxy}-4,5-dihydro-1-methyl-1H-thieno[3,4-g]indazole-6-carboxamide:

A solution of the compound (0.90 g) obtained in Example 385, acetyl chloride (0.16 ml) and triethylamine (0.32 ml) in anhydrous tetrahydrofuran (40 ml) was stirred under ice cooling for 4 hours. The reaction solution was poured into water and extracted with ethyl acetate. This extract was washed with water and an aqueous solution of sodium chloride, then dried (MgSO₄), and concentrated under reduced pressure. The thus-obtained brown solid was subjected to column chromatography on silica gel. From the fractions eluted with ethyl acetate-methanol (9:1), the title compound (0.30 g, 31% yield) was obtained as colorless prisms. Melting point: 139-140° C.

### EXAMPLE 388

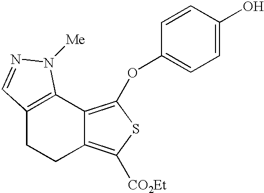

4,5-Dihydro-8-(4-hydroxyphenoxy)-1-methyl-1H-thieno[3,4-g]indazole-6-carboxylic Acid Ethyl Ester:

According to the same manner as that in Example 383, the title compound was obtained as colorless needles from the compound in Group A obtained in Example 150. Melting point: 245-246° C.

### EXAMPLE 389

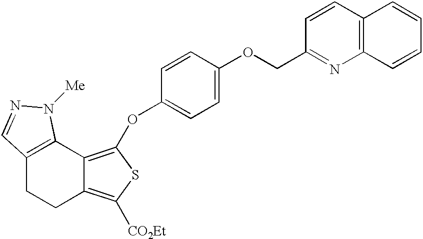

4,5-Dihydro-1-methyl-8-[(4-(2-quinolinylmethoxy)phenoxy]-1H-thieno[3,4-g]indazole-6-carboxylic Acid Ethyl Ester:

A solution of the compound (1.5 g) obtained in Example 388, 2-chloromethylquinoline hydrochloride (1.7 g), and potassium carbonate (1.7 g) in DMF (50 ml) was stirred at 70C for 10 hours. The reaction solution was concentrated under reduced pressure and the residue was made acid with an aqueous solution of citric acid. The resulting mixture was subjected to extraction with ethyl acetate, drying (MgSO₄), and concentration under reduced pressure. The thus-obtained brown oily substance was subjected to column chromatography on silica gel. From the fractions eluted with ethyl acetate-hexane (2:1), the title compound (1.9 g, 92% yield) was obtained as colorless prisms. Melting point: 121-122° C.

According to the same manner as that in Example 389, compounds in Examples 390 to 392 were obtained from the compound obtained in Example 388.

TABLE 38

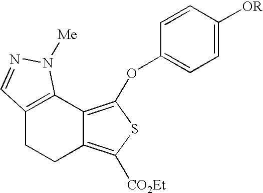

| Example | | Melting |
|---|---|---|
| No. | R | point (° C.) |
| 390 | 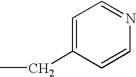 | Oily substance¹⁾ |
| 391 | 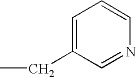 | 134-135 |
| 392 | 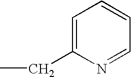 | 133-134 |

¹⁾ H NMR (δ ppm in CDCl₃): 1.32 (3H, t, J = 7.0 Hz), 2.70 (2H, t, J = 7.0 Hz), 3.25 (2H, t, J = 7.0 Hz), 4.09 (3H, s), 4.28 (2H, q, J = 7.0 Hz), 6.98 (2H, d, J = 9.2 Hz), 7.16 (2H, d, J = 9.2 Hz), 7.36 (1H, s), 7.37 (2H, d, J = 6.2 Hz), 8.64 (2H, d, J = 6.2 Hz).

According to the same manner as that in Example 10, compounds in Examples 393 to 396 were obtained.

TABLE 39

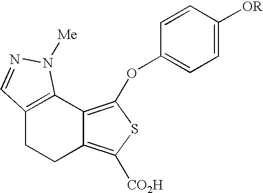

| Example No. | R | Meltingpoint (° C.) |
|---|---|---|
| 393 | 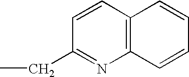 | 269-270 |
| 394 | 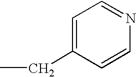 | 228-230 |
| 395 | 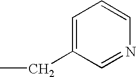 | 261-262 |
| 396 | 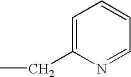 | 256-257 |

According to the same manner as that in Example 211, compounds in Examples 397 to 400 were obtained.

TABLE 40

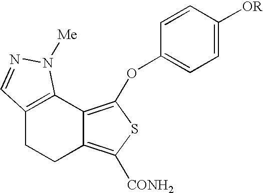

| Example No. | R | Melting point (° C.) |
|---|---|---|
| 397 | 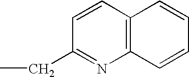 | 214-215 |
| 398 | 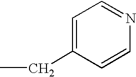 | 249-250 |
| 399 | 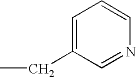 | 229-230 |
| 400 | 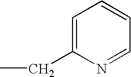 | 209-210 |

### EXAMPLE 401

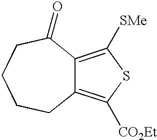

Production of 4,5,6,7-tetrahydro-3-methylsulfanyl-4-oxocyclohepta[c]thiophene-1-carboxylic Acid Ethyl Ester:

Into a three-necked flask (2 L) were charged potassium carbonate (anhydrous) (73 g), N,N-dimethylformamide (150 ml), and 1,3-cycloheptanedione (12.3 g) and the resulting solution was stirred at room temperature for 10 minutes. Thereto was added at once carbon disulfide (11.5 ml) and the resulting solution was stirred at room temperature for 20 minutes. A solution of ethyl chloroacetate (19.7 ml) in N,N-dimethylformamide (150 ml) was added dropwise below 35° C. over a period of 1 hour. After the reaction mixture was stirred at room temperature for 1 hour, methyl iodide (11 ml) was added dropwise and the reaction mixture was heated at 50° C. to react for 1 hour. The reaction solution was cooled, water (1 L) was added dropwise and the precipitated crystals were collected by filtration and washed with water to obtain 16.5 g (33%) of the title compound as light red needles.

¹H NMR (δ ppm in CDCl₃): 1.38 (3H, t, J=7.0 Hz), 1.81-1.92 (4H, m), 2.58 (3H, s), 3.39-3.45 (2H, m), 4.33 (2H, q, J=7.0 Hz).

### EXAMPLE 402

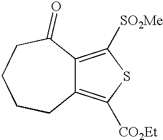

Production of 4,5,6,7-tetrahydro-3-methylsulfonyl-4-oxocyclohepta[c]thiophene-1-carboxylic Acid Ethyl Ester:

To a solution of the compound (3.33 g) obtained in Example 401 and dissolved in trifluoroacetic acid (30 ml) was added an aqueous 30% solution of hydrogen peroxide (8 ml) under ice cooling and the resulting solution was stirred at room temperature for 2 hours. The reaction solution was neutralized with an aqueous, saturated solution of sodium bicarbonate and extracted with ethyl acetate. The extract was washed with water and an aqueous, saturated solution of sodium chloride, then dried (MgSO₄), and evaporated to remove the solvent to obtain 3.52 g (95%) of the title compound as colorless crystals.

¹H NMR (δ ppm in CDCl₃): 1.39 (3H, t, J=7.0 Hz), 1.89 (4H, quint, J=3.0 Hz), 2.74-2.81 (2H, m), 3.30-3.36 (2H, m), 3.50 (3H, s), 4.38 (2H, q, J=7.0 Hz).

### EXAMPLE 403

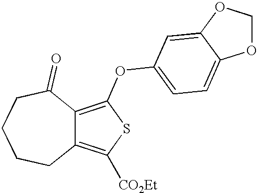

Production of 4,5,6,7-tetrahydro-3-(3,4-methylenedioxyphenoxy)-4-oxocyclohepta[c]thiophene-1-carboxylic Acid Ethyl Ester:

To a solution of the compound (3.33 g) obtained in Example 402 and sesamol (1.74 g) dissolved in THF (30 ml) was added sodium hydride (0.44 g) under ice cooling and the resulting solution was stirred at room temperature for 3 hours, then poured into water, and extracted with ethyl acetate. The extract was washed with water and an aqueous, saturated solution of sodium chloride, then dried (MgSO₄), and evaporated under reduced pressure to remove the solvent. The residual oily substance was subjected to column chromatography on silica gel. From the fractions eluted with ethyl acetate-hexane (1:4), 1.80 g (48%) of the title compound was obtained as colorless crystals.

¹H NMR (δ ppm in CDCl₃): 1.30 (3H, t, J=7.0 Hz), 1.87 (4H, quint, J=3.0 Hz), 2.71 (2H, t, J=6.2 Hz), 3.37 (2H, t, J=6.2 Hz), 4.23 (2H, q, J=7.0 Hz), 6.03 (2H, s), 6.71 (1H, dd, J=8.4, 2.6 Hz), 6.74 (1H, d, J=2.6 Hz), 6.81 (1H, d, J=8.4 Hz).

### EXAMPLE 404

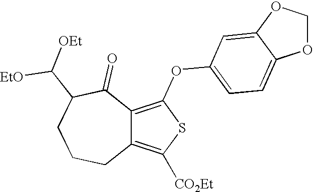

Production of 5-diethoxymethyl-4,5,6,7-tetrahydro-3-(3,4-methylenedioxyphenoxy)-4-oxocyclohepta[c]thiophene-1-carboxylic Acid Ethyl Ester:

A solution of boron trifluoride ether complex (1.81 ml) in dichloromethane (35 ml) was added dropwise to triethyl orthoformate (2.14 g) that was cooled at −50° C. This solution was stirred under ice cooling for 20 minutes and then cooled to −70° C. after addition of the compound (1.8 g) obtained in Example C. To this solution were added dropwise diisopropylethylamine (3.6 ml). After being stirred for 12 hours while returning the temperature to room temperature, the reaction solution was poured into an aqueous solution of citric acid and extracted with ethyl acetate. The organic layer was washed successively with water, dilute hydrochloric acid, and an aqueous, saturated solution of sodium chloride, then dried (MgSO₄), and concentrated under reduced pressure. The thus-obtained, residual oily substance was subjected to column chromatography on silica gel. From the fractions eluted with ethyl acetate-hexane (1:5), the title compound was obtained as a light yellow, oily substance (71%). ¹H NMR (δ ppm in CDCl₃): 1.17 (3H, t, J=7.0 Hz), 1.20 (3H, t, J=7.0 Hz), 1.30 (3H, t, J=7.4 Hz), 1.72-1.82 (2H, m), 1.93-2.17 (2H, m), 2.63-2.39 (1H, m), 3.00-3.12 (1H, m), 3.53-3.78 (4H, m), 3.86-3.99 (1H, m), 4.25 (2H, q, J=7.4 Hz), 5.00 (1H, d, J=7.0 Hz), 6.02 (2H, s), 6.69 (1H, dd, J=8.0, 2.4 Hz), 6.73 (1H, d, J=2.4 Hz), 6.79 (1H, d, J=8.0 Hz).

### EXAMPLE 405

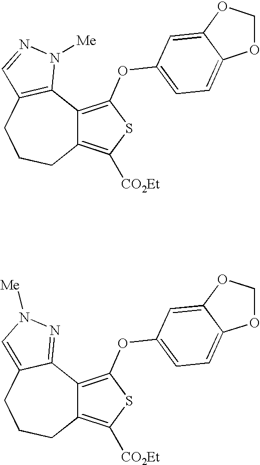

Production of 1,4,5,6-tetrahydro-9-(3,4-methylenedioxyphenoxy)-1-methylthieno[3′,41′:6,7]cyclohepta[1,2-c]pyrazole-7-carboxylic Acid Ethyl Ester and 1,4,5,6-tetrahydro-9-(3,4-methylenedioxyphenoxy)-2-methylthieno[3′,4′:6,7]cyclohepta[1,2-c]pyrazole-7-carboxylic Acid Ethyl Ester:

To a solution of the compound (1.62 g) obtained in Example 404 and dissolved in ethanol (10 ml) was added methanesulfonic acid (0.73 ml) and the resulting solution was stirred at room temperature for 10 minutes. Thereto was added methylhydrazine (0.72 ml) under ice cooling and the resulting solution was stirred at room temperature for 1 hour. The reaction solution was concentrated under reduced pressure. The residue was suspended in ethyl acetate and the suspension was washed successively with an aqueous, saturated solution of sodium hydrogen carbonate and an aqueous, saturated solution of sodium chloride. The organic layer was concentrated under reduced pressure and the residual oily substance was subjected to column chromatography on silica gel. From the fractions eluted with ethyl acetate-hexane (1:4), 0.43 g (31%) of 1,4,5,6-tetrahydro-9-(3,4-methylenedioxyphenoxy)-1-methylthieno[3′,4′:6,7]cyclohepta[1,2-c]pyrazole-7-carboxylic acid ethyl ester and 0.48 g (34%) of 1,4,5,6-tetrahydro-9-(3,4-methylenedioxyphenoxy)-2-methylthieno[3′,4′:6,7]cyclohepta[1,2-c]pyrazole-7-carboxylic acid ethyl ester were obtained, respectively as a colorless oily substance.

1,4,5,6-Tetrahydro-9-(3,4-methylenedioxyphenoxy)-1-methylthieno[3′,4′:6,7]cyclohepta[1,2-c]pyrazole-7-carboxylic Acid Ethyl Ester: ¹H NMR (δ ppm in CDCl₃): 1.33 (3H, t, J=7.4 Hz), 2.09 (2H, quint, J=7.0 Hz), 2.51 (2H, t, J=7.0 Hz), 2.99 (2H, brs), 3.94 (3H, s), 4.29 (2H, q, J=7.4 Hz), 6.01 (2H, s), 6.64 (1H, dd, J=8.6, 2.4 Hz), 6.69 (1H, d, J=2.4 Hz), 6.78 (1H, d, J=8.6 Hz), 7.38 (1H, s).

1,4,5,6-Tetrahydro-9-(3,4-methylenedioxyphenoxy)-2-methylthieno[3′,4′:6,7]cyclohepta[1,2-c]pyrazole-7-carboxylic Acid Ethyl Ester: ¹H NMR (δ ppm in CDCl₃): 1.32 (3H, t, J=7.4 Hz), 1.97-2.10 (2H, m), 2.67 (2H, t, J=7.0 Hz), 3.17-3.23 (2H, m), 3.87 (3H, s), 4.26 (2H, q, J=8.4 Hz), 5.97 (2H, s), 6.70 (1H, dd, J=8.4, 2.2 Hz), 6.71 (1H, d, J=8.4 Hz), 6.78 (1H, d, J=2.2 Hz), 7.19 (1H, s).

### EXAMPLE 406

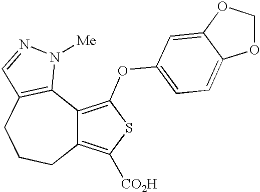

Production of 1,4,5,6-tetrahydro-9-(3,4-methylenedioxyphenoxy)-1-methylthieno[3′,4′:6,7]cyclohepta[1,2-c]pyrazole-7-carboxylic Acid:

A mixture of 1,4,5,6-tetrahydro-9-(3,4-methylenedioxyphenoxy)-1-methylthieno[3′,4′:6,7]cyclohepta[1,2-c]pyrazole-7-carboxylic acid ethyl ester (0.43 g) obtained in Example 405, an aqueous 0.4 N solution of potassium hydroxide (10 ml), THF (10 ml), and ethanol (10 ml) was stirred at room temperature for 24 hours. The reaction solution was concentrated under reduced pressure and then neutralized with 1 N hydrochloric acid to collect the precipitated crystals by filtration. A mixed solution of the thus-obtained crystals dissolved in THF-ethyl acetate was washed with an aqueous, saturated solution of sodium chloride and then evaporated to remove the solvent to obtain 0.33 g (83%) of the title compound as colorless crystals.

¹H NMR (δ ppm in CDCl₃): 2.06-2.29 (2H, m), 2.51 (2H, t, J=7.4 Hz), 2.93-3.06 (2H, brs), 3.96 (3H, s), 6.02 (2H, s), 6.66 (1H, dd, J=8.0, 2.2 Hz), 6.70 (1H, d, J=2.2 Hz), 6.79 (1H, d, J=8.0 Hz), 7.40 (1H, s).

### EXAMPLE 407

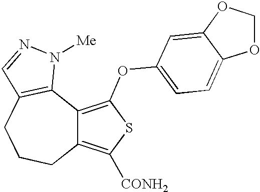

Production of 1,4,5,6-tetrahydro-9-(3,4-methylenedioxyphenoxy)-1-methylthieno[3′,4′:6,7]cyclohepta[1,2-c]pyrazole-7-carboxamide:

To a solution of the compound (0.30 g), which was obtained in Example 406, in DMF (10 ml) were added 1-ethyl-3-(3-dimetyhylaminopropyl) carbodiimide hydrochloride (WSC) (0.18 g) and 1-hydroxybenzotriazole-ammonia complex (HOBt-NH₃) (0.15 g) and the resulting solution was subjected to stirring at room temperature for 7 hours, then concentration, addition of water, and extraction with ethyl acetate. The organic layer was washed successively with an aqueous, saturated solution of sodium hydrogen carbonate and an aqueous, saturated solution of sodium chloride, then dried (MgSO₄), and evaporated to remove the solvent to obtain 0.19 g (63%) of the title compound as colorless crystals.

¹H NMR (δ ppm in CDCl₃): 2.05-2.17 (2H, m), 2.54 (2H, t, J=7.2 Hz), 2.82-2.93 (2H, m), 3.94 (3H, s), 5.57 (2H, brs), 6.01 (2H, s), 6.62 (1H, dd, J=8.4, 2.2 Hz), 6.67 (1H, d, J=2.2 Hz), 6.76 (1H, d, J=8.4 Hz), 7.40 (1H, s).

### EXAMPLE 408

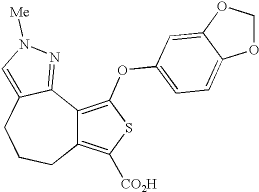

Production of 1,4,5,6-tetrahydro-9-(3,4-methylenedioxyphenoxy)-2-methylthieno[3′,4′:6,7]cyclohepta[1,2-c]pyrazole-7-carboxylic Acid:

A mixture of 1,4,5,6-tetrahydro-9-(3,4-methylenedioxyphenoxy)-2-methylthieno[3′,4′:6,7]cyclohepta[1,2-c]pyrazole-7-carboxylic acid ethyl ester (0.48 g), which was obtained in Example 405, an aqueous 0.4 N solution of potassium hydroxide (10 ml), and ethanol (10 ml) was stirred at room temperature for 14 hours. The reaction solution was concentrated, then neutralized with 1 N hydrochloric acid, and extracted with ethyl acetate. The organic layer was washed successively with water and an aqueous, saturated solution of sodium chloride, dried (MgSO₄), and then evaporated to remove the solvent to obtain 0.26 g (58%) of the title compound as a colorless oily material.

¹H NMR (δ ppm in CDCl₃): 2.00-2.12 (2H, m), 2.70 (2H, t, J=7.0 Hz), 3.13-3.21 (2H, m), 3.92 (3H, s), 5.99 (2H, s), 6.74-6.81 (3H, m), 7.21 (1H, s).

### EXAMPLE 409

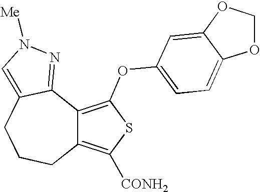

Production of 1,4,5,6-tetrahydro-9-(3,4-methylenedioxyphenoxy)-2-methylthieno[3′,4′:6,7]cyclohepta[1,2-c]pyrazole-7-carboxamide:

To a solution of the compound (0.26 g) obtained in Example 408 and dissovled in DMF (10 ml) were added 1-ethyl-3-(3-dimetyhylaminopropyl)carbodiimide hydrochloride (WSC) (0.23 g) and 1-hydroxybenzotriazole-ammonia complex (HOBt-NH₃) (0.18 g) and the resulting solution was subjected to stirring at room temperature for 14 hours, then concentration, addition of water, and extraction with ethyl acetate. The organic layer was washed successively with an aqueous, saturated solution of sodium hydrogen carbonate and an aqueous, saturated solution of sodium chloride, then dried (MgSO₄), and evaporated to remove the solvent. The thus-obtained residual oily substance was subjected to column chromatography on silica gel. From the fractions eluted with ethyl acetate-hexane (2:1), 0.16 g (36%) of the title compound was obtained as colorless crystals.

¹H NMR: 1.98-2.11 (2H, m), 2.69 (2H, t, J=7.0 Hz), 3.07-3.13 (2H, m), 3.87 (3H, s), 5.47 (2H, brs), 5.98 (2H, s), 6.68 (1H, dd, J=6.6, 2.2 Hz), 6.74 (1H, d, J=6.6 Hz), 6.76 (1H, d, J=2.2 Hz), 7.19 (1H, s).

According to the same manner as that in Example 13a hereinafter, compounds in Examples 410 to 430 were synthesized.

TABLE 41

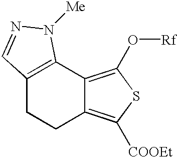

| Ex- | | |
|---|---|---|
| ample | | Melting |
| No. | —Rf | Point (° C.) |
| 410 | 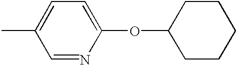 | 111-112 |
| 411 | 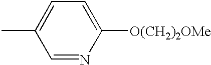 | Oily substance¹⁾ |
| 412 | 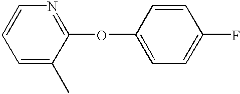 | 156-157 |
| 413 | 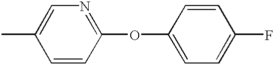 | Oily substance²⁾ |
| 414 | 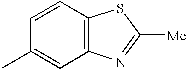 | 143-144 |
| 415 | 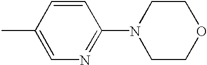 | Oily substance³⁾ |
| 416 | 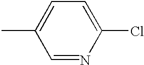 | 147-148 |
| 417 | 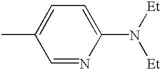 | 111-112 |
| 418 | 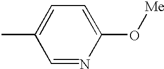 | 91-92 |
| 419 | 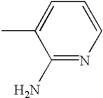 | Oily substance⁴⁾ |

TABLE 42

| | | |
|---|---|---|
| 420 | 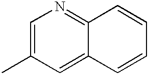 | 107-109 |
| 421 | 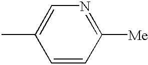 | 58-59 |
| 422 | 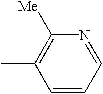 | 139-140 |
| 423 | 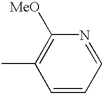 | 133-134 |
| 424 | 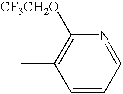 | 112-113 |
| 425 | 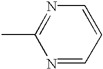 | 200-202 |
| 426 | 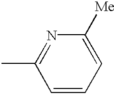 | 152-154 |
| 427 | 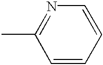 | 169-170 |
| 428 | 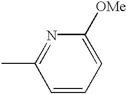 | 163-164 |
| 429 | 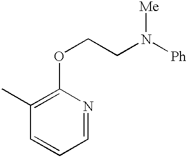 | Oily substance⁵⁾ |
| 430 | 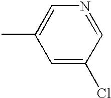 | |

1) ¹H NMR (δ ppm in CDCl₃): 1.33 (3H, t, J=7.2 Hz), 2.67-2.73 (2H, m), 3.21-3.28 (2H, m), 3.46 (3H, s), 3.74-3.78 (2H, m), 4.10 (3H, s), 4.29 (2H, q, J=7.2 Hz), 4.46-4.56 (2H, m), 6.86 (1H, d, J=8.8 Hz), 7.37 (1H, S), 7.48 (1H, dd, J=3.0 Hz, 8.8 Hz), 8.09 (1H, d, J=2.8 Hz).

2) ¹H NMR (δ ppm in CDCl₃): 1.34 (3H, t, J=7.2 Hz), 2.70 (2H, t, J=7.0 Hz), 3.26 (2H, t, J=7.0 Hz), 4.07 (3H, s), 4.30 (2H, q, J=7.2 Hz), 6.97 (1H, d, J=8.4 Hz), 7.10-7.13 (4H, m), 7.36 (1H, s), 7.56 (1H, dd, J=3.0 Hz, 9.0 Hz), 8.09 (1H, d, J=2.8 Hz).

3) ¹H NMR (δ ppm in CDCl₃): 1.32 (3H, t, J=7.0 Hz), 2.69 (2H, t, J=7.0 Hz), 3.25 (2H, t, J=6.9 Hz), 3,51 (4H, t, J=5.0 Hz), 3.84 (4H, t, J=4.8 Hz), 4.11 (3H, s), 4.28 (2H, q, J=7.0 Hz), 6.66 (1H, d, J=9.2 Hz), 7.36 (1H, s), 7.42 (1H, dd, J=3.0 Hz, 9.2 Hz), 8.18 (1H, d, J=2.6 Hz).

4) ¹H NMR (δ ppm in CDCl₃): 1.30 (3H, t, J=6.9 Hz), 2.57 (2H, t, J=7.0 Hz), 3.09 (2H, t, J=7.0 Hz), 3.33 (3H, s), 3.98 (3H, s), 4.26 (2H, q, J=7.0 Hz), 6.81 (1H, dd, J=7.8, 5.0 Hz), 7.11 (1H, dd, J=7.7, 1.5 Hz), 7.36 (1H, S), 7.81 (1H, dd, J=5.1, 1.5 Hz), 9.18 (1H, s).

5) ¹H NMR (δ ppm in CDCl₃): 1.32 (3H, t, J=7.2 Hz), 2.70 (2H, t, J=6.6 Hz), 2.85 (3H, s), 3.29 (2H, t, J=6.6 Hz), 3,66 (2H, t, J=6.0 Hz), 4.28 (2H, q, J=7.2 Hz), 4.55 (2H, t, J=6.0 Hz), 6.65-6.69 (3H, m), 6.94 (1H, dd, J=7.6, 4.8 Hz), 7.11-7.19 (2H, m), 7.37 (1H, s), 7.46 (1H, dd, J=7.6, 1.4 Hz), 8.06 (1H, dd, J=4.8, 1.4 Hz).

### EXAMPLE 431

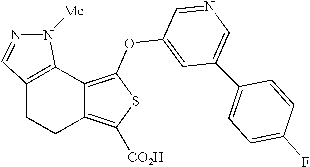

4,5-Dihydro-8-(5-(4-fluorophenyl)pyridin-3-yloxy)-1-methyl-1H-thieno[3,4-g]indazole-6-carboxylic Acid:

To a solution of 3-(4-fluorophenyl)-5-hydroxypyridine (1.35 g) in N-methylpyrrolidone (12 ml) was added potassium tert-butoxide (0.80 g) and the resulting solution was stirred at room temperature for 10 minutes. To this reaction solution was added the compound (2.04 g) obtained in Example 12a hereinafter and the resulting solution was stirred at 100° C. for 6 hours. The reaction solution was poured into water and extracted with ethyl acetate. The extract was washed with an aqueous, saturated solution of sodium chloride, then dried (MgSO₄), and evaporated to remove the solvent. The residue was subjected to column chromatography on silica gel and, from the fractions eluted with ethyl acetate-hexane (1:1), 1.27 g of 4,5-dihydro-8-(5-(4-fluorophenyl)pyridin-3-yloxy)-1-methyl-1H-thieno[3,4-g]indazole-6-carboxylic acid ethyl ester was obtained. To a solution of this compound dissolved in ethanol (6 ml) and THF (9 ml) was added an aqueous 2 N solution of potassium hydroxide (3 ml) and the resulting solution was stirred at 60° C. for 1 hour. The reaction solution was cooled and then neutralized with 1 N hydrochloric acid to collect the precipitated crystals by filtration, thereby obtaining the title compound (0.78 g, 31% yield) as colorless crystals. ¹H NMR (δ ppm in CDCl₃): 2.66 (2H, t, J=6.8 Hz), 3.18 (2H, t, J=6.8 Hz), 4.01 (3H, s), 7.32-7.40 (3H, m), 7.83-7.90 (2H, m), 8.21-8.23 (1H, m), 8.67 (1H, d, J=2.6 Hz), 8.87 (1H, d, J=2.2 Hz).

According to the same manner as that in Example 172, compounds in Examples 432 to 451 were synthesized.

TABLE 43

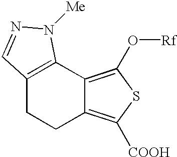

| Example | | Melting point |
|---|---|---|
| No. | —Rf | (° C.) |
| 432 | 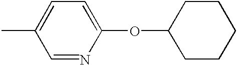 | 267-268 |
| 433 | 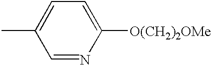 | 243-244 |
| 434 | 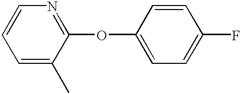 | 262-263 |
| 435 | 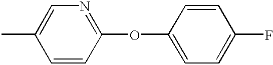 | 249-251 |
| 436 | 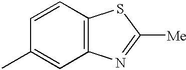 | 252-254 |
| 437 | 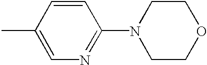 | 280-282 |
| 438 | 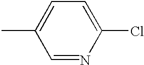 | 265-267 |
| 439 | 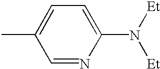 | 259-261 |
| 440 | 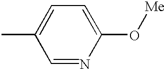 | 260-262 |
| 441 | 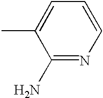 | Oily |
| | | substance¹⁾ |

TABLE 44

| | | |
|---|---|---|
| 442 | 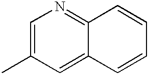 | 258-260 |
| 443 | 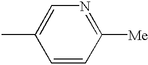 | 274-275 |
| 444 | 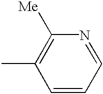 | 269-271 |
| 445 | 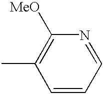 | 252-254 |
| 446 | 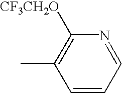 | 268-270 |
| 447 | 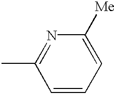 | 261-263 |
| 448 | 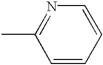 | 248-249 |
| 449 | 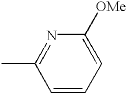 | 252-254 |
| 450 | 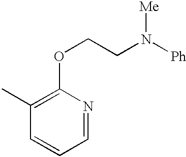 | 203-205 |
| 451 | 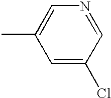 | 289-291 |

1) ¹H NMR (δ ppm in CDCl₃): 2.63 (2H, t, J=6.8 Hz), 3.16 (2H, t, J=6.8 Hz), 4.03 (3H, s), 6.34 (2H, brs), 6.58 (1H, dd, J=7.9, 4.9 Hz), 7.49 (1H, d, J=8.0 Hz), 7.88 (1H, dd, J=0.8, 1.4 Hz).

### EXAMPLE 452

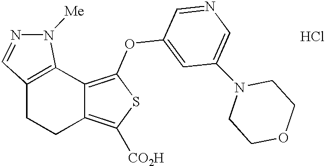

4,5-Dihydro-1-methyl-8-{[5-(4-morpholinyl)-3-pyridinyl]oxy}-1H-thieno[3,4-g]indazole-6-carboxylic Acid Hydrochloride:

To a solution of 5-(4-morpholinyl)-3-hydroxypyridine (0.85 g) in N-methylpyrrolidone (25 ml) was added potassium tert-butoxide (0.53 g) and the resulting solution was stirred at room temperature for 10 minutes. Thereto was added then the compound (1.61 g) that was obtained in Example 12a hereinafter and, after stirring at 90° C. for 7 hours, the reaction solution was poured into water and extracted with ethyl acetate. The extract was washed successively with water, an aqueous, saturated solution of sodium hydrogen carbonate, and an aqueous, saturated solution of sodium chloride, then dried (MgSO₄), and evaporated to remove the solvent. The residual oily substance was subjected to column chromatography on silica gel and, from the fractions eluted with ethyl acetate-hexane (1:3), 4,5-dihydro-1-methyl-8-(5-(4-morpholinyl)-3-pyridinyloxy)-1H-thieno[3,4-g]indazole-6-carboxylic acid ethyl ester was obtained as an oily substance. To a solution of this compound dissolved in ethanol (20 ml) was added an aqueous 2 N solution of potassium hydroxide (2 ml) and the resulting solution was stirred at room temperature for 12 hours. The reaction solution was subjected to addition of 2 N hydrochloric acid (3 ml), followed by concentration and collection of the precipitated crystals by filtration. Washing with water and ethyl acetate gave the title compound (0.67 g, 32% yield) as colorless needles. Melting point: 303-305° C.

### EXAMPLE 453

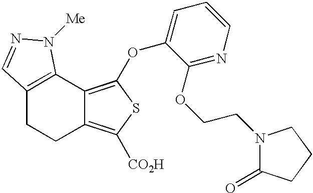

4,5-Dihydro-1-methyl-8-({2-[2-(2-oxo-1-pyrrolidinyl)ethoxy]-3-pyridinyl}oxy)-1H-thieno[3,4-g]indazole-6-carboxylic Acid:

To a solution of 2-[2-(2-oxo-1-pyrrolidinyl)ethoxy]-3-hydroxypyridine (1.98 g) in N-methylpyrrolidone (30 ml) was added potassium tert-butoxide (1.07 g) and the resulting solution was stirred at room temperature for 10 minutes. Then, the compound (2.88 g), which was obtained in Example 12a hereinafter, was added and, after stirring at 90° C. for 7 hours, the reaction solution was poured into water and extracted with ethyl acetate. The extract was washed successively with water, an aqueous solution of sodium hydrogen carbonate, and an aqueous, saturated solution of sodium chloride, then dried (MgSO₄), and evaporated under reduced pressure to remove the solvent. The residual oily substance was subjected to column chromatography on silica gel and, from the fractions eluted with ethyl acetate-hexane (1:3), 4,5-dihydro-1-methyl-8-({2-[2-(2-oxo-1-pyrrolidinyl)ethoxy]-3-pyridinyl}oxy)-1H-thieno[3,4-g]indazole-6-carboxylic acid ethyl ester was obtained as an oily substance. To a solution of this compound dissolved in ethanol (20 ml) and tetrahydrofuran (20 ml) was added an aqueous 2 N solution of potassium hydroxide (6 ml) and the resulting solution was stirred at room temperature for 12 hours. The reaction solution was subjected to addition of 2 N hydrochloric acid (7 ml), followed by concentration and collection of the precipitated crystals by filtration. Washing with water and ethyl acetate gave the title compound (2.13 g, 55% yield) as colorless needles. Melting point: 202-204° C.

According to the same manner as that in Example 211, compounds in Examples 454 to 473 were synthesized.

TABLE 45

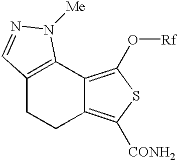

| Example | | Melting |
|---|---|---|
| No. | —Rf | point (° C.) |
| 454 | 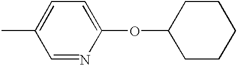 | 208-209 |
| 455 | 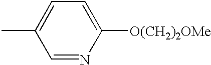 | 142-146 |
| 456 | 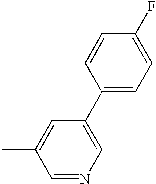 | 199-200 |
| 457 | 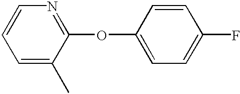 | 236-238 |
| 458 | 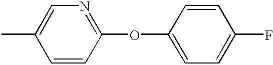 | 181-182 |
| 459 | 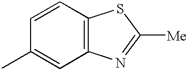 | 104-105 |
| 460 | 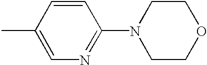 | 236-238 |
| 461 | 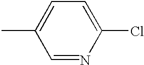 | 225-226 |
| 462 | 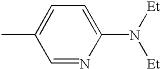 | 139-140 |
| 463 | 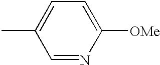 | 176-177 |

TABLE 46

| | | |
|---|---|---|
| 464 | 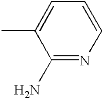 | 235-236 |
| 465 | 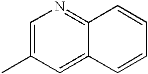 | 219-220 |
| 466 | 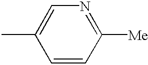 | 147-148 |
| 467 | 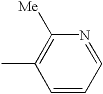 | 186-187 |
| 468 | 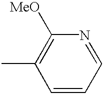 | 180-182 |
| 469 | 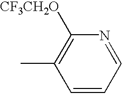 | 210-212 |
| 470 | 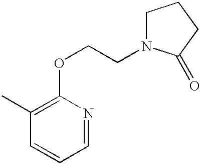 | 141-143 |
| 471 | 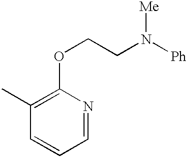 | 136-138 |
| 472 | 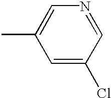 | 239-240 |
| 473 | 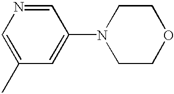 | 218-219 |

### EXAMPLE 474

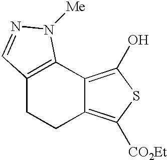

4,5-Dihydro-8-hydroxy-1-methyl-4,5-dihydro-1H-thieno[3,4-g]indazole-6-carboxylic Acid Ethyl Ester:

To a solution of 10 g of the compound obtained in Example 264 and dissolved in ethanol (200 ml) was added palladium carbon (2 g) and the resulting mixture was stirred under hydrogen pressure (0.5 Pa) for 4 hours. The insoluble material was filtered off and the filtrate was concentrated under reduced pressure to obtain crystals. Recrystallization from tetrahydrofuran gave the title compound (6.95 g, 46% yield) as colorless prisms. Melting point: 203-205° C.

### EXAMPLE 475

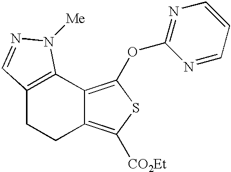

4,5-Dihydro-1-methyl-8-(pyrimidin-2-yloxy)-1H-thieno[3,4-g]indazole-6-carboxylic Acid Ethyl Ester:

A solution of the compound (1.82 g) obtained in Example 474 and sodium fluoride (0.75 g) in DMF (30 ml) was subjected to stirring at 90° C. for 30 minutes, then addition of 2-chloropyrimidine (0.75 g), and stirring at 90° C. for 20 hours. The reaction solution was poured into water and extracted with ethyl acetate. The extract was washed with an aqueous, saturated solution of sodium hydrogen carbonate, water, and an aqueous, saturated solution of sodium chloride, then dried (MgSO₄), and evaporated under reduced pressure to remove the solvent to obtain the title compound (0.75 g, 32%) as yellow needles. Melting point: 200-202° C.

### EXAMPLE 476

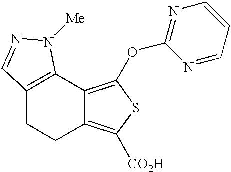

4,5-Dihydro-1-methyl-8-(pyrimidin-2-yloxy)-1H-thieno[3,4-g]indazole-6-carboxylic Acid:

A mixture of the compound (1.00 g) obtained in Example 475, an aqueous 2 N solution of potassium hydroxide (3.2 ml), ethanol (10 ml), and tetrahydrofuran (10 ml) was stirred at 50° C. for 12 hours. The reaction solution was made acid with 2 N hydrochloric acid and extracted with ethyl acetate. The extract was washed with water and an aqueous, saturated solution of sodium chloride, then dried (MgSO₄), and evaporated under reduced pressure to remove the solvent to obtain crystals. Recrystallization from tetrahydrofuran gave the title compound (0.50 g, 54% yield) as colorless needles. Melting point: 256-258° C.

### EXAMPLE 477

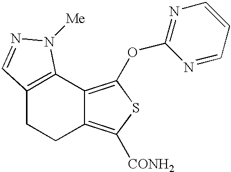

4,5-Dihydro-1-methyl-8-(pyrimidin-2-yloxy)-1H-thieno[3,4-g]indazole-6-carboxamide:

A solution of the compound (0.49 g) obtained in Example 476, HOBt-NH₃ (0.14 g), and WSC (0.17 g) in DMF (15 ml) was stirred at room temperature for 12 hours. The reaction solution was poured into water and extracted with ethyl acetate. The extract was washed with an aqueous solution of sodium hydrogen carbonate and an aqueous, saturated solution of sodium chloride and then dried (MgSO₄). Evaporation of the solvent under reduced pressure gave the title compound (0.12 g, 24%) as colorless prisms. Melting point: 265° C. (decomposition) (recrystallization solvent: AcOEt-isopropyl ether).

### EXAMPLE 478

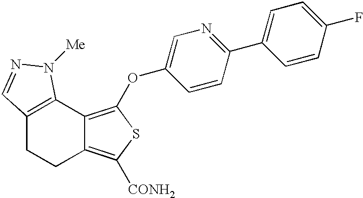

4,5-Dihydro-8-{[6-(4-fluorophenyl)-3-pyridinyl]oxy}-1-methyl-1H-thieno[3,4-g]indazole-6-carboxamide:

A mixture of the compound (1.08 g) obtained in Example 461, 4-fluorophenylboronic acid (0.50 g), tetrakis(triphenylphosphine)palladium (0.17 g), and 2 N sodium carbonate (3.6 ml), dimethoxyethane (12 ml), and dimethylacetamide (6 ml) was heated at reflux overnight and then poured into water to collect the precipitated crystals by filtration. Recrystallization from chloroform-ethyl acetate gave the title compound (0.71 g, 54% yield) as colorless crystals. Melting point: 236-237° C.

According to the same manner as that in Example 478, compounds in Examples 479 to 482 were synthesized.

TABLE 47

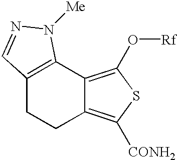

| Example | | Melting |
|---|---|---|
| No. | —Rf | point (° C.) |
| 479 | 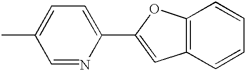 | 237-238 |
| 480 | 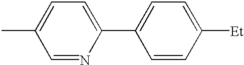 | 227-228 |
| 481 | 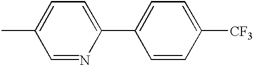 | 237-238 |
| 482 | 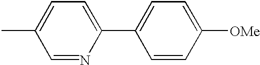 | 230-231 |

### EXAMPLE 483

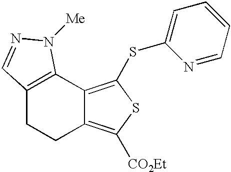

4,5-Dihydro-1-methyl-8-(2-pyridinylsulfanyl)-1H-thieno[3,4-g]indazole-6-carboxylic Acid Ethyl Ester:

To a solution of 2-pyridinethiol (1.72 g) in N-methylpyrrolidone (35 ml) was added potassium tert-butoxide (1.81 g) and the resulting solution was stirred at 70° C. for 30 minutes. Then, the compound (5.00 g), which was obtained in Example 12a hereinafter, was added and, after stirring at 100° C. for 4 hours, the reaction solution was poured into water and extracted with ethyl acetate. The extract was washed successively with water and an aqueous, saturated solution of sodium chloride, dried (MgSO₄), and then evaporated under reduced pressure to remove the solvent. The residual oily substance was subjected to column chromatography on silica gel and, from the fractions eluted with ethyl acetate-hexane (2:3), the title compound (5.08 g, 93%) was obtained as a colorless oily substance. ¹H NMR (δ ppm in CDCl₃): 1.38 (3H, t, J=7.4 Hz), 2.67 (2H, t, J=6.6 Hz), 3.27 (2H, t, J=6.6 Hz), 4.10 (3H, s), 4.36 (2H, q, J=7.4 Hz), 6.95 (1H, dt, J=8.0, 1.0 Hz), 7.02-7.09 (1H, m), 7.52 (1H, dt, J=8.0, 1.8 Hz), 8.42-8.46 (1H, m).

### EXAMPLE 484

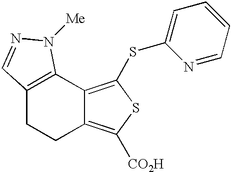

4,5-Dihydro-1-methyl-8-(2-pyridinylsulfanyl)-1H-thieno[3,4-g]indazole-6-carboxylic Acid:

To a mixture of 4,5-dihydro-1-methyl-8-(2-pyridinylsulfanyl)-1H-thieno[3,4-g]indazole-6-carboxylic acid ethyl ester (5.00 g) in ethanol (10 ml) and THF (10 ml) was added an aqueous 2 N solution of potassium hydroxide (30 ml) and the resulting solution was stirred at room temperature for 14 hours. The reaction solution was made acid with 2 N hydrochloric acid and extracted with a mixed solution of ethyl acetate-THF. The organic layer was washed with water and an aqueous, saturated solution of sodium chloride, then dried (MgSO₄), and evaporated to remove the solvent. The thus-obtained crystals were recrystallized from THF to obtain the title compound (2.73 g, 59% yield) as colorless prisms. Melting point: 231-232° C.

### EXAMPLE 485

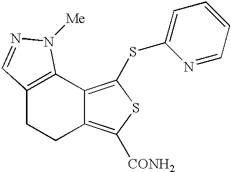

4,5-Dihydro-1-methyl-8-(2-pyridinylsulfanyl)-1H-thieno[3,4-g]indazole-6-carboxamide:

To a solution of the compound (1.5 g) obtained in Example 484 and dissolved in THF (50 ml) was added oxalyl chloride (1.2 ml) under ice cooling and the resulting solution was stirred for 30 minutes. The reaction solution was concentrated under reduced pressure and a solution of the residue dissolved in THF (30 ml) was mixed with an aqueous 25% ammonia solution (3 ml) and stirred at room temperature for 30 minutes. The reaction solution was poured into water and extracted with ethyl acetate. This organic layer was washed with water and an aqueous, saturated solution of sodium chloride, dried (MgSO₄), and evaporated to remove the solvent. The thus-obtained crystals were recrystallized from THF-IPE to obtain the title compound (0.71 g) as colorless prisms. Melting point: 234-236° C.

According to the same manner as that in Example 485, the following compounds were synthesized.

TABLE 48

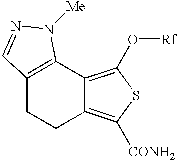

| Example No. | —Rf | Melting point (° C.) |
|---|---|---|
| 486 | 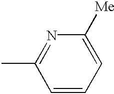 | 230-231 |
| 487 | 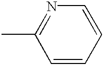 | 253-254 |
| 488 | 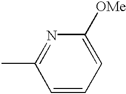 | 183-185 |

### EXAMPLE 489

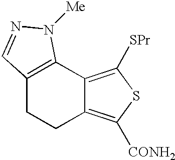

1-Methyl-8-(propylsulfanyl)-1H-thieno[3,4-g]indazole-6-carboxamide:

A solution of the compound (0.33 g) obtained in Reference Example 52, and manganese dioxide (1.5 g) in dichloromethane (30 ml) was heated at reflux for 4 days. An insoluble material was removed by filtration and the filtrate was concentrated under reduced pressure. The thus-obtained residue was subjected to column chromatography on silica gel. From the fractions eluted with ethyl acetate, the title compound (39 mg, 12%) was obtained as light brown crystals. Melting point: 165-166° C.

### EXAMPLE 490

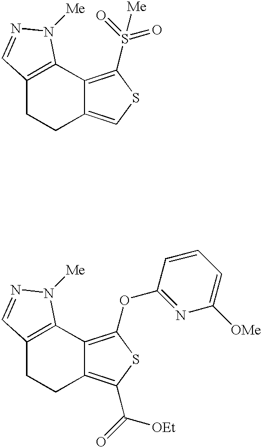

1-Methyl-8-methylsulfonyl-4,5-dihydro-1H-thieno[3,4-g]indazole and 8-(6-methoxypyridin-2-yloxy)-1-methyl-4,5-dihydro-1H-thieno[3,4-g]indazole-6-carboxylic Acid Ethyl Ester

To a solution of 2-hydroxy-6-methoxypyridine (2.76 g) dissolved in 1-methyl-2-pyrrolidone (50 ml) was added potassium t-butoxide (2.69 g) and the mixture was stirred at 70° C. for 20 minutes. The compound obtained in Reference Example 78 (7.51 g) was added thereto and the mixture was stirred at 120° C. for 8 hours. The reaction mixture was poured into water and the precipitated crystals were filtered off. This solid was dissolved in ethyl acetate, washed with water and saturated saline succesively and dried (MgSO₄), and then the solvent was removed under reduced pressure. The residue was subjected to silica gel chromatography. From the fraction eluted with ethyl acetate-hexane (1:4), 1-methyl-8-methylsulfonyl-4,5-dihydro-1H-thieno[3,4-g]indazole (1.60 g) and 8-(6-methoxypyridin-2-yloxy)-1-methyl-4,5-dihydro-1H-thieno[3,4-g]indazole-6-carboxylic acid ethyl ester (1.79 g, 21%) were obtained.

1-Methyl-8-methylsulfonyl-4,5-dihydro-1H-thieno[3,4-g]indazole: melting point: 192-193° C. ¹H-NMR (δ ppm in CDCl₃): 2.60-2.27 (4H, m), 3.25 (3H, s), 4.13 (3H, s), 7.45 (1H, d, J=7.8 Hz).

8-(6-Methoxypyridin-2-yloxy)-1-methyl-4,5-dihydro-1H-thieno[3,4-g]indazole-6-carobxylic acid ethyl ester: melting point: 163-164° C. ¹H-NMR (δ ppm in CDCl₃): 1.38 (3H, t, J=7.2 Hz), 2.68 (2H, t, J=6.6 Hz), 3.25 (2H, t, J=6.6 Hz), 3.97 (3H, s), 4.11 (3H, s), 4.34 (2H, q, J=7.2 Hz), 6.57-7.00 (2H, m), 7.37 (1H, s), 7.66 (1H, t, J=8.1 Hz).

### EXAMPLE 491

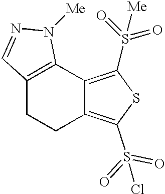

1-Methyl-8-methylsulfonyl-4,5-dihydro-1H-thieno[3,4-g]indazole-6-sulfonyl Chloride

The compound obtained in Example 490 (0.50 g) was added little by little to fuming sulfuric acid (2.4 ml) cooled to −10° C. The mixture was stirred for 1 hour with slowly warming to 8° C. To this solution was added thionyl chloride (9.6 ml). The mixture was heated under reflux for 1 hour and thionyl chloride was distilled off under reduced pressure. The residue was pourd into ice-water and extracted with dichloromethane. The extract was washed with water and saturated saline, and dried (MgSO₄). The solvent was removed under reduced pressure. The residue was recrystallized from ethyl acetate to obtain the title compound as pale yellow needles (0.46 g, 67%). Melting point 81-82° C. ¹H-NMR (δ ppm in CDCl₃): 2.78 (2H, dd, J=8.4, 6.2 Hz), 3.16 (2H, dd, J=8.4, 6.2 Hz), 3.32 (3H, s), 7.53 (1H, s).

### EXAMPLE 492

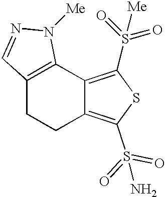

1-Methyl-8-methylsulfonyl-4,5-dihydro-1H-thieno[3,4-g]indazole-6-sulfonamide

To a solution of the compound obtained in Example 491 (0.20 g) dissolved in THF (10 ml) was added a 25% aqeuous ammonia solution (1 ml) with ice-cooling and the mixture was stirred at room temperature for 30 minutes. The mixture was extracted with ethyl acetate and the extract was washed with water and saturated saline and dried (MgSO₄). The solvent was removed under reduced pressure to obtain the title compound (0.17 g, 89%). Melting point: 228-229° C. ¹H-NMR (δ ppm in CDCl₃): 2.61 (2H, t, J=7.2 Hz), 2.93 (2H, t, J=7.2 Hz), 3.61 (3H, s), 4.00 (3H, s), 7.51 (1H, s), 8.17 (2H, s).

### EXAMPLE 493

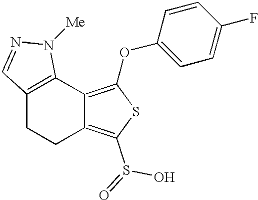

8-(4-Fluorphenoxy)-1-methyl-4,5-dihydro-1H-thieno[3,4-g]indazole-6-carboxylic Acid

According to the same manner as that in Example 172, the title compound (yield: 100%) was obtained as colorless prisms from the compound obtained in Example 292. Melting point: 266-268° C.

### EXAMPLE 494

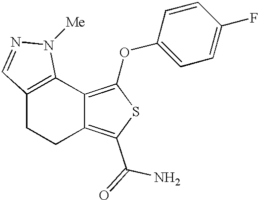

8-(4-Fluorophenoxy)-1-methyl-4,5-dihydro-1H-thieno[3,4-g]indazole-6-carboxamide

According to the same manner as that in Example 211, the title compound (yield: 95%) was obtained as colorless prisms from the compound obtained in Example 493. Melting point: 188-189° C.

### EXAMPLE 1a

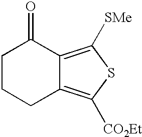

Production of 4,5,6,7-tetrahydro-3-methylthio-4-oxobenzo[c]thiophene-1-carboxylic Acid Ethyl Ester:

Into a four-necked flask (5 L) were charged potassium carbonate (anhydrous) (210 g, 1.50 mol), N,N-dimethylformamide (90 ml), and 1,3-cyclohexanedione (12.3 g, 0.55 mol, 1.1 eq) under argon atmosphere and the resulting solution was stirred at room temperature (29° C.) for 12 minutes. Thereto was added at once carbon disulfide (36 ml, 0.60 mol, 1.2 eq) and the resulting solution was stirred at room temperature for 15 minutes. A solution of ethyl chloroacetate (61.5 g, 0.50 mol) in N,N-dimethylformamide (500 ml) was added dropwise below 35° C. over a period of 2 hours and 40 minutes with washing the container with N,N-dimethylformamide (25 ml). After the reaction mixture was stirred at room temperature for 20 minutes and then cooled with ice, methyl iodide (34 ml, 0.55 mol) was added dropwise in 12 minutes (4° C. to 9° C.). The reaction mixture was heated at 50±2° C. to react for 4 hours. The reaction solution was cooled and water (2.5 L) was added dropwise in 30 minutes (6° C. to 18° C.). The resulting mixture was stirred overnight at room temperature. The precipitated crystals were collected by filtration and washed with water (1 L×2) Drying under vacuum at 40° C. for 16 hours gave light brown, crude crystals (111.3 g, 82.3% yield). A solution of these crystals (111.3 g) dissolved in ethyl acetate (1.35 L) was washed with water (1.0 L). The ethyl acetate layer was mixed with activated carbon (13.5 g, Shirasagi A) and stirred at room temperature for 30 minutes. The activated carbon was filtered off and washed with ethyl acetate (750 ml). The filtrate (red color) and the washing were combined and concentrated to dryness. The residue was dissolved in methanol (650 ml) with heating (60° C.). The resulting solution was gradually cooled to room temperature (crystals precipitated at about 47° C.). The resulting mixture was cooled with ice and stirred below 5° C. for 1 hour. The crystals were collected by filtration and washed with ice-cooled methanol (200 ml). Drying at 40° C. under vacuum for 18 hours gave the title compound (101.7 g, 75.2% yield) as light yellow crystals.

¹H NMR (300 MHz, CDCl₃) δ: 1.37 (3H, t, J=7.1 Hz), 2.05 (2H, quint, J=6.7 Hz), 2.54 (2H, t, J=6.7 Hz), 2.60 (3H, s), 3.19 (2H, t, J=6.7 Hz), 4.33 (2H, q, J=7.1 Hz).

### EXAMPLE 2a

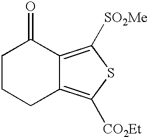

Production of 4,5,6,7-tetrahydro-3-methylsulfonyl-4-oxobenzo[c]thiophene-1-carboxylic Acid Ethyl Ester:

To a solution of the compound (10.8 g, 39.9 mmol) obtained in Example 1a and dissolved in dichloromethane (300 ml) was gradually added 70% metachloroperbenzoic acid (21.7 g) under ice cooling (3° C. to 9° C.). The resulting solution was stirred at room temperature overnight. The reaction solution was washed with an aqueous, saturated solution of sodium bicarbonate (200 ml×2) and water (200 ml). After the organic layer was dried using magnesium sulfate, the magnesium sulfate was filtered off and washed with dichloromethane (30 ml). The filtrate and the washing were combined and concentrated to dryness. Drying under vacuum at 40° C. for 5 hours gave the title compound (12.3 g) as a brownish-white, crystalline powder.

¹H NMR (300 MHz, CDCl₃) δ: 1.64 (3H, t, J=7.1 Hz), 2.39 (2H, quint, J=6.5 Hz), 2.92 (2H, t, J=6.5 Hz), 3.52 (2H, t, J=6.5 Hz), 3.78 (3H, s), 4.63 (2H, q, J=7.1 Hz).

### EXAMPLE 3a

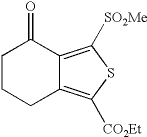

Production of 4,5,6,7-tetrahydro-3-methylsulfonyl-4-oxobenzo[c]thiophene-1-carboxylic Acid Ethyl Ester:

The compound (10.8 g, 40.0 mmol) obtained in Example 1a was dissolved in trifluoroacetic acid (92 ml, 1.20 mol, 30 eq). Thereto was added dropwise an aqueous 30% solution of hydrogen peroxide (14 g, 123 mmol, 3 eq) under ice cooling. The resulting solution was stirred at room temperature for 2 hours and 40 minutes. After the reaction, the cooled reaction solution was neutralized by addition of an aqueous 8 M solution of sodium hydroxide (161 ml). The precipitation mixture was loosened by addition of water (150 ml) and stirred at room temperature for 1 hour. The crystals were collected by filtration and washed with water (100 ml). Drying under vacuum at 40° C. for 3 hours gave the title compound (8.53 g, 70.6% yield) as yellow-brown crystals.

¹H NMR (300 MHz, CDCl₃) δ: 1.64 (3H, t, J=7.1 Hz), 2.39 (2H, quint, J=6.5 Hz), 2.92 (2H, t, J=6.5 Hz), 3.52 (2H, t, J=6.5 Hz), 3.78 (3H, s), 4.63 (2H, q, J=7.1 Hz).

### EXAMPLE 4a

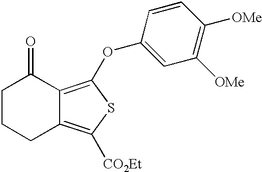

Production of 3-(3,4-dimethoxyphenoxy)-4,5,6,7-tetrahydro-4-oxobenzo[c]thiophene-1-carboxylic Acid Ethyl Ester:

To a solution of 3,4-dimethoxyphenol (10.2 g, 66.2 mmol, 1.1 eq) in tetrahydrofuran (anhydrous) (728 ml) was added potassium t-butoxide (7.42, 66.2 mmol, 1.1 eq) (25° C. to 29° C.). After stirring at room temperature for 30 minutes, the compound (18.2 g, 60.1 mmol) obtained in Example 2a was added gradually. The reaction was carried out at room temperature for 4 hours. The reaction solution was concentrated to obtain a residue (69.5 g). To the thus-obtained concentrated paste residue was added tetrahydrofuran (107 ml). Water (292 ml) was added dropwise over a period of 20 minutes. The resulting mixture was stirred at room temperature for 1 hour. The crystals were collected by filtration and washed with tetrahydrofuran-water (1:3) (90 ml) and water (90 ml). Drying under vacuum at 40° C. for 9 hours gave the title compound (20.1 g, 88.7% yield) as brownish-white crystals.

¹H NMR (300 MHz, CDCl₃) δ:: 1.30 (3H, t, J=7.1 Hz), 2.07 (2H, quint, J=6.5 Hz), 2.57 (2H, t, J=6.5 Hz), 3.22 (2H, t, J=6.5 Hz), 3.87 (3H, s), 3.91 (3H, s), 4.26 (2H, q, J=7.1 Hz), 6.83-6.91 (3H, m).

### EXAMPLE 5a

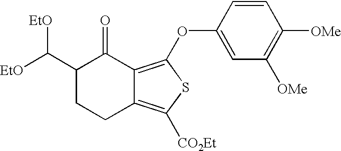

Production of 5-diethoxymethyl-3-(3,4-dimethoxyphenoxy)-4,5,6,7-tetrahydro-4-oxobenzo[c]thiophene-1-carboxylic Acid Ethyl Ester:

Triethyl orthoformate (9.2 ml, 8.27 g, d 0.896, 2.0 eq) and dichloromethane (31 ml) were mixed under argon atmosphere and cooled at −40° C. Thereto was added dropwise boron trifluoride diethyl ether complex (8.7 ml, 9.89 g, d 1.137, 69.7 mmol, 2.5 eq). The resulting solution was warmed to 0° C. and stirred for 15 minutes and then cooled to −70° C. To this solution was added dropwise a solution of the compound (10.5 g, 27.9 mmol), which was obtained in Example 4a, in dichloromethane (32 ml) in 14 minutes (the reaction temperature was raised to −64° C. at highest) with washing the container with dichloromethane (10 ml). Then, N-ethyldiisopropylamine (7.6 ml, 10.82 g, d 1.416, 83.7 mmol, 3.0 eq) was added dropwise in 10 minutes (the reaction temperature was raised to −65° C. at highest). The reaction was carried out at −60 to −70° C. for 3 hours. The reaction solution was poured into an aqueous, saturated solution of sodium bicarbonate (300 ml). After stirring, the organic layer was separated and washed with 1 N hydrochloric acid (100 ml) and purified water (300 ml). The organic layer was dried using magnesium sulfate. The magnesium sulfate was filtered off and washed with dichloromethane (30 ml). The combined filtrate and washing were concentrated and the residue was dissolved by addition of tetrahydrofuran (30 ml). Water (150 ml) was added and the resulting mixture was stirred at room temperature overnight (after the addition of water, a syrupy material precipitated and was gradually solidified). The crystals were collected by filtration and washed with water (40 ml×2). Drying under vacuum at 40° C. for 13 hours gave the title compound (12.72 g, 95.3% yield) as pale yellow-brown, crystalline powder.

¹H NMR (300 MHz, CDCl₃) δ: 1.18 (3H, t, J=7.0 Hz), 1.26 (3H, t, J=7.0 Hz), 1.30 (3H, t, J=7.1 Hz), 2.05-2.35 (2H, m), 2.71 (1H, ddd, J=3.9, 4.1, 10.3 Hz), 2.90 (1H, ddd, J=5.0, 11.0, 18.0 Hz), 3.50-4.00 (5H, m), 3.87 (3H, s), 3.91 (3H, s), 4.26 (2H, q, J=7.1 Hz), 5.16 (1H, d, J=3.2 Hz), 6.82-6.90 (3H, m).

### EXAMPLE 6a

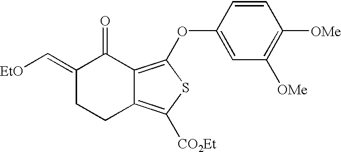

Production of 3-(3,4-dimethoxyphenoxy)-5-ethoxymethylidene-4,5,6,7-tetrahydro-4-oxobenzo[c]thiophene-1-carboxylic Acid Ethyl Ester:

To the compound (437 mg, 0.913 mmol) obtained in Example 5a was added methanesulfonic acid (1.7 ml, 29.5 eq) and the resulting solution was stirred at room temperature for 10 minutes. After cooling with ice, ethanol (5.1 ml) was added dropwise (crystals precipitated to give a light yellow brown suspension). The suspension was stirred under ice cooling for 1 hour. The crystals were collected by filtration and washed with ice-cooled ethanol (3 ml). Drying under vacuum at 40° C. for 7 hours gave the title compound (345 mg, 87.4% yield) as pale brown crystals.

¹H NMR (300 MHz, CDCl₃) δ: 1.30 (3H, t, J=7.1 Hz), 1.37 (3H, t, J=7.1 Hz), 2.68 (2H, t, J=6.3 Hz), 3.21 (2H, t, J=6.9 Hz), 3.86 (3H, s), 3.91 (3H, s), 4.14 (2H, q, J=7.1 Hz), 4.25 (2H, q, J=7.1 Hz), 6.83-6.88 (3H, m), 7.54 (1H, s).

### EXAMPLE 7a

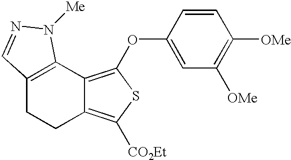

Production of 8-(3,4-dimethoxyphenoxy)-4,5-dihydro-1-methyl-1H-thieno[3,4-g]indazole-6-carboxylic Acid Ethyl Ester:

To the compound (12.7 g, 26.5 mmol) obtained in Example 5a added methanesulfonic acid (31.8 ml) and the resulting solution was stirred at room temperature for 10 minutes (a dark brown solution). After cooing with ice, ethanol (159 ml) was added dropwise over a period of 15 minutes (exothermic, where crystals precipitated during the period to give a light yellowish brown suspension). Thereto was added dropwise a solution of methylhydrazine (5.6 ml, 106 mmol, 4 eq) in ethanol (31.8 ml) under ice cooling over a period of 33 minutes (the precipitate increased further). The reaction solution was heated to 50-60° C. and stirred for 1 hour. After cooling, the reaction solution was concentrated under reduced pressure. A suspension of the residue in ethyl acetate (190 ml) was gradually poured into a suspension of sodium bicarbonate (45.3 g, 0.539 mol, 1.1 eq for methanesulfonic acid) in water (254 ml) (bubbled vigorously). The organic layer was separated and washed with an aqueous, saturated solution of sodium chloride (254 ml). The organic layer was concentrated under reduced pressure and a mixture of the residue (a deep brown, oily substance) and acetonitrile (33 ml) was heated (at reflux) to dissolve. Crystals precipitated soon after gradual cooling. After cooling to room temperature, the resulting mixture was stirred for 1 hour under ice cooling. The crystals were collected by filtration and washed with ice-cooled acetonitrile (9 ml). Drying under vacuum at 40° C. for 9 hours gave the title compound (9.24 g, 84.0% yield) as pale brown crystals.

¹H NMR (300 MHz, CDCl₃) δ: 1.32 (3H, t, J=7.1 Hz), 2.70 (2H, t, J=7.0 Hz), 3.25 (2H, t, J=7.0 Hz), 3.88 (3H, s), 3.90 (3H, s), 4.11 (3H, s), 4.28 (2H, q, J=7.1 Hz), 6.75-6.88 (3H, m), 7.36 (1H, s)

### EXAMPLE 8a

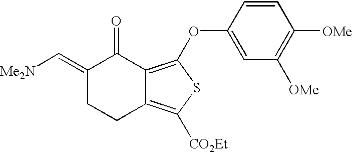

Production of 3-(3,4-dimethoxyphenoxy)-5-dimethylaminomethylidene-4,5,6,7-tetrahydro-4-oxobenzo[c]thiophene-1-carboxylic Acid Ethyl Ester:

To the compound (5.0 g, 13.28 mmol) obtained in Example 4a were added N,N-dimethylformamide (30 ml), N,N-dimethylformamide dimethyl acetal (17.8 ml, d 0.89, 15.8 g, 133 mmol, 10 eq) at room temperature to make a suspension (a light violet color). The reaction solution was heated at 100° C. and stirred for 4 hours. N,N-dimethylformamide dimethyl acetal (3.6 ml, 2 eq) was added additionally and the resulting solution was further stirred at 100° C. for 2 hours. The reaction solution was cooled, mixed with water (100 ml) and extracted with ethyl acetate (50 ml×2). The combined organic layers were washed with an aqueous, saturated solution of sodium chloride (100 ml). The organic layer was mixed with activated carbon (1.0 g) and filtered. The activated carbon was washed with ethyl acetate (20 ml) and the combined filtrate and washing were concentrated under reduced pressure. A mixture of the residue and ethyl acetate (20 ml) was heated at reflux to make a suspension. The resulting suspension was cooled and stirred under ice cooling for 1 hour. The crystals were collected by filtration and washed with ice-cooled ethyl acetate (5 ml). Drying at 40° C. under vacuum for 15 hours gave the title compound (2.58 g, 45.0% yield) as yellow crystals.

¹H NMR (300 MHz, CDCl₃) δ: 1.30 (3H, t, J=7.1 Hz), 2.87 (2H, t, J=6.7 Hz), 3.13 (6H, s), 3.18 (2H, t, J=6.7 Hz), 3.85 (3H, s), 3.89 (3H, s), 4.25 (2H, q, J=7.1 Hz), 6.75-6.95 (3H, m), 7.63 (1H, s).

### EXAMPLE 9a

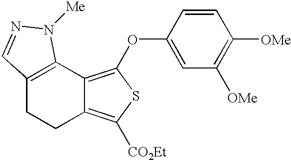

Production of 8-(3,4-dimethoxyphenoxy)-4,5-dihydro-1-methyl-1H-thieno[3,4-g]indazole-6-carboxylic Acid Ethyl Ester:

The compound (0.50 g, 1.16 mmol) obtained in Example 8a was suspended in ethanol (5 ml). Thereto was added methanesulfonic acid (0.25 ml, d 1.48, 370 mg, 3.85 mmol, 3.3 eq) under ice cooling. The suspension was dissolved to obtain a brown and clear solution. Under ice cooing, to this was added dropwise a solution of methylhydrazine (0.06 ml, d 0.87, 53.4 mg, 464 mmol, 4 eq) in ethanol (1.25 ml) below 10° C. A white fume was formed to precipitate crystals. The resulting mixture was heated to 50-60° C. and stirred for 1.5 hours. After cooling, the reaction solution was adjusted to pH 8 with an aqueous 5 N solution of sodium hydroxide. Ethyl acetate (5 ml) and water (10 ml) were added and the resulting mixture was stirred. After standing, the organic layer was separated and the aqueous layer was further extracted with ethyl acetate (5 ml). The combined organic layers were washed with an aqueous, saturated solution of sodium chloride (10 ml). The organic layer was concentrated under reduced pressure and a mixture of the residue (0.54 g) and acetonitrile (1 ml) was heated (at reflux) to dissolve. After gradual cooling, the resulting mixture was stirred for 1 hour under ice cooling. The crystals were collected by filtration and washed with ice-cooled acetonitrile (1 ml). Drying under vacuum at 40° C. for 8 hours gave the title compound (0.365 g, 75.9% yield) as crystals.

¹H NMR (300 MHz, CDCl₃) δ: 1.32 (3H, t, J=7.1 Hz), 2.70 (2H, t, J=7.0 Hz), 3.25 (2H, t, J=7.0 Hz), 3.88 (3H, s), 3.90 (3H, s), 4.11 (3H, s), 4.28 (2H, q, J=7.1 Hz), 6.75-6.88 (3H, m), 7.36 (1H, s).

### EXAMPLE 10a

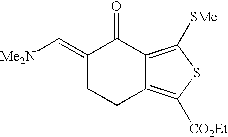

Production of 5-dimethylaminomethylidene-4,5,6,7-tetrahydro-3-methylthio-4-oxobenzo[c]thiophene-1-carboxylic Acid Ethyl Ester:

To the compound (82.0 g, 0.303 mol) obtained in Example 1a were added N,N-dimethylformamide (328 ml) and then trisdimethylaminomethane (161 ml, d 0.82, 132.0 g, 0.909 mol, 3 eq). Upon the addition of trisdimethylaminomethane, the yellow reaction suspension was almost dissolved. The resulting solution was heated at 70±5° C. and stirred for 3 hours. The reaction solution was cooled with ice. At this time, a large quantity of crystals precipitated to solidify the mixture. At this time, the color was red brown. Water (1.15 L) was added dropwise in 50 minutes. Upon the addition of water, a white fume was formed with exothermic generation. The reaction solution became a suspension. The resulting suspension was stirred under ice cooling for 2 hours. The crystals were collected by filtration and washed with water (550 ml). Drying at 40° C. under vacuum for 9 hours gave the title compound (94.1 g, 95.3% yield) as brown crystals.

¹H NMR (300 MHz, CDCl₃) δ: 1.37 (3H, t, J=7.1 Hz), 2.58 (3H, s), 2.87 (2H, t, J=6.7 Hz), 3.11 (6H, s), 3.17 (2H, t, J=6.7 Hz), 4.32 (2H, q, J=7.1 Hz), 7.58 (1H, s).

### EXAMPLE 11a

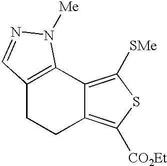

Production of 4,5-dihydro-1-methyl-8-methylsulfanyl-1H-thieno[3,4-g]indazole-6-carboxylic Acid Ethyl Ester:

A mixture of the compound (74.0 g, 0.227 mmol) obtained in Example 10a and ethanol (740 ml) was stirred at room temperature. A suspension was formed. Thereto was added methylhydrazine (25.5 ml, d 0.87, 21.0 g, 0.455 mol, 2 eq). After cooling, 4 M hydrochloric acid (171 ml, 0.682 mol, 3 eq) was added dropwise. An exothermic reaction occurred with generation of a white fume and the temperature was raised to 42° C. A portion of the suspension was dissolved to form a red brown suspension. The suspension was heated to 50-60° C. and stirred for 1 hour. A red-brown, clear solution was formed. After cooling, the reaction solution was adjusted to pH 7 with an aqueous 5 M solution of sodium hydroxide (45 ml). Water (894 ml) was added dropwise and the resulting mixture was stirred under ice cooling for 1.5 hours. The crystals were collected by filtration and washed with ice-cooled ethanol-water (1:2) (200 ml). Drying under vacuum at 40° C. for 9 hours gave the title compound (68.2 g, 97.3% yield) as dark-brown crystals.

¹H NMR (300 MHz, CDCl₃) δ: 1.39 (3H, t, J=7.1 Hz), 2.59 (3H, s), 2.61 (2H, t, J=6.7 Hz), 3.17 (2H, t, J=6.7 Hz), 4.16 (3H, s), 4.37 (2H, q, J=7.1 Hz), 7.40 (1H, s).

### EXAMPLE 12a

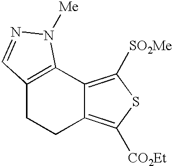

Production of 4,5-dihydro-1-methyl-8-methylsulfonyl-1H-thieno[3,4-g]indazole-6-carboxylic Acid Ethyl Ester:

Concentrated sulfuric acid (6 ml) was added dropwise to acetic acid (24 ml) with ice-cooling. The compound (10.0 g, 32.4 mmol) obtained in Example 11a was added gradually with ice-cooling. Under ice cooling, an aqueous 30% solution of hydrogen peroxide (11.0 g, 97.0 mmol, 3 eq) was added dropwise in 30 minutes. The resulting solution was gradually heated and stirred at 50-55° C. for 3 hours. After the reaction, the ice-cooled reaction solution was mixed with ethanol (30 ml) and adjusted to pH 7 by dropwise addition of an aqueous 5 M solution of sodium hydroxide (115 ml). Water (24 ml) was added to loosen the precipitation mixture and the resulting mixture was stirred at room temperature for 1 hour. The crystals were collected by filtration and washed with ethanol-water (1:4) (15 ml) and water (60 ml). Drying under vacuum at 40° C. for 6 hours gave the title compound (10.4 g, 94.3% yield) as light yellow crystals.

¹H NMR (300 MHz, CDCl₃) δ: 1.41 (3H, t, J=7.1 Hz), 2.66 (2H, t, J=6.7 Hz), 3.18 (2H, t, J=6.7 Hz), 3.26 (3H, s), 4.09 (3H, s), 4.40 (2H, q, J=7.1 Hz), 7.47 (1H, s)

### EXAMPLE 13a

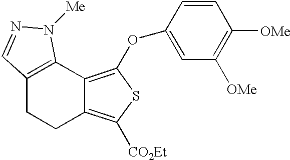

Production of 8-(3,4-dimethoxyphenoxy)-4,5-dihydro-1-methyl-1H-thieno[3,4-g]indazole-6-carboxylic Acid Ethyl Ester:

To a solution of 3,4-dimethoxyphenol (2.49 g, 16.16 mmol, 1.1 eq) in N-methylpyrrolidone (80 ml) was added potassium t-butoxide (1.81 g, 16.16 mmol, 1.1 eq). After stirring at room temperature for 30 minutes (the color was changed from clear violet to dark brown), the compound (5.0 g, 14.69 mmol) obtained in Example 12a was added. The resulting solution was stirred at room temperature for 30 minutes and then at 80-85° C. for 4 hours. After cooling to room temperature, water (160 ml) was added dropwise over a period of 20 minutes. Upon the addition of water, crystals precipitated. The resulting mixture was stirred under ice cooling for 1 hour. The crystals were collected by filtration and washed with water (80 ml). Drying under vacuum at 40° C. gave the title compound (4.37 g, 71.8% yield) as pale brown crystals.

¹H NMR (300 MHz, CDCl₃) δ: 1.32 (3H, t, J=7.1 Hz), 2.70 (2H, t, J=7.0 Hz), 3.25 (2H, t, J=7.0 Hz), 3.88 (3H, s), 3.90 (3H, s), 4.11 (3H, s), 4.28 (2H, q, J=7.1 Hz), 6.75-6.88 (3H, m). 7.36 (1H, s).

### EXAMPLE 14a

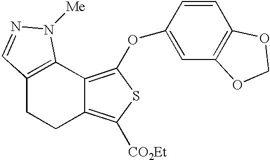

Production of 4,5-dihydro-1-methyl-8-(3,4-methylenedioxyphenoxy)-1H-thieno[3,4-g]indazole-6-carboxylic Acid Ethyl Ester:

According to the same manner as that in Example 13a, the title compound (1.54 g, 65.8% yield) was obtained from the compound (2.0 g) obtained in Example 12a, and 3,4-methylenedioxyphenol.

¹H NMR (300 MHz, CDCl₃) δ: 1.31 (3H, t, J=7.1 Hz), 2.68 (2H, t, J=7.0 Hz), 3.23 (2H, t, J=7.0 Hz), 4.07 (3H, s), 4.27 (2H, q, J=7.1 Hz), 6.00 (2H, s), 6.64-6.79 (3H, m), 7.34 (1H, s).

### EXAMPLE 15a

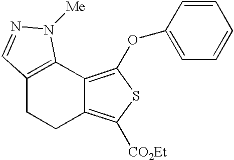

Production of 4,5-dihydro-1-methyl-8-phenoxy-1H-thieno[3,4-g]indazole-6-carboxylic Acid Ethyl Ester:

According to the same manner as that in Example 13a, the title compound (115 mg, 51.4% yield) was obtained from the compound (250 mg) obtained in Example 12a and phenol.

¹H NMR (300 MHz, CDCl₃) δ: 1.32 (3H, t, J=7.1 Hz), 2.71 (2H, t, J=7.0 Hz), 3.26 (2H, t, J=7.0 Hz), 4.06 (3H, s), 4.29 (2H, q, J=7.1 Hz), 7.17-7.43 (5H, m), 7.35 (1H, s).

### EXAMPLE 16a

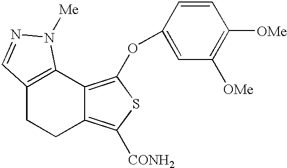

Production of 8-(3,4-dimethoxyphenoxy)-4,5-dihydro-1-methyl-1H-thieno[3,4-g]indazole-6-carboxamide:

A mixture of the compound (1.0 g, 2.413 mmol) obtained in Example 13a, formamide (8 ml, 8 v/w), and sodium methoxide (521 mg, 9.652 mmol, 4.0 eq) was heated at 100° C. for 4 hours. The color of the reaction solution was changed from yellow brown to dark brown. To the reaction solution that was cooled at 75° C., N-methylpyrrolidone (5 ml), an aqueous 5 M solution of sodium hydroxide (0.72 ml, 1.5 eq), and water (0.28 ml) were added. The resulting solution was stirred at 80° C. for 1 hour and 30 minutes and then cooled to room temperature. Upon cooling, crystals precipitated. The reaction mixture was mixed with water (9 ml), methyl ethyl ketone (20 ml), and ethyl acetate (10 ml) and separated. The aqueous layer was extracted three times with ethyl acetate (10 ml). The combined organic layers were washed successively with an aqueous 0.5 M solution of sodium hydroxide (10 ml), an aqueous, saturated solution of sodium chloride (10 ml), 0.1 M hydrochloric acid (10 ml), water (10 ml), and an aqueous, saturated solution of sodium chloride (10 ml). The organic layer was mixed with activated carbon (100 mg, Shirasagi P), stirred at room temperature, and then filtered. The activated carbon was washed with ethyl acetate (5 ml). The combined filtrate and washing were concentrated under reduced pressure to obtain the title compound (0.87 g).

¹H NMR (300 MHz, CDCl₃) δ: 2.70 (2H, t, J=6.7 Hz), 3.15 (2H, t, J=6.7 Hz), 3.86 (3H, s), 3.87 (3H, s), 4.08 (3H, s), 5.79 (2H, broad s), 6.65-6.95 (3H, m). 7.34 (1H, s).

### EXAMPLE 17a

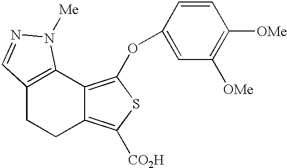

Production of 8-(3,4-dimethoxyphenoxy)-4,5-dihydro-1-methyl-1H-thieno[3,4-g]indazole-6-carboxylic Acid:

A mixture of the compound (4.3 g, 10.4 mmol) obtained in Example 7a and ethanol (17.2 ml) was stirred at room temperature (a suspension). Thereto was added an aqueous 5 N solution of sodium hydroxide (4.2 ml, 20.7 mmol, 2.0 eq). The reaction mixture was heated at 60° C. and stirred for 30 minutes. Upon heating, the suspension was gradually dissolved to form a brown solution. After cooling, 2 N hydrochloric acid (13.0 ml, 2.5 eq) was added dropwise to the reaction solution. Fine crystals were precipitated from the reaction solution to form a highly viscous slurry (pH 1.4). The resulting mixture was stirred under ice cooling for 30 minutes. The crystals were collected by filtration and washed with ethanol-water (9:1) (5 ml). Drying under vacuum at 40° C. gave the title compound (3.89 g, 97.0% yield) as white crystals.

¹H NMR (300 MHz, CDCl₃) δ: 2.73 (2H, t, J=6.7 Hz), 3.25 (2H, t, J=6.7 Hz), 3.88 (3H, s), 3.89 (3H, s), 4.13 (3H, s), 6.97-7.19 (3H, m), 7.46 (1H, s).

### EXAMPLE 18a

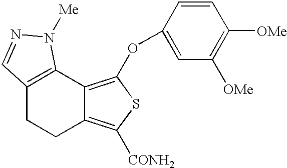

Production of 8-(3,4-dimethoxyphenoxy)-4,5-dihydro-1-methyl-1H-thieno[3,4-g]indazole-6-carboxamide:

The compound (1.0 g, 2.59 mmol) obtained in Example 17a was suspended in N,N-dimethylformamide (8 ml). 1-Hydroxybenzotriazole ammonium salt (0.59 g, 3.88 mmol, 1.5 eq) was added. The suspension was almost dissolved to form a yellow solution. Then, N-ethyl-N′-3-dimethylaminopropylcarbodiimide hydrochloride (0.74 g, 3.88 mmol, 1.5 eq) was added. A suspension was formed without dissolution. This suspension was stirred at room temperature for 3 hours and 15 minutes. The reaction mixture was mixed with water (5 ml) and an aqueous 5 N solution of sodium hydroxide (2 ml, 4 eq) and extracted with ethyl acetate (8 ml×6 times). The combined organic layers were evaporated under reduced pressure to remove the solvent. A remaining N,N-dimethylformamide was removed to dryness. A mixture of the residue, ethanol (20 ml), and water (1 ml) was heated (at reflux) to dissolve. After cooling to the room temperature, the resulting suspension was stirred under ice cooling for 30 minutes. The crystals were collected by filtration and washed with ice-cooled ethanol (3 ml). Drying at 40° C. under vacuum gave the title compound (0.80 g, 79.9% yield) as white crystals.

¹H NMR (300 MHz, CDCl₃) δ: 2.70 (2H, t, J=6.7 Hz), 3.15 (2H, t, J=6.7 Hz), 3.86 (3H, s), 3.87 (3H, s), 4.08 (3H, s), 5.79 (2H, broad s), 6.65-6.95 (3H, m), 7.34 (1H, s).

### EXAMPLE 19a

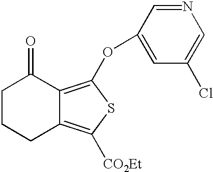

Production of 3-(5-chloro-3-pyridyloxy)-4,5,6,7-tetrahydro-4-oxobenzo[c]thiophene-1-carboxylic Acid Ethyl Ester:

A mixture of the compound (20.0 g, 66.14 mmol) obtained in Example 3a, 3-chloro-5-hydroxypyridine (9.43 g, 72.76 mmol, 1.1 eq), anhydrous potassium carbonate (45.7 g, 330.7 mmol, 5.0 eq), toluene (240 ml), and ethyl acetate (160 ml) was stirred at reflux for 3 hours. The reaction solution was cooled to room temperature, mixed with water (200 ml) and 2-butanone (200 ml), and stirred foe 30 minutes. After standing followed by separation, the organic layer was washed successively with water (200 ml) and an aqueous, saturated solution of sodium chloride (200 ml). The organic layer was concentrated under reduced pressure to dryness to obtain the title compound (21.81 g, 93.7% yield).

¹H NMR (300 MHz, CDCl₃) δ: 1.33 (3H, t, J=7.1 Hz), 2.07 (2H, t, J=6.3 Hz), 2.53 (2H, t, J=6.3 Hz), 3.24 (2H, t, J=6.3 Hz), 4.30 (2H, q, J=7.1 Hz), 7.50-8.46 (4H, m)

### EXAMPLE 20a

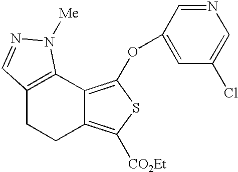

Production of 8-(5-chloro-3-pyridyloxy)-4,5-dihydro-1-methyl-1H-thieno[3,4-g]indazole-6-carboxylic Acid Ethyl Ester:

To a solution of the compound (21.81 g) obtained in Example 19a and dissolved in acetonitrile (80 ml) was added dropwise a solution of N,N-dimethylformamide diisopropyl acetal (23.5 g, 134 mmol, 2.0 eq) in acetonitrile (20 ml) in 4 minutes. The resulting solution was stirred at reflux for 2 hours and 15 minutes. To the reaction solution was added dropwise methanesulfonic acid (18.9 g, 197 mmol, 3.0 eq) with ice-cooling. Then, methylhydrazine (68.0 mmol, 1.0 eq) was added dropwise. The resulting solution was stirred at 50° C. for 1 hour, cooled to room temperature, mixed with water (200 ml) and ethyl acetate (200 ml), and separated. The organic layer was washed with water (200 ml) and an aqueous, saturated solution of sodium chloride (200 ml). The organic layer was mixed with activated carbon (4.0 g), which was then filtered off and washed with ethyl acetate (20 ml). The combined filtrate and washing were concentrated under reduced pressure and a mixture of the residue and ethanol (60 ml) was heated to dissolve. An aqueous 1 M solution of sodium hydroxide (13.2 ml, 0.20 eq) was added dropwise at 50° C. and the resulting mixture was stirred for 30 minutes. After cooling to room temperature, water (67 ml) was added dropwise. After stirring for 30 minutes under ice cooling, the crystals were collected by filtration and washed with ice-cooled ethanol-water (1:2) (15 ml) and water (15 ml). Drying under vacuum at 50° C. gave the title compound (14.97 g, 58.0% yield) as a dark brown solid.

¹H NMR (300 MHz, CDCl₃) δ: 1.34 (3H, t, J=7.1 Hz), 2.71 (2H, t, J=6.9 Hz), 3.26 (2H, t, J=6.9 Hz), 4.00 (3H, s), 4.31 (2H, q, J=7.1 Hz), 7.35 (1H, s), 7.44-8.42 (3H, m).

### EXAMPLE 21a

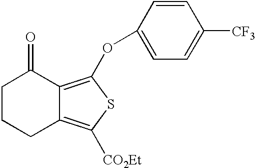

Production of 4,5,6,7-tetrahydro-4-oxo-3-(4-trifluoromethylphenoxy)benzo[c]thiophene-1-carboxylic Acid Ethyl Ester:

A mixture of the compound (15.12 g, 0.050 mol) obtained in Example 3a, 4-hydroxybenzotrifluoride (8.92 g, 0.055 mol, 1.1 eq), anhydrous potassium carbonate (20.72 g, 0.150 mol, 3.0 eq), and ethyl acetate (378 ml) was stirred at reflux for 3 hours and 25 minutes, then mixed additionally with anhydrous potassium carbonate (6.91 g, 1.0 eq), and stirred further for 2 hours. The reaction solution was cooled to room temperature and mixed with water (150 ml). After separation, the aqueous layer was extracted with ethyl acetate (75 ml). The combined organic layers were washed with water (100 ml) and an aqueous, saturated solution of sodium chloride (100 ml×2). The organic layer was concentrated under reduced pressure and a mixture of the residue (19.35 g) and ethanol (31 ml) was heated at reflux to dissolve. Water (15 ml) was added dropwise to form crystals. The resulting mixture was stirred under ice cooling for 1 hour. The crystals were collected by filtration and washed with ice-cooled ethanol-water (1:2) (20 ml) and water (30 ml). Drying under vacuum at 50° C. gave the title compound as yellow-brown crystals (15.7 g, 81.7% yield).

¹H NMR (300 MHz, CDCl₃) δ: 1.32 (3H, t, J=7.1 Hz), 2.09 (2H, t, J=6.3 Hz), 2.53 (2H, t, J=6.3 Hz), 3.24 (2H, t, J=6.3 Hz), 4.29 (2H, q, J=7.1 Hz), 7.27-7.69 (4H, m).

### EXAMPLE 22a

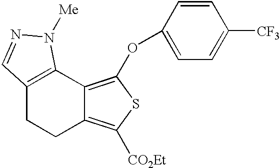

Production of 4,5-dihydro-1-methyl-8-(4-trifluoromethylphenoxy)-1H-thieno[3,4-g]indazole-6-carboxylic Acid Ethyl Ester:

A mixture of the compound (1.0 g, 2.60 mmol), which was obtained in Example 21a, and toluene (6 ml) was heated at 100° C. and thereto was added dropwise a solution of N,N-dimethylformamide diisopropyl acetal (1.37 g, 7.87 mmol, 3 eq) in toluene (6 ml) in 40 minutes. The resulting solution was stirred at 95-100° C. for 1.5 hours. After cooling, to the reaction solution were added successively ethanol (4 ml), methanesulfonic acid (1.5 g, 15.6 mmol, 6 eq), and methylhydrazine (0.13 ml, 2.86 mmol, 1.1 eq). The resulting solution was stirred at 50° C. for 50 minutes. After cooling, water (10 ml) was added and the aqueous layer was separated. It was extracted with toluene (4 ml) and the combined organic layers were washed with water (10 ml) and an aqueous, saturated solution of sodium chloride (100 ml). The organic layer was concentrated under reduced pressure and addition of diisopropyl ether (2 ml) followed by stirring at room temperature resulted in precipitation of crystals. After stirring under ice cooling for 1 hour, the crystals were collected by filtration and washed with ice-cooled diisopropyl ether (2 ml). Drying at 40° C. under vacuum gave the title compound (0.47 g, 43% yield) as light yellow crystals.

¹H NMR (300 MHz, CDCl₃) δ: 1.35 (3H, t, J=7.1 Hz), 2.72 (2H, t, J=7.0 Hz), 3.29 (2H, t, J=7.0 Hz), 4.00 (3H, s), 4.32 (2H, q, J=7.1 Hz), 7.23-7.67 (4H, m), 7.36 (1H, s).

### EXAMPLE 23a

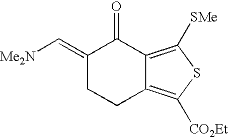

Production of 5-dimethylaminomethylidene-4,5,6,7-tetrahydro-3-methylthio-4-oxobenzo[c]thiophene-1-carboxylic Acid Ethyle Ester:

The compound obtained in Example 1a (1.0 g, 3.70 mmol), N,N-dimethylformamide (4 ml) and N,N-dimethylformamidodiisopropyl acetal (2.32 ml, d 0.84, 1.95 g, 11.1 mmol, 3 eq) were mixed, heated to 100±5° C. and stirred for 6 hours. The reaction mixture was black. When the reaction mixture was cooled, crystals were deposited. When water (8 ml) was added dropwise, an exotherm reaction occurred and further crystals were deposited. The reaction mixture was stirred at room temperature for 2 hours and furhter 1 hour with ice-cooling. The crystals were filtered off and washed with water (3 ml). The crystals were dried at 40° C. under vacuum for 5 hours to obtain the title compound (1.114 g, yield: 92.5%) as dark mud yellow crystals.

¹H-NMR (300 MHz, CDCl₃) δ: 1.37 (3H, t, J=7.1 Hz), 2.58 (3H, s), 2.87 (2H, t, J=6.7 Hz), 3.11 (6H, s), 3.17 (2H, t, J=6.7 Hz), 4.32 (2H, q, J=7.1 Hz), 7.58 (1H, s).

### EXAMPLE 24a

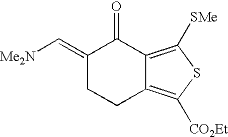

Production of 5-dimethylaminomethylidene-4,5,6,7-tetrahydro-3-methylthio-4-oxobenzo[c]thiophene-carboxylic Acid Ethyl Ester:

The compound obtained in Example 1a (1.0 g, 3.70 mmol), N,N-dimethylformamide (4 ml) and N,N-dimethylformamidodimethyl acetal (1.5 ml, d 0.89, 1.32 g, 11.1 mmol, 3 eq) were mixed, heated to 100±5° C. and stirred for 8 hours. The reaction mixture was black. The reaction mixture was cooled. When water (8 ml) was added dropwise, an exotherm reaction occurred and crystals were deposited. The reaction mixture was stirred for 1 hour with ice-cooling. The crystals were filtered off and washed with water (3 ml). The crystals were dried at 50° C. under vacuum for 9 hours to obtain the title compound (1.105 g, yield: 91.8%) as dark brown crystals.

¹H-NMR (300 MHz, CDCl₃) δ: 1.37 (3H, t, J=7.1 Hz), 2.58 (3H, s), 2.87 (2H, t, J=6.7 Hz), 3.11 (6H, s), 3.17 (2H, t, J=6.7 Hz), 4.32 (2H, q, J=7.1 Hz), 7.58 (1H, s)

### EXAMPLE 25a

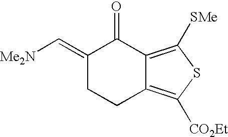

The compound obtained in Example 1a (5.0 g, 18.5 mmol), N,N-dimethylformamide (15 ml) and N,N-dimethylformamidodi-n-propyl acetal (11.4 ml, d 0.89, 9.72 g, 55.5 mmol, 3 eq) were mixed, heated to 100±5° C. and stirred for 8 hours. The reaction mixture was black. The reaction mixture was cooled. When water (8 ml) was added dropwise, an exotherm reaction occurred and crystals were deposited. The reaction mixture was stirred at room temperature for 2 hours. The crystals were filtered off and washed with water (20 ml). The crystals were dried at 40° C. under vacuum for 7 hours to obtain the title compound (5.455 g, yield: 90.6%) as dark brown crystals.

¹H-NMR (300 MHz, CDCl₃) δ: 1.37 (3H, t, J=7.1 Hz), 2.58 (3H, s), 2.87 (2H, t, J=6.7 Hz), 3.11 (6H, s), 3.17 (2H, t, J=6.7 Hz), 4.32 (2H, q, J=7.1 Hz), 7.58 (1H, s).

### TEST EXAMPLE 1

Assay of Chondromodulin-I (ChM-I) mRNA:

ATDC5, a substrain derived from mouse teratocarcinoma cell line AT805, was used. ATDC5 cells were cultured at 37° C. for 7 days in the presence of the test compound using a 1:1 mixed medium of Dulbecco's modified Eagle's medium/Ham's F-12 medium containing 5% heat-inactivated fetal bovine serum and 20 μg/ml gentamycin under a condition of 5% carbon dioxide and 95% air. After the cultivation was completed, cells were collected, from which RNA was recovered by using an RNeasy mini kit (manufactured by Quiagen Company), followed by DNAase treatment using MessageClean Kit (manufactured by Genhunter Company) to obtain the total RNA. The amount of ChM-I mRNA in the total RNA was determined by the RT-PCR method. First, cDNA was synthesized from the RNA preparation by reverse transcription reaction using TaKaRa PCR Kit (AMV) ver. 2.1 and random 9-mers (manufactured by Takara Shuzo Co., Ltd.) according to the instruction for the kit. Next, ChM-I gene was amplified specifically by using HotStarTaq DNA polymerase (manufactured by Quiagen company). The following primers were used for the amplification of ChM-I gene:

| | |
|---|---|
| 1: | 5′-CTAAAATCCTTGAACTCTCTGGCGACCTC-3′ |
| 2: | 5′-CTGCGTCGTCCTGAACATTGGGTCTGG-3′ |

The amplification reaction was carried out in a GeneAmp PCR System 2400 (manufactured by Perkin Elmer Inc.) after warming at 95° C. for 15 minutes, which was followed by 32 repeating reaction cycles of 95° C. for 20 seconds, 58° C. for 10 seconds and 72° C. for 30 seconds. The thus-obtained PCR product was separated by electrophoresis on 2% agarose gel containing ethidium bromide, and the fluorescence intensity of the DNA fragment, which appeared by ultraviolet irradiation at the location corresponding the size (212 bp) expected from the base sequence of mouse ChM-I, was determined. The activity of the test compound to enhance ChM-I expression is expressed as the density of ChM-I band as determined by bioimaging (Table 49).

The cell line “ATDC” used in this experiment has been listed in page 187 of RIKEN GENEBANK General Catalog No. 3, May 1997 Issue that was published by Institute of Physical and Chemical Research (RIKEN), as Cell No. =RCN565, Cell name =ATDC5.

TABLE 49

| | ChM-I expression- |
|---|---|
| | enhancing activity |
| Experiment No. | 10⁻⁶ M |
| 70 | 47 |
| 212 | 20 |
| 216 | 73 |
| 218 | 61 |
| 234 | 39 |
| 249 | 58 |
| 232 | 31 |
| 255 | 106 |
| 251 | 87 |
| No test | 0 |
| compound | |
| added | |

### TEST EXAMPLE 2

Action to Promote Osteogenesis:

The action was determined in interstitial cells prepared from the bone marrow of the femur of normal rats by using alkaline phosphatase activity as the measure for osteogenesis. In other words, interstitial cells were prepared from the bone marrow of the femur of 7-week old male Sprague-Dawley rats according to the method of Maniatopoulos et al. [Cell Tissue Research, vol. 254, p. 317 (1988)], and cultivated in α-MEM (minimum essential medium) containing dexamethasone (10⁻⁷ M) and β-glycerophosphoric acid (10⁻² M) to facilitate formation of the calcified osteoid tissue. After one week, the primary culture cells that reached the confluence were recovered by treating with a solution containing 0.25% trypsin and 0.2% EDTA and were subcultured in culture plates at a cell density of 1.6×10⁻⁴ cells/cm² (day 0 of the cultivation). Starting from day 2 of the cultivation, the test compound (10⁻⁵ M) was added to the above culture fluid and the cells were cultivated for additional 5 days. After the cells were washed with a phosphate buffer solution, 0.2% Nonidet P-40 was added to the cells, which were homogenized and centrifuged at 3000 rpm for 10 minutes, and the supernatant was subjected to the determination of alkaline phosphatase activity according to the method of Lowry et al. [Journal of Biological Chemistry, vol. 207, p. 19 (1954)]. The measured values are expressed in mean±SE as shown in Table 50. Student's t-test was used for the statistical treatment.

TABLE 50

Alkaline phosphatase activity (A₄₀₅)

| Experiment No. | 10 − 7 M | No addition |
|---|---|---|
| 212 | 1.05 ± 0.07** | 0.35 ± 0.02 |
| 70 | 0.25 ± 0.02** | 0.16 ± 0.01 |
| 216 | 0.95 ± 0.08** | 0.32 ± 0.00 |
| 218 | 1.02 ± 0.04** | 0.30 ± 0.01 |
| 234 | 1.60 ± 0.05** | 0.68 ± 0.03 |
| 249 | l.32 ± 0.02** | 0.68 ± 0.03 |
| 273 | 1.96 ± 0.06** | 0.69 ± 0.05 |
| 232 | 1.74 ± 0.01** | 0.50 ± 0.02 |
| 255 | 1.82 ± 0.06** | 0.63 ± 0.02 |
| 251 | 1.33 ± 0.13** | 0.57 ± 0.02 |
| 284 | 0.96 ± 0.05** | 0.35 ± 0.01 |

Mean ± S. E. (n = 4),
**p (0.01 vs. control) Student's t-test

As described hereinabove the compound represented by general formula (I) and a salt thereof of the present invention have, for examples, an action to induce the differentiation of and an action to promote the differentiation of undifferentiated cells such as osteoblast precursor cells, chondrocyte precursor cells and the like and are also expected to an action to enhance the BMP action so that they are useful as prophylactic/therapeutic drugs for metabolic bone diseases including osteoporosis. Also, cell differentiation inducers that have these activities may be applied, for example, as promoters of osteogenesis or chondrogenesis to the prevention/treatment of bone diseases such as bone fracture, bone loss, degenerative articular disease, and the like in orthopedics, or of articular diseases (for examples, osteoarthritis and the like) represented by chondropathy. Furthermore, in the dentistry field, the compounds are expected to be effective in the repair of defect in the periodontal tissue caused by periodontal disease, stabilization of artificial dental root, alveolar crest formation, repair of cleft palate, and the like. Also, since compounds and salts thereof, which are represented by general formula (I) of the present invention, are expected to have activity to enhance the action of neurotrophic factors, they are expected to be useful in the treatment and prevention of diseases that are caused by a variety of nerve degenerations, such as Alzheimer's dementia, senile dementia in general, motor neuron dysfunction (for examples, amyotrophic lateral sclerosis and the like), diabetic peripheral neuropathy, and so on. Furthermore, since compounds and salts thereof, which are represented by general formula (I) of the present invention, are expected to have an anti-MMP activity, they are expected to be useful in the treatment and prevention of diseases, in which MMP is involved, such as osteoarthritis, chronic rheumatoid arthritis, arteriosclerosis, tumor metastasis, and the like.

In addition, according to the production processes of the present invention, fused, heterocyclic derivatives possessing an action to promote osteogenesis or synthetic intermediates thereof can be synthesized with a position selectivity, so that the objective compounds can be produced with an industrial advantage without the use of tedious purification operations.

Furthermore, by the use of “an optionally substituted amino group” as R¹³ of the general formula (IX), a compound such as boron trifluoride ether complex, which has a corrosion problem, is not employed in the production of compounds (I) so that the objective compounds can be produced with an industrial advantage.

1. A compound represented by the general formula (I): 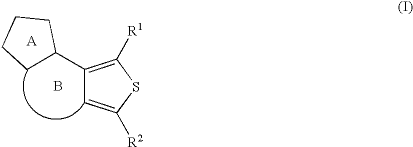
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 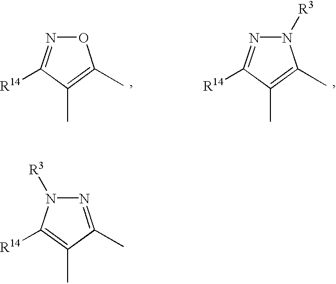
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than the compound represented by the formula: 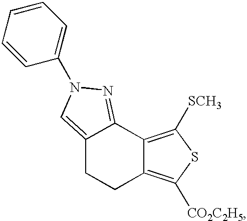
and a compound wherein, when ring A is 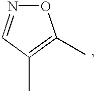
Z¹ of -Z¹-Z² is —CO— and Z² thereof is optionally substituted amino group, or a salt thereof.

2. The compound according to claim 1, wherein ring B represents a 5- to 7-membered hydrocarbon ring having 1 to 3 C₁₋₈ alkyl groups.

3. The compound according to claim 1, wherein ring B represents an unsubstituted 5- to 7-membered hydrocarbon ring.

4. The compound according to claim 1, wherein ring B represents an unsubstituted 6-membered hydrocarbon ring.

5. The compound according to claim 1, wherein ring A is 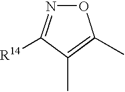
wherein R¹⁴ is as defined in claim 1.

6. The compound according to claim 1, wherein ring A is 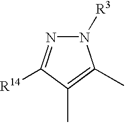
wherein each symbol is as defined in claim 1.

7. The compound according to claim 1, wherein ring A is 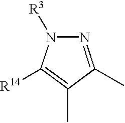
wherein each symbol is as defined in claim 1.

8. The compound according to claim 1, wherein R³ is (1) hydrogen atom, (2) a C₁₋₈ alkyl group optionally substituted with 1 to 3 substituents selected from the group consisting of C₁₋₆ alkoxy, carboxyl, C₁₋₆ alkoxy-carbonayl, halogen and hydroxyl, (3) a C₇₋₁₄ aralkyl group, (4) a C₂₋₈ alkanoyl group, (5) a C₁₋₆ alkoxy-carbonyl group,
(6) carbamoyl group optionally substituted with C₁₋₆ alkyl,
(7) C₁₋₈ alkylsulfinyl or (8) C₁₋₈ alkylsulfonyl, and R¹⁴ is (1) hydrogen atom, (2) halogen, (3) C₁₋₈ alkyl or (4) i) C₆₋₁₀ aryl, ii) a 5- to 7-membered heterocyclic group containing one sulfur atom, one nitrogen atom or one oxygen atom, iii) a 5- to 6-membered heterocyclic group containing 2 to 4 nitrogen atoms, iv) a 5- to 6-membered heterocyclic group containing 1 to 2 nitrogen atoms and one sulfur atom or oxygen atom, or v) a heterocyclic group of ii) to iv) which is fused with a 5- to 6-membered heterocyclic ring containing 2 or less nitrogen atom, benzene ring or a 5-membered heterocyclic ring, each of which may have 1 to 3 substituents selected from the group consisting of C₁₋₆ alkyl, C₁₋₆ haloalkyl, C₁₋₆ alkoxy, C₁₋₆ haloalkoxy, C₁₋₆ alkoxy-carbonyl, amino and C₁₋₆ alkylthio.

9. The compound according to claim 1, wherein R³ is a C₁₋₈ alkyl group and R¹⁴ is hydrogen atom.

10. The compound according to claim 1, wherein R¹ is an optionally substituted hydrocarbon group.

11. The compound according to claim 1, wherein R¹ is an optionally substituted heterocyclic group.

12. The compound according to claim 1, wherein R¹ is an optionally substituted sulfinyl group, an optionally substituted sulfonyl group or an optionally substituted thiol group.

13. The compound according to claim 1, wherein R¹ is an optionally substituted hydroxyl group.

14. The compound according to claim 1, wherein R¹ is an optionally substituted amino group.

15. The compound according to claim 1, wherein R¹ is:
(1) sulfinyl group, sulfonyl group or thiol group each of which may be substituted with C₁₋₈ alkyl or C₇₋₁₄ aralkyl,
(2) i) a C₆₋₁₀ aryloxy group,
ii) an aromatic 5- or 6-membered heterocyclic-oxy group having 1 to 4 hetero atoms selected from the group consisting of nitrogen atom, oxygen atom and sulfur atom, or
iii) a fused bicyclic heterocyclic-oxy group formed by aromatic 5- or 6-membered heterocyclic ring having 1 to 3 hetero atoms selected from the group consisting of nitrogen atom, oxygen atom and sulfur atom fused with benzene ring or aromatic 5- or 6-membered heterocyclic ring having 1 to 3 hetero atoms selected from the group consisting of nitrogen atom, oxygen atom and sulfur atom,
each of which may be substituted with 1 or 2 substituents selected from the group consisting of C₁₋₈ alkyl, C₆₋₁₄ aryl, C₇₋₁₄ aralkyl, hydroxyl, C₁₋₆ alkoxy, C₁₋₆ alkylenedioxy, C₆₋₁₄ aryloxy, C₁₋₆ alkylthio, morpholinosulfonyl, formyl, C₁₋₆ alkyl-carbonyl, carboxyl, C₁₋₆ alkoxy-carbonyl, C₆₋₁₄ aryloxy-carbonyl, C₇₋₁₄ aralkyloxy-carbonxyl, amino, mono- or di-C₁₋₆ alkylamino, mono- or di-C₁₋₆ alkyl-carbonylamino, toluenesulfonylamino, C₁₋₆ alkylaminothiocarbonylamino, nitro, halogen, C₁₋₆ haloalkyl, C₁₋₆ haloalkoxy, C₁₋₆ alkoxy-C₆₋₁₄ aryl, 2-amino-3-(4-morpholinyl)-3-oxo-C₁₋₆ alkyl, C₁₋₆ alkyl having one or two di(C₁₋₆ alkyoxy)phosphoryl, an aromatic 5- or 6-membered heterocyclic group having 1 to 4 hetero atoms selected from the group consisting of nitorgen atom, oxygen atom and sulfur atom, an aromatic 5- or 6-membered heterocyclic-oxy group having 1 to 4 hetero atoms selected from the group consisting of nitrogen atom, oxygen atom and sulfur atom, and a fused bicyclic heterocyclic-oxy group formed by an aromatic 5- or 6-membered heterocyclic ring having 1 to 3 hetero atoms selected from the group consisting of nitorgen atom, oxygen atom and sulfur aton fused with benzene ring or an aromatic 5- or 6-membered heterocyclic ring having 1 to 3 hetero atoms selected from the group consisting of nitrogen atom, oxygen atom and sulfur atom,
(3) a C₁₋₈ alkyloxy group, a C₃₋₇ cycloalkyloxy group, a C₇₋₁₄ aralkyloxy group or dihydrobenzofuranyloxy group,
(4) amino group which may be substituted with 1 or 2 substituents selected from the group consisting of C₁₋₈ alkyl, C₆₋₁₀ aryl, C₇₋₁₄ aralkyl, halo C₆₋₁₀ aryl, C₁₋₆ alkyl-carbonyl, C₆₋₁₀ aryl-carbonyl and C₇₋₁₄ aralkyl-oxycarbonyl, or
(5) C₁₋₈ alkyl or C₇₋₁₄ aralkyl.

16. The compound according to claim 1, wherein R¹ is:
(1) sufinyl group, sulfonyl group or thiol group each of which is substituted with C₁₋₈ alkyl,
(2) (a) C₁₋₆ alkylenedioxy, (b) di (C₁₋₆ alkoxy)phosphoryl-C₁₋₆ alkyl, (c) C₁₋₆ haloalkyl, (d) C₇₋₁₄ aralkyloxy, (e) aromatic 5- or 6-membered heterocyclic-oxy having 1 to 4 hetero atoms selected from the group consisting of nitrogen atom, oxygen atom and sulfur atom or (f) a C₆₋₁₀ aryloxy group which may be subsituted with fused bicyclic heterocyclic-oxy formed by an aromatic 5- or 6-membered heterocyclic ring having 1 to 3 hetero atoms selected from the group consisting of nitrogen atom, oxygen atom and sulfur atom fused with benzene ring or an aromatic 5- or 6-membered heterocyclic ring having 1 to 3 hetero atoms selected from the group consisting of nitrogen atom, oxygen atom and sulfur atom,
(3) an aromatic 5- or 6-membered heterocyclic-oxy group having 1 to 4 hetero atoms selected from the group consisting of nitrogen atom, oxygen atom and sulfur atom, or
(4) a concensed bicyclic heterocyclic-oxy group formed by an aromatic 5- or 6-membered heterocyclic ring having 1 to 3 hetero atoms selected from the group consisting or nitrogen atom, oxygen atom and sulfur atom fused with benzene ring or an aromatic 5- or 6-membered heterocyc ring having 1 to 3 hetero atom selected from the group consisting of nitrogen atom, oxygen atom and sulfur atom.

17. The compound according to claim 1, wherein R² is represented by
—CO-Z^{2′}
wherein Z^{2′} represents hydrogen atom or an optionally substituted hydrocarbon group, an optionally substituted heterocyclic group, an optionally substituted amino group or an optionally substituted hydroxyl group.

18. The compound according to claim 17, wherein Z^{2′} is (1) amino group which may be substituted with 1 or 2 substituents selected from the group consisting of C₁₋₈ alkyl, C₆₋₁₄ aryl, C₇₋₁₄ aralkyl, hydroxyl, C₁₋₆ alkoxy, amino, mono- or di-C₁₋₆ alkyl amino, C₁₋₆ alkoxy-carbonylamino, an aromatic 5- or 6-membered heterocyclic group having 1 to 4 hetero atoms selected from the group consisting of nitorgen atom, oxygen atom and sulfur atom, C₁₋₆ haloalkyl, carboxy C₁₋₆ alkyl, C₁₋₆ alkoxy-carbonyl-C₁₋₆ alkyl, carbamoyl C₁₋₆ alkyl, aromatic 5- or 6-membered heterocyclic-C₁₋₆ alkyl having 1 to 4 hetero atoms selected from the group consisting or nitrogen atom, oxygen atom and sulfur atom, aromatic 5- or 6-membered heterocyclic-C₁₋₆ alkyl-C₆₋₁₄ aryl having 1 to 4 hetero atoms selected from the group consisting of nitrogen atom, oxygen atom and sulfur atom, and mono- or di-C₁₋₆ alkylamino C₁₋₆ alkyl, or (2) morpholino group.

19. The compound according to claim 17, wherein Z² is amino group optionally substituted with 1 or 2 C₁₋₆ alkyls.

20. The compound according to claim 1, wherein R¹⁴ is hydrogen atom.

21. The compound according to claim 1, wherein R¹ is:
(1) sulfinyl group, sulfonyl group or thiol group each of which may be substituted with C₁₋₈ alkyl or C₇₋₁₄ aralkyl,
(2) i) a C₆₋₁₀ aryloxy group,
ii) an aromatic 5- or 6-membered heterocyclic-oxy group having 1 to 4 hetero atoms selected from the group consisting of nitrogen atom, oxygen atom and sulfur atom, or
iii) a fused bicyclic heterocyclic-oxy group formed by aromatic 5- or 6-membered heterocyclic ring having 1 to 3 hetero atoms selected from the group consisting of nitrogen atom, oxygen atom and sulfur atom fused with benzene ring or aromatic 5- or 6-membered heterocyclic ring having 1 to 3 hetero atoms selected from the group consisting of nitrogen atom, oxygen atom and sulfur atom,
each of which may be substituted with 1 to 2 substituents selected from the group consisting of C₁₋₈ alkyl, C₆₋₁₄ aryl, C₇₋₁₄ aralkyl, hydroxyl, C₁₋₆ alkoxy, C₁₋₆ alkylenedioxy, C₆₋₁₄ aryloxy, C₁₋₆ alkylthio, morpholinosulfonyl, formyl, C₁₋₆ alkyl-carbonyl, carboxyl, C₁₋₆ alkoxy-carbonyl, C₆₋₁₄ aryloxy-carbonyl, C₇₋₁₄ aralkyloxy-carbonxyl, amino, mono- or di-C₁₋₆ alkylamino, mono- or di-C₁₋₆ alkyl-carbonylamino, toluenesulfonylamino, C₁₋₆ alkylaminothiocarbonylamino, nitro, halogen, C₁₋₆ haloalkyl, C₁₋₆ haloalkoxy, C₁₋₆ alkoxy-C₆₋₁₄ aryl, 2-amino-3-(4-morpholinyl)-3-oxo-C₁₋₆ alkyl, C₁₋₆ alkyl having one or two di(C₁₋₆ alkyoxy)phosphoryl, an aromatic 5- or 6-membered heterocyclic group having 1 to 4 hetero atoms selected from the group consisting of nitorgen atom, oxygen atom and sulfur atom, an aromatic 5- or 6-membered heterocyclic-oxy group having 1 to 4 hetero atoms selected from the group consisting of nitrogen atom, oxygen atom and sulfur atom, and a fused bicyclic heterocyclic-oxy group formed by an aromatic 5- or 6-membered heterocyclic ring having 1 to 3 hetero atoms selected from the group consisting of nitorgen atom, oxygen atom and sulfur aton fused with benzene ring or an aromatic 5- or 6-membered heterocyclic ring having 1 to 3 hetero atoms selected from the group consisting of nitrogen atom, oxygen atom and sulfur atom,
(3) a C₁₋₈ alkyloxy group, a C₃₋₇ cycloalkyloxy group, a C₆₋₁₄ aryloxy group, a C₇₋₁₄ aralkyloxy group or dihydrobenzofuranyloxy group,
(4) amino group which may be substituted with 1 or 2 substituents selected from the group consisting of C₁₋₈ alkyl, C₆₋₁₀ aryl, C₇₋₁₄ aralkyl, halo C₆₋₁₀ aryl, C₁₋₆ alkyl-carbonyl, C₆₋₁₀ aryl-carbonyl and C₇₋₁₄ aralkyl-oxycarbonyl, or
(5) C₁₋₈ alkyl or C₇₋₁₄ aralkyl
R² is —CO-Z^{2′} and Z^{2′} is:
(1) amino group each of which may be substituted with 1 or 2 substituents selected from the group consisting of C₁₋₈ alkyl, C₆₋₁₄ aryl, C₇₋₁₄ aralkyl, hydroxyl, C₁₋₆ alkoxy, amino, mono- or di-C₁₋₆ alkyl amino, C₁₋₆ alkoxy-carbonylamino, an aromatic 5- or 6-membered heterocyclic group having 1 to 4 hetero atoms selected from the group consisting of nitorgen atom, oxygen atom and sulfur atom, C₁₋₆ haloalkyl, carboxy C₁₋₆ alkyl, C₁₋₆ alkoxy-carbonyl-C₁₋₆ alkyl, carbamoyl C₁₋₆ alkyl, aromatic 5- or 6-membered heterocyclic-C₁₋₆ alkyl having 1 to 4 hetero atoms selected from the group consisting or nitrogen atom, oxygen atom and sulfur atom, aromatic 5- or 6-membered heterocyclic-C₁₋₆ alkyl-C₆₋₁₄ aryl having 1 to 4 hetero atoms selected from the group consisting of nitrogen atom, oxygen atom and sulfur atom, and mono- or di-C₁₋₆ alkylamino C₁₋₆ alkyl, or (2) morpholino group,
R³ is:
(1) hydrogen atom, (2) a C₁₋₈ alkyl group optionally substituted with 1 to 3 substituents selected from the group consisting of C₁₋₆ alkoxy, carboxyl, C₁₋₆ alkoxy-carbonayl, halogen and hydroxyl, (3) a C₇₋₁₄ aralkyl group, (4) a C₂₋₈ alkanoyl group, (5) a C₁₋₆ alkoxy-carbonyl group, (6) carbamoyl group optionally substituted with C₁₋₆ alkyl, (7) C₁₋₈ alkylsulfinyl or (8) C₁₋₈ alkylsulfonyl, and R¹⁴ is (1) hydrogen atom, (2) halogen, (3) C₁₋₈ alkyl or (4) i) C₆₋₁₀ aryl, ii) a 5- to 7-membered heterocyclic group containing one sulfur atom, one nitrogen atom or one oxygen atom, iii) a 5- to 6-membered heterocyclic group containing 2 to 4 nitrogen atoms, iv) a 5- to 6-membered heterocyclic group containing 1 to 2 nitrogen atoms and one sulfur atom or oxygen atom, or v) a heterocyclic group of ii) to iv) which is fused with a 5- to 0.6-membered heterocyclic ring containing 2 or less nitrogen atom, benzene ring or a 5-membered heterocyclic ring, each of which may have 1 to 3 substituents selected from the group consisting of C₁₋₆ alkyl, C₁₋₆ haloalkyl, C₁₋₆ alkoxy, C₁₋₆ haloalkoxy, C₁₋₆ alkoxy-carbonyl, amino and C₁₋₆ alkylthio, and
ring B is a 5- to 7-membered hydrocarbon ring optionally substituted with 1 to 3 C₁₋₈ alkyl groups.

22. The compound according to claim 1, wherein R¹ is (a) C₁₋₆ alkylenedioxy, (b) di(C₁₋₆ alkoxy)phosphoryl-C₁₋₆ alkyl, (c) C₁₋₆ haloalkyl, (d) C₇₋₁₄ aralkyloxy, (e) aromatic 5- or 6-membered heterocyclic-oxy having 1 to 4 hetero atoms selected from the group consisting of nitrogen atom, oxygen atom and sulfur atom or (f) a C₆₋₁₀ aryloxy group which may be subsituted with fused bicyclic heterocyclic-oxy formed by an aromatic 5- or 6-membered heterocyclic ring having 1 to 3 hetero atoms selected from the group consisting of nitrogen atom, oxygen atom and sulfur atom fused with benzene ring or an aromatic 5- or 6-membered heterocyclic ring having 1 to 3 hetero atoms selected from the group consisting of nitrogen atom, oxygen atom and sulfur atom,
R² is —CO-Z^{2′} and Z^{2′} is amino group optionally substituted with 1 or 2 C₁₋₆ alkyl groups,
R³ is a C₁₋₈ alkyl group,
R¹⁴ is hydrogen atom, and
ring B is cyclohexane ring.

23. The compound according to claim 1, wherein
9-methylthio-4,5-dihydro-6H-1-oxa-8-thia-2-aza-cyclopenta[e]azulene-7-carboxamide,
4,5-dihydro-8-methylthio-4-phenylisoxazolo[4,5-e]benzo[c]thiophene-6-carboxamide,
4,5-dihydro-4-methyl-8-(methylthio)isoxazolo[4,5-e]benzo[c]thiophene-6-carboxamide, and
4,5-dihydro-4,4-dimethyl-8-(methylthio)isoxazolo[4,5-e]benzo[c]thiophene-6-carboxamide are further excluded from the compound represented by the formula (I).

24. The compound according to claim 1, wherein a compound wherein, when ring B is a 6-membered hydrocarbon ring substituted with a lower alkyl group or phenyl, R¹ is a lower alkylthio group, and a compound wherein, when ring B is an unsubstituted 7-membered hydrocarbon ring, R¹ is a lower alkylthio group are further excluded from the compound represented by the formula (I).

25. The compound according to claim 1, wherein the compound represented by the formula (I) is:
4,5-dihydro-1-methyl-8-propylsulfanyl-1H-thieno[3,4-g]indazole-6-carboxamide;
4,5-dihydro-1-methyl-8-propylsulfinyl-1H-thieno [3,4-g]indazole-6-carboxamide;
4,5-dihydro-1-methyl-8-propylsulfonyl-1H-thieno [3,4-g]indazole-6-carboxamide;
4,5-dihydro-8-(3,4-methylenedioxyphenoxy)-1-methyl-1H-thieno[3,4-g]indazole-6-carboxamide;
4,5-dihydro-8-phenoxy-1-methyl-1H-thieno[3,4-g]indazole-6-carboxamide;
4,5-dihydro-8-(3,4-methylenedioxyphenoxy)thieno-[3,4-g]-1,2-benzisoxazole-6-carboxamide;
8-[4-[(diethoxyphosphoryl)methyl]phenoxy]-4,5-dihydro-2-methyl-2H-thieno [3,4-g]indazole-6-carboxamide;
8-[4-[(diethoxyphosphoryl)methyl]phenoxy]-4,5-dihydro-1-methyl-1H-thieno[3,4-g]indazole-6-carboxamide;
N-ethyl-4,5-dihydro-8-(3,4-methylenedioxy-phenoxy)-1-methyl-1H-thieno[3,4-g]indazole-6-carboxamide;
4,5-dihydro-1-methyl-8-(4-trifluoromethyl-phenoxy)-1H-thieno[3,4-g]indazole-6-carboxamide;
4,5-dihydro-1-methyl-8-(6-quinolinyloxy)-1H-thieno[3,4-g]indazole-6-carboxamide;
4,5-dihydro-1-methyl-8-(3-pyridinyloxy)-1H-thieno[3,4-g]indazole-6-carboxamide;
8-[4-(benzyloxy)phenoxy]-4,5-dihydro-1-methyl-1H-thieno[3,4-g]indazole-6-carboxamide; or
4,5-dihydro-1-methyl-8-[4-(2-quinolinylmethoxy)phenoxy]-1H-thieno[3,4-g]indazole-6-carboxamide.

26. A prodrug of the compound according to claim 1 or a salt thereof.

27. A pharmaceutical composition which comprises a compound represented by the general formula (I): 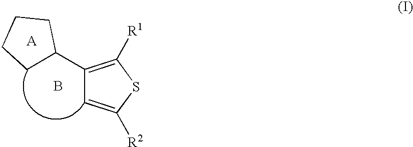
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 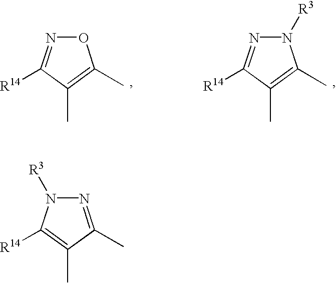
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than, when ring A is 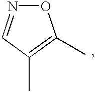
Z¹ of -Z¹-Z² is —CO— and Z² thereof is optionally substituted amino group, or a pharmaceutically acceptable salt thereof or a prodrug thereof.

28. The composition according to claim 27, wherein
9-methylthio-4,5-dihydro-6H-1-oxa-8-thia-2-aza-cyclopenta[e]azulene-7-carboxamide,
4,5-dihydro-8-methylthio-4-phenylisoxazolo[4,5-e]benzo[c]thiophene-6-carboxamide,
4,5-dihydro-4-methyl-8-(methylthio)isoxazolo[4,5-e]benzo[c]thiophene-6-carboxamide, and
4,5-dihydro-4,4-dimethyl-8-(methylthio)isoxazolo[4,5-e]benzo[c]thiophene-6-carboxamide are further excluded from the compound represented by the formula (I).

29. The composition according claim 27, wherein a compound wherein, when ring B is a 6-membered hydrocarbon ring substituted with a lower alkyl group or phenyl, R¹ is a lower alkylthio group, and a compound wherein, when ring B is an unsubstituted 7-membered hydrocarbon ring, R¹ is a lower alkylthio group are further excluded from the compound represented by the formula (I).

30. A cell differentiation inducing drug which comprises a compound represented by the general formula 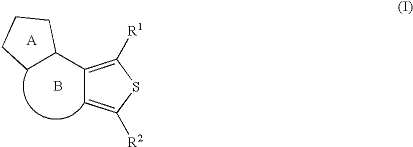
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 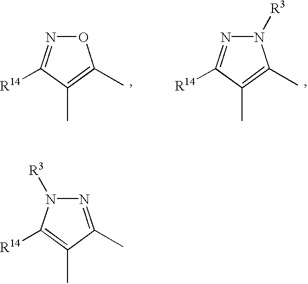
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula: 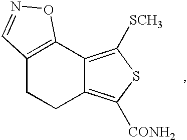
or a pharmaceutically acceptable salt thereof or a prodrug thereof.

31. The cell differentiation inducing drug according to claim 30, wherein 9-methylthio-4,5-dihydro-6H-1-oxa-8-thia-2-aza-cyclopenta[e]azulene-7-carboxamide,
4,5-dihydro-8-methylthio-4-phenylisoxazolo[4,5-e]benzo[c]thiophene-6-carboxamide,
4,5-dihydro-4-methyl-8-(methylthio)isoxazolo[4,5-e]benzo[c]thiophene-6-carboxamide, and
4,5-dihydro-4,4-dimethyl-8-(methylthio)isoxazolo[4,5-e]benzo[c]thiophene-6-carboxamide are further excluded from the compound represented by the formula (I).

32. The cell differentiation inducing drug according to claim 30, wherein a compound wherein, when ring B is a 6-membered hydrocarbon ring substituted with a lower alkyl group or phenyl, R¹ is a lower alkylthio group, and a compound wherein, when ring B is an unsubstituted 7-membered hydrocarbon ring, R¹ is a lower alkylthio group are further excluded from the compound represented by the formula (I).

33. A a prophylactic or therapeutic drug for bone or articular diseases which comprises a compound represented by the general formula (I): 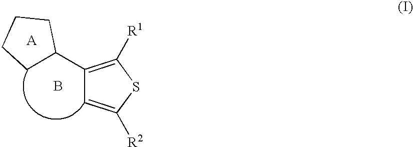
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 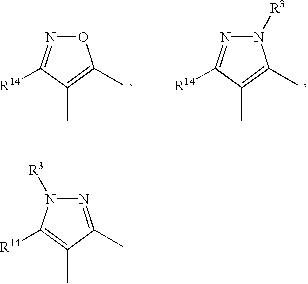
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula: 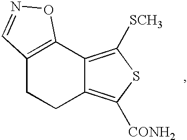
or a pharmaceutically acceptable salt thereof or a prodrug thereof.

34. The prophylactic or therapeutic drug for bone or articular diseases according to claim 33, wherein the bone or articular diseases are osteoporosis, bone fracture, osteoarthritis or chronic rheumatoid arthritis.

35. A compound represented by general formula (II): 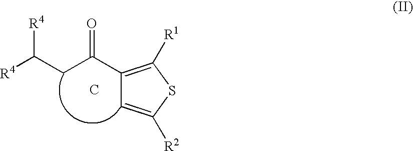
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R⁴ represents a substituted hydroxyl group; and ring C represents an optionally substituted 5- to 7-membered hydrocarbon ring, or a salt thereof.

36. A compound represented by the general formula (IX): 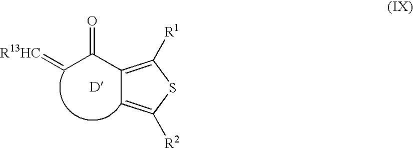
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group, or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group, or an optionally substituted amino group); R¹³ represents an optionally substituted amino group or an optionally substituted hydroxyl group; and ring D′ represents an optionally substituted 5- to 7-membered hydrocarbon ring, or a salt thereof.

37. A process for producing a compound represented by the general formula (IX-1): 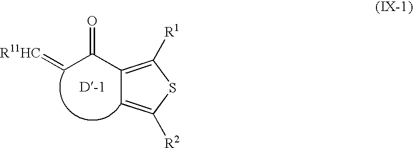
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R” represents an optionally substituted amino group; ring D′-1 represents an optionally substituted 5- to 7-membered hydrocarbon, or a salt thereof, which comprises reacting a compound of the general formula (IV) 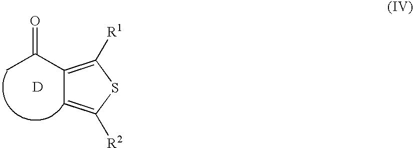
wherein ring D represents an optionally substituted 5- to 7-membered hydrocarbon ring; and R¹ and R² are as defined above, with an amide acetal.

38. A process for producing a compound of the general formula (IX-2): 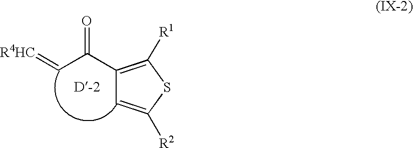
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group; R² represents cyano group, formyl group, thioformyl group or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); R⁴ represents an optionally substituted hydroxyl group; ring D′-2 represents an optionally substituted 5- to 7-membered hydrocarbon ring, or a salt thereof, which comprises subjecting a compound of the general formula (II): 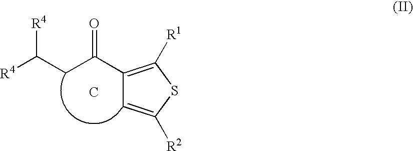
wherein ring C represents an optionally substituted 5- to 7-membered hydrocarbon ring; and R¹, R² and R⁴ are as defined above, to a de-alcoholization reaction.

39. A process for producing a compound represented by the general formula (I): 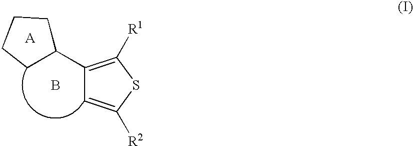
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 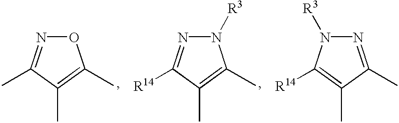
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring, or a salt thereof, which comprises subjecting a compound represented by the general formula (IX) 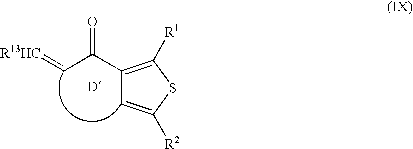
wherein R¹³ represents an optionally substituted amino group or an optionally substituted hydroxyl group; ring D′ represents an optionally substituted 5- to 7-membered hydrocarbon ring; and R¹ and R² are as defined above, or a salt thereof, and hydroxylamine or its salt, or a compound represented by the formula: R^{3′}NHNH₂ (wherein R^{3′} represents hydrogen atom or an optionally substituted hydrocarbon group, an optionally substituted heterocyclic group, an optionally substituted sulfonyl group, or an optionally substituted acyl group), or a salt thereof to a cyclization reaction, if desired, followed by conversion into an optionally substituted hydroxyl group or an optionally substituted amino group.

40. A process for producing a compound represented by the general formula (I-II): 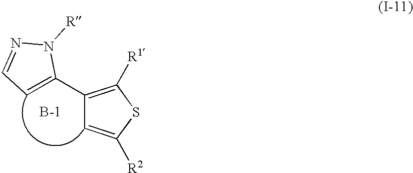
wherein R^{1′} represents, among an optionally substituted hydrocarbon group, an optionally substituted heterocyclic group, an optionally substituted sulfinyl group, an optionally substituted sulfonyl group, an optionally substituted hydroxyl group, an optionally substituted thiol group, or an optionally substituted amino group, a group represented by the formula: —X′—W′ (wherein X′ represents a bond, an optionally substituted carbon atom, an optionally substituted nitrogen atom, oxygen atom or an optionally oxidized sulfur atom; R^{3″} represents an optionally substituted hydrocarbon group, an optionally substituted heterocyclic group, an optionally substituted sulfonyl group or an optionally substituted acyl group; and W′ represents an optionally substituted cyclic group or carbon or nitrogen atom having 2 or more substituents); R² represents cyano group, formyl group, thioformyl group, or a group represented by the formula: -Z¹-Z² (wherein Z¹ represents —CO—, —CS—, —SO—, or —SO₂—; and Z² represents an optionally substituted hydrocarbon group, an optionally substituted heterocyclic group, an optionally substituted hydroxyl group or an optionally substituted amino group); ring B-1 represents an optionally substituted 5- to 7-membered hydrocarbon ring, or a salt thereof, which comprises subjecting a compound represented by the general formula (IX-3): 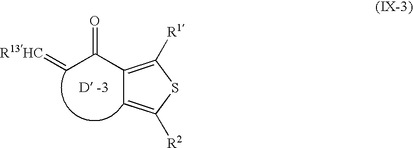
wherein R^{13′} represents an optionally substituted amino group or an optionally substituted hydroxyl group; ring D′-3 represents an optionally substituted 5- to 7-membered hydrocarbon ring; and R^{1′} and R² are as defined above, or a salt thereof, and a compound resented by formula: R^{3″}NHNH₂ (wherein R^{3″} is as defined above), or a salt thereof to a cyclization reaction in the presence of an acid under substantially anhydrous conditions.

41. The process according to claim 40, wherein R^{1′} is the group represented by —X′—W′, wherein, X′ represents oxygen atom or an optionally oxidized sulfur atom; and W′ represents an optionally substituted cyclic group.

42. A method for inducing cell differentiation in a mammal in need thereof which comprises administering an effective amount of a compound represented by the general formula (I): 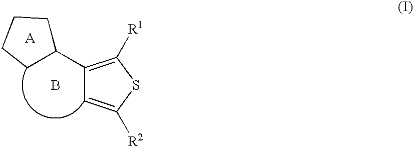
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 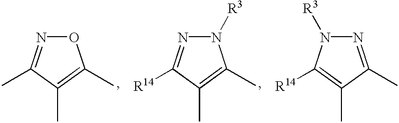
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula: 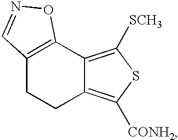
or a pharmaceutically acceptable salt thereof or a prodrug thereof, to the mammal.

43. A method for preventing or treating bone or articular diseases in a mammal in need thereof which comprises administering an effective amount of a compound represented by the general formula (I): 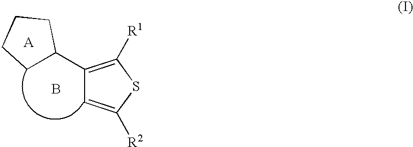
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 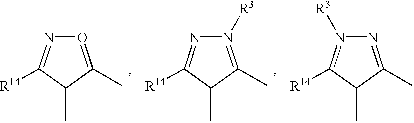
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula: 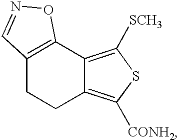
or a pharmaceutically acceptable salt thereof or a prodrug thereof, to the mammal.

44. Use of a compound represented by the general formula (I): 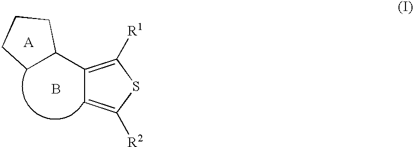
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 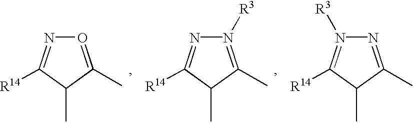
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula: 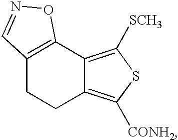
or a pharmaceutically acceptable salt thereof or a prodrug thereof, for manufacturing a cell defferentiation inducing drug.

45. Use of a compound represented by the general formula (I): 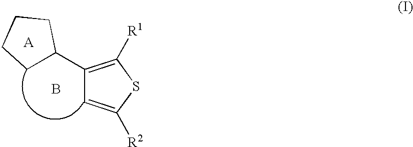
wherein R¹ represents an optionally substituted hydrocarbon group, an optionally substituted heterocyclic group, an optionally substituted sulfinyl group, an optionally substituted sulfonyl group, an optionally substituted hydroxyl group, an optionally substituted thiol group or an optionally substituted amino group; R² represents cyano group, formyl group, thioformyl group, or a group represented by the formula: -Z¹-Z² (wherein Z¹ represents —CO—, —CS—, —SO— or —SO₂—; and Z² represents an optionally substituted hydrocarbon group, an optionally substituted heterocyclic group, an optionally substituted hydroxyl group or an optionally substituted amino group); ring A represents an aromatic 5-membered heterocyclic ring represented by any of 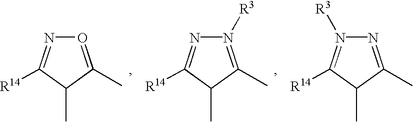
(wherein R³ represents hydrogen atom, an optionally substituted hydrocarbon group, an optionally substituted heterocyclic group, an optionally substituted hydroxyl group, an optionally substituted amino group, an optionally substituted sulfonyl group or an optionally substituted acyl group; R¹⁴ represents hydrogen atom, a halogen atom, an optionally substituted hydrocarbon group, an optionally substituted heterocyclic group, an optionally substituted hydroxyl group, an optionally substituted amino group, an optionally substituted sulfonyl group or an optionally substituted acyl group); and ring B represents an optionally substituted 5- to 7-membered hydrocarbon ring; provided that the compound is other than that represented by the formula: 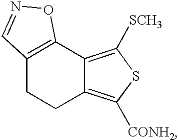
or a pharmaceutically acceptable salt thereof or a prodrug thereof, for manufacturing a prophylactic or therapeutic drug for bone or articular diseases which comprises.
